(19) United States
(12) Patent Application Publication
(10) Pub. No.: US 2020/0289611 A1
(43) Pub. Date: Sep. 17, 2020

(54) Methods and Compositions for the Inhibition of Quorum Sensing in Bacterial Infections

(71) Applicants: Cedric Pearce, Chapel Hill, NC (US); University of North Carolina at Greensboro, Greensboro, NC (US); University of Iowa Research Foundation, Iowa City, IA (US); The United State Government as Represented by the Department of Veterans, Washington, DC (US)

(72) Inventors: Nicholas Oberlies, Greensboro, NC (US); Cedric Pearce, Chapel Hill, NC (US); Alexander Horswill, North Liberty, IA (US); Jeffrey Kavanaugh, Iowa City, IA (US); Corey Parlet, Cedar Rapids, IA (US); Tamam El-Elimat, Mafraq (JO)

(21) Appl. No.: 16/301,426

(22) PCT Filed: May 12, 2017
(86) PCT No.: PCT/US2017/032474

§ 371 Date: Nov. 13, 2018

Related U.S. Application Data
(60) Provisional application No. 62/336,174, filed on May 13, 2016.

(51) Int. Cl. A61K 38/13; A61K 9/00

(52) U.S. Cl. CPC A61K 38/13; A61K 9/0019; A61K 9/0014

(57) ABSTRACT

This disclosure is directed to novel apicidin methods and compositions for the inhibition of quorum sensing in bacterial infections and novel apicidin methods and compositions for treating a staphylococcal infection.

### STATEMENT REGARDING FEDERALLY SPONSORED RESEARCH OR DEVELOPMENT

This invention was made with government support under Grant No. T32AI007511-19, awarded by the National Institute of Allergy and Infectious Diseases, an institute of the National Institutes of Health; Grant No. AT007052, awarded by the National Center for Complementary and Integrative Health, an institute of the National Institutes of Health; Grant Nos. AI007511 and AI00734, each awarded by the National Institutes of Health; Merit Award No. I01 BX002711, awarded by the Department of Veteran Affairs; and Grant No. AI083211 (Project 3), awarded by the National Institutes of Health. The United States Government has certain rights in the invention.

### 1. FIELD

The invention relates generally to the discovery of novel apicidin methods and compositions for the inhibition of quorum sensing in bacterial infections, and novel apicidin methods and compositions for treating a staphylococcal infection.

### 2. BACKGROUND

Antibiotic resistant pathogens are a global health threat. In particular, as the leading cause of infectious mortality in the United States, Staphylococcus aureus-induced disease represents a major healthcare problem. The alarming rise of infections caused by virulent, antibiotic resistant strains, such as emerging methicillin-resistant S. aureus (MRSA) isolates, highlight the need for new interventions that inhibit MRSA pathogenicity and potentiate host defense responses. In particular, there is a need for small molecule therapeutics that inhibit bacterial virulence as alternatives and/or adjuncts to conventional antibiotics, as they may limit pathogenesis and increase bacterial susceptibility to host killing.

### 3. SUMMARY OF THE INVENTION

The presently disclosed subject matter provides a pharmaceutical composition comprising: apicidin, or a pharmaceutically acceptable derivative thereof, in an amount effective to inhibit quorum sensing in a microbe; and a pharmaceutically acceptable carrier. In some embodiments, the apicidin is a natural analog of apicidin. In some embodiments, the apicidin is a synthetic analog of apicidin. In some embodiments, the microbe is Staphylococcus aureus.

The presently disclosed subject matter also provides a pharmaceutical composition comprising: apicidin, or a pharmaceutically acceptable derivative thereof, in an amount effective to treat a skin and soft tissue infection (SSTI); and a pharmaceutically acceptable carrier. In some embodiments, the apicidin is a natural analog of apicidin. In some embodiments, the apicidin is a synthetic analog of apicidin. In some embodiment, the pharmaceutical composition is a pharmaceutically acceptable topical formulation.

The presently disclosed subject matter also provides a method of treating a skin and soft tissue infection (SSTI) comprising administering to a subject in need thereof a therapeutically effective amount of apicidin or a pharmaceutically acceptable derivative thereof, and a pharmaceutically acceptable carrier. In some embodiments, the apicidin is a natural analog of apicidin. In some embodiments, the apicidin is a synthetic analog of apicidin. In some embodiments, the administering step is topical administration.

The presently disclosed subject matter also provides a pharmaceutical composition comprising: apicidin, or a pharmaceutically acceptable derivative thereof, in an amount effective to treat a staphylococcal infection; and a pharmaceutically acceptable carrier. In some embodiments, the apicidin is a natural analog of apicidin. In some embodiments, the apicidin is a synthetic analog of apicidin. In some embodiments, the staphylococcal infection is a skin and soft tissue infection (SSTI), staphylococcal pneumonia, a staphylococcal bone infection, a staphylococcal joint infection, staphylococcal sepsis, staphylococcal endocarditis, staphylococcal osteomyelitis, or staphylococcal meningitis. In some embodiments, the staphylococcal infection is a methicillin-resistant Staphylococcus aureus infection or a methicillin-sensitive Staphylococcus aureus infection.

The presently disclosed subject matter also provides a method of treating a staphylococcal infection comprising administering to a subject in need thereof a therapeutically effective amount of apicidin or a pharmaceutically acceptable derivative thereof, and a pharmaceutically acceptable carrier. In some embodiments, the apicidin is a natural analog of apicidin. In some embodiments, the apicidin is a synthetic analog of apicidin. In some embodiments, the administering step is intravenous administration. In some embodiments, the staphylococcal infection is a skin and soft tissue infection (SSTI), staphylococcal pneumonia, a staphylococcal bone infection, a staphylococcal joint infection, staphylococcal sepsis, or staphylococcal phendocarditis. In some embodiments, the staphylococcal infection is a methicillin-resistant Staphylococcus aureus infection or a methicillin-sensitive Staphylococcus aureus infection.

### 4. BRIEF DESCRIPTION OF THE FIGURES

FIG. 1A depicts quorum quenching activity of apicidin against an LAC agr reporter with minimal growth inhibition. Top graphs show optical density (OD) measurements over time; bottom graphs show relative fluorescence unit (RFU) measurements over time (hours). Micromolar concentrations of DMSO or apicidin are displayed to the right of the graphs.

FIG. 1B are tables summarizing quorum quenching activity of apicidin for both agr reporter (top) and hemolytic (bottom) activity assays.

FIG. 1C are graphs of in vitro quorum quenching activity of apicidin. Apicidin mediated suppression of agr-P3 reporters (inhibition extends to all 4 agr types). Left) Time course showing quorum quenching activity of apicidin against an LAC agr reporter with minimal growth inhibition, micromolar concentrations of vehicle (DMSO) or apicidin are displayed to the right of the graphs. FIG. 2 is a graph of the percentage of apicidin or vehicle exposed LAC recovered after 1 hr culture in human whole blood.

FIG. 3 is a graph with steps of an intradermal skin infection model.

FIG. 4A are representative images of tissue injury in C57BL/6 mice infected with WT LAC+/−apicidin (left) or Δagr+/−apicidin (right).

FIG. 4B are graphs depicting skin lesion size (left) and weight loss (right) measurements following infection for the indicated groups. Error bars represent SEM. Post test P value (*)=<0.05, (**)=<0.01.

FIG. 4C are representative images of tissue injury following infection with WT LAC+/−apicidin or Δagr apicidin and corresponding graphs of skin lesion size and weight loss and measurements following infection for the indicated groups in BALB/c (right) and C57BL/6 mice (left). Error bars represent SEM. Post test p value (*)=<0.05, (**)=<0.01

FIG. 5A are images of agrP3 reporter activity (bioluminescence) 3 hrs post infection.

FIG. 5B is a graph depicting kinetics of agr activation in apicidin and vehicle control treated mice after infection.

FIG. 5C is a graph depicting skin lesion size measurements (left) and representative images (right) at the indicated time points after infection. Error bars represent SEM. Post test P value (*)=<0.05, (**)=<0.01.

FIG. 5D is a graph depicting skin lesion size measurements (left) and representative images (right) at the indicated time points after infection. Error bars represent SEM. Post test p value (*)=<0.05, (**)=<0.01.

FIGS. 6A and 6B are images and a graph, respectively, of noninvasive, longitudinal measurements of MRSA bioluminescence following skin infection with Lux*MRSA.

FIG. 6C are graphs depicting CFUs recovered from BALB/c skin lesions 1 day after infection (left) and corresponding lesion size measurements (right). Error bars represent SEM. Post test P value (*)=<0.05, (**)=<0.01. FIG. 6D are corresponding representative images.

FIG. 6E includes (I.) a graph of skin lesion measurements following infection with an agr type II invasive MRSA isolate (Error bars represent SEM. Post test p value (*)=<0.05, (**)=<0.01.); (II.) representative images of tissue injury following infection with agr type II+/−apicidin; (III.) a graph of kinetics of agr activation in apicidin and vehicle control treated mice after infection (Error bars represent SEM. Post test p value (*)=<0.05, (**)=<0.01.); and (IV.) a graph of corresponding skin lesion size measurements at the indicated time point following infection (Error bars represent SEM. Post test p value (*)=<0.05.).

FIGS. 7A and 7B are graphs depicting PMN accumulation assessments by flow cytometry for skin, PB, and LN cell suspensions generated from LAC infected mice. Error bars represent SEM. Post test P value (*)=<0.05. Gating strategies are shown to the left of the bar graphs.

FIGS. 7C, 7D, and 7E each depict gating strategies (Left) and PMN accumulation values (Right) from the indicated tissues one day after intradermal MRSA challenge (+/−apicidin). Error bars represent SEM. Post test p value (*)=<0.05. (***)=<0.005.

FIGS. 7F and 7G each depict gating strategies (Left) and enumerated phagocytic PMNs (Right) one day after intradermal MRSA challenge with GFP-MRSA (Top) or DsRed-MRSA (Bottom) Error bars represent SEM. Post test p value (*)=<0.05. (***)=<0.005.

FIG. 7H is a graph with data from a whole blood killing assay, following four hours of culture in the presence of 100 μm apicidin or vehicle control, heparinized human whole blood was inoculated with MRSA organisms cultured (4 hrs) in the presence of 100 μm apicidin or vehicle. After one hours CFUs from inoculated whole blood were plated out and compared with the starting inoculum to score for percent killing. Error bars represent SEM. Post test p value (*)=<0.05.

FIGS. 8A depicts gating strategies, and 8B are the corresponding graphs depicting phagocyte accumulation assessments by flow cytometry one day after infection for skin cell suspensions generated from mice infected with 2×107 CFUs LAC engineered for constitutive expression of red fluorescent protein+/−5 μg apicidin. Error bars represent SEM. Post test P value (*)=<0.05.

FIG. 9A is a graph of an assessment of apicidin mediated agr-inhibition using a constitutive AgrC mutant. FIG. 9B. is a graph of mass spectrometric measurements of AIP-I production by a USA300 MRSA isolate. FIG. 9C is a graph of effect of increasing concentrations of apicidin upon agrA reporter activation using an agr null strain expressing a plasmid for agr.

FIG. 10 is a Venn diagram using LAC+vehicle as a baseline and showing the number of genes surpassing the four-fold change threshold in Δagr and apicidin treated groups, as well as the number overlapping transcriptional targets (left); and a table listing MRSA virulence factors that are commonly repressed in Δagr and apicidin treated cells (right).

FIG. 11A are graphs of data from cytokine array analysis of supernatants from infected skin tissues collected one day after infection. FIG. 11B depicts flow cytometric analysis of apoptosis among phagocytic and non-phagocytic PMNs recovered from skin lesions of apicidin or vehicle treated animals one day after infection. Error bars represent SEM. Post test p value (*)=<0.05, (**)=<0.01.

FIG. 12A is a representative prep-HPLC chromatogram of fraction #4 (MSX53644). FIG. 12B is a representative prep-HPLC chromatogram of fraction #5 (G134). Method: Gradient, MeCN: H2O/0.1 formic acid, 40 to 70 over 30 min to 100, no hold, 21.24 mL/min, 254 nm. Column: Phenomenex Gemini-NX, 5 μm, C18, 110A, AX. 250×21.20 mm.

FIG. 13A is a schematic of agr system; and FIG. 13B is a flow chart for isolation of apicidin from G134/G137 preparative chromatogram (left) and apicidin structures (right).

FIGS. 14A-D are (+)-HRESIMS spectra of Apicidin L, Apicidin, Apicidin A, and Apicidin D₂.

FIGS. 15A-C are (A) overlay of chromatographic peaks of apicidin, G134, and G137; (B) (−)-HRESIMS of apicidin; and (C) MS MS CID fragmentation spectra of apicidin.

FIG. 16A is ¹H and ¹³C NMR spectra of compound 1 [500 MHz for 1H and 125 MHz for 13C, CDCl3]. FIG. 16B is ¹H and 13C NMR spectra of compound 2 [500 MHz for ¹H and 125 MHz for ¹³C, CDCl₃]. FIG. 16C is ¹H NMR spectrum of compound 3 [500 MHz, CDCl₃]. FIG. 16D is ¹H NMR spectrum of compound 4 [500 MHz, CDCl3].

### 5. DETAILED DESCRIPTION OF REPRESENTATIVE EMBODIMENTS

The presently disclosed subject matter now will be described more fully hereinafter. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the descriptions and the associated figures. Therefore, it is to be understood that the presently disclosed subject matter is not to be limited to the specific embodiments disclosed and that modifications and other embodiments are intended to be included within the scope of the appended claims.

Unless defined otherwise, all technical and scientific terms used herein have the same meanings as commonly understood by one of ordinary skill in the art to which this disclosure belongs. Preferred methods, devices, and materials are described, although any methods and materials similar or equivalent to those described herein can be used in the practice or testing of the present disclosure. All references cited herein are incorporated by reference in their entirety. Numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The presently disclosed subject matter provides pharmaceutical compositions comprising apicidin for inhibiting quorum sensing in bacterial infections and methods of inhibiting quorum sensing in bacterial infections comprising administering apicidin. Apicidin is a fungal metabolite produced by certain isolates of Fusarium semitectum and has the following chemical structure:

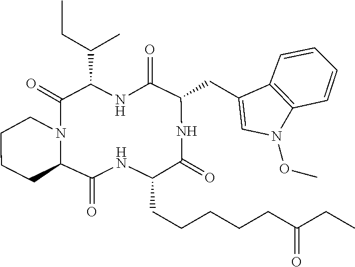

Apicidin has previously been characterized within multiple contexts relevant to human health. To date, the applicability of apicidin as a drug candidate has been primarily attributed to its action as a histone deacetylase (HDAC) inhibitor. The epigenetic effects of apicidin treatment has been shown to induce growth arrest and apoptosis in multiple tumor cell lines (45-47). In addition to its capacity to interfere with the transcriptional regulation underlying multiple malignant processes, apicidin's HDAC activity has also been shown to inhibit the infective mechanisms of several apicomplexan parasites (38, 39, 48). In contrast to these reports that highlight the capacity of apicidin to alter gene expression by influencing chromatin structure within eukaryotic cells, the present application describes the potent bioactivity of this compound within a clinically relevant prokaryotic system. Like other prokaryotes, S. aureus lacks histones and therefore the ability to apicidin to interfere with staphylococcal quorum sensing occurs independently of its HDAC activity. On the other hand, genome wide inquiry into the transcriptional regulation of immune response pathways has revealed that HDAC inhibitors play an important role in host defense (49-51). To date numerous HDAC inhibitors, including apicidin have been shown to enhance cationic antimicrobial peptide (CAMP) production from epithelial cells in vitro (51). Given that CAMPs play key elements of innate defense against bacterial pathogens, it stands to reason that an apicidin-mediated augmentation of CAMP production could benefit host defense directly.

This disclosure describes apicidin as a suppressor of quorum sensing, which is a cell-to-cell and density-dependent communication system in which pathogenic bacteria control the expression of certain genes, e.g. virulence factors important for invasive infection and pathogenesis. Quorum quenching is the disruption of the quorum sensing mechanism of pathogens and can limit pathogenesis in the host and/or serve as adjuncts to extend the utility of existing antibiotics.

Staphylococcus aureus (S. aureus) remains one of the most frequent causes of both hospital and community acquired infection (22). The propensity of S. aureus to acquire antibiotic resistance is evidenced by the reoccurring clinical pattern whereby epidemics caused by resistant isolates ensue rapidly after a new antibiotic is introduced for infection control (23). While S. aureus is classified as an opportunistic pathogen, the capacity of highly aggressive USA300 lineages of methicillin resistant S. aureus (MRSA) to inflict disease among “healthy” community-dwelling individuals has reached pandemic proportions (23-25). The hyper-virulent nature of “community associated” MRSA (CA-MRSA) strains has been attributed to heightened expression of genome-encoded virulence factors such as α-hemolysin and phenol soluble modulins (PSMs), which subvert host defense by exerting cytolytic effects upon immune effector cells (6, 24).

Staphylococcus aureus is a major cause of invasive skin and soft tissue infections (SSTIs) in both the hospital and community, and is also becoming increasingly antibiotic resistant. Many of the virulence factors contributing to SSTIs are regulated by the accessory gene regulator (agr). Also, the expression of methicillin-resistant S. aureus (MRSA) virulence factors is a function of agr-driven quorum sensing. Because many S. aureus virulence factors antagonize the host innate immune response, inhibiting bacterial virulence can itself augment host defense. Specifically, disruption of S. aureus quorum-sensing-dependent virulence can not only limit pathogenesis, but also can reduce inflammation and result in enhanced bacterial clearance. Disruption of agr-signaling by mutagenesis, monoclonal antibodies, and/or host-factors limits S. aureus infection and reduces pathogenesis (6, 7-12).

The agr system uses a small, secreted autoinducing peptide (AIP) to activate a receptor histidine kinase, AgrC, in the bacterial cell membrane. AgrC phosphorylates the transcription factor AgrA, which in turn activates transcription at the P2 and P3 promoters of the operon. P3 activation drives production of the effector of the operon, RNAIII, which regulates expression of over 200 virulence genes that contribute to invasive infection (6). S. aureus isolates have one of four agr alleles (agr-I, agr-II, agr-III, or agr-IV), each encoding factors that secrete a unique AIP (AIP1, AIP2, AIP3, or AIP4, respectively) which is detected by a cognate AgrC histidine kinase. S. aureus isolates that possesses any one of the four alleles can cause human disease (13, 14).

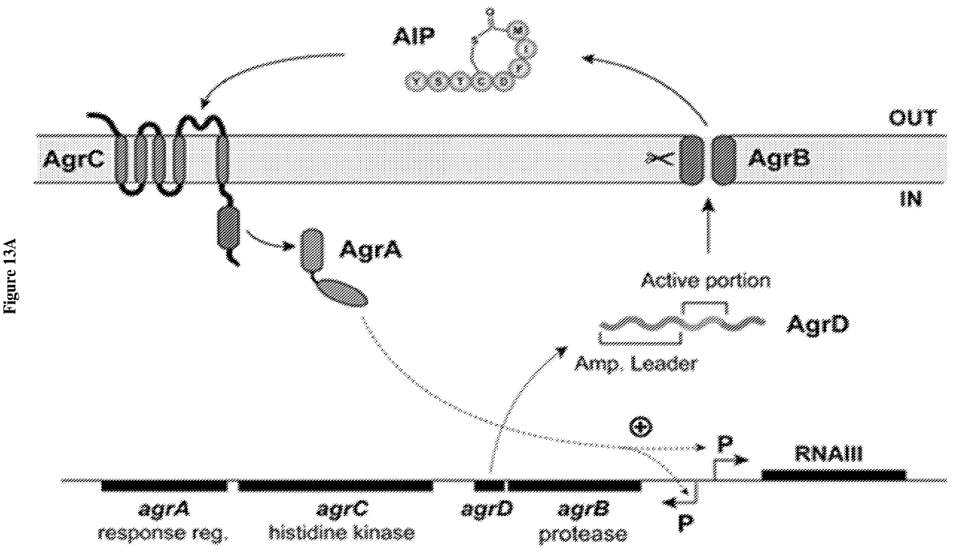

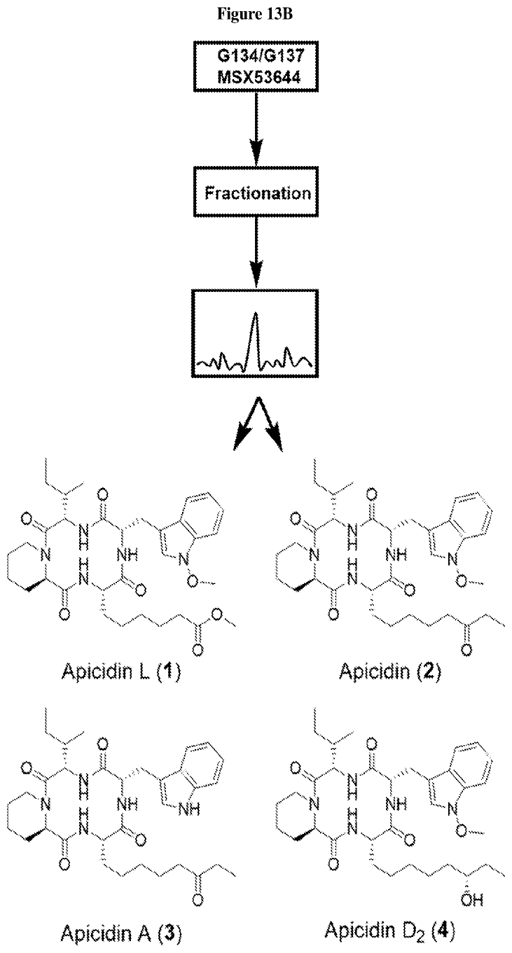

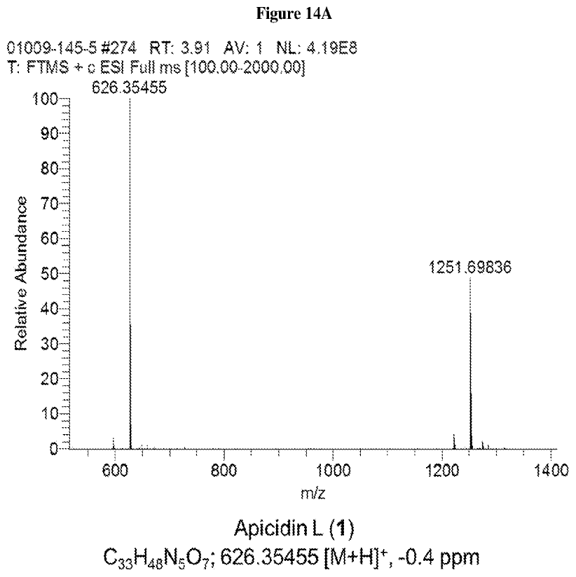

Like other S. aureus strains, MRSA utilizes quorum sensing to synchronize virulence factor induction in proportion to prevailing cell-density (26, 27). Encoded by the agrBDCA operon (FIG. 13A), the quorum sensing signaling apparatus achieves maximal activity at high cell densities when ambient agrB/agrD derived autoinducing peptides (AIPs) reach a concentration threshold necessary to activate the AgrC-A two component signal transduction system. The respective ligand/receptor interaction between AIP and the sensor histidine kinase AgrC, initiates the signaling cascade via phospho-transfer mediated activation of the response regulator AgrA. The DNA binding capability of AgrA mediates distinct transcriptional pathways from the systems' two oppositely oriented promoter units (27). Whereas the P2 promoter activates an auto-induction circuit via agrBDCA expression, the P3 promoter, exponentially increases the system's major effector molecule, RNAIII, which regulates >200 virulence factor genes for the purpose of countering host defense and promoting tissue invasion (26-28). Within the context of acute infection, agr-regulated quorum sensing coordinates an explosive outburst of virulence factors that are manifestly harmful to host (29-30). As such, the therapeutic potential of quorum sensing inhibitors (aka quorum quenchers) have been intensively investigated, yet there are presently no quorum quenching therapies that have received FDA approval for clinical use (30-35).

This disclosure describes novel apicidin compositions and methods to inhibit quorum sensing in an exemplary model infectious microbe, S. aureus and demonstrates in vivo efficacy against S. aureus quorum sensing-dependent virulence; and novel apicidin compositions and methods to treat a staphylococcal infection. As used herein, the term “apicidin” refers to apicidin:

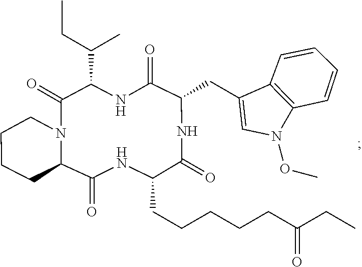

natural analogs of apicidin, wherein a “natural analog of apicidin” is an analog of apicidin biosynthesized by fungi, including but not limited to the following natural analogs of apicidin:

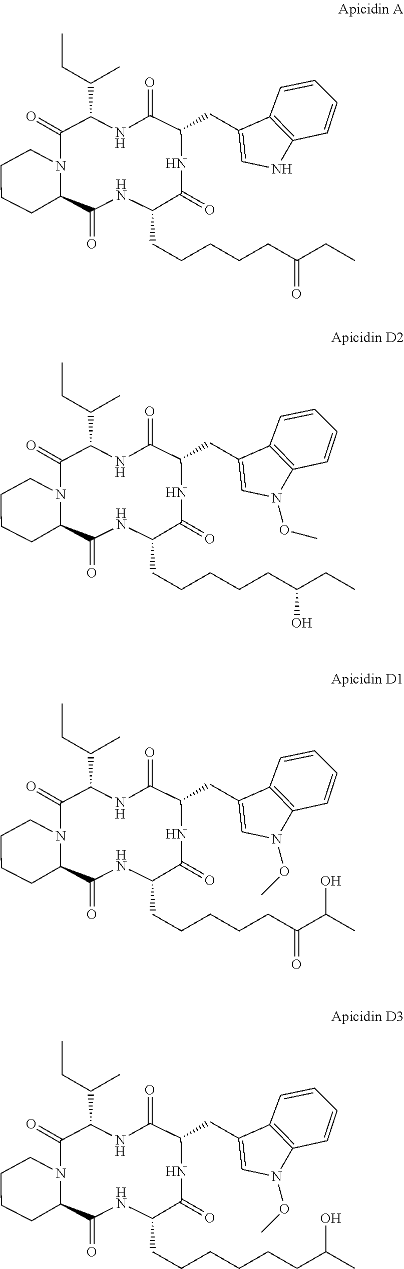 [SMILES: CCC(=O)CCCCC[C@@H]1NC(=O)[C@H]2CCCCN2C(=O)[C@H](C(C)CC)NC(=O)[C@H](Cc2c[nH]c3ccccc23)NC1=O ; CCC(C)[C@@H]1NC(=O)[C@H](Cc2cn(OC)c3ccccc23)NC(=O)[C@H](CCCCC[C@@H](O)CC)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCC(C)[C@@H]1NC(=O)[C@H](Cc2cn(OC)c3ccccc23)NC(=O)[C@H](CCCCCCC(C)O)NC(=O)[C@H]2CCCCN2C1=O]

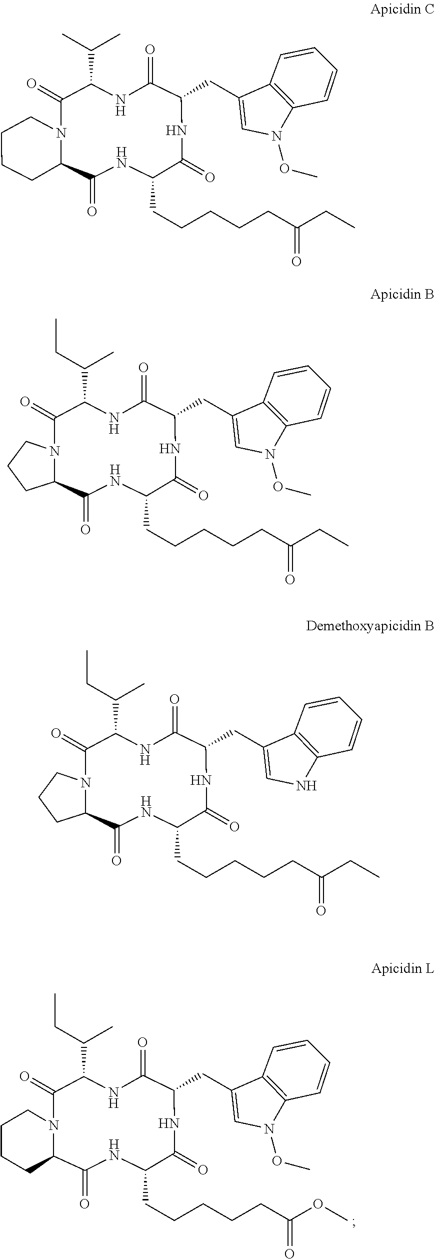 [SMILES: CCC(=O)CCCCC[C@@H]1NC(=O)[C@H]2CCCCN2C(=O)[C@H](C(C)C)NC(=O)[C@H](Cc2cn(OC)c3ccccc23)NC1=O ; CCC(=O)CCCCC[C@@H]1NC(=O)[C@H]2CCCN2C(=O)[C@H](C(C)CC)NC(=O)[C@H](Cc2cn(OC)c3ccccc23)NC1=O ; CCC(=O)CCCCC[C@@H]1NC(=O)[C@H]2CCCN2C(=O)[C@H](C(C)CC)NC(=O)[C@H](Cc2c[nH]c3ccccc23)NC1=O ; CCC(C)[C@@H]1NC(=O)[C@H](Cc2cn(OC)c3ccccc23)NC(=O)[C@H](CCCCCC(=O)OC)NC(=O)[C@H]2CCCCN2C1=O]

and synthetic analogs of apicidin, wherein a “synthetic analog of apicidin” is an analog of apicidin that is synthesized de novo or synthesized by modification to apicidin or a natural analog of apicidin, such synthetic analogs of apicidin including, but not limited to, the following synthetic analogs of apicidin:

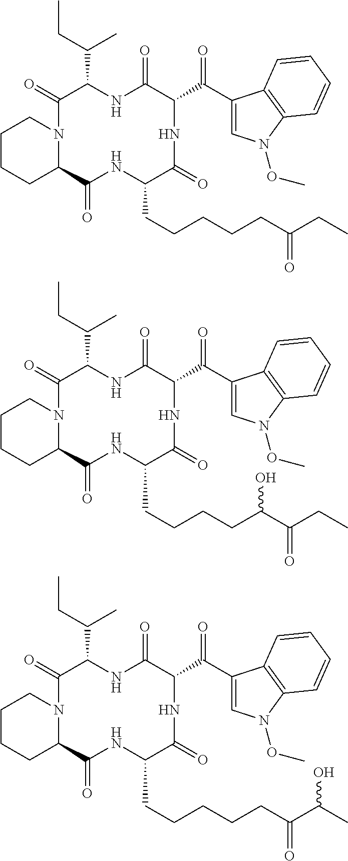 [SMILES: CCC(=O)CCCCC[C@@H]1NC(=O)[C@H]2CCCCN2C(=O)[C@H](C(C)CC)NC(=O)[C@H](C(=O)c2cn(OC)c3ccccc23)NC1=O ; CCC(=O)C(O)CCCC[C@@H]1NC(=O)[C@H]2CCCCN2C(=O)[C@H](C(C)CC)NC(=O)[C@H](C(=O)c2cn(OC)c3ccccc23)NC1=O ; CCC(C)[C@@H]1NC(=O)[C@H](C(=O)c2cn(OC)c3ccccc23)NC(=O)[C@H](CCCCCC(=O)C(C)O)NC(=O)[C@H]2CCCCN2C1=O]

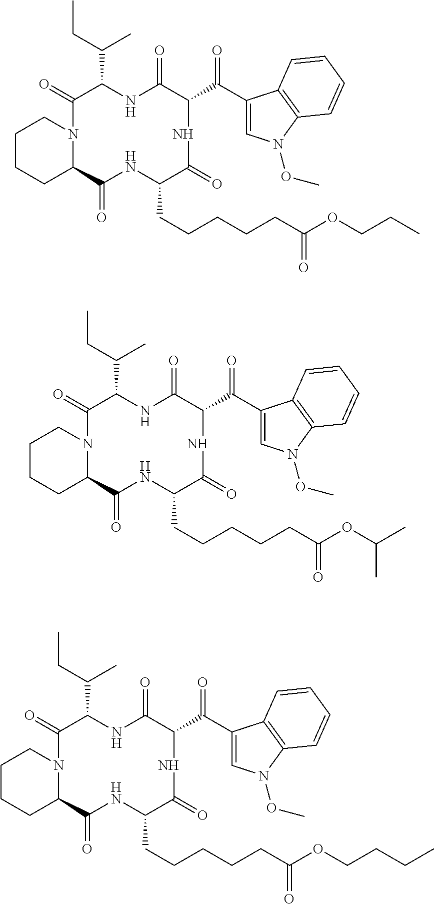 [SMILES: CCCOC(=O)CCCCC[C@@H]1NC(=O)[C@H]2CCCCN2C(=O)[C@H](C(C)CC)NC(=O)[C@H](C(=O)c2cn(OC)c3ccccc23)NC1=O ; CCC(C)[C@@H]1NC(=O)[C@H](C(=O)c2cn(OC)c3ccccc23)NC(=O)[C@H](CCCCCC(=O)OC(C)C)NC(=O)[C@H]2CCCCN2C1=O ; CCCCOC(=O)CCCCC[C@@H]1NC(=O)[C@H]2CCCCN2C(=O)[C@H](C(C)CC)NC(=O)[C@H](C(=O)c2cn(OC)c3ccccc23)NC1=O]

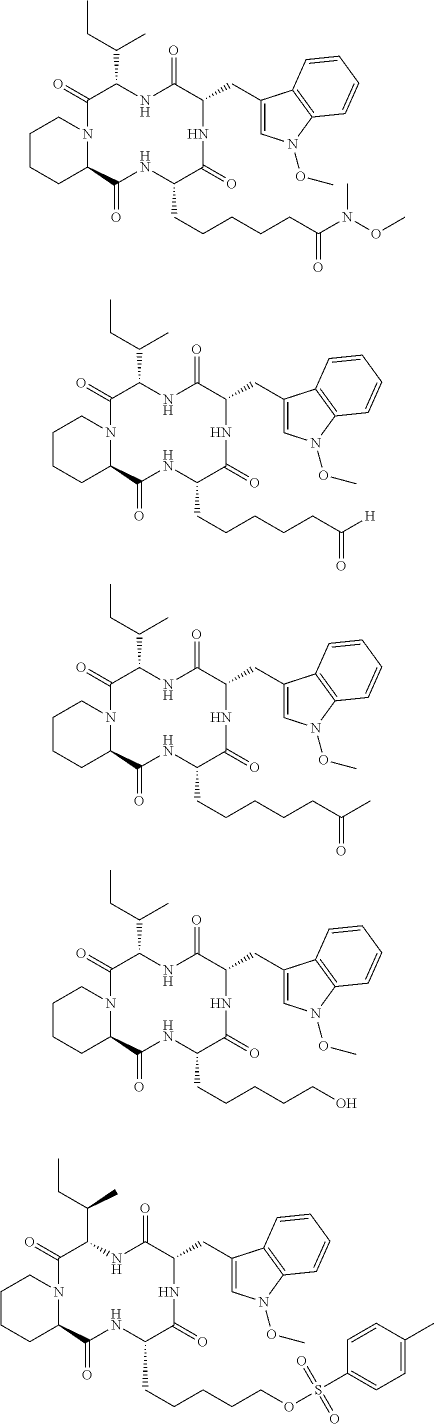 [SMILES: CCC(C)[C@@H]1NC(=O)[C@H](Cc2cn(OC)c3ccccc23)NC(=O)[C@H](CCCCCC(=O)N(C)OC)NC(=O)[C@H]2CCCCN2C1=O ; CCC(C)[C@@H]1NC(=O)[C@H](Cc2cn(OC)c3ccccc23)NC(=O)[C@H](CCCCCC=O)NC(=O)[C@H]2CCCCN2C1=O ; CCC(C)[C@@H]1NC(=O)[C@H](Cc2cn(OC)c3ccccc23)NC(=O)[C@H](CCCCCC(C)=O)NC(=O)[C@H]2CCCCN2C1=O ; CCC(C)[C@@H]1NC(=O)[C@H](Cc2cn(OC)c3ccccc23)NC(=O)[C@H](CCCCCO)NC(=O)[C@H]2CCCCN2C1=O ; CC[C@@H](C)[C@@H]1NC(=O)[C@H](Cc2cn(OC)c3ccccc23)NC(=O)[C@H](CCCCCOS(=O)(=O)c2ccc(C)cc2)NC(=O)[C@H]2CCCCN2C1=O]

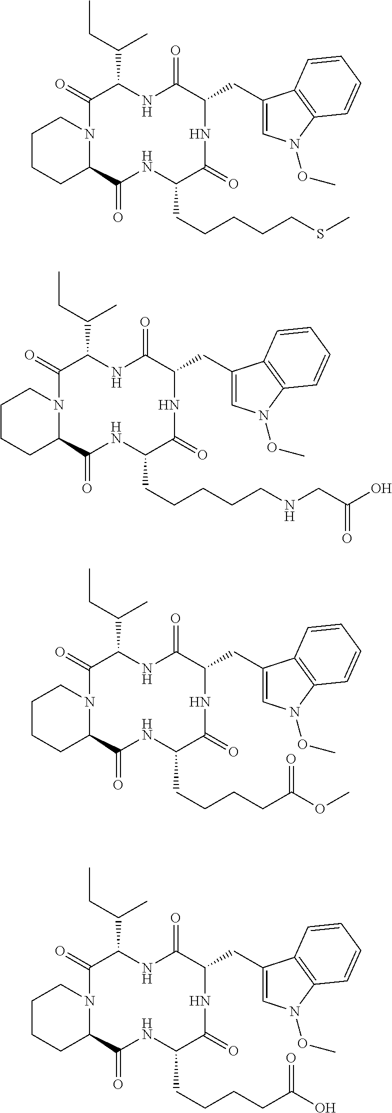 [SMILES: CCC(C)[C@@H]1NC(=O)[C@H](Cc2cn(OC)c3ccccc23)NC(=O)[C@H](CCCCCSC)NC(=O)[C@H]2CCCCN2C1=O ; CCC(C)[C@@H]1NC(=O)[C@H](Cc2cn(OC)c3ccccc23)NC(=O)[C@H](CCCCCNCC(=O)O)NC(=O)[C@H]2CCCCN2C1=O ; CCC(C)[C@@H]1NC(=O)[C@H](Cc2cn(OC)c3ccccc23)NC(=O)[C@H](CCCCC(=O)OC)NC(=O)[C@H]2CCCCN2C1=O ; CCC(C)[C@@H]1NC(=O)[C@H](Cc2cn(OC)c3ccccc23)NC(=O)[C@H](CCCCC(=O)O)NC(=O)[C@H]2CCCCN2C1=O]

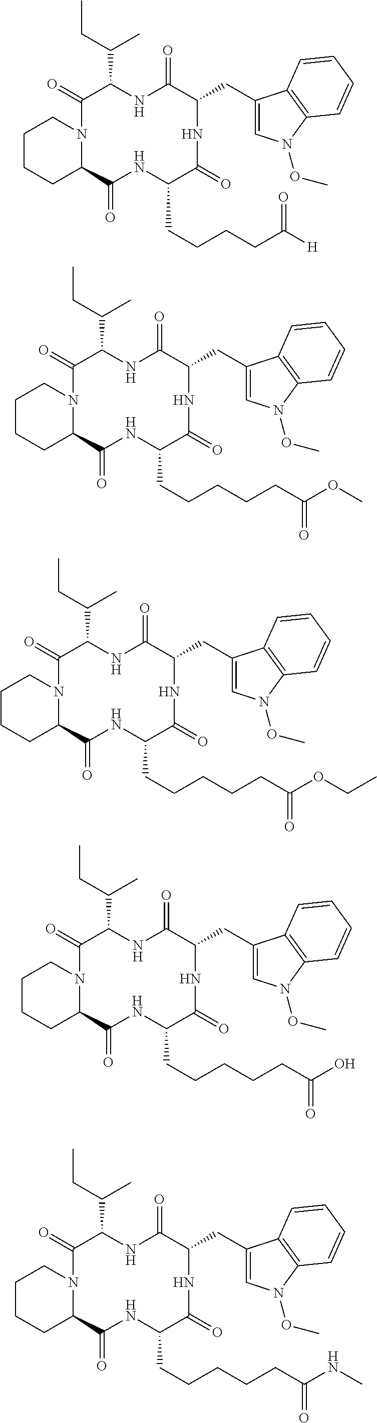 [SMILES: CCC(C)[C@@H]1NC(=O)[C@H](Cc2cn(OC)c3ccccc23)NC(=O)[C@H](CCCCC=O)NC(=O)[C@H]2CCCCN2C1=O ; CCC(C)[C@@H]1NC(=O)[C@H](Cc2cn(OC)c3ccccc23)NC(=O)[C@H](CCCCCC(=O)OC)NC(=O)[C@H]2CCCCN2C1=O ; CCOC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)NC)NC(=O)[C@H]2CCCCN2C1=O]

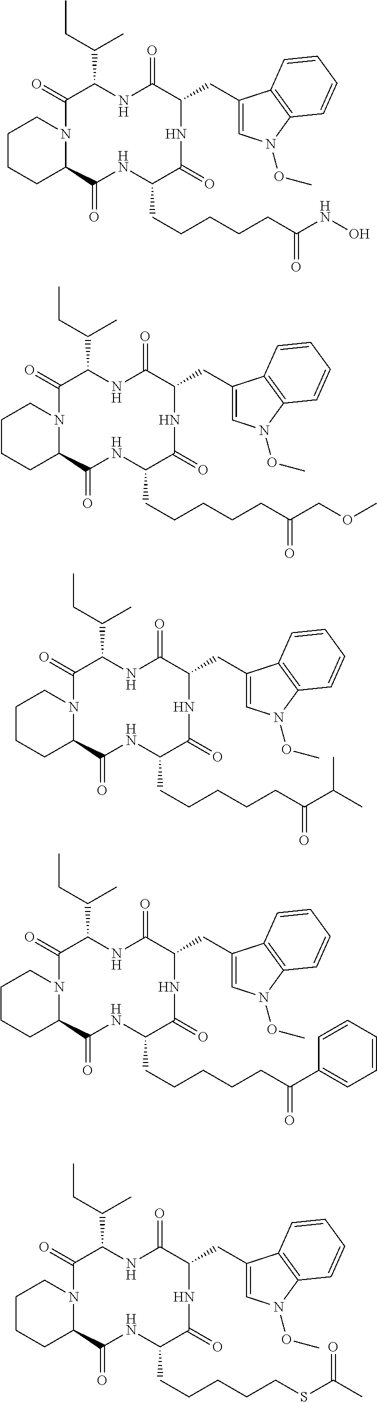 [SMILES: CCC(C)[C@@H]1NC(=O)[C@H](Cc2cn(OC)c3ccccc23)NC(=O)[C@H](CCCCCC(=O)NO)NC(=O)[C@H]2CCCCN2C1=O ; CCC(C)[C@@H]1NC(=O)[C@H](Cc2cn(OC)c3ccccc23)NC(=O)[C@H](CCCCCC(=O)COC)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)C)NC(=O)[C@H]2CCCCN2C1=O ; CCC(C)[C@@H]1NC(=O)[C@H](Cc2cn(OC)c3ccccc23)NC(=O)[C@H](CCCCCC(=O)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCCSC(C)=O)NC(=O)[C@H]2CCCCN2C1=O]

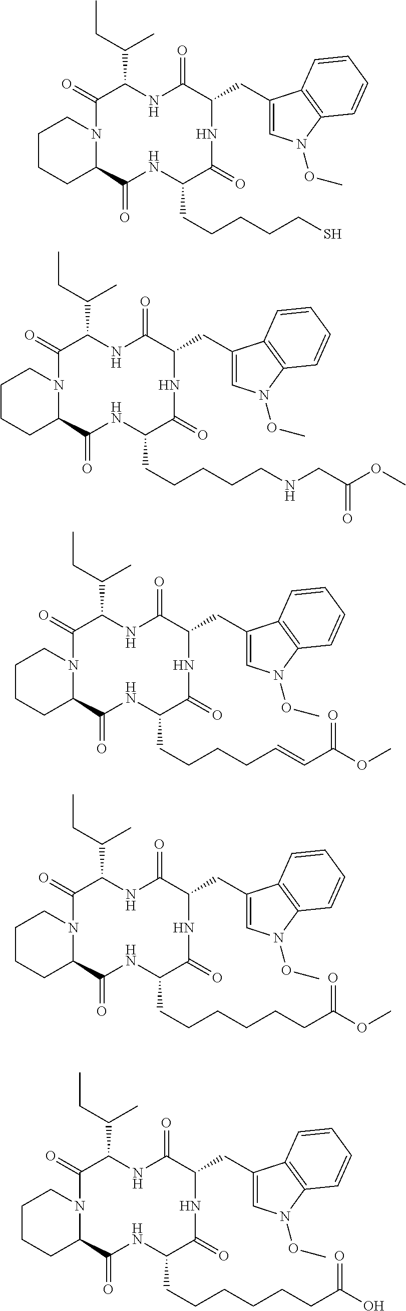 [SMILES: CCC(C)[C@@H]1NC(=O)[C@H](Cc2cn(OC)c3ccccc23)NC(=O)[C@H](CCCCCS)NC(=O)[C@H]2CCCCN2C1=O ; CCC(C)[C@@H]1NC(=O)[C@H](Cc2cn(OC)c3ccccc23)NC(=O)[C@H](CCCCCNCC(=O)OC)NC(=O)[C@H]2CCCCN2C1=O ; CCC(C)[C@@H]1NC(=O)[C@H](Cc2cn(OC)c3ccccc23)NC(=O)[C@H](CCCC/C=C/C(=O)OC)NC(=O)[C@H]2CCCCN2C1=O ; CCC(C)[C@@H]1NC(=O)[C@H](Cc2cn(OC)c3ccccc23)NC(=O)[C@H](CCCCCCC(=O)OC)NC(=O)[C@H]2CCCCN2C1=O ; CCC(C)[C@@H]1NC(=O)[C@H](Cc2cn(OC)c3ccccc23)NC(=O)[C@H](CCCCCCC(=O)O)NC(=O)[C@H]2CCCCN2C1=O]

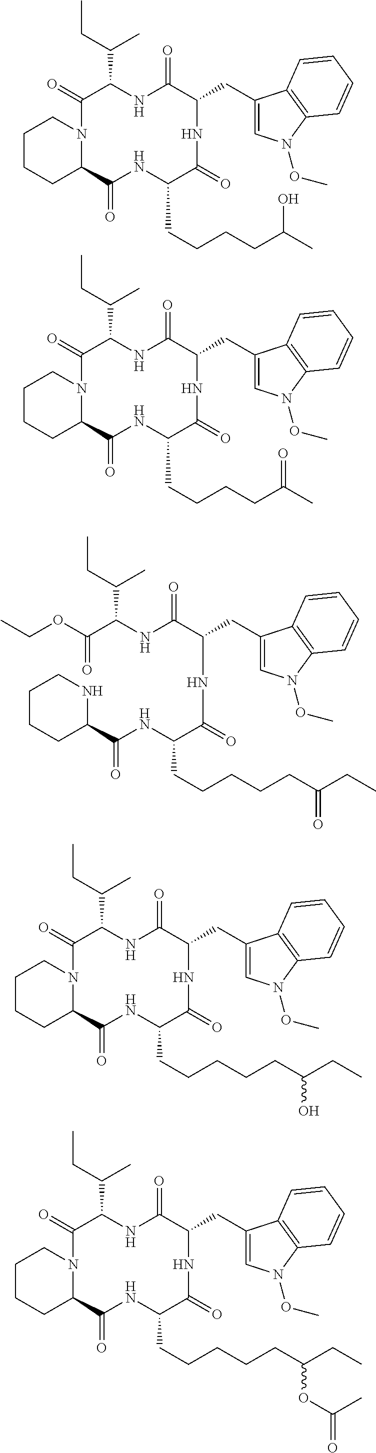 [SMILES: CCC(C)[C@@H]1NC(=O)[C@H](Cc2cn(OC)c3ccccc23)NC(=O)[C@H](CCCCC(C)O)NC(=O)[C@H]2CCCCN2C1=O ; CCC(C)[C@@H]1NC(=O)[C@H](Cc2cn(OC)c3ccccc23)NC(=O)[C@H](CCCCC(C)=O)NC(=O)[C@H]2CCCCN2C1=O ; CCOC(=O)[C@@H](NC(=O)[C@H](Cc1cn(OC)c2ccccc12)NC(=O)[C@H](CCCCCC(=O)CC)NC(=O)[C@H]1CCCCN1)C(C)CC ; CCC(O)CCCCC[C@@H]1NC(=O)[C@H]2CCCCN2C(=O)[C@H](C(C)CC)NC(=O)[C@H](Cc2cn(OC)c3ccccc23)NC1=O ; CCC(CCCCC[C@@H]1NC(=O)[C@H]2CCCCN2C(=O)[C@H](C(C)CC)NC(=O)[C@H](Cc2cn(OC)c3ccccc23)NC1=O)OC(C)=O]

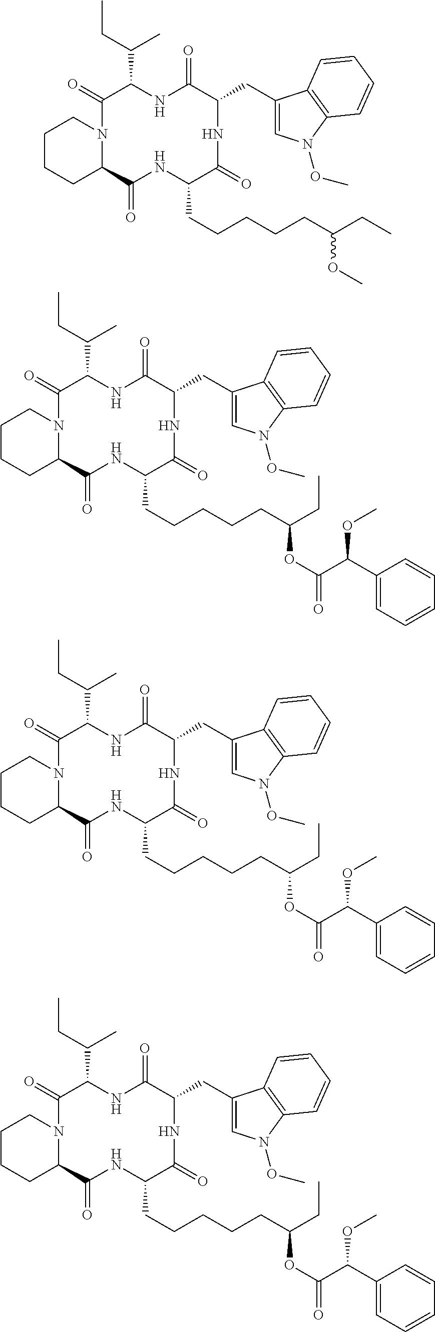 [SMILES: CCC(CCCCC[C@@H]1NC(=O)[C@H]2CCCCN2C(=O)[C@H](C(C)CC)NC(=O)[C@H](Cc2cn(OC)c3ccccc23)NC1=O)OC ; CCC(C)[C@@H]1NC(=O)[C@H](Cc2cn(OC)c3ccccc23)NC(=O)[C@H](CCCCC[C@@H](CC)OC(=O)[C@@H](OC)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCC[C@H](CC)OC(=O)[C@H](OC)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCC[C@@H](CC)OC(=O)[C@H](OC)c2ccccc2)NC(=O)[C@H]2CCCCN2C1=O]

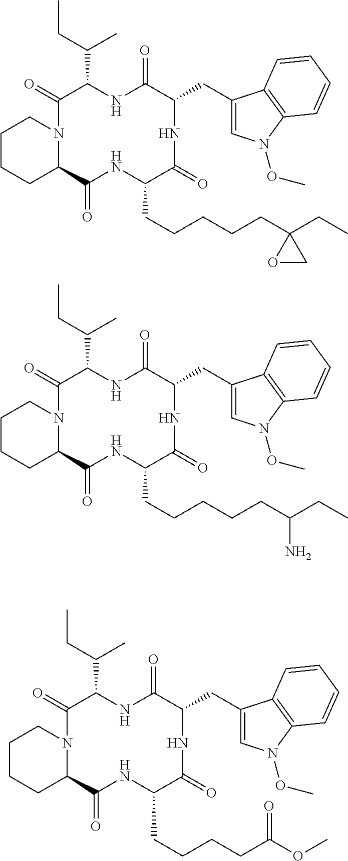 [SMILES: CCC(C)[C@@H]1NC(=O)[C@H](Cc2cn(OC)c3ccccc23)NC(=O)[C@H](CCCCCC2(CC)CO2)NC(=O)[C@H]2CCCCN2C1=O ; CCC(N)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCC(=O)OC)NC(=O)[C@H]2CCCCN2C1=O]

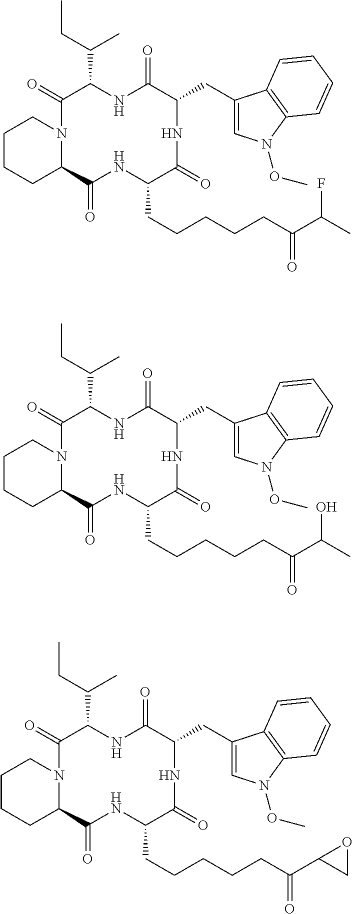 [SMILES: CCC(C)[C@@H]1NC(=O)[C@H](Cc2cn(OC)c3ccccc23)NC(=O)[C@H](CCCCCC(=O)C(C)F)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)C2CO2)NC(=O)[C@H]2CCCCN2C1=O]

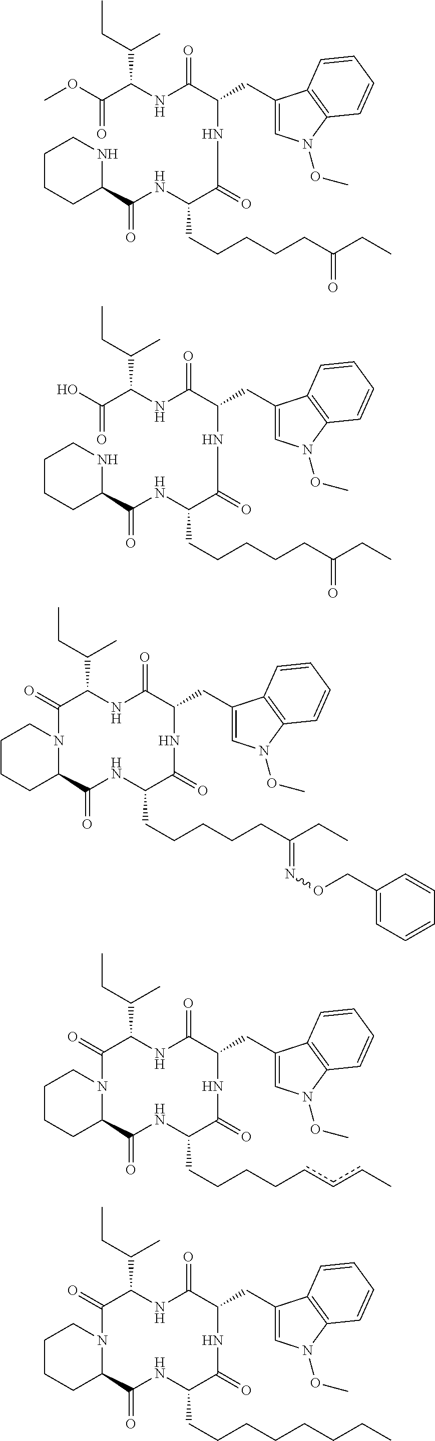

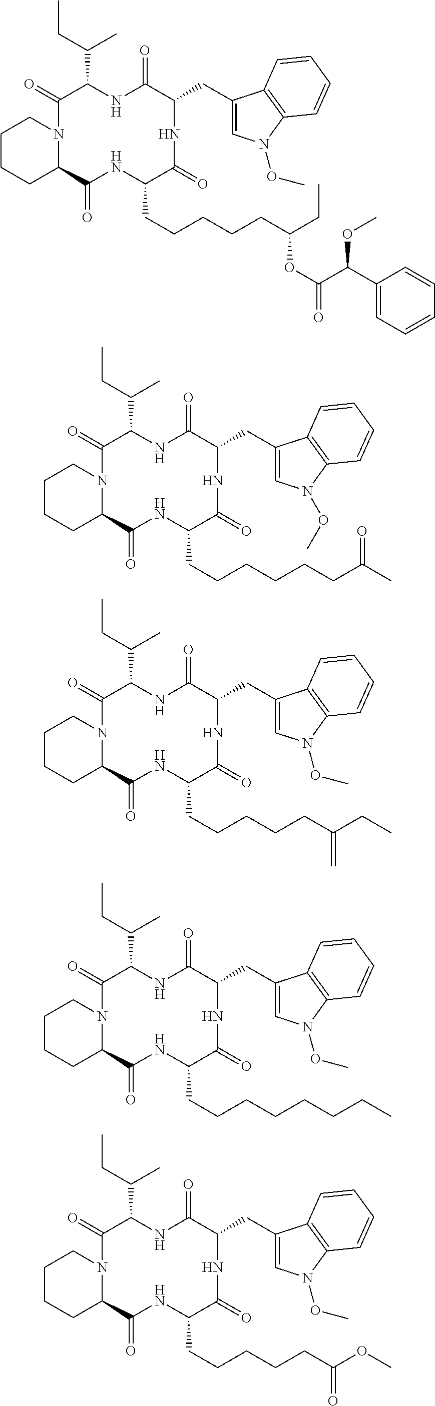

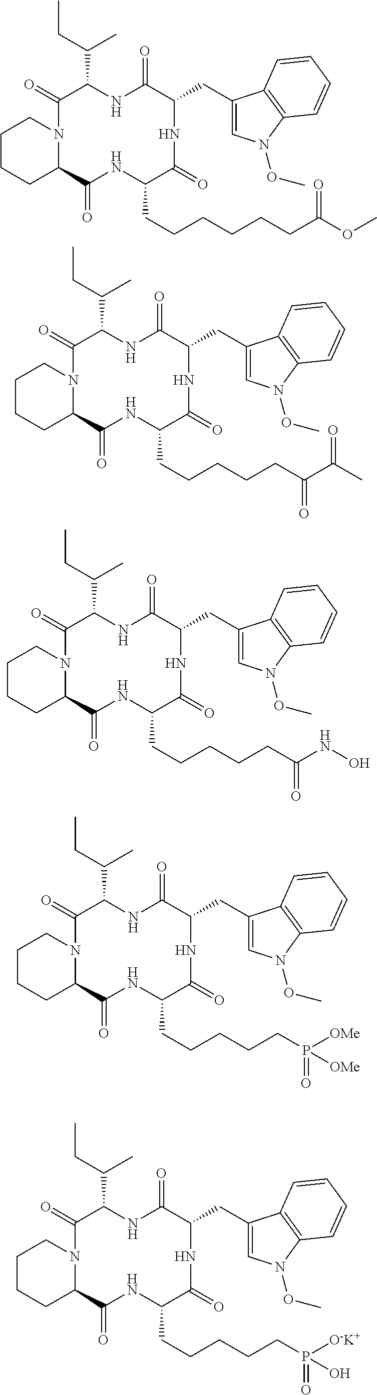

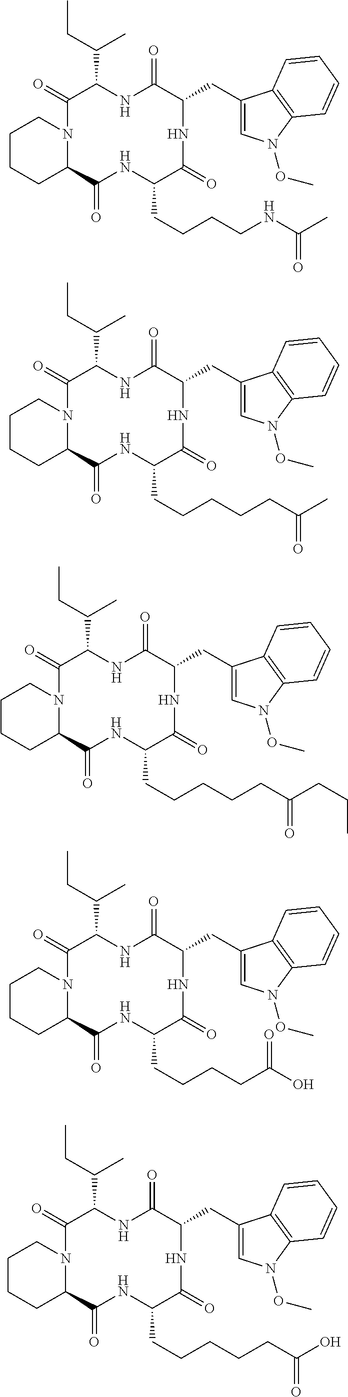 [SMILES: CCC(C)[C@@H]1NC(=O)[C@H](Cc2cn(OC)c3ccccc23)NC(=O)[C@H](CCCCNC(C)=O)NC(=O)[C@H]2CCCCN2C1=O ; CCC(C)[C@@H]1NC(=O)[C@H](Cc2cn(OC)c3ccccc23)NC(=O)[C@H](CCCCCC(C)=O)NC(=O)[C@H]2CCCCN2C1=O ; CCCC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCC(=O)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)O)NC(=O)[C@H]2CCCCN2C1=O]

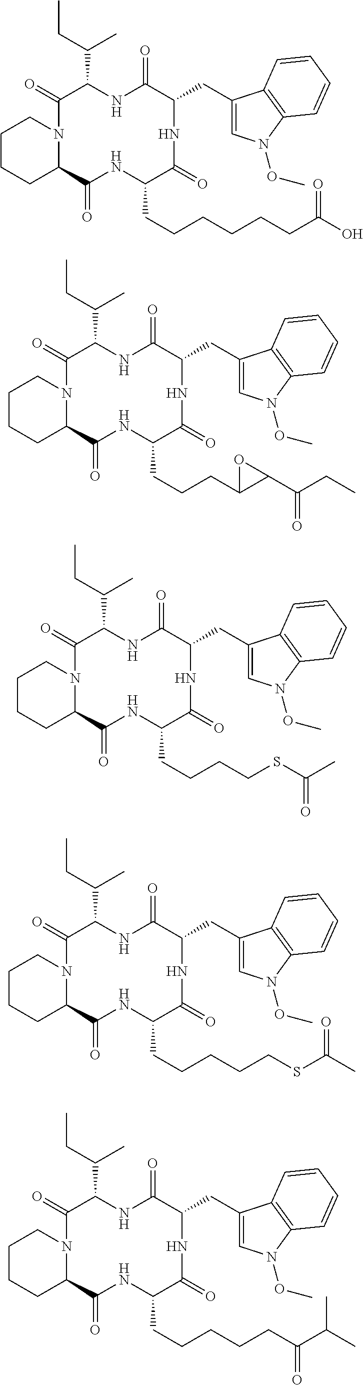 [SMILES: CCC(C)[C@@H]1NC(=O)[C@H](Cc2cn(OC)c3ccccc23)NC(=O)[C@H](CCCCCCC(=O)O)NC(=O)[C@H]2CCCCN2C1=O ; CCC(=O)C1OC1CCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCSC(C)=O)NC(=O)[C@H]2CCCCN2C1=O ; CCC(C)[C@@H]1NC(=O)[C@H](Cc2cn(OC)c3ccccc23)NC(=O)[C@H](CCCCCSC(C)=O)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)C)NC(=O)[C@H]2CCCCN2C1=O]

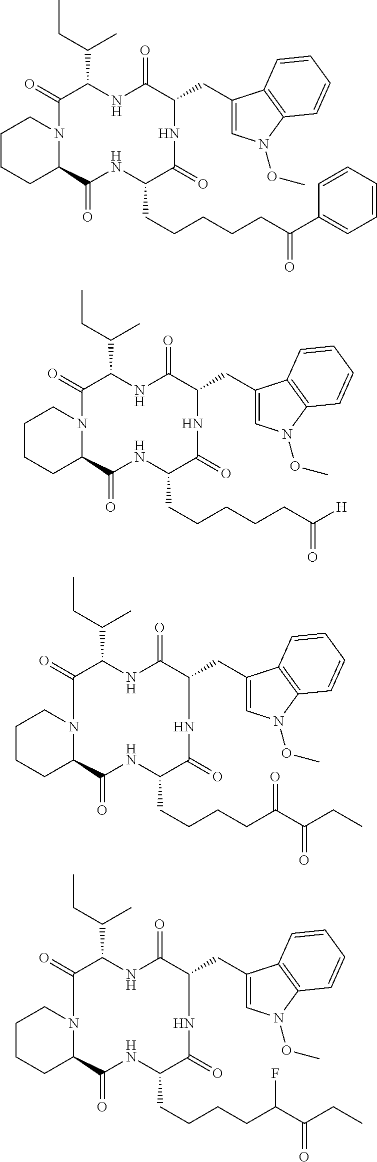 [SMILES: CCC(C)[C@@H]1NC(=O)[C@H](Cc2cn(OC)c3ccccc23)NC(=O)[C@H](CCCCCC(=O)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCCC=O)NC(=O)[C@H]2CCCCN2C1=O ; CCC(=O)C(=O)CCCC[C@@H]1NC(=O)[C@H]2CCCCN2C(=O)[C@H](C(C)CC)NC(=O)[C@H](Cc2cn(OC)c3ccccc23)NC1=O ; CCC(=O)C(F)CCCC[C@@H]1NC(=O)[C@H]2CCCCN2C(=O)[C@H](C(C)CC)NC(=O)[C@H](Cc2cn(OC)c3ccccc23)NC1=O]

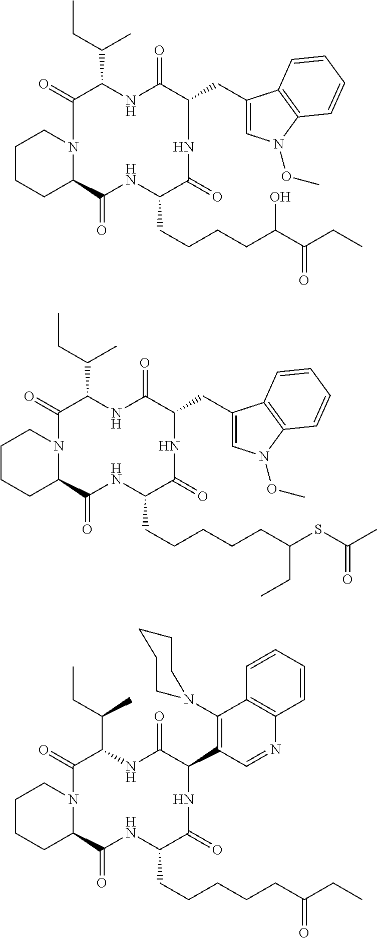 [SMILES: CCC(=O)C(O)CCCC[C@@H]1NC(=O)[C@H]2CCCCN2C(=O)[C@H](C(C)CC)NC(=O)[C@H](Cc2cn(OC)c3ccccc23)NC1=O ; CCC(CCCCC[C@@H]1NC(=O)[C@H]2CCCCN2C(=O)[C@H](C(C)CC)NC(=O)[C@H](Cc2cn(OC)c3ccccc23)NC1=O)SC(C)=O ; CCC(=O)CCCCC[C@@H]1NC(=O)[C@H]2CCCCN2C(=O)[C@H]([C@H](C)CC)NC(=O)[C@@H](c2cnc3ccccc3c2N2CCCCC2)NC1=O]

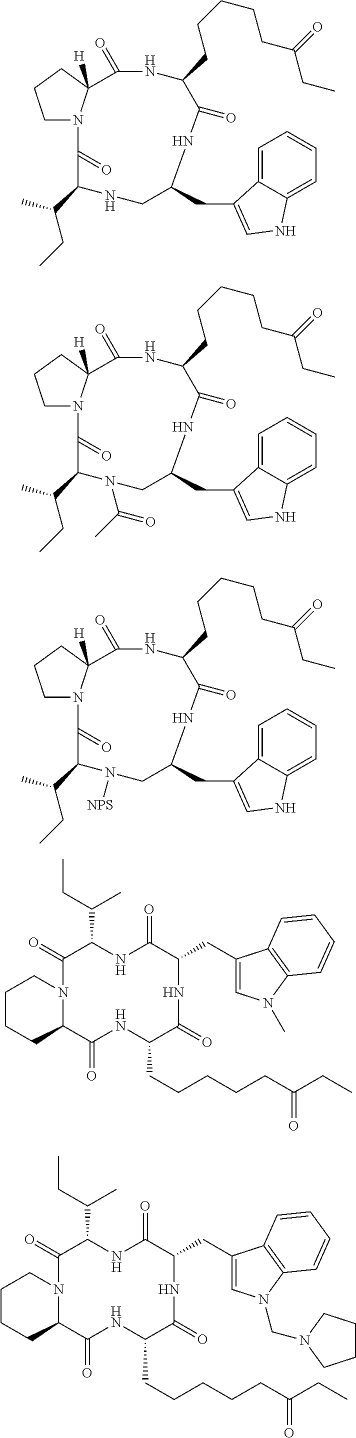

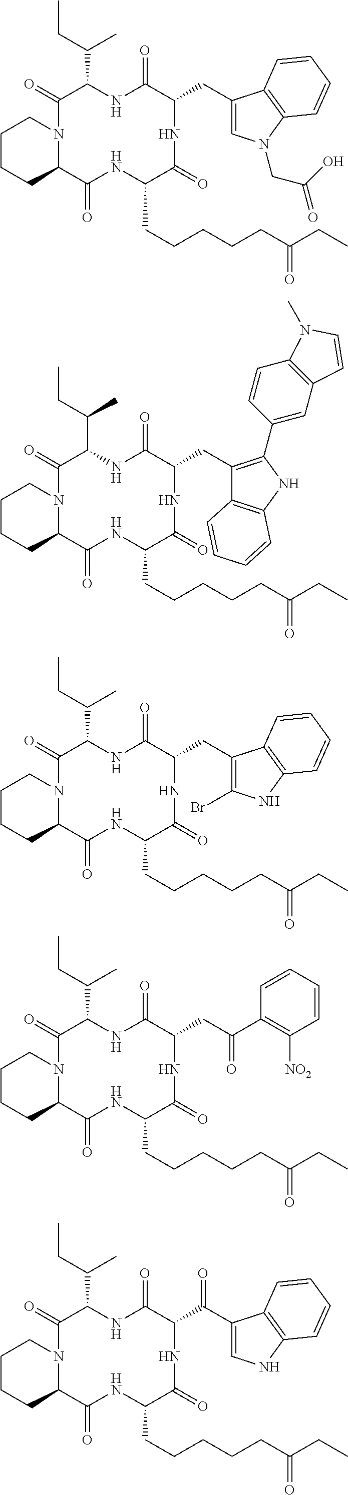

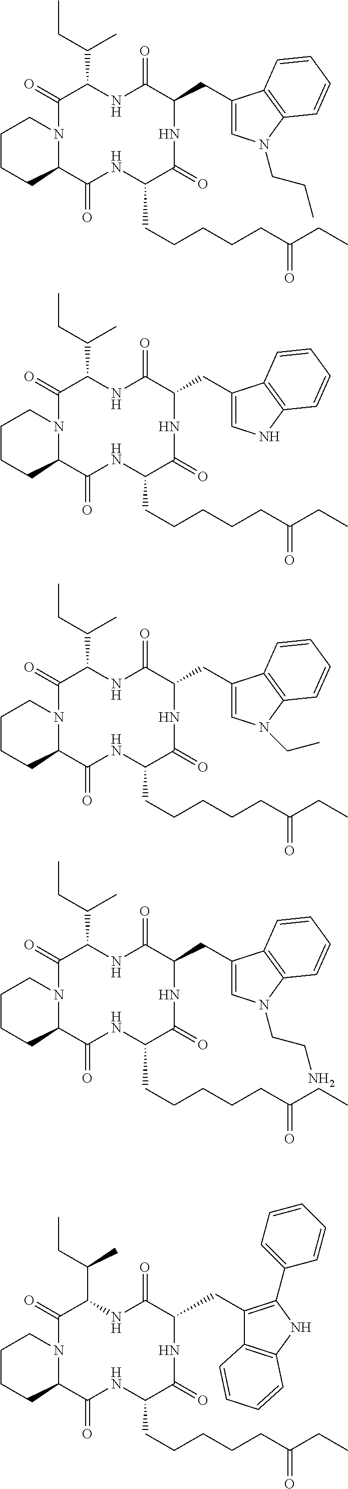 [SMILES: CCCn1cc(C[C@H]2NC(=O)[C@H](CCCCCC(=O)CC)NC(=O)[C@H]3CCCCN3C(=O)[C@H](C(C)CC)NC2=O)c2ccccc21 ; CCC(=O)CCCCC[C@@H]1NC(=O)[C@H]2CCCCN2C(=O)[C@H](C(C)CC)NC(=O)[C@H](Cc2c[nH]c3ccccc23)NC1=O ; CCC(=O)CCCCC[C@@H]1NC(=O)[C@H]2CCCCN2C(=O)[C@H](C(C)CC)NC(=O)[C@H](Cc2cn(CC)c3ccccc23)NC1=O ; CCC(=O)CCCCC[C@@H]1NC(=O)[C@H]2CCCCN2C(=O)[C@H](C(C)CC)NC(=O)[C@@H](Cc2cn(CCN)c3ccccc23)NC1=O ; CCC(=O)CCCCC[C@@H]1NC(=O)[C@H]2CCCCN2C(=O)[C@H]([C@H](C)CC)NC(=O)[C@H](Cc2c(-c3ccccc3)[nH]c3ccccc23)NC1=O]

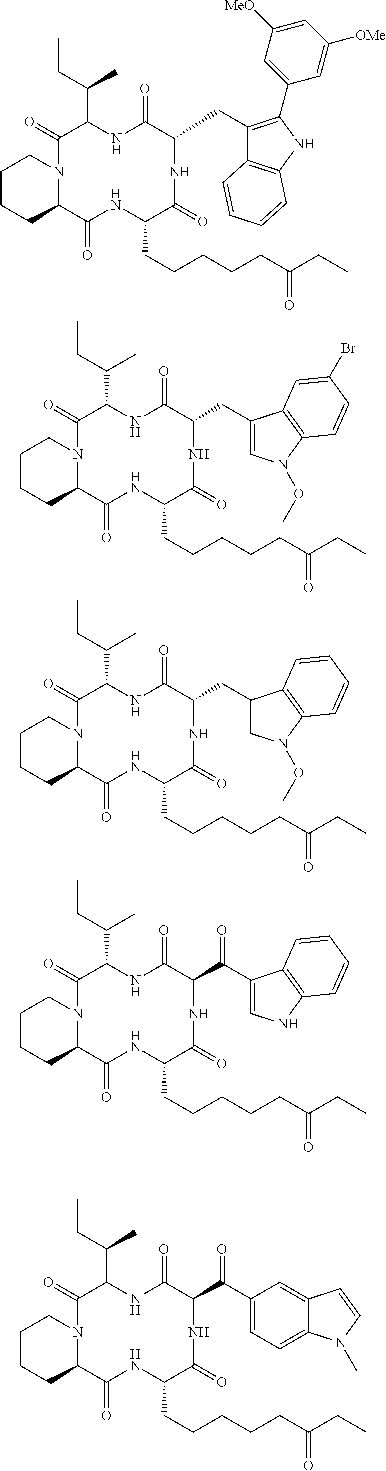 [SMILES: CCC(=O)CCCCC[C@@H]1NC(=O)[C@H]2CCCCN2C(=O)C([C@H](C)CC)NC(=O)[C@H](Cc2c(-c3cc(OC)cc(OC)c3)[nH]c3ccccc23)NC1=O ; CCC(=O)CCCCC[C@@H]1NC(=O)[C@H]2CCCCN2C(=O)[C@H](C(C)CC)NC(=O)[C@H](Cc2cn(OC)c3ccc(Br)cc23)NC1=O ; CCC(=O)CCCCC[C@@H]1NC(=O)[C@H]2CCCCN2C(=O)[C@H](C(C)CC)NC(=O)[C@H](CC2CN(OC)c3ccccc32)NC1=O ; CCC(=O)CCCCC[C@@H]1NC(=O)[C@H]2CCCCN2C(=O)[C@H](C(C)CC)NC(=O)[C@@H](C(=O)c2c[nH]c3ccccc23)NC1=O ; CCC(=O)CCCCC[C@@H]1NC(=O)[C@H]2CCCCN2C(=O)C([C@H](C)CC)NC(=O)[C@@H](C(=O)c2ccc3c(ccn3C)c2)NC1=O]

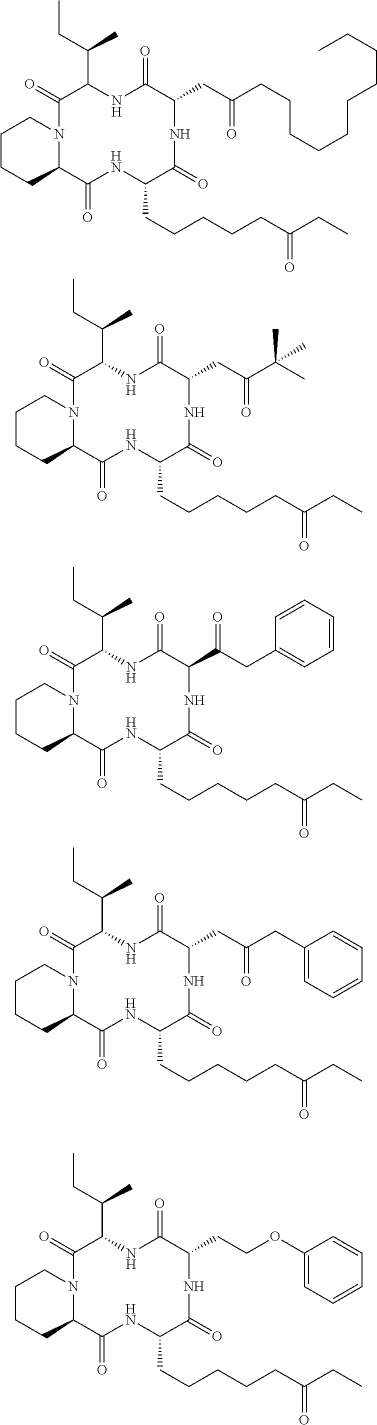

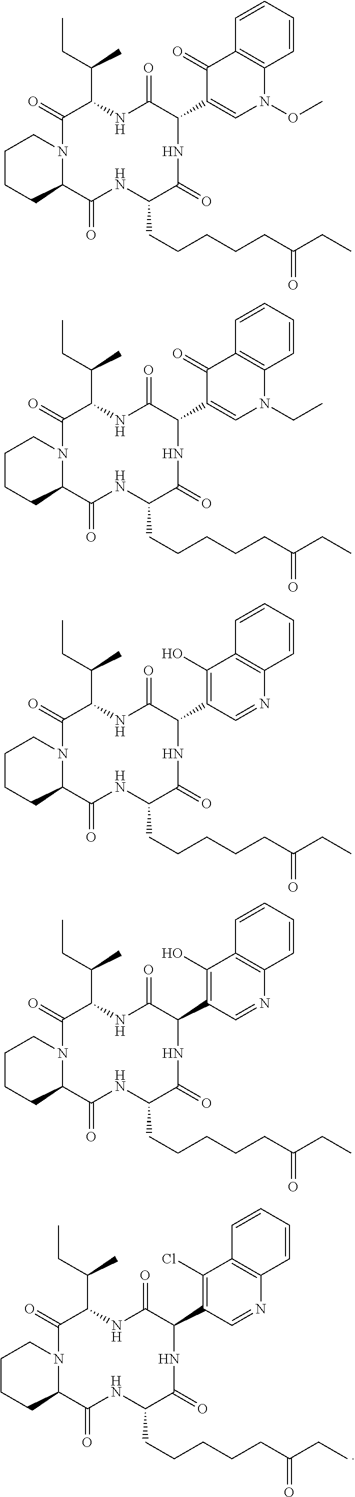

Exemplary methods to obtain natural analogs of apicidin are disclosed in (1) Darkin-Rattray, S. J.; Gurnett, A. M.; Myers, R. W.; Dulski, P. M.; Crumley, T. M.; Allocco, J. J.; Cannova, C.; Meinke, P. T.; Colletti, S. L.; Bednarek, M. A.; Singh, S. B.; Goetz, M. A.; Dombrowski, A. W.; Polishook, J. D.; Schmatz, D. M. Apicidin: A novel antiprotozoal agent that inhibits parasite histone deacetylase. Proceedings of the National Academy of Sciences 1996, 93, 13143-13147; (2) Singh, S. B.; Zink, D. L.; Liesch, J. M.; Mosley, R. T.; Dombrowski, A. W.; Bills, G. F.; Darkin-Rattray, S. J.; Schmatz, D. M.; Goetz, M. A. Structure and Chemistry of Apicidins, a Class of Novel Cyclic Tetrapeptides without a Terminal α-Keto Epoxide as Inhibitors of Histone Deacetylase with Potent Antiprotozoal Activities. J. Org. Chem. 2002, 67, 815-825; and (3) Singh, S. B.; Zink, D. L.; Liesch, J. M.; Dombrowski, A. W.; Darkin-Rattray, S. J.; Schmatz, D. M.; Goetz, M. A. Structure, Histone Deacetylase, and Antiprotozoal Activities of Apicidins B and C, Congeners of Apicidin with Proline and Valine Substitutions. Org. Lett. 2001, 3, 2815-2818.

Exemplary methods to obtain synthetic analogs of apicidin are disclosed in (1) Kuriyama, W.; Kitahara, T. Synthesis of Apicidin. Heterocycles 2001, 55, 1-4; (2) Mou, L.; Singh, G. Synthesis of (S)-2-amino-8-oxodecanoic acid (Aoda) and apicidin A. Tetrahedron Lett. 2001, 42, 6603-6606; (3) Berst, F.; Ladlow, M.; Holmes, A. B. Solid-phase synthesis of apicidin A and a cyclic tetrapeptoid analogue. Chem. Commun. 2002, 508-509; (4) Singh, S. B.; Zink, D. L.; Liesch, J. M.; Mosley, R. T.; Dombrowski, A. W.; Bills, G. F.; Darkin-Rattray, S. J.; Schmatz, D. M.; Goetz, M. A. Structure and Chemistry of Apicidins, a Class of Novel Cyclic Tetrapeptides without a Terminal α-Keto Epoxide as Inhibitors of Histone Deacetylase with Potent Antiprotozoal Activities. J. Org. Chem. 2002, 67, 815-825; (5) Meinke, P. T.; Colletti, S. L.; Ayer, M. B.; Darkin-Rattray, S. J.; Myers, R. W.; Schmatz, D. M.; Wyvratt, M. J.; Fisher, M. H. Synthesis of side chain modified apicidin derivatives: potent mechanism-based histone deacetylase inhibitors. Tetrahedron Lett. 2000, 41, 7831-7835; (6) Colletti, S. L.; Myers, R. W.; Darkin-Rattray, S. J.; Gurnett, A. M.; Dulski, P. M.; Galuska, S.; Allocco, J. J.; Ayer, M. B.; Li, C.; Lim, J.; Crumley, T. M.; Cannova, C.; Schmatz, D. M.; Wyvratt, M. J.; Fisher, M; (7) Singh et al. Structure, Histone Deacetylase, and Antiprotozoal Activities of Apicidins B and C, Congeners of Apicidin with Proline and Valine Substitutions. Org Lett. 2001 Sep. 6; 3(18):2815-8.

I. Compositions

Bacterial infections caused by staphylococcus bacteria (i.e., a “staph infection” or “a staphylococcal infection”) are very common in the general population. About 25% of individuals commonly carry staphylococcus bacteria on their skin or in their nose. Most of the time, these bacteria do not cause or problem or may cause a relatively minor skin infection. However, staph infections can become deadly if the bacteria invade deeper into an individual's body, for example, entering the bloodstream, joints, bones, lungs or heart. In the past, a lethal staph infection might have occurred in a person who was hospitalized or had a chronic illness or weakened immune system. Now, it is increasingly common for an otherwise healthy individual to develop a life-threatening staph infection. Many staph infections have become recalcitrant to antibiotic treatment due to infection with strains that exhibit true antibiotic resistance or reduced susceptibility to existing antibiotics. Such reductions in antibiotic effectiveness are typically more pronounced in patients with weakened immune systems due to immune senescence, co-morbidities, or co-administered pharmaceutical agents or other medical procedures. Staphylococcus aureus, often referred to as “staph” or “S. aureus,” is a major human pathogen, producing a multitude of virulence factors making it able to cause several types of infection, from superficial lesions to toxinoses and life-threatening systemic conditions such as endocarditis, osteomyelitis, pneumonia, meningitis and sepsis (reviewed in Miller and Cho, “Immunity Against Staphylococcus aureus Cutaneous Infections,” Nat. Rev. Immunol. 11:505-518 (2011)). The staphylococcal infection may be caused by any staphylococcal species. In one aspect, the staphylococcal infection is caused by Staphylococcal infection aureus, including methicillin-resistant S. aureus (MRSA) and methicillin-sensitive S. aureus (MSSA).

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

As used herein throughout, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

Some of the foregoing compounds can comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of the invention may be prepared by crystallization under different conditions and may exist as one or a combination of polymorphs. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present invention encompasses inventive compounds, their derivatives, their tautomeric forms, their stereoisomers, their polymorphs, their pharmaceutically acceptable salts their pharmaceutically acceptable solvates and pharmaceutically acceptable compositions containing them.

The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference.

The pharmaceutical compositions of the present invention additionally comprise a pharmaceutically acceptable carrier, which, as used herein, includes any and all solvents, diluents, or other liquid vehicle, vehicle, coating, dispersion or suspension medium or aids, surface active agents, antibacterial and/or antifungal agent, isotonic agents, thickening or emulsifying agents, preservatives, solid binders, lubricants, absorption delaying agent, buffer, carrier solution, suspension, colloid, and the like, as suited to the particular dosage form desired. The use of such media and/or agents for pharmaceutical active substances is well known in the art. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In certain embodiments, these compositions optionally further comprise one or more additional therapeutic agents. Alternatively, a compound of this invention may be administered to a patient in need thereof in combination with the administration of one or more other therapeutic agents. For example, additional therapeutic agents for conjoint administration or inclusion in a pharmaceutical composition with a compound of this invention may be an immunomodulatory agent, antibiotic agent, or anticancer agent. It will also be appreciated that certain of the compounds of present invention can exist in free form for treatment, or where appropriate, as a pharmaceutically acceptable derivative thereof.

The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A pharmaceutical composition also can be administered via a sustained or delayed release.

It will be appreciated that the inventive compound may be administered systemically in dosage forms, formulations or e.g. suitable delivery devices or implants containing conventional, non-toxic pharmaceutically acceptable carriers and adjuvants such that the compound effectiveness is optimized. For example, the inventive compound may be formulated together with appropriate excipients into a pharmaceutical composition, which, upon administration of the composition to the subject, systemically releases the active substance in a controlled manner. Alternatively, or additionally, compound dosage form designs may be optimized so as to increase the compound effectiveness upon administration. The above strategies (i.e., dosage form design and rate control of drug input), when used alone or in combination, can result in a significant increase in compound effectiveness and are considered part of the invention.

The pharmaceutical composition may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

II. Methods of Treatment

The method involves the administration of a therapeutically effective amount of the compound or pharmaceutically acceptable derivative thereof to a subject (including, but not limited to a human or animal) in need of it. As used herein, “therapeutically effective amount” or an “effective amount” indicates an amount that results in a desired pharmacological and/or physiological effect for the condition. The effect may be prophylactic in terms of completely or partially preventing a condition or symptom thereof and/or may be therapeutic in terms of a partial or complete cure for the condition and/or adverse effect attributable to the condition. The exact amount required will vary from subject to subject, depending on the species, age, and general condition of the subject, the severity of the diseases, its mode of administration, and the like. The compounds of the invention are preferably formulated in dosage unit form for ease of administration and uniformity of dosage. The expression dosage unit form as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disease or indication being treated and the severity of the disease or indication; the specific compound; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, Tenth Edition, A. Gilman, J. Hardman and L. Limbird, eds., McGraw-Hill Press, 155-173, 2001, which is incorporated herein by reference in its entirety). Those of ordinary skill in the art can readily determine the appropriate amount with consideration of relevant factors.

In some embodiments, frequency of administration may be, for example, from a single dose to multiple doses per week, although in some embodiments the method can be performed by administration at a frequency outside this range. In certain embodiments, administration may be from about once per month to about five times per week. In other embodiments, administration may be on an as needed basis. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

Administration to a subject may be before or after the subject manifests a symptom or clinical sign of infection by a microbe. “Symptom” refers to any subjective evidence of disease or of a patient's condition. “Sign” or “clinical sign” refers to an objective physical finding relating to a particular condition capable of being found by one other than the patient. Treatment that is initiated before a subject manifests a symptom or clinical sign of infection can be considered prophylactic treatment of a subject “at risk” of infection by the microbe. As used herein, the term “at risk” refers to a subject that may or may not actually possess the described risk. Thus, for example, a subject “at risk” of infectious condition is a subject present in an area where other individuals have been identified as having the infectious condition and/or is likely to be exposed to the infectious agent even if the subject has not yet manifested any detectable indication of infection by the microbe and regardless of whether the subject may harbor a subclinical amount of the microbe.

Accordingly, administration of a composition can be performed before, during, or after the subject first exhibits a symptom or clinical sign of the condition or, alternatively, before, during, or after the subject first comes in contact with the infectious agent. Treatment initiated before the subject first exhibits a symptom or clinical sign associated with the condition may result in decreasing the likelihood that the subject experiences clinical evidence of the condition compared to an animal to which the composition is not administered, decreasing the severity of symptoms and/or clinical signs of the condition, and/or completely resolving the condition. Treatment initiated after the subject first exhibits a symptom or clinical sign associated with the condition can be considered therapeutic treatment of the subject, and may result in decreasing the severity of symptoms and/or clinical signs of the condition compared to an animal to which the composition is not administered, and/or completely resolving the condition.

Furthermore, after formulation with an appropriate pharmaceutically acceptable carrier or diluent in a desired dosage, the pharmaceutical compositions of this invention can be administered to humans and other animals orally, rectally, parenterally, intracisternally, intravaginally, intraperitoneally, topically (as by powders, ointments, creams or drops), bucally, as an oral or nasal spray, or the like, depending on the severity of the infection being treated. In certain embodiments, the compounds of the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally. The dose may be calculated using actual body weight obtained just prior to the beginning of a treatment course. For the dosages calculated in this way, body surface area (m₂) is calculated prior to the beginning of the treatment course using the Dubois method: m₂=30 (wt kg_{0.425}×height cm_{0.725})×0.007184.

In certain embodiments, an effective amount of the active ingredient for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg.

In certain embodiments, the active ingredient may be administered at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions, which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner Examples of embedding compositions, which can be used, include polymeric substances and waxes.

The present invention encompasses pharmaceutically acceptable topical formulations of inventive compounds. The term “pharmaceutically acceptable topical formulation”, as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate) and N-methyl pyrrolidone.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

In certain embodiments, after application of the topical formulation to the epidermis, the area may be covered with a dressing. The term “dressing”, as used herein, means a covering designed to protect a topically applied drug formulation. “Dressing” includes coverings such as a bandage, which may be porous or non-porous and various inert coverings, e.g., a plastic film wrap or other non-absorbent film. The term “dressing” also encompasses non-woven or woven coverings, particularly elastomeric coverings, which allow for heat and vapor transport. These dressings allow for cooling of the treated area, which provides for greater comfort.

In certain exemplary embodiments, pharmaceutically acceptable topical formulations of the invention are contained in a patch that is applied adjacent to the area of skin to be treated. As used herein a “patch” comprises at least a topical formulation and a covering layer, such that, the patch can be placed over the area of skin to be treated. Preferably, but not necessarily, the patch is designed to maximize drug delivery through the stratum corneum and into the epidermis or dermis, reduce lag time, promote uniform absorption, and/or reduce mechanical rub-off. In certain embodiments, when the intended use comprises the treatment of a skin condition (e.g., psoriasis), the patch is designed to minimize absorption into the circulatory system. Preferably, the patch components resemble the viscoelastic properties of the skin and conform to the skin during movement to prevent undue shear and delamination. Advantages of a patch comprising the topical formulation of the invention over conventional methods of administration include (i) that the dose is controlled by the patch's surface area, (ii) constant rate of administration, (iii) longer duration of action (the ability of to adhere to the skin for 1, 3, 7 days or longer), (iv) improved patient compliance, (v) non-invasive dosing, and (vi) reversible action (i.e., the patch can simply be removed).

In certain embodiments, a patch suitable for use with the invention contains at least: (1) a backing layer and (2) a carrier formulated with a compound of the invention. Examples of patch systems suitable for practicing the invention include, but are not limited to, matrix-type patches; reservoir-type patches; multi-laminate drug-in-adhesive-type patches; and monolithic drug-in-adhesive type-patch. See, for example Ghosh, T. K.; Pfister, W. R.; Yum, S. I. Transdermal and Topical Drug Delivery Systems, Interpharm Press, Inc. p. 249-297, which is incorporated herein by reference in its entirety. These patches are well known in the art and generally available commercially.

The matrix patch comprises matrix containing an inventive compound, an adhesive backing film overlay, and preferably, but not necessarily, a release liner. In some cases, it may be necessary to include a impermeable layer to minimize drug migration into the backing film (e.g., U.S. Pat. No. 4,336,243, incorporated herein by reference). In certain embodiments, the matrix containing the inventive compound is held against the skin by the adhesive overlay. Examples of suitable matrix materials include but are not limited to lipophilic polymers, such as polyvinyl chloride, polydimethylsiloxane, and hydrophilic polymers like polyvinylpyrrolidone, polyvinyl alcohol, hydrogels based on gelatin, or polyvinylpyrrolidone/polyethylene oxide mixtures. Suitable release liners include but are not limited to occlusive, opaque, or clear polyester films with a thin coating of pressure sensitive release liner (e.g., silicone-fluorosilicone, and perfluorocarbon based polymers.

It will also be appreciated that the pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another immunomodulatory agent, antibiotic agent, or anticancer agent), or they may achieve different effects (e.g., control of any adverse effects).

### 6. Examples

The following Examples further illustrate the disclosure and are not intended to limit the scope. In particular, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims. Various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

I. Materials and Methods

A. General

NMR data were collected using a JEOL ECA-500 NMR spectrometer operating at 500 MHz for ¹H and 125 MHz for ¹³C (JEOL Ltd., Tokyo, Japan). Residual solvent signals were utilized for referencing. High resolution mass spectra (HRMS) were obtained using a Thermo LTQ Orbitrap XL mass spectrometer equipped with an electrospray ionization source (Thermo Fisher Scientific, San Jose, Calif., USA). Phenomenex Gemini-NX C₁₈ analytical (5 μm; 250×4.6 mm) and preparative (5 μm; 250×21.2 mm) columns (Phenomenex, Torrance, Calif., USA) were used on a Varian Prostar HPLC system equipped with ProStar 210 pumps and a Prostar 335 photodiode array detector (PDA), with data collected and analyzed using Galaxie Chromatography Workstation software (version 1.9.3.2, Varian Inc.). Flash chromatography was conducted on a Teledyne ISCO CombiFlash Rf using Silica Gold columns and monitored by UV and evaporative light-scattering detectors (both from Teledyne Isco, Lincoln, Nebr., USA).

B. Fungal Strain MSX53644 Isolation and Identification

Fermentation, Extraction and Isolation

Fungal strain MSX53644 from Mycosynthetix library was stored, fermented, and extracted as reported previously. Briefly, a 2.8-L Fernbach flask (Corning, Inc., Corning, N.Y., USA) containing 150 g rice and 300 mL H₂O was inoculated with a seed culture of fungal strain MSX53644 that was grown in YESD medium. After incubation for 14 days at r.t., the solid culture was extracted by addition of a 500 mL mixture of 1:1 MeOH/CHCl₃. Using a spatula, the culture was chopped into small pieces and left to shake at 125 rpm at r.t., followed by filtration. The solid residues were then washed with 100 mL of 1:1 MeOH/CHCl₃. To the combined filtrates, 900 mL CHCl₃ and 1500 mL H₂O were added so that the final ratio of CHCl₃/MeOH/H₂O was 4:1:5 and left to stir for 30 min. The mixture was then transferred into a separatory funnel and the organic bottom layer was drawn off and evaporated to dryness. The dried organic phase was then re-constituted in 100 mL of 1:1 MeOH/CH₃CN and 100 mL of hexanes and transferred into a separatory funnel. The MeOH/CH₃CN layer was drawn off and evaporated to dryness under vacuum. The defatted crude material (1.2 g) was dissolved in a mixture of CHCl₃/MeOH, adsorbed onto Celite 545, and fractionated via normal phase flash chromatography using a gradient solvent system of hexane/CHCl₃/MeOH at a 40 mL/min flow rate and 53.3 column volumes over 63.9 min to afford five fractions. Fraction 4 (300 mg) was subjected to preparative reversed-phase HPLC over a Phenomenex Gemini-NX C18 preparative column using a gradient system of 40:60 to 70:30 over 30 min of CH₃CN/H²O (acidified with 0.1% formic acid) at a flow rate of 21.24 mL/min (FIG. S1A, Supporting Information) to yield compounds 1 (4.1 mg) and 2 (3.0 mg) which eluted at 18.0 and 19.5 min, respectively.

Plant Material

Plant material of yerba mansa [Anemopsis californica (Nutt.) Hook. & Arn. (Saururaceae)] was collected with permission by Amy Brown of Apache Creek Ranch in Santa Fe, N.M. (35° 35′ 56.40″N, 105° 50′ 27.22″W). A voucher specimen (NCU602027) was deposited in the University of North Carolina Herbarium. The specimen was authenticated by Amy Brown.

Fungal Endophyte Strains G134 and G137 Isolation and Identification

The endophytic fungal strains G134 and G137 were isolated from surface sterilized fresh roots of yerba mansa using methods reported previously. G134 and G137 were found to be two isolates for the same strain and were identified as a Fusarium sp. by sequencing the internal transcribed spacer region of the ribosomal RNA gene (ITS) using molecular methods reported previously. The ITS sequences for G134 and G137 were deposited in GenBank (accession no. KM816766 for G134 and KM816768 for G137).

Fermentation, Extraction and Isolation

Endophyte fungal strains G134 and G137 were fermented and extracted as reported previously and as outlined above for fungal strain MSX53644. The extracts from G134 and G137 were combined as the LC-MS analysis of the extracts showed similar chemical profiles, the two cultures showed similar morphological characteristics, and decisively, the two isolates showed similar sequences of the internal transcribed spacer region of the ribosomal RNA gene (ITS). The combined defatted extracts of G134 and G137 (125 mg) were then fractionated using normal phase flash chromatography using a gradient solvent system of hexane/CHCl₃/MeOH at a 18 mL/min flow rate and 68.1 column volumes over 18.2 min to afford five fractions. Fraction 5 (35 mg) was found to contain apicidin as evidenced by LC-MS analysis and hence was subjected to preparative reversed-phase HPLC purification over a Phenomenex Gemini-NX C₁₈ preparative column using a gradient system of 40:60 to 70:30 over 30 min of CH₃CN/H₂O (acidified with 0.1% formic acid) at a flow rate of 21.24 mL/min to yield compounds 2 (8.9 mg), 3 (1.7 mg), and 4 (1.5 mg), which eluted at 19.5, 15.5, and 17.5, respectively.

C. Bacterial culture conditions and strains.

| Strain | Description | Ref |
|---|---|---|
| AH1263 | LAC WT | Boles, 2010 |
| AH1292 | LAC agr deletion mutant | Keid, 2011 |
| AH1677 | agr I LAC (agr::P3yfp) | Hall, 2013 |
| AH430 | agr II 502a (agr::P3yfp) | Hall, 2013 |
| AH1747 | agr III MW2 (agr::P3yfp) | Hall, 2013 |
| AH1872 | agr IV MN TG (agr::P3yfp) | Hall, 2013 |
| AH2759 | agr I LAC (agr::P31ux) | This work |
| AH3700 | LAC Xen29: lux | This work |
| AH3868 | LAC Xen291ux + PHC48 | This work |
| AH3048 | Δagr + EPSAS (agr Ac) | Sully, 2014 |
| AH3468 | | This work |
| AH3469 | | This work |
| AH3490 | | This work |

II. Experimental Procedures

Quenching Assays with Reporter Strains.

Apicidin was tested for quorum quenching activity against all four agr types using P3-YFP reporter strains AH1677 (type I), AH430 (type II) AH1747 (type III), and AH1872 (type IV). (28) Overnight cultures of reporter strains that were grown in TSB supplemented with Cam were inoculated at a dilution of 1:250 into fresh TSB containing Cam. 100 μL, aliquots were added to 96-well microtiter plates containing 100 μL aliquots of TSB containing Cam and 2-fold serial dilutions of apicidin. Readings were recorded at 30 min increments using a Tecan Systems Infinite M200 plate reader.

Evaluation of Skin and Blood Immune Cells:

For skin: abdominal skin ulcerations were excised and incubated in trypsin (0.6% in PBS) for 75 min at 37° C.; then cut into small pieces and incubated in Collagenase type II (0.5 mg/ml RPMI) for 90 min at 37° C. Skin cell suspensions were generated by serial passage of skin fragments through 18 and 20 gauge syringes. IN cell suspensions were generated mincing inguinal LNs with a razor blade before by serial passage of skin fragments through 18 and 20 gauge syringes. AU tissue preps were passed through a 70 am filter before immune cell staining. For whole blood: blood was collected, washed and resuspended in Tris-buffered ammonium chloride to lyse red blood cells.

Hemolytic Activity.

Overnight cultures of agr strain types I, II, III, and IV were inoculated 1:500 into 5 ml of TSB (in 17×150 mm culture tubes) containing apicidin at concentrations of 100, 50, 25, 12.5, and 6.25 μg mL (28). All cultures were incubated at 37° C. with shaking (250 rpm), and growth was monitored by periodically transferring 100 μL of culture to a 96-well microtiter plate and reading OD600 in a Tecan Systems Infinite M200 plate reader. Following incubation, 600 μL of each culture was filter sterilized using cellulose acetate SpinX 0.22 am filters. To quantify hemolytic activity, the filter sterilized culture supernatants from apicidin treated cultures were serially in TSB, and 50 μL aliquots were dispensed in quadruplicate into 96-well microtiter plates. Rabbit erythrocytes, were added to the microtiter plates at 50 μL per well. The erythrocytes and culture supernatants were mixed and incubated statically at room temperature for 2 hr. Hemolysis was detected by the loss of turbidity as measured at OD630 using a Tecan Infinite M200 plate reader.

Human Whole Blood Killing Assay.

For whole blood killing, overnight cultures of LAC were inoculated 1:100 into 200 μl of TSB+/−100 μM apicidin and subcultured for 4 hours (in 96-well microtiter plate at 37° C. with shaking at 250 rpm). Following subculture, 200 μl of human whole blood was inoculated with ≈1.0×106 LAC and incubated for 1 hour in 96-well microtiter plate at 37° C. with shaking at 250 rpm. To calculate whole blood killing, the CFUs recovered after blood culture were compared to the bacterial load of the inoculum.

Whole Blood Killing and Phagocytosis Assays.

For killing assays: PB was collected in sodium heparin tubes, transferred into round bottomed 96 well plates (Corning, N.Y.) LAC cultures were grown in the presence of apicidin (100 □M) or DMSO for 4 hr at 37° C. with shaking (250 rpm). After culture, cells were washed in saline and resuspended to a concentration of □ 1×105 CFUs/□L. Cells exposed to Apicidin or control were then mixed with heparinized PB in at a concentration of 1×106 CFUs/150 μL PB. To whole blood killing, CFUs DsRed LAC/150 μL of PB. For peripheral blood (PB) phagocytosis assays: PB was collected in sodium heparin tubes, transferred into round bottomed 96 well plates (Corning, N.Y.) and inoculated with 1×106 DsRed LAC/150 μL of PB. The infected blood was incubated at 37° C. in a 37° C. with shaking (250 rpm). After 1 h, serial dilutions in saline were performed to determine the endpoint numbers of CFU, which were compared to the initial bacterial load to determine the viable percentage of the initial inoculum. For phagocytosis assays, the PB/DsRed+ LAC mixtures were washed in saline, and incubated in tris ammonium chloride for 20 min at room temperature to lyse red blood cells. After washing twice in FACs buffer, cells were stained for flow cytometry.

RNA Seq:

Cultures of LAC and LAC [symbol] agr were grown in TSB with DMSO alone or 100 □M apicidin (diluted in neat DMSO). All four cultures were grown to optical density of 1.4 at 600 nm, and the RNA was purified. The samples were subjected to DNase treatment and sample was quality was affirmed via Bioanalyzer (Agilent). rDNA was depleted with a Ribo-Zero rRNA Removal Kit from gram positive bacteria (Illumina). cDNA libraries were generated at the University of Iowa Genomics Division using the TruSeq Stranded mRNA Library Prep kit (Illumina). Sample were barcoded, pooled and sequenced in a 100×100 pared end reads using HiSeq_FPR3757 genome sequence using SeqMan NGen (DNASTAR) and the alignment were analyzed using ArrayStar (DNASTAR). Genes were considered differentially expressed if they showed a >4-fold change in expression with 95% confidence as evaluated the student's t-test with a false discovery rate (FDR) correction applied for multiple t-tests.

S. aureus Skin Infections

At D0, age, strain and sex matched mice were anesthetized with isoflurane, abdominal skin was carefully shaved and exposed skin was cleansed by wiping with an alcohol prep pad. For inoculum preparation, a USA 300 MRSA strain (AH1263), its deletion mutant, LAC strains engineered for constitutive bioluminescence via lux operon insertion (AH3700) or the Lux+ LAC engineered to constitutively express red fluorescent protein (AH 3868), green fluorescent protein (AH3669) were grown to mid-log-phase, pelleted and resuspended in DPBS to achieve 50 μl inoculum mixtures that contained either 2×107 CFUs+/−5 μg apicidin (diluted in neat DMSO). Apicidin or DMSO containing inoculums were injected intradermally into abdominal skin. Baseline body weights of mice were measured before infection and every day thereafter for a period of 7 days. For determination of lesion size, digital photos of skin lesions were taken daily and analyzed via ImageJ software.

Measurement of Quorum Quenching In Vivo.

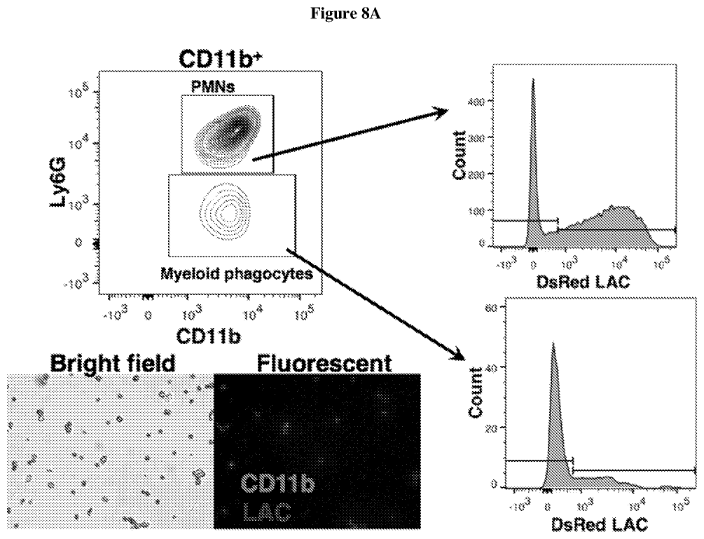

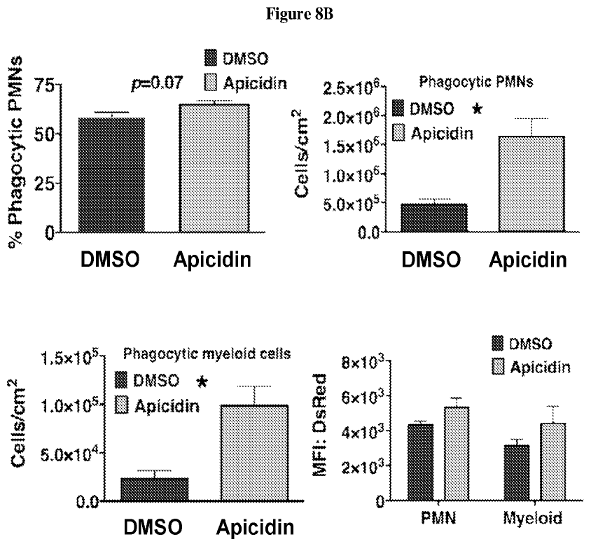

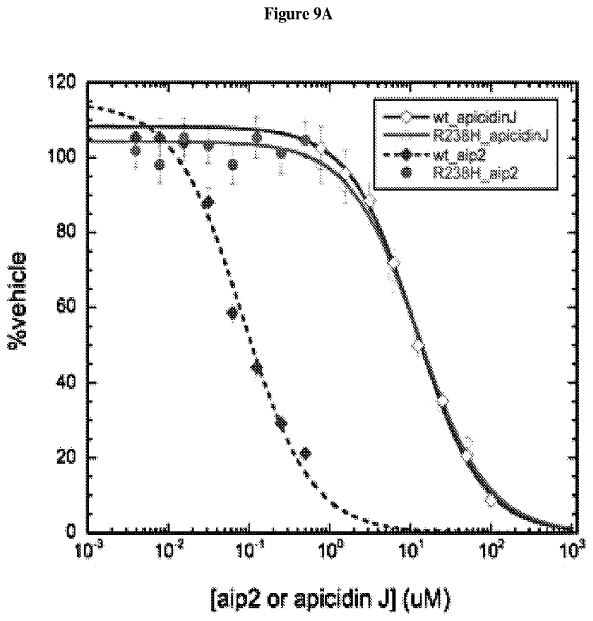

Inoculum preparation for assessing quorum quenching in vivo: An LAC reporter strain engineered to couple agr activation with bioluminescence, agr-P3 lux (AH2759) was grown in TSB medium+chloramphenicol, overnight at 37° C. in a shaking incubator set to 200 rpm. Overnight cultures were diluted 1:100 TSB+chloramphenicol and subcultured to an optical density of 0.1 at 600 nm hr). Bacterial cells were the pelleted and resuspended in sterile saline. 50 μL it inoculum suspensions containing 1×107 CFUs and 5 μg of apicidin diluted in DMSO or DMSO alone were injected intradermally into abdominal skin using 0.3 mL/31 gauge insulin syringe (as a technical control several mice were injected in the same manner with ␣L of sterile saline only. For all infections, challenge dose was confirmed by plating serial dilutions of inoculum on TSA and counting ensuing colonies after overnight culture. Beginning immediately after infection, mice were imaged under isoflurane inhalation anesthesia (2%). Photons emitted from luminescent bacteria were collected during a 2 min exposure using the Xenogen IVIS Imaging System and living image software (Xenogen, Alameda, Calif.). Bioluminescent image data are presented on a pseudocolor scale (blue representing least intense and red representing the most intense signal) overlaid onto a gray-scale photographic image. Using the image analysis tools in living image software, circular analysis windows (of uniform area) were overlaid onto abdominal regions of interest (as depicted in FIG. 8e) and the corresponding bioluminescence values (total flux) were measured and plotted vs. time after infection.

Flow Cytometry:

For staining of skin, LN and whole blood preparations, the following antibodies: Anti-Ly6G (AI8), Anti-Ly6C (HK1.4), -CD11b (M1/70), CD45 (30-F11) were purchased from BioLegend. To block nonspecific binding, cells were incubated with rat anti-mouse CD16/32 FcγRIII/II (2.4G2) and vortexed prior to surface staining. In all experiments, LN or blood cells were collected on a FACS LSR using Cellquest software, and analyzed via FlowJo software. Dead cells were excluded by low forward-scatter and side-light scatter. Spectral overlaps between fluorochrome channels were corrected by automated compensation on singly stained, positive controls for each fluorochrome. In general, 50,000 cells were collected/tube. Flow cytometric analyses of agr-interference in bacterial cultures were conducted.

Statistics:

The P values for comparisons between experimental groups, were calculated by use of an unpaired Student's t-test.

Cytokine Measurements from Tissue Homogenates.

Biopsy punches (4 mm), obtained from the center of skin ulcerations 1 day after infection, were homogenized with in sterile RPMI with proteinase inhibitor cocktail. The supernatants from these preparations were incubated with a multiplex bead array for IL-1β, G-CSF, KC (CXCL1), and CXCL2. Data were collected on a Luminex 200 (Luminex, Austin, Tex., USA) and analyzed with

III. Results

The studies identified apicidin as a novel quorum quencher with efficacy as anti-MRSA interventions. A library of terrestrial, freshwater, and endophytic fungal metabolites was screened and apicidin was found to be produced from both a terrestrial (MSX53644) and an endophytic (G134/137) fungal strains. In a non-bactericidal manner, apicidin inhibited quorum sensing activity across S. aureus isolates, achieving low micromolar MICs. Furthermore, whole transcriptome analysis demonstrated that the transcriptional signature of apicidin is narrowly focused upon the agr virulon. The translatability of apicidin-mediated quorum quenching in vitro to efficacy in vivo was confirmed in a cutaneous challenge model that also revealed quorum sensing interference within the infectious environment. Consistent with the latter finding, apicidin treatment enhanced polymorphonuclear neutrophil (PMN) accumulation and function at cutaneous sites of infection.

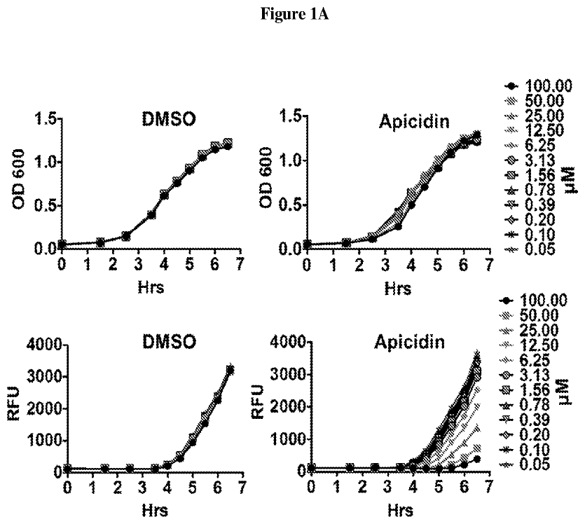

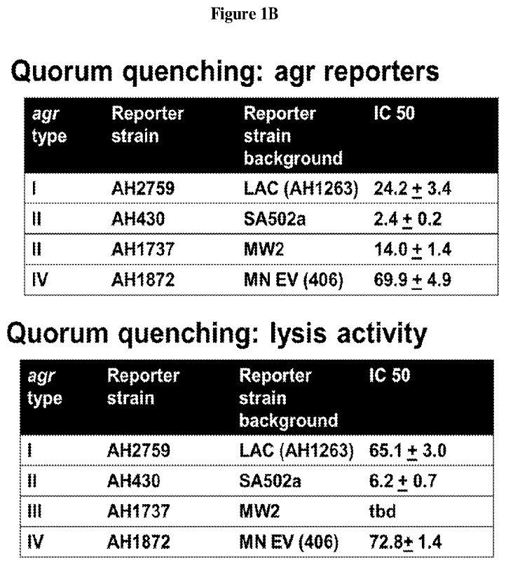

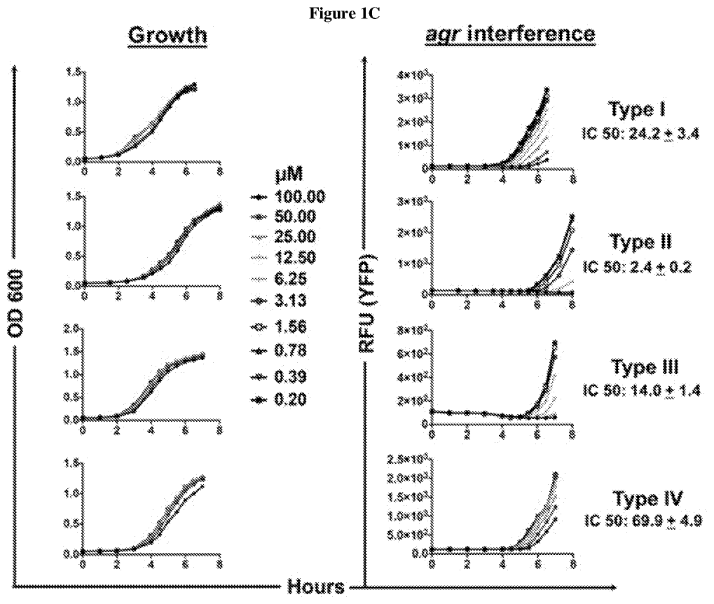

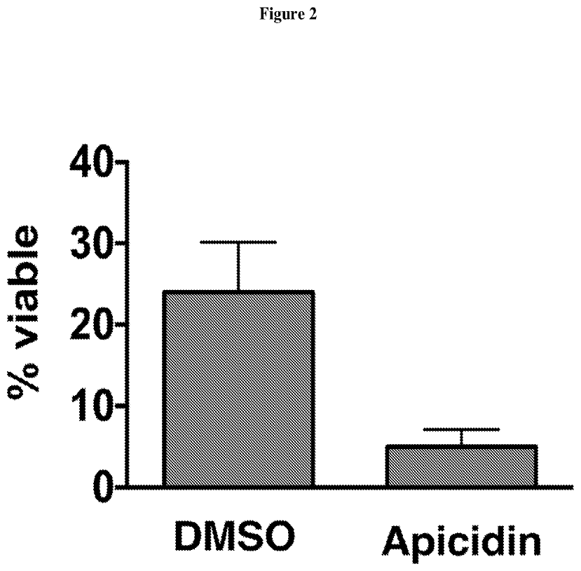

FIG. 1A depicts quorum quenching activity of apicidin against an LAC agr reporter with minimal growth inhibition. Top graphs show optical density (OD) measurements over time; bottom graphs show relative fluorescence unit (RFU) measurements over time (hours). Micromolar concentrations of DMSO or apicidin are displayed to the right of the graphs.

FIG. 1B are tables summarizing quorum quenching activity of apicidin for both agr reporter (top) and hemolytic (bottom) activity assays.

FIG. 1C are graphs of in vitro quorum quenching activity of apicidin. Apicidin mediated suppression of agr-P3 reporters (inhibition extends to all 4 agr types). Left) Time course showing quorum quenching activity of apicidin against an LAC agr reporter with minimal growth inhibition, micromolar concentrations of vehicle (DMSO) or apicidin are displayed to the right of the graphs.

FIG. 2 is a graph of the percentage of apicidin or vehicle exposed LAC recovered after 1 hr culture in human whole blood.

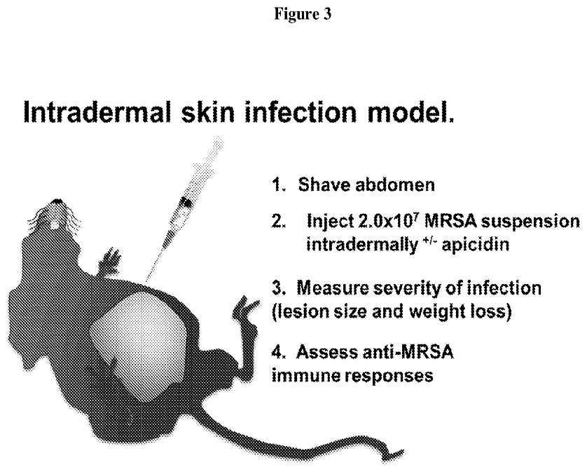

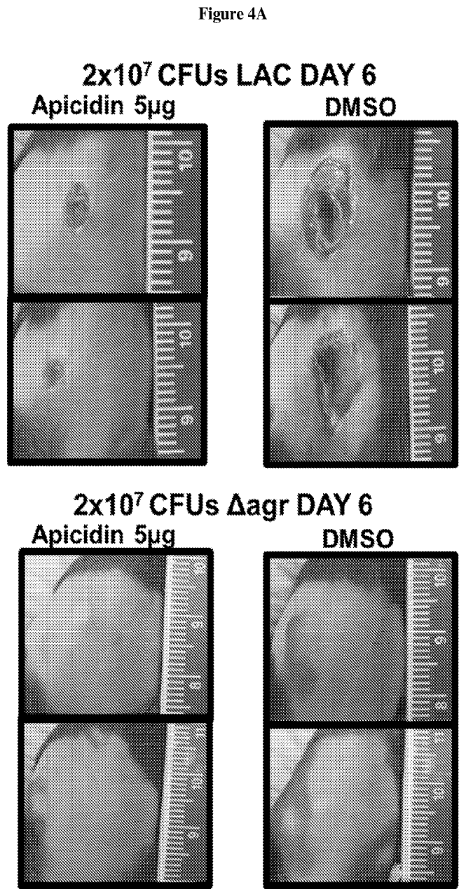

FIG. 3 is a graph with steps of an intradermal skin infection model.

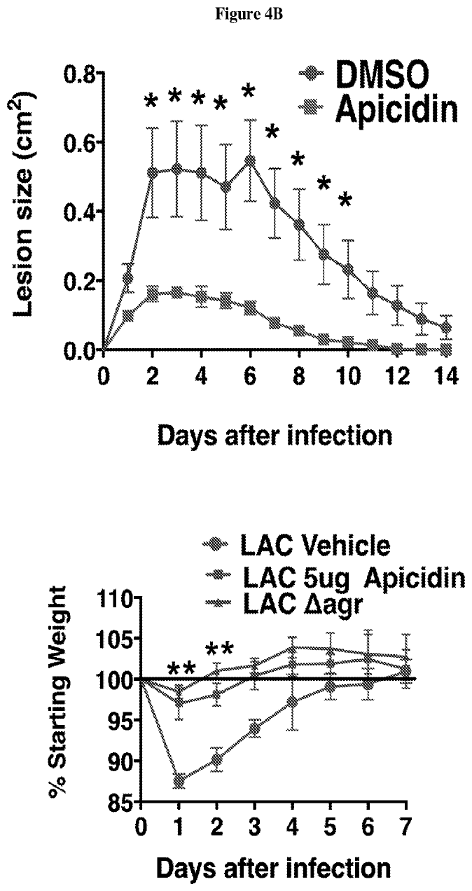

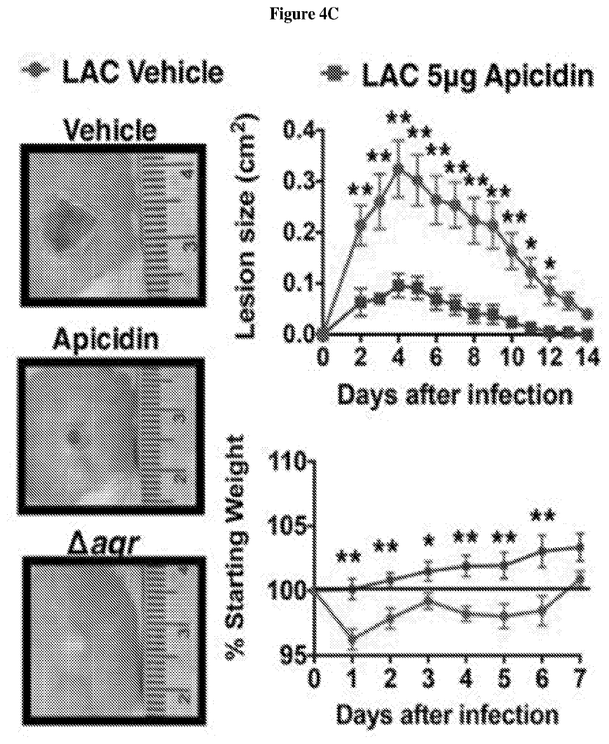

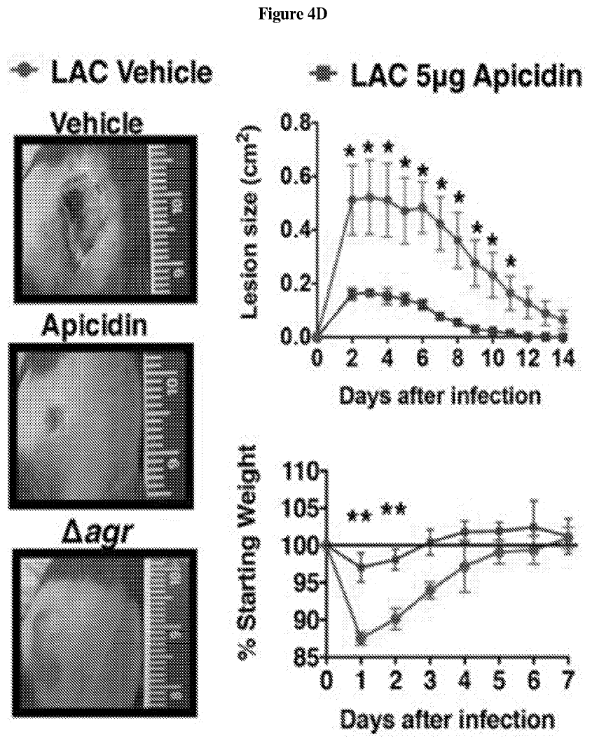

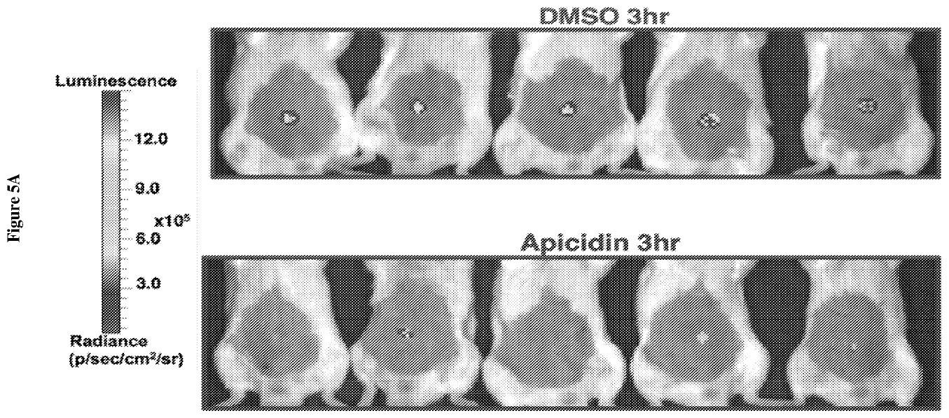

FIG. 4A are representative images of tissue injury in C57BL/6 mice infected with WT LAC+/−apicidin (left) or Δagr+/−apicidin (right).

FIG. 4C are representative images of tissue injury following infection with WT LAC+/−apicidin or Δagr apicidin and corresponding graphs of skin lesion size and weight loss and measurements following infection for the indicated groups in BALB/c (right) and C57BL/6 mice (left). Error bars represent SEM. Post test p value (*)=<0.05, (**)=<0.01

FIG. 4B are graphs depicting skin lesion size (left) and weight loss (right) measurements following infection for the indicated groups. Error bars represent SEM. Post test P value (*)=<0.05, (**)=<0.01.

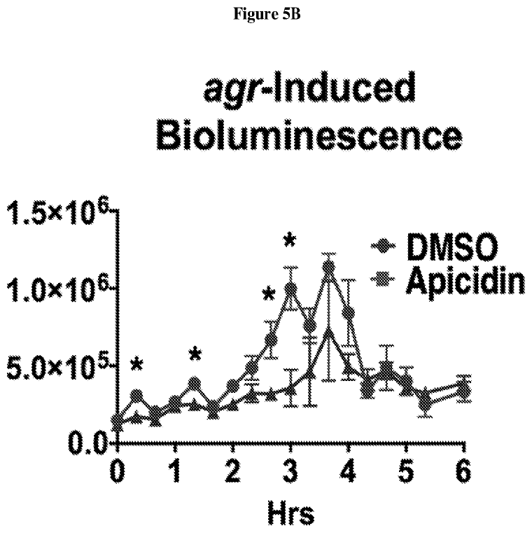

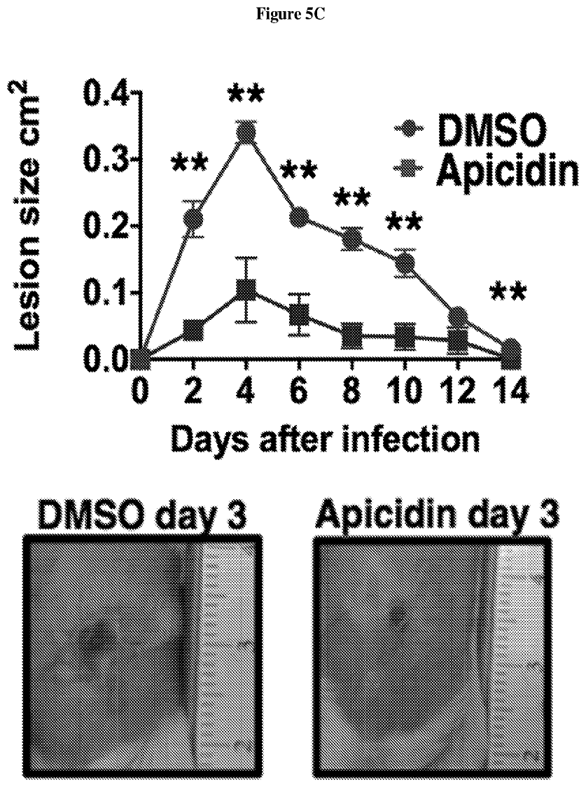

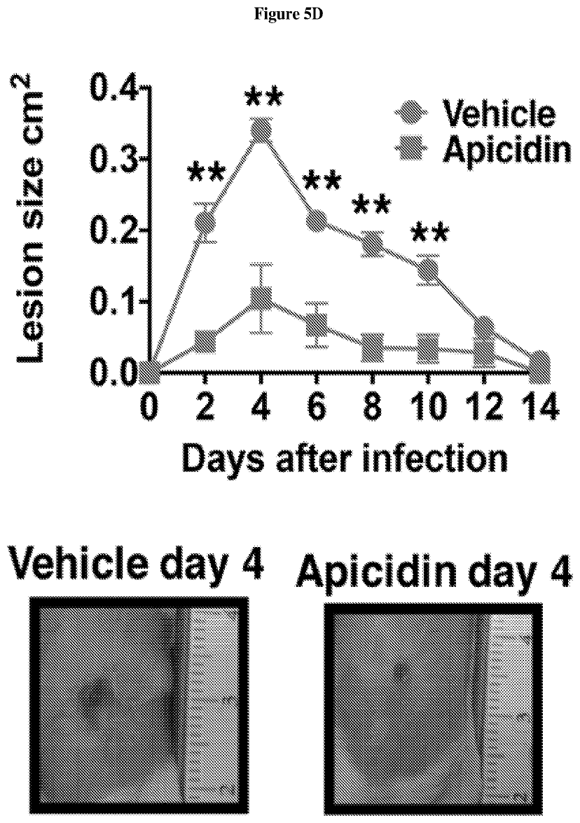

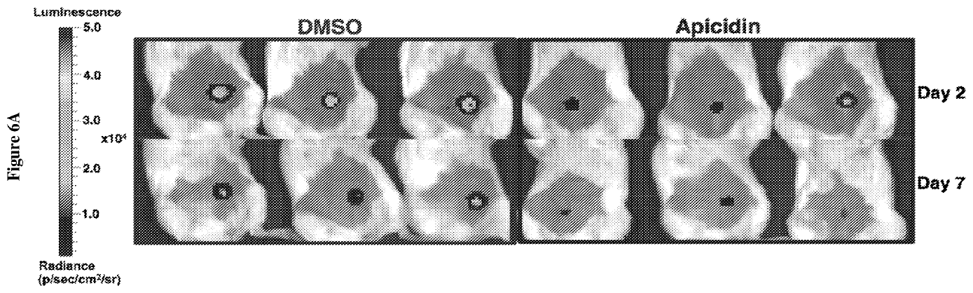

FIG. 5A are images of agrP3 reporter activity (bioluminescence) 3 hrs post infection.

FIG. 5B is a graph depicting kinetics of agr activation in apicidin and vehicle control treated mice after infection.

FIG. 5C is a graph depicting skin lesion size measurements (left) and representative images (right) at the indicated time points after infection. Error bars represent SEM. Post test P value (*)=<0.05, (**)=<0.01.

FIG. 5D is a graph depicting skin lesion size measurements (left) and representative images (right) at the indicated time points after infection. Error bars represent SEM. Post test p value (*)=<0.05, (**)=<0.01.

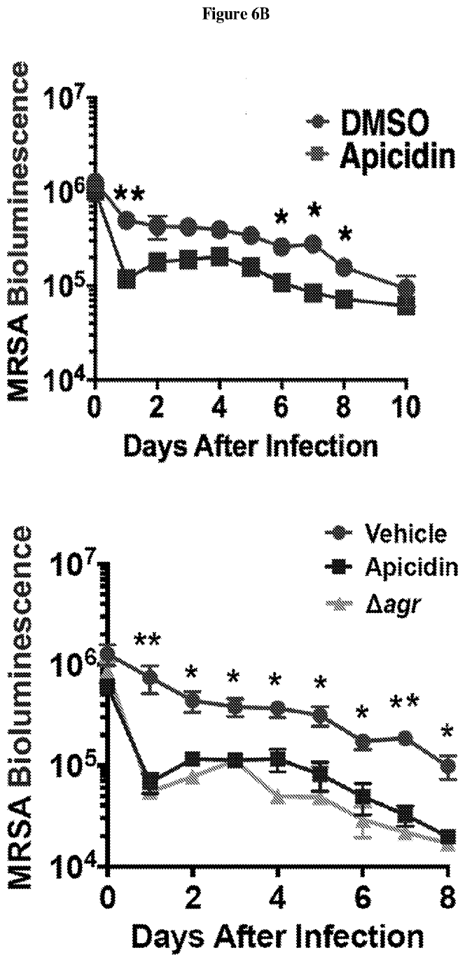

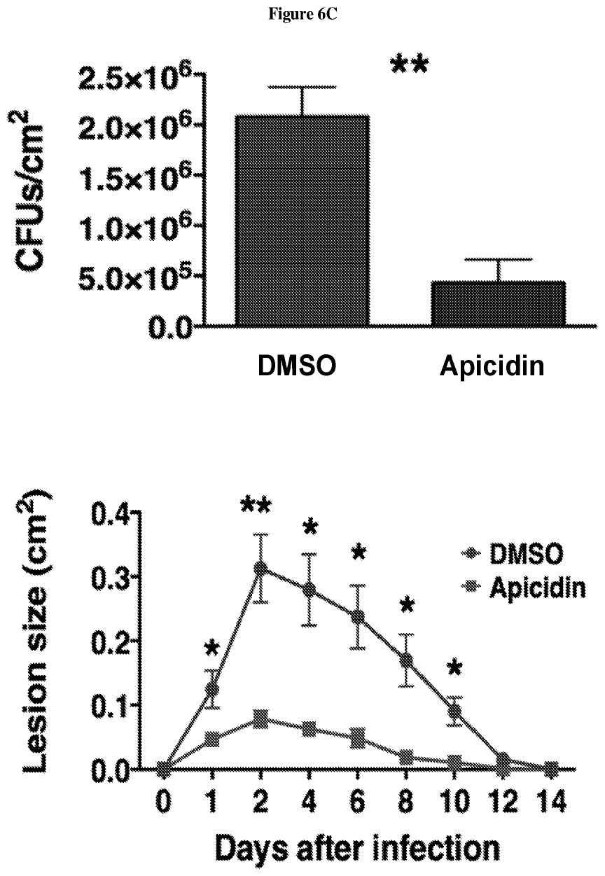

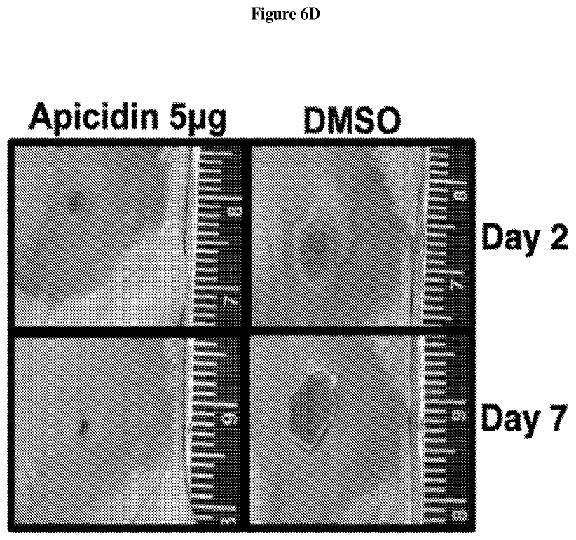

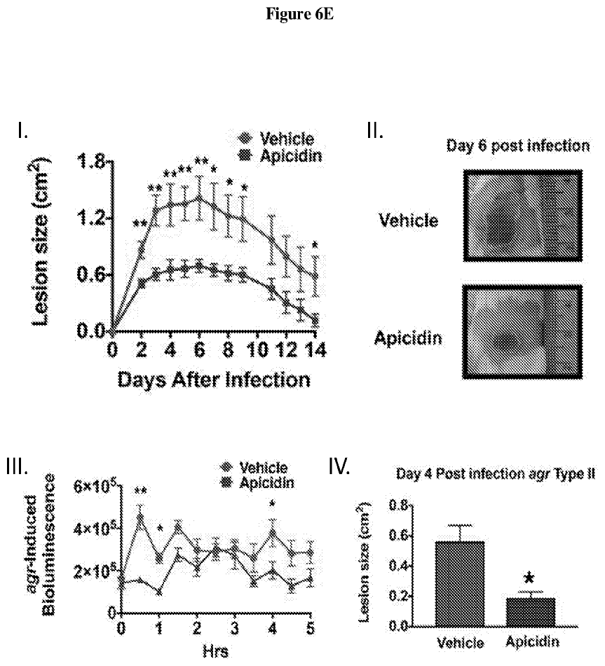

FIGS. 6A and 6B are images and a graph, respectively, of noninvasive, longitudinal measurements of MRSA bioluminescence following skin infection with Lux*MRSA.

FIG. 6C are graphs depicting CFUs recovered from BALB/c skin lesions 1 day after infection (left) and corresponding lesion size measurements (right). Error bars represent SEM. Post test P value (*)=<0.05, (**)=<0.01. FIG. 6D are corresponding representative images.

FIG. 6E includes (I.) a graph of skin lesion measurements following infection with an agr type II invasive MRSA isolate (Error bars represent SEM. Post test p value (*)=<0.05, (**)=<0.01.); (II.) representative images of tissue injury following infection with agr type II+/−apicidin; (III.) a graph of kinetics of agr activation in apicidin and vehicle control treated mice after infection (Error bars represent SEM. Post test p value (*)=<0.05, (**)=<0.01.); and (IV.) a graph of corresponding skin lesion size measurements at the indicated time point following infection (Error bars represent SEM. Post test p value (*)=<0.05.).

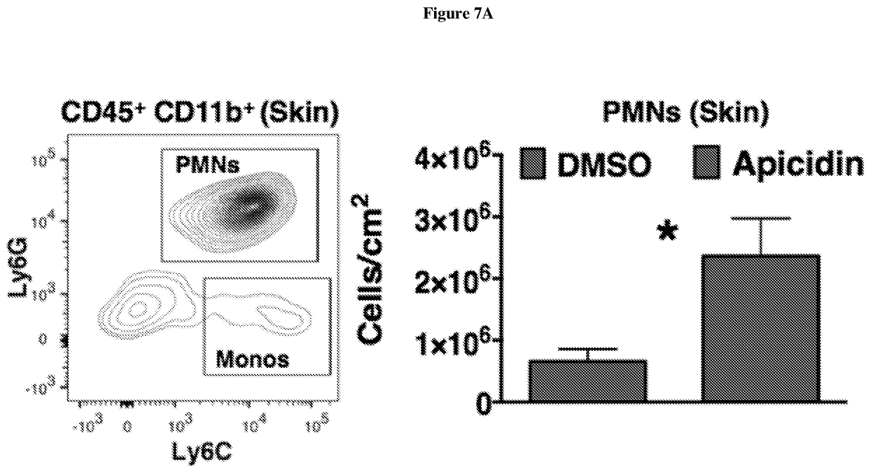

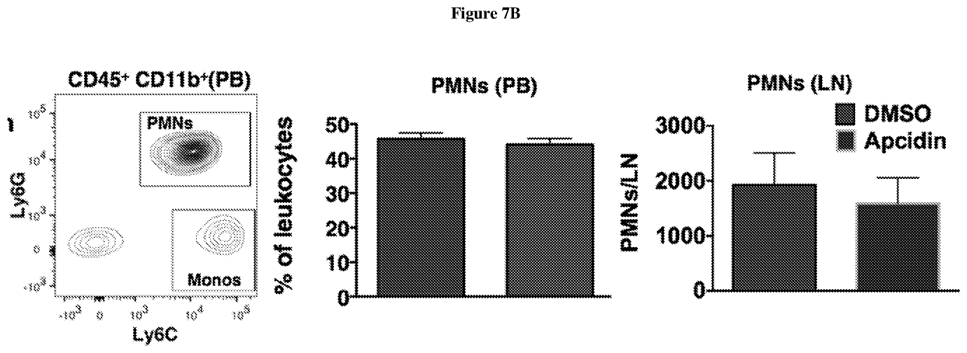

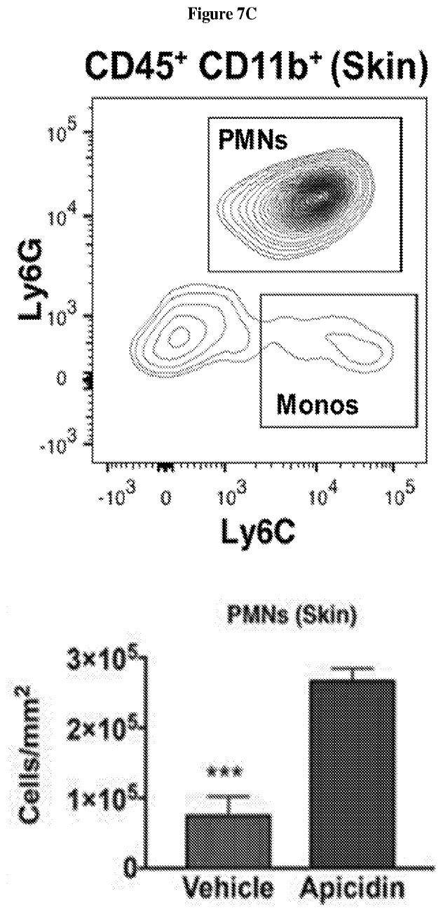

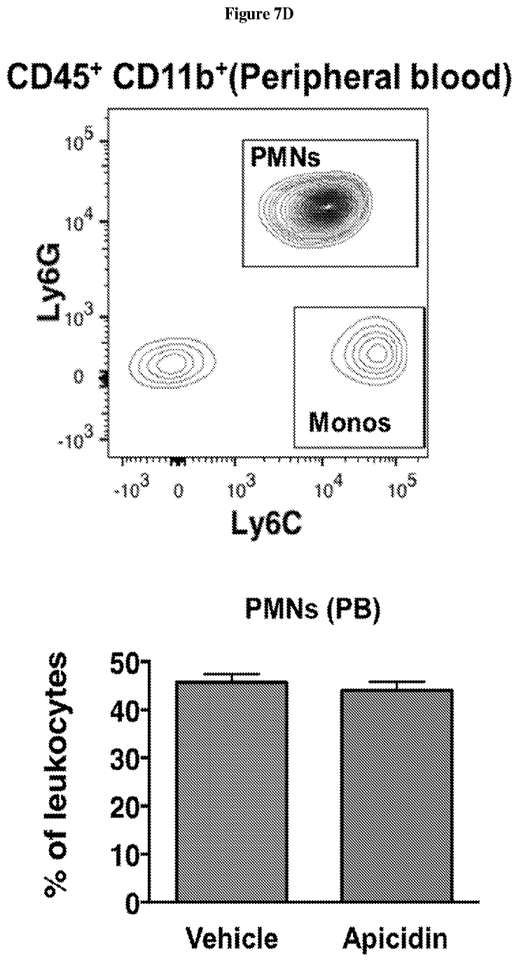

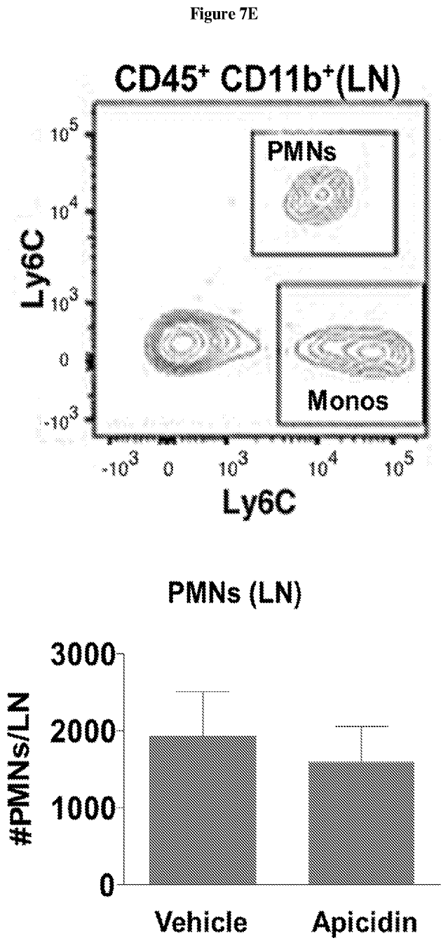

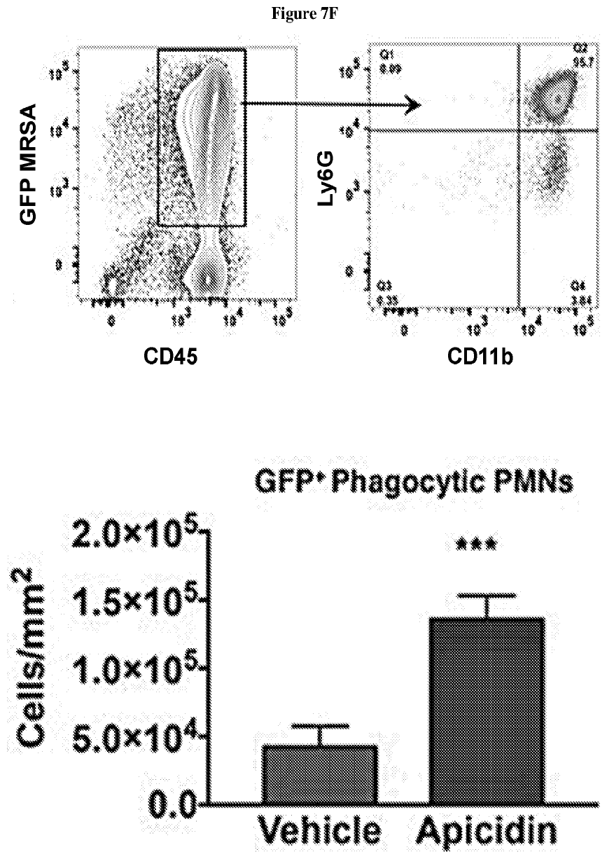

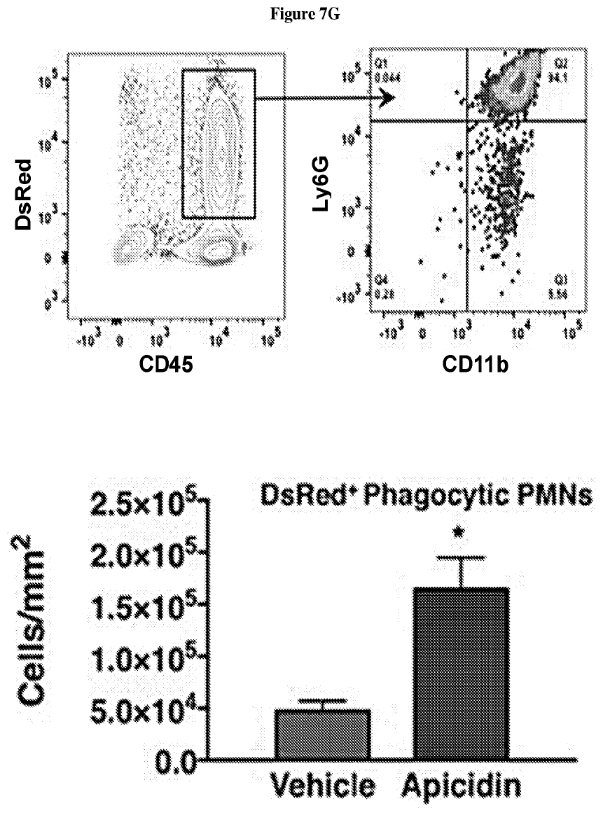

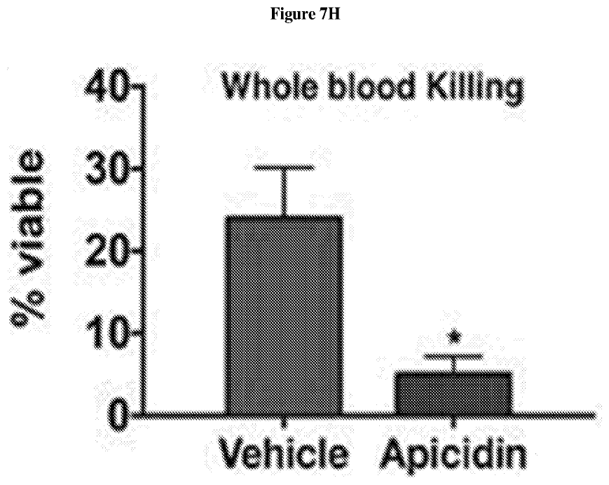

FIGS. 7A and 7B are graphs depicting PMN accumulation assessments by flow cytometry for skin, PB, and LN cell suspensions generated from LAC infected mice. Error bars represent SEM. Post test P value (*)=<0.05. Gating strategies are shown to the left of the bar graphs.

FIGS. 7C, 7D, and 7E. each depict gating strategies (Left) and PMN accumulation values (Right) from the indicated tissues one day after intradermal MRSA challenge (+/−apicidin). Error bars represent SEM. Post test p value (*)=<0.05. (***)=<0.005.

FIGS. 7F and 7G each depict gating strategies (Left) and enumerated phagocytic PMNs (Right) one day after intradermal MRSA challenge with GFP-MRSA (Top) or DsRed-MRSA (Bottom) Error bars represent SEM. Post test p value (*)=<0.05. (***)=<0.005.

FIG. 7H is a graph with data from a whole blood killing assay, following four hours of culture in the presence of 100 μm apicidin or vehicle control, heparinized human whole blood was inoculated with MRSA organisms cultured (4 hrs) in the presence of 100 μm apicidin or vehicle. After one hours CFUs from inoculated whole blood were plated out and compared with the starting inoculum to score for percent killing. Error bars represent SEM. Post test p value (*)=<0.05.

FIGS. 8A depicts gating strategies, and 8B are the corresponding graphs depicting phagocyte accumulation assessments by flow cytometry one day after infection for skin cell suspensions generated from mice infected with 2×107 CFUs LAC engineered for constitutive expression of red fluorescent protein+/−5 μg apicidin. Error bars represent SEM. Post test P value (*)=<0.05.

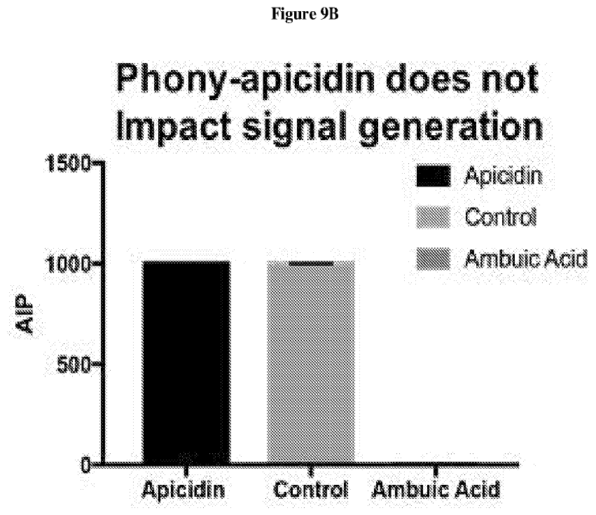

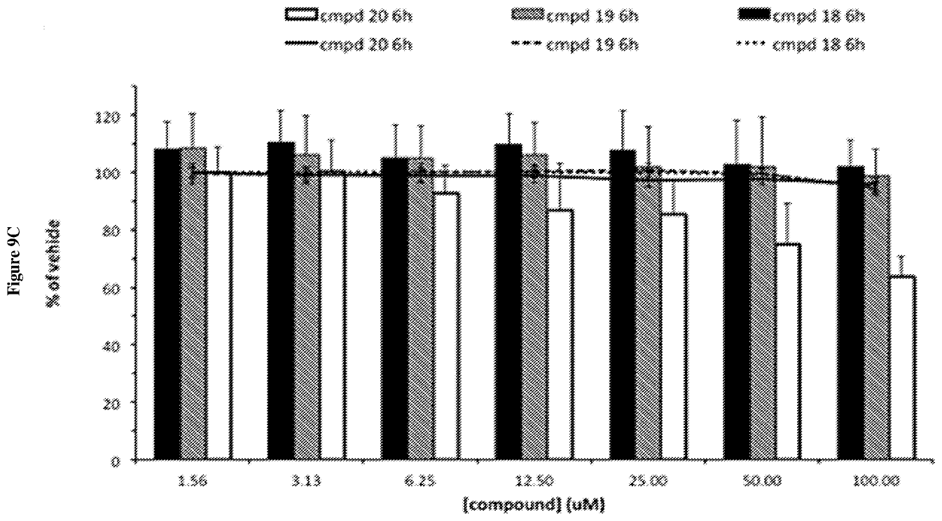

FIG. 9A is a graph of an assessment of apicidin mediated agr-inhibition using a constitutive AgrC mutant. FIG. 9B. is a graph of mass spectrometric measurements of AIP-I production by a USA300 MRSA isolate. FIG. 9C is a graph of effect of increasing concentrations of apicidin upon agrA reporter activation using an agr null strain expressing a plasmid for agr.

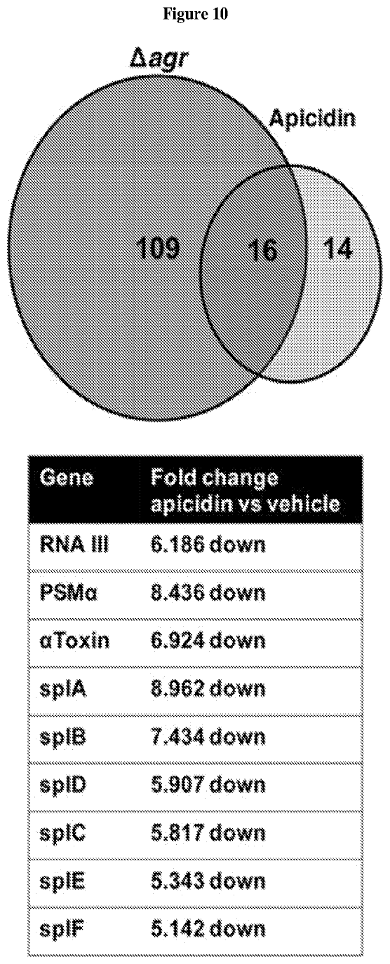

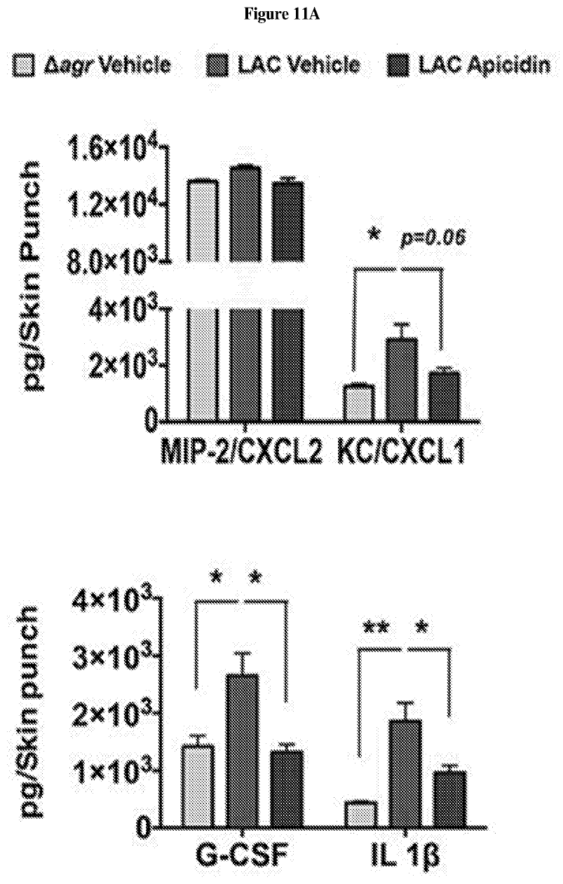

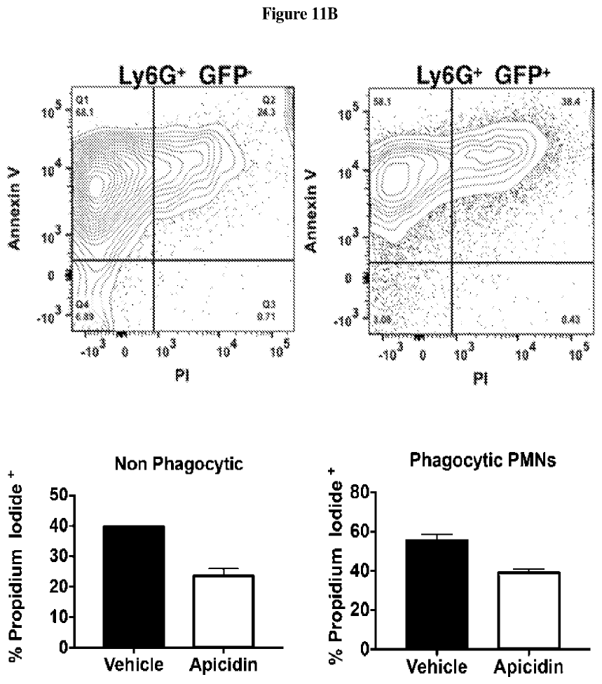

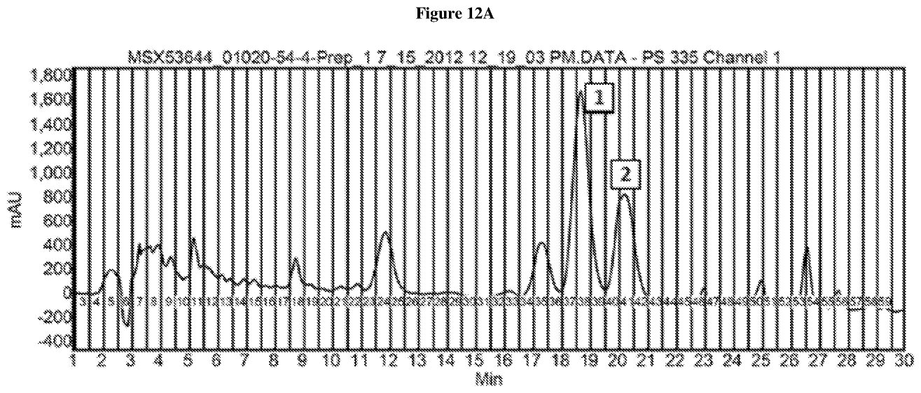

FIG. 10 is a Venn diagram using LAC+vehicle as a baseline and showing the number of genes surpassing the four-fold change threshold in Δagr and apicidin treated groups, as well as the number overlapping transcriptional targets (left); and a table listing MRSA virulence factors that are commonly repressed in Δagr and apicidin treated cells (right).

FIG. 11A are graphs of data from cytokine array analysis of supernatants from infected skin tissues collected one day after infection. FIG. 11B depicts flow cytometric analysis of apoptosis among phagocytic and non-phagocytic PMNs recovered from skin lesions of apicidin or vehicle treated animals one day after infection. Error bars represent SEM. Post test p value (*)=<0.05, (**)=<0.01.

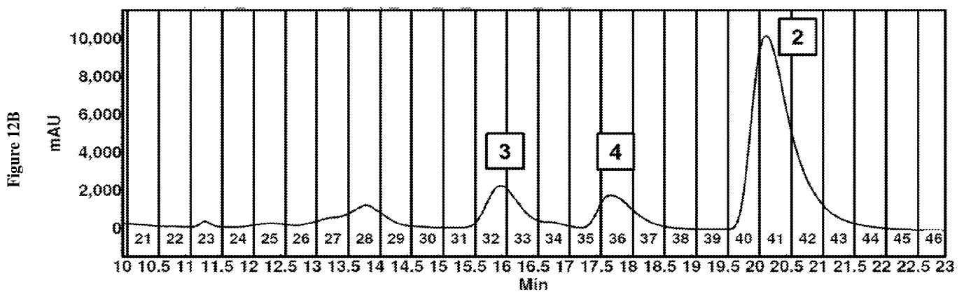

FIG. 12A is a representative prep-HPLC chromatogram of fraction #4 (MSX53644). FIG. 12B is a representative prep-HPLC chromatogram of fraction #5 (G134). Method: Gradient, MeCN: H₂O/0.1 formic acid, 40 to 70 over 30 min to 100, no hold, 21.24 mL/min, 254 nm. Column: Phenomaenex Gemini-NX, 5 μm, C18, 110A, AX. 250×21.20 mm.

FIG. 13A is a schematic of agr system; and FIG. 13B is a flow chart for isolation of apicidin from G134/G137 preparative chromatogram (left) and apicidin structures (right).

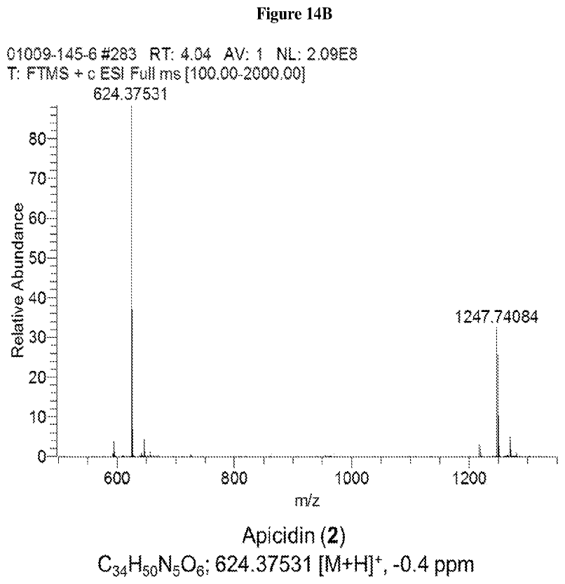

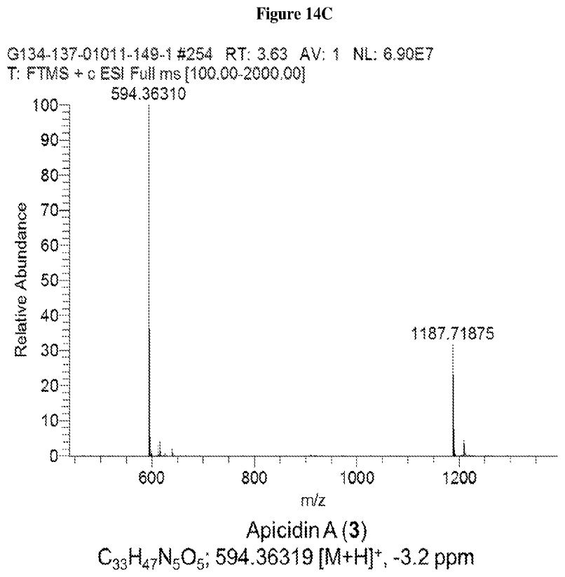

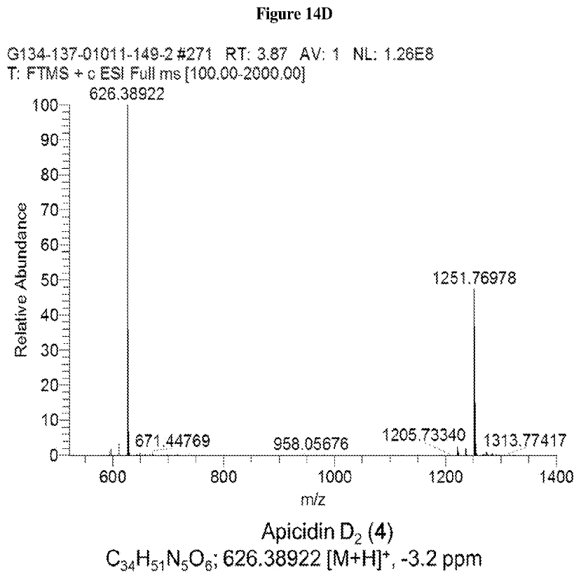

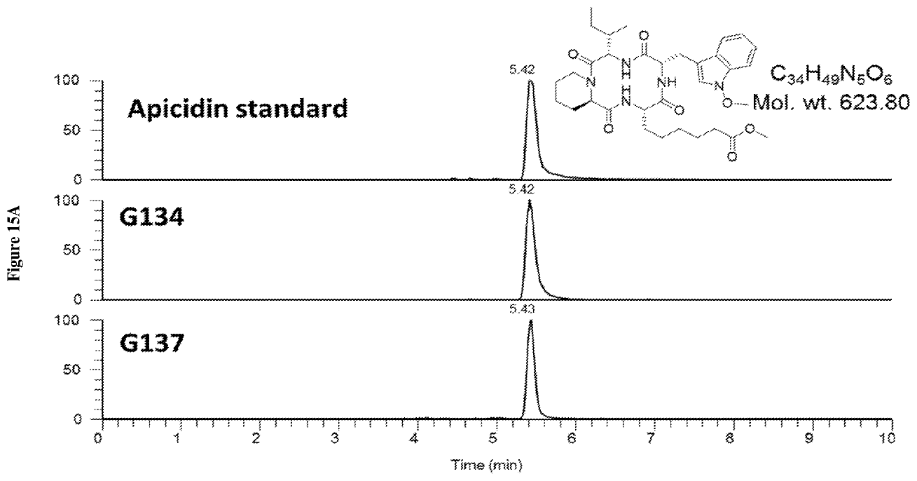

FIGS. 14A-D are (+)-HRESIMS spectra of Apicidin L, Apicidin, Apicidin A, and Apicidin D₂.

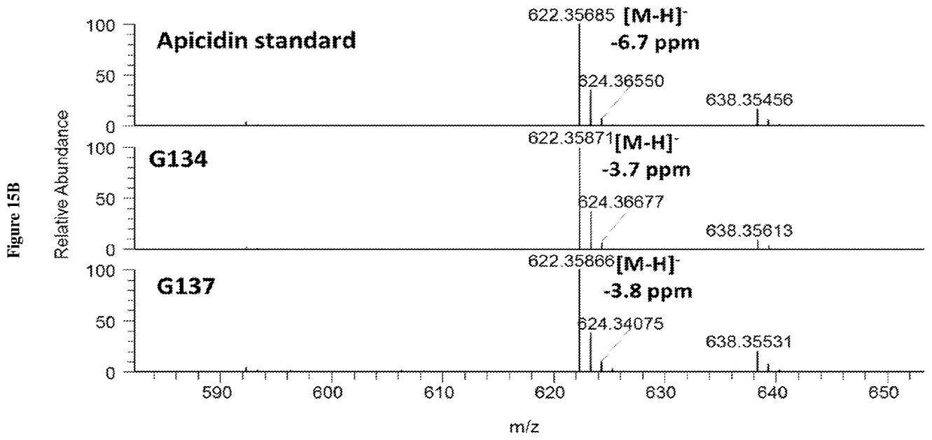

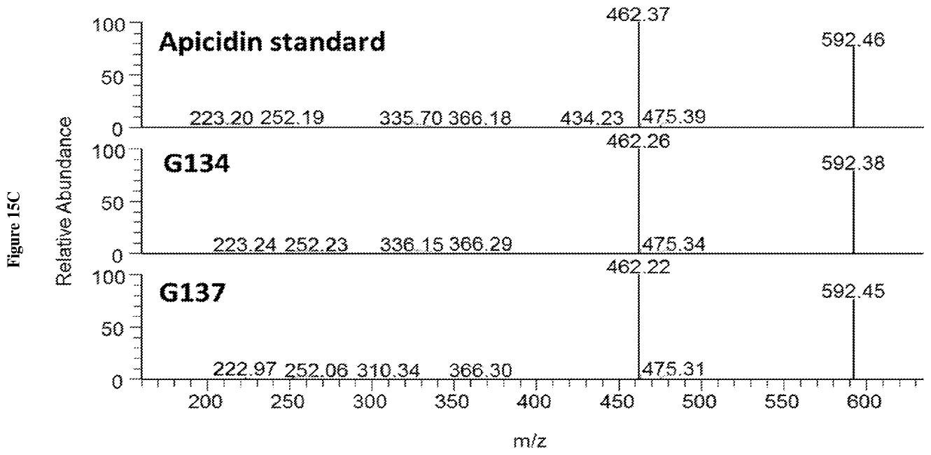

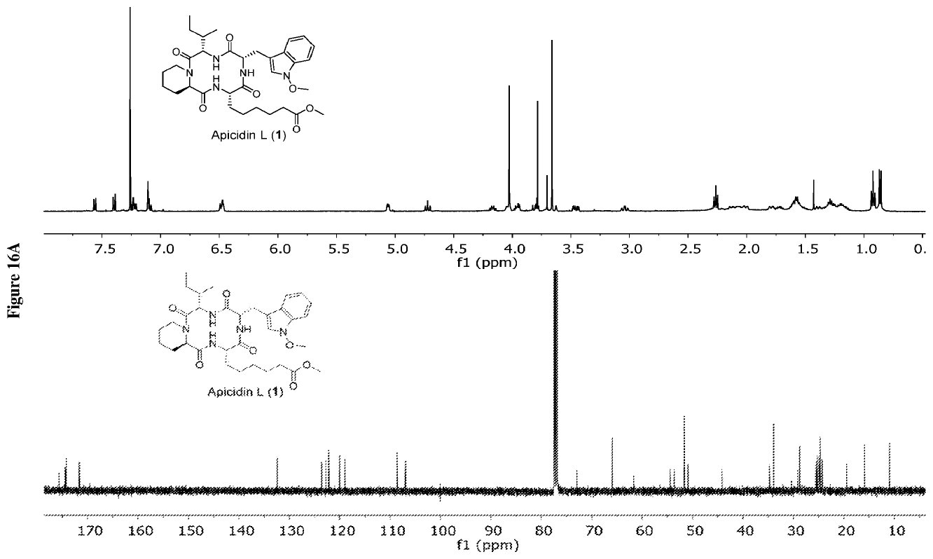

FIGS. 15A-C are (A) overlay of chromatographic peaks of apicidin, G134, and G137; (B) (−)-HRESIMS of apicidin; and (C) MS MS CID fragmentation spectra of apicidin.

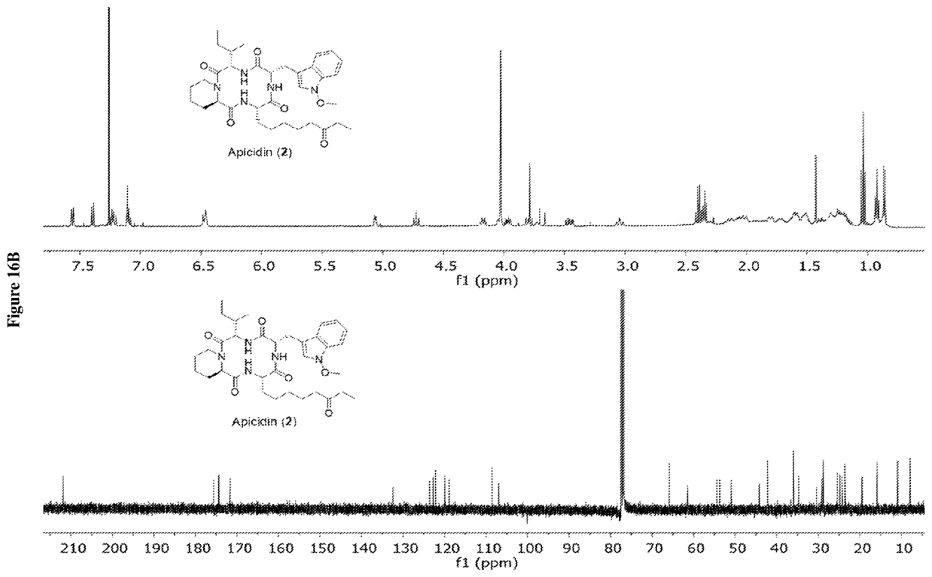

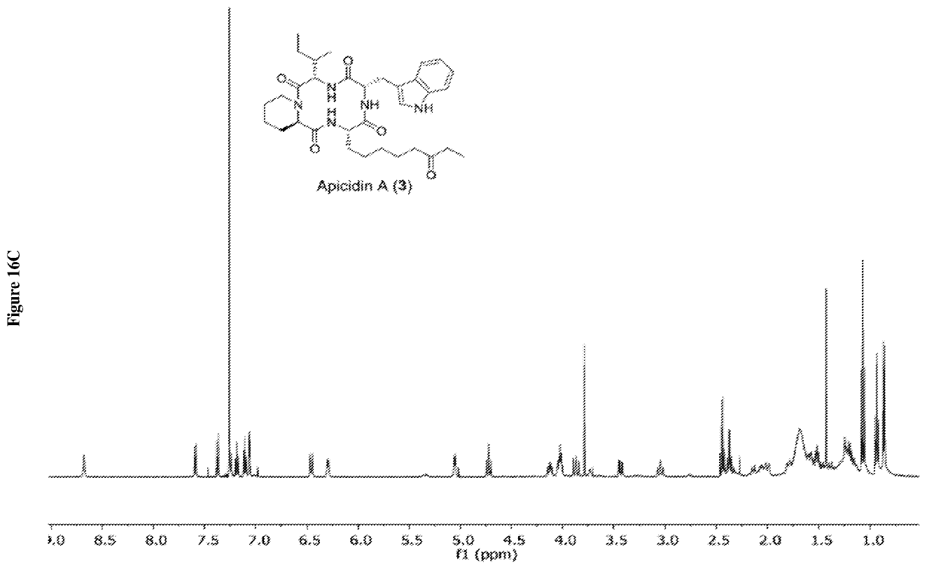

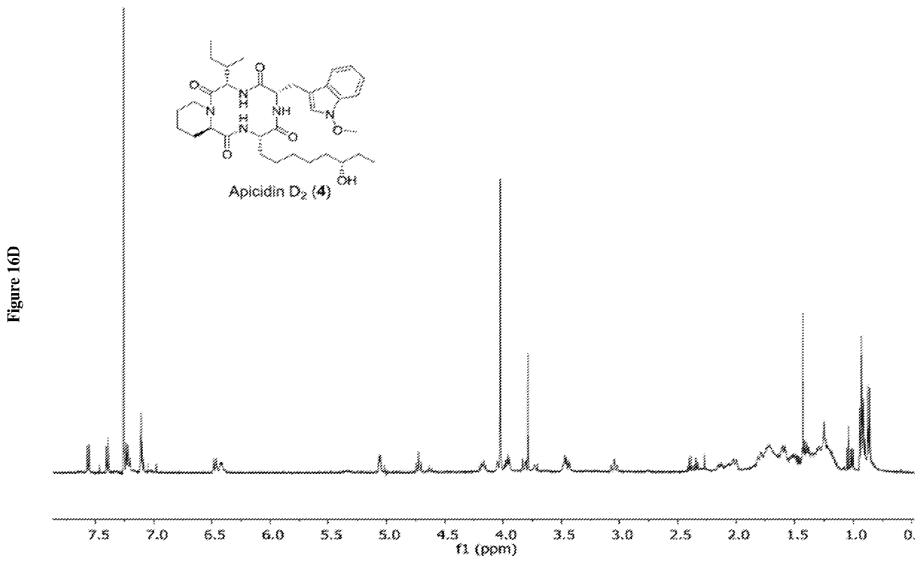

FIG. 16A is ¹H and ¹³C NMR spectra of compound 1 [500 MHz for 1H and 125 MHz for 13C, CDCl₃]. FIG. 16B is ¹H and ¹³C NMR spectra of compound 2 [500 MHz for ¹H and 125 MHz for ¹³C, CDCl₃]. FIG. 16C is ¹H NMR spectrum of compound 3 [500 MHz, CDCl3]. FIG. 16D is ¹H NMR spectrum of compound 4 [500 MHz, CDCl3].

The results conveyed in the figures above demonstrate, through agr-reporter-based screens, that a class of compounds, called apicidins inhibit quorum-sensing activity across MRSA isolates.

In particular, to test the efficacy of apicidin in vivo, mice were challenged intradermally with 2×107 MRSA (+/−) 5 μg apicidin. The abatement MRSA virulence in the apicidin-treated group was demonstrated by reduced: weight loss, dermonecrosis and cutaneous bacterial burden. By challenging mice with an agr-reporter strain, it was demonstrated that the apicidin-mediated attenuation of MRSA pathogenesis corresponded with reduced quorum sensing activity in vivo.

Moreover, to evaluate apicidin's impact upon anti-MRSA effector responses, polymorphonuclear neutrophil (PMN) accumulation and function at cutaneous sites of infection was assessed. Flow cytometric analysis revealed that apicidin increased the density of PMNs within infected wounds 24 hours post infection. In addition, the number of PMNs that phagocytosed MRSA organisms in vivo increased in lesional skin preparations from apicidin treated mice.

Further description of the results follows:

Isolation of Apicidin and Related Analogues from MSX53644 and G134/G137

The organic extract of a fungal culture of strain MSX53644 that was grown over rice was subjected to purification using normal phase silica gel flash chromatography to yield five fractions. The fourth fraction showed potent cytotoxic activity, and hence was further purified using reversed-phase preparative HPLC (FIG. S1A, Supporting information) to yield two cyclic tetrapeptides that were identified using HRESIMS and NMR as the known apicidin (2) and a new natural product analogue to apicidin to which the trivial name apicidin L (1) was assigned (FIG. 13, FIGS. 14A-D, 16A, and 16B). The compounds were added to our in-house library of fungal seconadry metabolites for dereplication studies.

The aim of the project was to study the chemical mycology of endophytic fungi of a number of medicinal plants, including milk thistle, goldenseal, pawpaw, and herba mensa. A total of thirteen endophytic fungal strains (eleven unique genotypes) were identified from the roots of yerba mansa [Anemopsis californica (Nutt.) Hook. & Arn. (Saururaceae)]. Yerba mansa harbors diverse endophyte populations, which yield antimicrobial compounds under the proper cultivation conditions (43). Crude extracts prepared from Yerba mansa have been used as a folk remedy to treat skin infections. Here, the extracts prepared from these endophytic fungi were dereplicated using our in-house LC-MS dereplication protocol, where the organic extracts of two fungal isolates, both identified as Fusarium sp. (strains G134 and G137), showed the presence of apicidin by matching retention time, HRESIMS, and MS/MS data (FIGS. 15A-C). Considering our interest in apicidin and in an attempt to isolate more structurally related analogues, the organic extracts of G134 and G137 were combined and purified using silica gel flash chromatography to yield five fractions. Fraction five was found to contain apicidin as evidenced by LC-MS and hence was further purified using reversed-phase preparative HPLC to yield apicidin (2) and two structurally related known apicidin analogues, apicidin A (3) and apicidin D2 (4) (FIG. 13).

Apicidin Broadly Inhibits Agr Systems of S. aureus.

Evolutionary divergence at the agr locus is evidenced by the four distinct allelic variants that have been identified among S. aureus isolates (27). Although, agr-I alleles, which encompass USA300 isolates, are the most pervasive and problematic clinically, all four agr-types represent a source of human disease. Thus, the applicability of any quorum quenching-based therapy is extended by its capacity to inhibit multiple agr-types. Well-established agr P3 reporter based assays (9, 40-41) were used and showed that apicidin and its three analogues inhibited the quorum sensing responses of multiple agr types (FIG. 1C). Further corroboration of the apicidins' broad quorum quenching effects was achieved in parallel experiments showing a similar inhibition of red blood cell (RBC) lysis assays. Relative to its analogues, apicidin (1) showed the most impressive actions as an agr inhibitor and therefore became the focus of subsequent efforts to further certify its capacity to function as a nontoxic, pan inhibitor of agr activation. To this end, the fluorescent reporter strains derived from all four agr-types were employed in time course experiments that carefully compared dose effects of apicidin (1) upon agr P3 driven YFP expression and bacterial growth (FIG. 1C) (9, 15). Using this approach, the quorum quenching potency of apicidin was found to be agr-type dependent, with types II and IV representing the most sensitive and resistant respectively (FIG. 1C). This relation between apicidin's quorum quenching effectiveness and agr-type was further affirmed in RBC lysis assays. It should be noted however, that in all cases, apicidin-mediated agr-inhibition achieved IC50 values at μM concentrations that were sub-inhibitory for growth. Finally, the capacity of apicidin to inhibit the agr systems of other Gram-positive staphylococci was assessed. agr-P3 reporter strains derived from Staphylococcus epidermidis (S. epidermidis) isolates of each agr-type were used to show that the quorum quenching activity of apicidin extend to multiple staphylococcal species (FIGS. 14 A-D) (35).

Mechanism of Apicidin Mediated Quorum Quenching

To investigate the mechanisms by which apicidin inhibits quorum sensing, a series of in vitro assays were conducted to systematically assess the relative contribution of each of the core genetic elements of the agrBDCA operon (FIG. 13A). The membrane embedded peptidase AgrB, is responsible for processing the AgrD pro-peptide (immature AIP) for extracellular release. To determine if apicidin inhibits agr signaling at the level of AIP signal generation, an established system was used whereby the agrBD genes (under sarA P1 promoter regulation), were integrated into an agr null strain derived from the USA300 isolate LAC (40). This construct enables alterations to AIP signal biosynthesis to be clearly delineated from regulation stemming from signal integration. Mass spectrometric measurements of AIP production in apicidin treated and vehicle control cultures showed that apicidin-mediated quorum quenching is achieved through a mechanism that does not target AIP signal biosynthesis (FIG. 9A). Next, the possibility that apicidin interferes with the sensory activity of AIP receptor AgrC was explored. For this, a R235H construct bearing a point mutation that confers constitutive AgrC activity, and thereby enables phospho-activation of AgrA to occur independently of AIP binding, was employed. In RBC lysis assays using this strain, apicidin imposed a dose dependent inhibition of lytic activity that was highly reminiscent of previous assays using WT cultures (FIG. 9B). Given that the results from the constitutive AgrC mutant strain suggest that apicidin mediates its effects downstream of AgrA phosphorylation. Next, it was determined if AgrA serves as the target of apicidin. To this end, an agr null reporter construct, whereby agrP3 lux activation can only be achieved through the constitutive production of plasmid encoded AgrA (ref), was employed. The dose dependent inhibition of agr-P3 driven bioluminescence shows that apicidin specifically interferes with AgrA-dependent quorum sensing activation (FIG. 9C). Together, these mechanistic studies provide evidence that AgrA serves as the molecular target of apicidin.

Apicidin Targets Agr-Dependent Transcriptional Regulation of Signature Virulence Factors

To evaluate the impact of apicidin upon global transcriptional regulation RNA-seq analysis upon WT LAC cultures grown in the presence of apicidin or vehicle was conducted. For purposes of comparing the effects of apicidin upon the MRSA transcriptome with those resulting from agr deletion, parallel RNA seq-analysis of WT and Δagr preparations were performed. A four-fold cutoff as the threshold for differential gene expression was used, and apicidin altered the expression of thirty transcripts, with >50% of these representing prototypical agr targets such hla, RNAIII, psms, and spl genes (FIG. 10). Altogether, these data show that apicidin has a transcriptional signature that is largely confined to agr-regulated transcripts.

Apicidin Abates MRSA Pathogenesis

The potent in vitro activity of apicidin encouraged an assessment of its efficacy in vivo. To this end, an intradermal challenge model was employed by delivering a single 5 μg dose of apicidin along as part of the inoculum suspension containing 2×107 MRSA organisms. Measurement of the resultant skin ulcerations over the course of 14 days, showed that apicidin significantly reduced MRSA-induced dermatopathology in both C57BL/6 and BALB/c mice. In addition to attenuated cutaneous injury, apicidin treated animals experienced a more severe and protracted period of weight loss in both strains (FIG. 4C). Together these data show that when applied as an anti-infective, apicidin impressively attenuates clinical disease following MRSA challenge.

Apicidin Mediates Quorum Quenching at In Vivo Sites of Infection

Having demonstrated that apicidin attenuated important indicia of ineffective illness (e.g., tissue damage, weight loss, bacterial burden), it was next to be determined if these therapeutic effects corresponded with quorum sensing interference in vivo. To this end, mice with an agr-P3 lux reporter strain were challenged and carefully monitored the kinetics of agr activation over a six-hour period immediately following infection. Here, attenuation of MRSA virulence in apicidin treated animals occurred alongside a significant interference of agr activation in vivo (FIGS. 5A-D). To further demonstrate that the level of agr interference mediated by apicidin corresponded with a hypo-virulent infectious phenotype, the ensuing skin ulcers in these animals over 14-day period were measured and a dramatic reduction in dermatopathology in apicidin treated animals was found. Altogether, these data demonstrate that the apicidin-mediated attenuation of MRSA pathogenesis corresponds with quorum quenching activity both in vitro and in vivo.

Apicidin Treatment Improves Bacterial Clearance Following MRSA Skin Challenge.

To determine if the apicidin mediated attenuation of MRSA pathogenesis occurred alongside a decrease in cutaneous bacterial burden, challenge experiments were conducted with a Lux+ MRSA strain. An advantage of this approach is the non-invasive and longitudinal manner with which bacterial burden can be measured. While an apicidin-induced decrease in bacterial burden was observed throughout the experiment, this effect was most impressively evident one day following infection (FIG. 6). Corroborating the apicidin mediated enhancement of bacterial clearance via IVIS imaging, significantly fewer CFUs were recovered from lesional skin one day after infection in apicidin treated mice relative to controls. In addition, parallel analysis of lesion development in the same apicidin and control mice showed a correspondence between MRSA driven bioluminescence and dermatopathology.

Apicidin Treatment Enhances PMN Responses Following MRSA Skin Challenge.

The recruitment of PMNs to sites of cutaneous S. aureus infection is critical for pathogen clearance (41, 42). Abundant in the circulation, PMNs orchestrate protective anti-S. aureus cutaneous immune responses by extravasating proximal to the focus of infection where they accumulate, phagocytose and ultimately clear S. aureus organisms (41). The present study demonstrates that single administration of apicidin at the time of challenge, alters the PMN dynamics at MRSA infected loci by augmenting their accumulation (FIG. 6E). It was important to address whether the PMNs present within apicidin-exposed infectious environments are functionally altered. For this purpose, mice were challenged with DsRed+ MRSA and apicidin's impact upon MRSA uptake was measured by flow cytometry. Though not significantly, a trend showing increased frequency of PMN able to phagocytose MRSA in preparations from apicidin treated mice (FIG. 6E). Given that the amassment of PMNs was greater in apicidin treated animals, there was overall enhancement MRSA uptake within the entire network of cutaneous PMNs. Importantly, these findings show that the increased MRSA clearance in apicidin treated mice corresponds with enhanced PMN effector responses.

Apicidin Mediated Enhancement of PMN Responses Corresponds with Inflammation and Apoptosis at Cutaneous Sites of Infection.

To explore mechanistic basis of the increased PMN density at cutaneous sites of infection, the cytokine milieu of lesional tissue one day after infection was assessed. Interestingly, the apicidin-induced increase in cutaneous PMNs did not correspond with a commensurate increase in prototypical chemoattractant or granulopoiesis factors (FIGS. 7C-H). In fact, preparations from apicidin treated animals showed a decrease in the production of CXCL-2 and G-CSF and relative to controls suggesting that apicidin enhanced PMN responses through alternative mechanisms. Chief among these is the possibility that the agr interference occurring in vivo inhibits the secretion of cytolytic enzymes thereby favors prolonged PMN survival. To explore the contribution of enhanced PMN persistence within the infectious environment, the frequency of PMNs in late stage apoptosis/necrosis within lesional skin preparations was assessed. There was decrease in the frequency of apoptotic PMNs (FIGS. 7C-H), which is consistent with our previous observation that agr-regulated virulence actor suppression represents a major repercussion of apicidin treatment

To assess apicidin's impact upon signal integration, an agr-P3 reporter strain endowed with constitutive AgrC activity was used. In this construct, the phosphor-activation and subsequent DNA binding activity of AgrA occurs in manner that is impervious to any perturbation upon AgrC's function as an AIP sensor/receptor. Therefore, the maintenance of potent quorum quenching activity against this strain suggests that apicidin orchestrates agr interference through the abatement of AgrA mediated signal transduction. In support of this, apicidin inhibited agr-P3 reporter activation in an engineered system where AgrA is solely responsible for P3 activation (20). Working from the premise that AgrC mediated phosphor-transfer is a prerequisite for AgrA-mediated P3 activation, the reporter activity should be unachievable in a AgrC deficient construct. The conundrum of reporter P3 activity in the constitutive AgrA strain is likely due to the ability of unphosphorylated AgrA engage the P3 promoter albeit at much lower affinity. It is likely that with even in without upstream regulation from other components of the agr system, AgrA overexpression permits reporter signal activation and thus the delineation of the apicidin/AgrA interactions. Altogether, these data indicate that apicidin-mediated quorum quenching does not alter the release of AIP signals nor their interaction with cognate receptors but rather interferes the activity of the response regulator AgrA.

Apicidin-mediates quorum sensing inhibition across S. epidermidis agr types. S. epidermidis is a well-recognized opportunistic pathogen. S. epidermidis is dominant commensal that has been shown to play an integral role in the induction and maintenance of key cellular components of the cutaneous immune system (44). Furthermore, S. epidermidis produces AIPs that are cross-inhibitory to multiple agr types and thus within the context of S. aureus infection, apicidin's inhibition of S. epidermidis-quorum sensing may in effect, suppress the direct and indirect contributions of S. epidermidis to host defense and therefore benefit the more dangerous pathogen. To begin to address these questions, potency of the efficacy of apicidin the context of cutaneous challenge was explored.

Using an experimental framework that assesses key host defense circuits, apicidin-mediated quorum quenching is shown to attenuate disease and significantly enhance effector responses. Apicidin reduced cutaneous bacterial burden following infection, which indicates that host innate effector mechanisms underlying bacterial clearance are potentiated as consequence of quorum sensing inhibition. The increased bacterial clearance was found to occur alongside an augmentation of the following parameters of anti-MRSA host defense: enhanced PMN survival, increased PMN density, and an overall gain in the phagocytic capability of the entire cutaneous PMNs network. When viewed together with the evidence of apicidin-mediated quorum sensing in vivo, these results are consistent with the interpretation that by suppression the agr-regulated virulon, which in turn, induces the secretion of virulence factors that are noxious to host immune cells (e.g., PSMs and α-toxin) the host's anti-MRSA effector mechanisms can be more effectively and efficiently executed, the manifestations of which are attenuated tissue damage and accelerated bacterial clearance.

Specificity and selectivity of the quorum quenching compounds against agr-systems are addressed with evidence of apicidin-mediated quorum quenching during the course of infection in vivo. Assessment of apicidin as an anti-infective demonstrated the proof of concept that by quenching S. aureus quorum sensing, it can also inhibit the severity of necrosis at the infection site. Based on previous work by Wright et al, which showed that the extent of S. aureus-induced dermatopathology is proportional to the magnitude of agr activation during the first four hours of infection, this key time period was selected for rigorous tracking of apicidin's effects upon quorum sensing in vivo (30). Results here show the high level of agr interference resulting from a single dose of apicidin corresponds with a marked attenuation of MRSA's infectious phenotype following cutaneous challenge. Altogether, these results are the first to unequivocally show small molecule interference of the agr-system within the context of in vivo infection and furthermore show that this magnitude of this interference within the most proximal hours of challenge corresponds with a markedly attenuated infectious phenotype.

Apicidin, a natural product isolated from both terrestrial and endophytic fungi, disables S. aureus quorum sensing and the resultant suppression of immune-teratogenic virulence factors indirectly potentiates key immune effector responses that mediate pathogen clearance. The antivirulence provides a means of disarming bacterial virulence mechanisms while simultaneously potentiating host defense.

The above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

### 7. REFERENCES

1. Newman D J, Cragg G M. 2012. Natural Products As Sources of New Drugs over the 30 Years from 1981 to 2010. J. Nat. Prod. 75:311-335.
2. LuoM, QiuJ, ZhangY, WangJ, DongJ, LiH, LengB, ZhangQ, DaiX, NiuX, ZhaoS, Deng X. 2012. alpha-cyperone alleviates lung cell injury caused by Staphylococcus aureus via attenuation of alpha-hemolysin expression. J. Microbiol. Biotechnol. 22:1170-1176.
3. Nakayama J, Uemura Y, Nishiguchi K, Yoshimura N, Igarashi Y, Sonomoto K. 2009. Ambuic acid inhibits the biosynthesis of cyclic peptide quormones in gram-positive bacteria. Antimicrob. Agents Chemother. 53:580-586.
4. Cech N B, Junio H A, Ackermann L W, Kavanaugh J S, Horswill A R. 2012. Quorum quenching and antimicrobial activity of goldenseal (Hydrastis canadensis) against methicillin-resistant Staphylococcus aureus (MRSA). Planta Med. 78:1556-1561.
5. Quave C L, Plano L R, Bennett B C. 2011. Quorum sensing inhibitors of Staphylococcus aureus from Italian medicinal plants. Planta Med. 77:188-195.
6. Cheung G Y, Wang R, Khan B A, Sturdevant D E, Otto M. 2011. Role of the accessory gene regulator agr in community-associated methicillin-resistant Staphylococcus aureus pathogenesis. Infect. Immun. 79:1927-1935.
7. Montgomery C P, Boyle-Vavra S, Daum R S. 2010. Importance of the global regulators Agr and SaeRS in the pathogenesis of CA-MRSA USA300 infection. PLoS One. 5:e15177.
8. Rothfork J M, Timmins G S, Harris M N, Chen X, Lusis A J, Otto M, Cheung A L, Gresham H D. 2004. Inactivation of a bacterial virulence pheromone by phagocyte-derived oxidants: new role for the NADPH oxidase in host defense. Proc. Natl. Acad. Sci. U.S.A. 101:13867-13872.
9. Hall P R, Elmore B O, Spang C H, Alexander S M, Manifold-Wheeler B C, Castleman M J, Daly S M, Peterson M M, Sully E K, Fending J K, Otto M, Horswill A R, Timmins G S, Gresham H D. 2013. Nox2 Modification of LDL Is Essential for Optimal Apolipoprotein B-mediated Control of agr Type III Staphylococcus aureus Quorum-sensing. PLoS Pathog. 9.
10. Nishimura H, Mayama M, Komatsu Y, Kato H, Shimaoka N, Tanaka Y. 1964. Showdomycin, a New Antibiotic from a Streptomyces Sp. J. Antibiot. (Tokyo). 17:148-155.
11. Schlievert P M, Case L C, Nemeth K A, Davis C C, Sun Y, Qin W, Wang F, Brosnahan A J, Mleziva J A, Peterson M L, Jones B E. 2007. Alpha and beta chains of hemoglobin inhibit production of Staphylococcus aureus exotoxins. Biochemistry. 46:14349-14358.
12. Park J, Jagasia R, Kaufmann G F, Mathison J C, Ruiz D I, Moss J A, Meijler M M, Ulevitch R J, Janda K D. 2007. Infection control by antibody disruption of bacterial quorum sensing signaling. Chem. Biol. 14:1119-1127.
13. Jacobsson G, Gustafsson E, Andersson R. 2008. Outcome for invasive Staphylococcus aureus infections. Eur. J. Clin. Microbiol. Infect. Dis. 27:839-848.
14. Jarraud S, Mougel C, Thioulouse J, Lina G, Meugnier H, Forey F, Nesme X, Etienne J, Vandenesch F. 2002. Relationships between Staphylococcus aureus genetic background, virulence factors, agr groups (Alleles), and human disease. Infect. Immun. 70:631-641.
15. Laxminarayan R, et al. (2013) Antibiotic resistance—the need for global solutions. Lancet Infect Dis. 13(12).
16. Medina E & Pieper D H (2016) Tackling Threats and Future Problems of Multidrug-Resistant Bacteria. Curr Top Microbiol Immunol. [Epub ahead of print].
17. CDC (2013) Antibiotic resistance threats in the United States. Atlanta Ga.::Centers for Disease Control listed] Na (2016) Jim O'Neill. Nat Rev Drug Discov 15(8):526-526.
18. Spellberg B, Srinivasan A, & Chambers H F (2016) New Societal Approaches to Empowering Antibiotic Stewardship. JAMA. 315(12):1229-1230.
19. Disease NIoAaI (2014) NIAD's Antibacterial Resistance Program: Current Status and Future Directions.1-13.
20. Cegelski L, Marshall G R, Eldridge G R, & Hultgren S J (2008) The biology and future prospects of antivirulence therapies. Nat Rev Microbiol. 6(1):17-27.
21. Rasko D A & Sperandio V (2010) Anti-virulence strategies to combat bacteria-mediated disease. Nat Rev Drug Discov. 9(2):117-118.
22. Lowy F D (1998) Staphylococcus aureus infections. N Engl J Med. 339(8):520-532.
23. Chambers H F & Deleo F R (2009) Waves of resistance: Staphylococcus aureus in the antibiotic era. Nat Rev Microbiol. 7(9):629-641.
24. Otto M (2010) Basis of virulence in community-associated methicillin-resistant Staphylococcus aureus. Annu Rev Microbiol. 2010(64):143-162.
25. DeLeo F R, Otto M, Kreiswirth B N, & Chambers H F (2010) Community-associated meticillin-resistant Staphylococcus aureus. Lancet. 375(9725):1557-1568.
26. Novick R P, et al. (1993) Synthesis of staphylococcal virulence factors is controlled by a regulatory RNA molecule. EMBO J. 12(10):3967-3975.
27. Novick R P & Geisinger E (2008) Quorum sensing in staphylococci. Annu Rev Genet. 2008(42):541-564.
28. Kavanaugh J S & Horswill A R (2016) Impact of Environmental Cues on Staphylococcal Quorum Sensing and Biofilm Development. J Biol Chem. 291(24):12556-12564.
29. Mayville P, et al. (1999) Structure-activity analysis of synthetic autoinducing thiolactone peptides from Staphylococcus aureus responsible for virulence. Proc Natl Acad Sci USA. 96(4):1218-1223.
30. Wright J S, Jin R, & Novick R P (2005) Transient interference with staphylococcal quorum sensing blocks abscess formation. Proc Natl Acad Sci USA. 102(5):1691-1696.
31. Nielsen A, et al. (2014) Solonamide B Inhibits Quorum Sensing and Reduces Staphylococcus aureus Mediated Killing of Human Neutrophils. PLoS One. 9(1):e84992. doi: 84910.81371/journal.pone.0084992.
32. Sully E K, et al. (2014) Selective chemical inhibition of agr quorum sensing in Staphylococcus aureus promotes host defense with minimal impact on resistance. PLoS Pathog. 10(6):e1004174. doi: 1004110.1001371/journal.ppat.1004174. eCollection 1002014.
33. Quave C L, et al. (2015) Castanea sativa (European Chestnut) Leaf Extracts Rich in Ursene and Oleanene Derivatives Block Staphylococcus aureus Virulence and Pathogenesis without Detectable Resistance. PLoS One. 10((8))::e0136486. doi: 0136410.0131371/journal.pone.0136486.
34. Figueroa M, et al. (2014) Polyhydroxyanthraquinones as quorum sensing inhibitors from the guttates of Penicillium restrictum and their analysis by desorption electrospray ionization mass spectrometry. J Nat Prod. 77(6):1351-1358.
35. Daly S M, et al. (2015) ω-Hydroxyemodin limits Staphylococcus aureus quorum sensing-mediated pathogenesis and inflammation. Antimicrob Agents Chemother. 59(4):2223-2235.
36. Kusari P, Kusari S, Spiteller M, & Kayser O (2015) Implications of endophyte-plant crosstalk in light of quorum responses for plant biotechnology. Appl Microbiol Biotechnol. 99(13):5383-5390.
37. El-Elimat T, et al. (2013) High-resolution MS, MS/MS, and UV database of fungal secondary metabolites as a dereplication protocol for bioactive natural products.76. J Nat Prod. 9(1709-1716.).
38. Singh S B, et al. (2002) Structure and Chemistry of Apicidins, a Class of Novel Cyclic Tetrapeptides without a Terminal r-Keto Epoxide as Inhibitors of Histone Deacetylase with Potent Antiprotozoal Activities. J Org Chem. 67(3):815-825.
39. Darkin-Rattray S J, et al. (1996) Apicidin: a novel antiprotozoal agent that inhibits parasite histone deacetylase. Proc Natl Acad Sci USA. 93(23):13143-13147.
40. Junio H A, et al. (2013) Quantitative analysis of autoinducing peptide I (AIP-I) from Staphylococcus aureus cultures using ultrahigh performance liquid chromatography-high resolving power mass spectrometry. J Chromatogr B Analyt Technol Biomed Life Sci. 930(doi: 10.1016/j.jchromb.2013.04.019.).
41. Spaan A N, Surewaard B G, Nijland R, & van Strijp J A (2013) Neutrophils Versus Staphylococcus aureus: A Biological Tug of War. Annu Rev Microbiol. 2013(67):629-650.
42. Miller L S & Cho J S (2011) Immunity against Staphylococcus aureus cutaneous infections. Nat Rev Immunol. 11(8):505-518.
43. Bussey R Or, et al. (2015) Comparison of the chemistry and diversity of endophytes isolated from wild-harvested and greenhouse-cultivated yerba mansa (Anemopsis californica). ytochem Lett. March; 11:202-208.
44. Myles I A, et al. (2013) Signaling via the IL-20 receptor inhibits cutaneous production of IL-1β and IL-17A to promote infection with methicillin-resistant Staphylococcus aureus. Nat Immunol. 14(8):804-811.
45. Han J W, et al. (2000) Apicidin, a histone deacetylase inhibitor, inhibits proliferation of tumor cells via induction of p21WAF1/Cip1 and gelsolin. Cancer Res. 60(21):6068-6074.
46. You J S, et al. (2008) Histone deacetylase inhibitor apicidin downregulates DNA methyltransferase 1 expression and induces repressive histone modifications via recruitment of corepressor complex to promoter region in human cervix cancer cells. Oncogene. 27(10):1376-1386.
47. Lai J P, et al. (2009) Additive effect of apicidin and doxorubicin in sulfatase 1 expressing hepatocellular carcinoma in vitro and in vivo. J Hepatol. 50(6):1112-1121.
48. von Bargen K W, et al. (2013) Structure elucidation and antimalarial activity of apicidin F: an apicidin-like compound produced by Fusarium fujikuroi. J Nat Prod. 76(11):2136-2140.
49. CRUFT H J, HINDLEY J, MAURITZEN C M, & STEDMAN E (1957) Amino-acid composition of the six histones of calf thymocytes. Nature. 180(4595):1107-1109.
50. CRUFT H J & LEAVER J L (1961) Isolation of histones(?) from Staphylococcus aureus. Nature. November 11(192):556-557.
51. Yedery R D & Jerse A E (2015) Augmentation of Cationic Antimicrobial Peptide Production with Histone Deacetylase Inhibitors as a Novel Epigenetic Therapy for Bacterial Infections. Antibiotics (Basel). 4(1):44-61.
52. Darkin-Rattray, S. J.; Gurnett, A. M.; Myers, R. W.; Dulski, P. M.; Crumley, T. M.; Allocco, J. J.; Cannova, C.; Meinke, P. T.; Colletti, S. L.; Bednarek, M. A.; Singh, S. B.; Goetz, M. A.; Dombrowski, A. W.; Polishook, J. D.; Schmatz, D. M. Apicidin: A novel antiprotozoal agent that inhibits parasite histone deacetylase. Proceedings of the National Academy of Sciences 1996, 93, 13143-13147.
53. Singh, S. B.; Zink, D. L.; Liesch, J. M.; Mosley, R. T.; Dombrowski, A. W.; Bills, G. F.; Darkin-Rattray, S. J.; Schmatz, D. M.; Goetz, M. A. Structure and Chemistry of Apicidins, a Class of Novel Cyclic Tetrapeptides without a Terminal α-Keto Epoxide as Inhibitors of Histone Deacetylase with Potent Antiprotozoal Activities. J. Org. Chem. 2002, 67, 815-825.
54. Singh, S. B.; Zink, D. L.; Liesch, J. M.; Dombrowski, A. W.; Darkin-Rattray, S. J.; Schmatz, D. M.; Goetz, M. A. Structure, Histone Deacetylase, and Antiprotozoal Activities of Apicidins B and C, Congeners of Apicidin with Proline and Valine Substitutions. Org. Lett. 2001, 3, 2815-2818.
55. Kuriyama, W.; Kitahara, T. Synthesis of Apicidin. Heterocycles 2001, 55, 1-4.
56. Mou, L.; Singh, G. Synthesis of (S)-2-amino-8-oxodecanoic acid (Aoda) and apicidin A. Tetrahedron Lett. 2001, 42, 6603-6606.
57. Berst, F.; Ladlow, M.; Holmes, A. B. Solid-phase synthesis of apicidin A and a cyclic tetrapeptoid analogue. Chem. Commun. 2002, 508-509.
58. Singh, S. B.; Zink, D. L.; Liesch, J. M.; Mosley, R. T.; Dombrowski, A. W.; Bills, G. F.; Darkin-Rattray, S. J.; Schmatz, D. M.; Goetz, M. A. Structure and Chemistry of Apicidins, a Class of Novel Cyclic Tetrapeptides without a Terminal α-Keto Epoxide as Inhibitors of Histone Deacetylase with Potent Antiprotozoal Activities. J. Org. Chem. 2002, 67, 815-825.
59. Meinke, P. T.; Colletti, S. L.; Ayer, M. B.; Darkin-Rattray, S. J.; Myers, R. W.; Schmatz, D. M.; Wyvratt, M. J.; Fisher, M. H. Synthesis of side chain modified apicidin derivatives: potent mechanism-based histone deacetylase inhibitors. Tetrahedron Lett. 2000, 41, 7831-7835.
60. Colletti, S. L.; Myers, R. W.; Darkin-Rattray, S. J.; Gurnett, A. M.; Dulski, P. M.; Galuska, S.; Allocco, J. J.; Ayer, M. B.; Li, C.; Lim, J.; Crumley, T. M.; Cannova, C.; Schmatz, D. M.; Wyvratt, M. J.; Fisher, M.
61. Singh et al. Structure, Histone Deacetylase, and Antiprotozoal Activities of Apicidins B and C, Congeners of Apicidin with Proline and Valine Substitutions. Org Lett. 2001 September 6; 3(18):2815-8.

All references, articles, publications, patents, patent publications, and patent applications cited herein are incorporated by reference in their entireties for all purposes. In the event that any inconsistency exists between the disclosure of the present application and the disclosure(s) of any document incorporated herein by reference, the disclosure of the present application shall govern. However, mention of any reference, article, publication, patent, patent publication, and patent application cited herein is not, and should not be taken as an acknowledgment or any form of suggestion that they constitute valid prior art. It is to be understood that, while the disclosure has been described in conjunction with the detailed description, thereof, the foregoing description is intended to illustrate and not limit the scope. Other aspects, advantages, and modifications are within the scope of the claims set forth below.

What is claimed:

1. A pharmaceutical composition comprising: apicidin, or a pharmaceutically acceptable derivative thereof, in an amount effective to inhibit quorum sensing in a microbe; and a pharmaceutically acceptable carrier.

2. The pharmaceutical composition of claim 1, wherein the apicidin has a structure selected from:

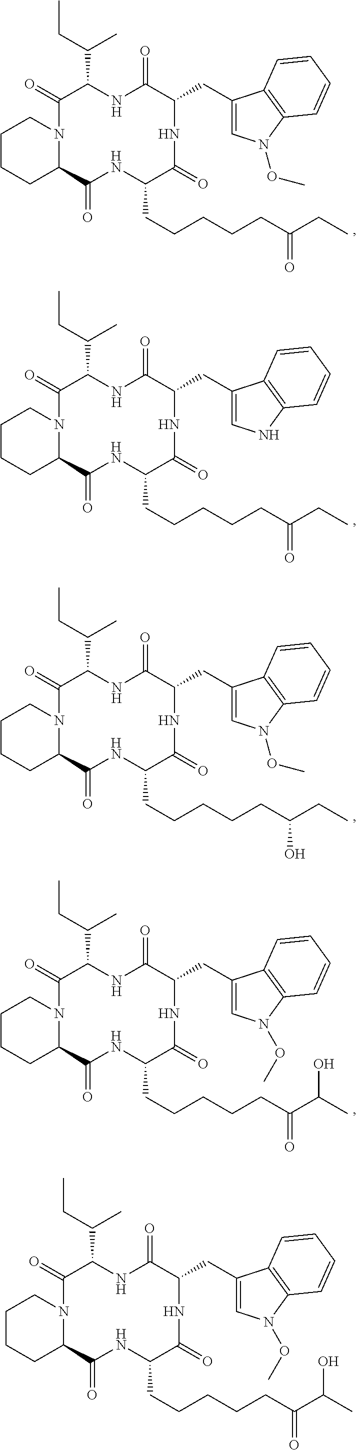 [SMILES: CCC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(=O)CCCCC[C@@H]1NC(=O)[C@H]2CCCCN2C(=O)[C@H](C(C)CC)NC(=O)[C@H](Cc2c[nH]c3ccccc23)NC1=O ; CCC(C)[C@@H]1NC(=O)[C@H](Cc2cn(OC)c3ccccc23)NC(=O)[C@H](CCCCC[C@@H](O)CC)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O]

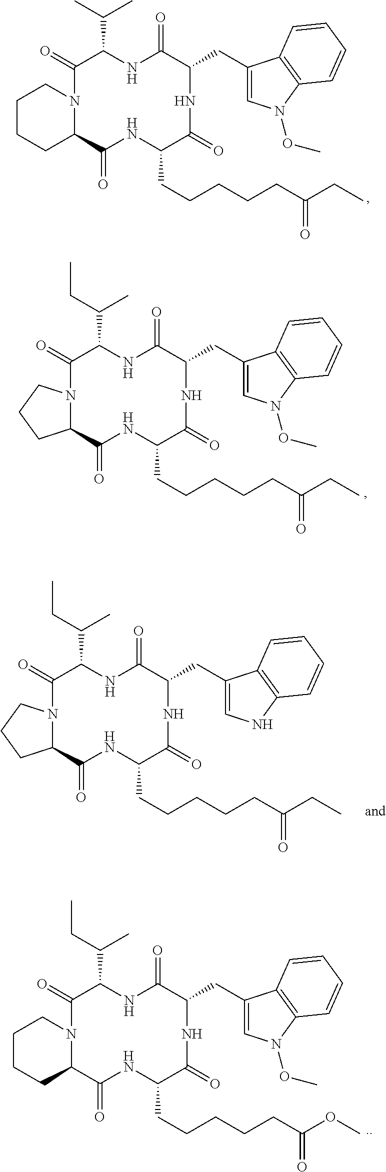 [SMILES: CCC(=O)CCCCC[C@@H]1NC(=O)[C@H]2CCCCN2C(=O)[C@H](C(C)C)NC(=O)[C@H](Cc2cn(OC)c3ccccc23)NC1=O ; CCC(=O)CCCCC[C@@H]1NC(=O)[C@H]2CCCN2C(=O)[C@H](C(C)CC)NC(=O)[C@H](Cc2cn(OC)c3ccccc23)NC1=O ; CCC(=O)CCCCC[C@@H]1NC(=O)[C@H]2CCCN2C(=O)[C@H](C(C)CC)NC(=O)[C@H](Cc2c[nH]c3ccccc23)NC1=O ; CCC(C)[C@@H]1NC(=O)[C@H](Cc2cn(OC)c3ccccc23)NC(=O)[C@H](CCCCCC(=O)OC)NC(=O)[C@H]2CCCCN2C1=O]

3. The pharmaceutical composition of claim 1, wherein apicidin has a structure selected from:

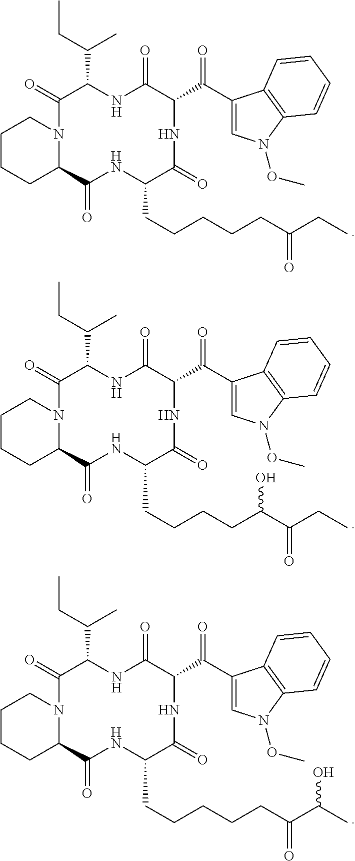 [SMILES: CCC(=O)CCCCC[C@@H]1NC(=O)[C@H]2CCCCN2C(=O)[C@H](C(C)CC)NC(=O)[C@H](C(=O)c2cn(OC)c3ccccc23)NC1=O ; CCC(=O)C(O)CCCC[C@@H]1NC(=O)[C@H]2CCCCN2C(=O)[C@H](C(C)CC)NC(=O)[C@H](C(=O)c2cn(OC)c3ccccc23)NC1=O ; CCC(C)[C@@H]1NC(=O)[C@H](C(=O)c2cn(OC)c3ccccc23)NC(=O)[C@H](CCCCCC(=O)C(C)O)NC(=O)[C@H]2CCCCN2C1=O]

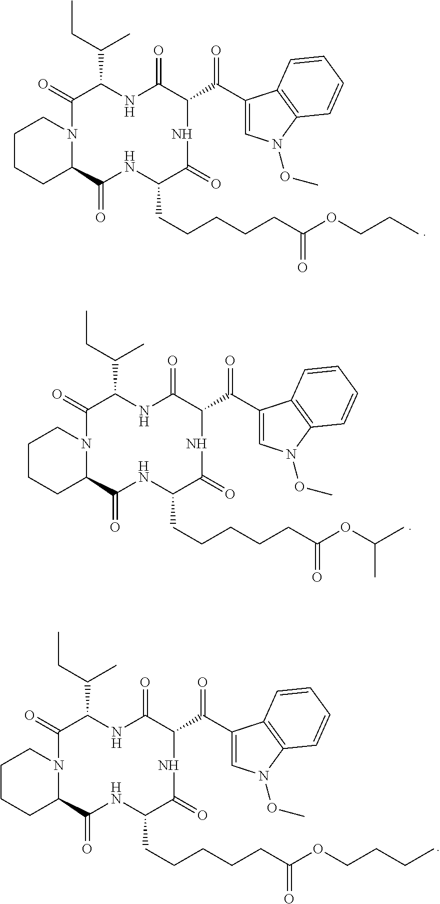 [SMILES: CCCOC(=O)CCCCC[C@@H]1NC(=O)[C@H]2CCCCN2C(=O)[C@H](C(C)CC)NC(=O)[C@H](C(=O)c2cn(OC)c3ccccc23)NC1=O ; CCC(C)[C@@H]1NC(=O)[C@H](C(=O)c2cn(OC)c3ccccc23)NC(=O)[C@H](CCCCCC(=O)OC(C)C)NC(=O)[C@H]2CCCCN2C1=O ; CCCCOC(=O)CCCCC[C@@H]1NC(=O)[C@H]2CCCCN2C(=O)[C@H](C(C)CC)NC(=O)[C@H](C(=O)c2cn(OC)c3ccccc23)NC1=O]

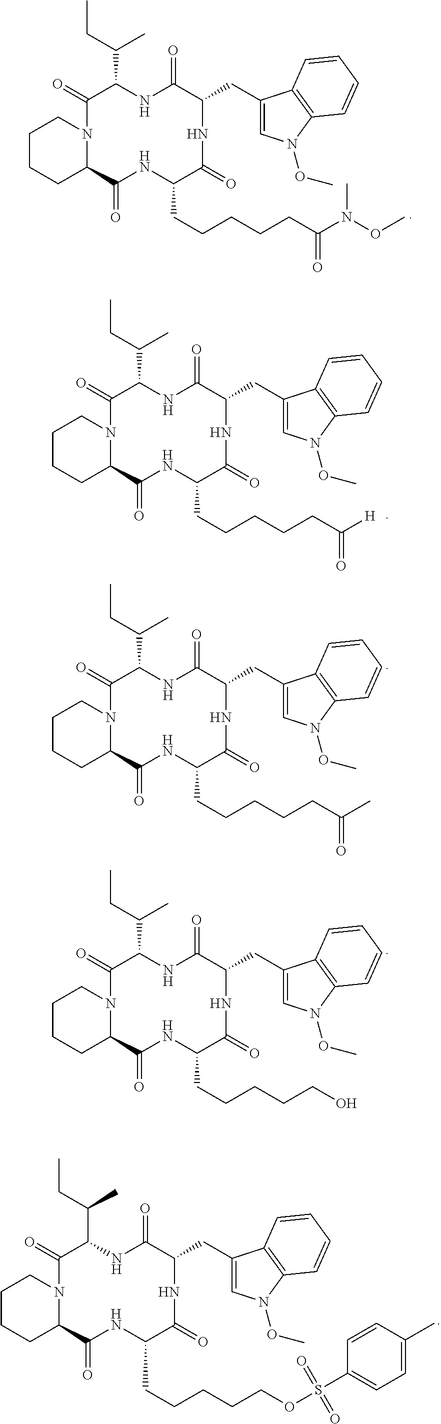 [SMILES: CCC(C)[C@@H]1NC(=O)[C@H](Cc2cn(OC)c3ccccc23)NC(=O)[C@H](CCCCCC(=O)N(C)OC)NC(=O)[C@H]2CCCCN2C1=O ; CCC(C)[C@@H]1NC(=O)[C@H](Cc2cn(OC)c3ccccc23)NC(=O)[C@H](CCCCCC=O)NC(=O)[C@H]2CCCCN2C1=O ; CCC(C)[C@@H]1NC(=O)[C@H](Cc2cn(OC)c3ccccc23)NC(=O)[C@H](CCCCCC(C)=O)NC(=O)[C@H]2CCCCN2C1=O ; CCC(C)[C@@H]1NC(=O)[C@H](Cc2cn(OC)c3ccccc23)NC(=O)[C@H](CCCCCO)NC(=O)[C@H]2CCCCN2C1=O ; CC[C@@H](C)[C@@H]1NC(=O)[C@H](Cc2cn(OC)c3ccccc23)NC(=O)[C@H](CCCCCOS(=O)(=O)c2ccc(C)cc2)NC(=O)[C@H]2CCCCN2C1=O]

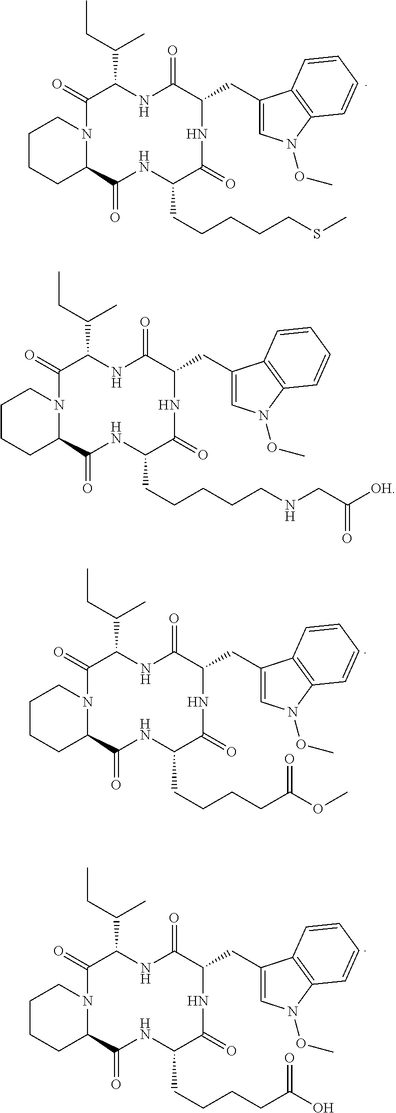 [SMILES: CCC(C)[C@@H]1NC(=O)[C@H](Cc2cn(OC)c3ccccc23)NC(=O)[C@H](CCCCCSC)NC(=O)[C@H]2CCCCN2C1=O ; CCC(C)[C@@H]1NC(=O)[C@H](Cc2cn(OC)c3ccccc23)NC(=O)[C@H](CCCCCNCC(=O)O)NC(=O)[C@H]2CCCCN2C1=O ; CCC(C)[C@@H]1NC(=O)[C@H](Cc2cn(OC)c3ccccc23)NC(=O)[C@H](CCCCC(=O)OC)NC(=O)[C@H]2CCCCN2C1=O ; CCC(C)[C@@H]1NC(=O)[C@H](Cc2cn(OC)c3ccccc23)NC(=O)[C@H](CCCCC(=O)O)NC(=O)[C@H]2CCCCN2C1=O]

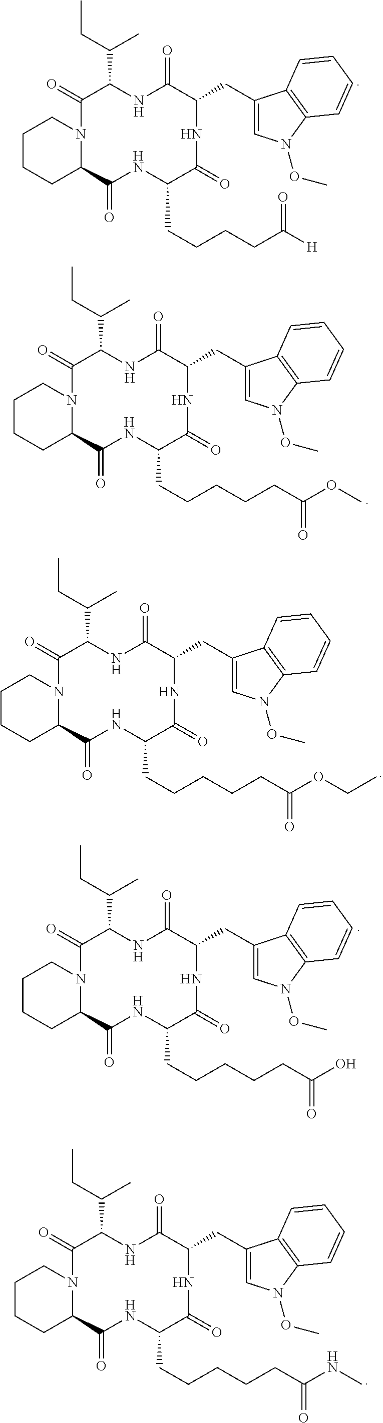 [SMILES: CCC(C)[C@@H]1NC(=O)[C@H](Cc2cn(OC)c3ccccc23)NC(=O)[C@H](CCCCC=O)NC(=O)[C@H]2CCCCN2C1=O ; CCC(C)[C@@H]1NC(=O)[C@H](Cc2cn(OC)c3ccccc23)NC(=O)[C@H](CCCCCC(=O)OC)NC(=O)[C@H]2CCCCN2C1=O ; CCOC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)NC)NC(=O)[C@H]2CCCCN2C1=O]

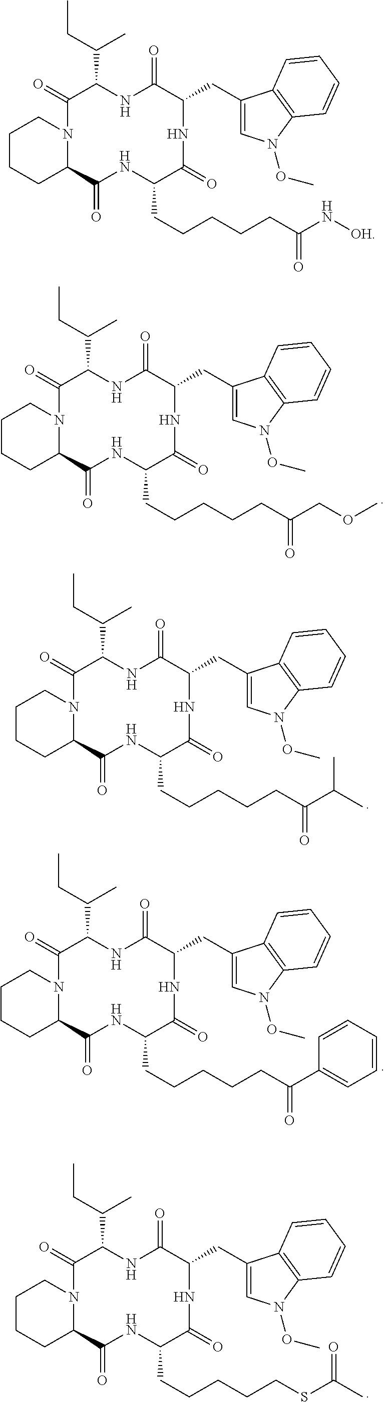 [SMILES: CCC(C)[C@@H]1NC(=O)[C@H](Cc2cn(OC)c3ccccc23)NC(=O)[C@H](CCCCCC(=O)NO)NC(=O)[C@H]2CCCCN2C1=O ; CCC(C)[C@@H]1NC(=O)[C@H](Cc2cn(OC)c3ccccc23)NC(=O)[C@H](CCCCCC(=O)COC)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)C)NC(=O)[C@H]2CCCCN2C1=O ; CCC(C)[C@@H]1NC(=O)[C@H](Cc2cn(OC)c3ccccc23)NC(=O)[C@H](CCCCCC(=O)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCCSC(C)=O)NC(=O)[C@H]2CCCCN2C1=O]

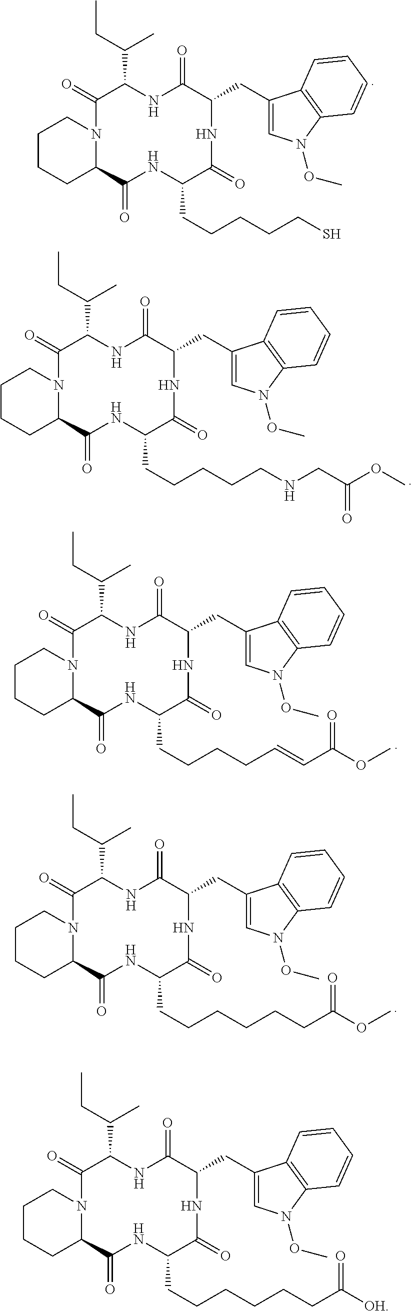 [SMILES: CCC(C)[C@@H]1NC(=O)[C@H](Cc2cn(OC)c3ccccc23)NC(=O)[C@H](CCCCCS)NC(=O)[C@H]2CCCCN2C1=O ; CCC(C)[C@@H]1NC(=O)[C@H](Cc2cn(OC)c3ccccc23)NC(=O)[C@H](CCCCCNCC(=O)OC)NC(=O)[C@H]2CCCCN2C1=O ; CCC(C)[C@@H]1NC(=O)[C@H](Cc2cn(OC)c3ccccc23)NC(=O)[C@H](CCCC/C=C/C(=O)OC)NC(=O)[C@H]2CCCCN2C1=O ; CCC(C)[C@@H]1NC(=O)[C@H](Cc2cn(OC)c3ccccc23)NC(=O)[C@H](CCCCCCC(=O)OC)NC(=O)[C@H]2CCCCN2C1=O ; CCC(C)[C@@H]1NC(=O)[C@H](Cc2cn(OC)c3ccccc23)NC(=O)[C@H](CCCCCCC(=O)O)NC(=O)[C@H]2CCCCN2C1=O]

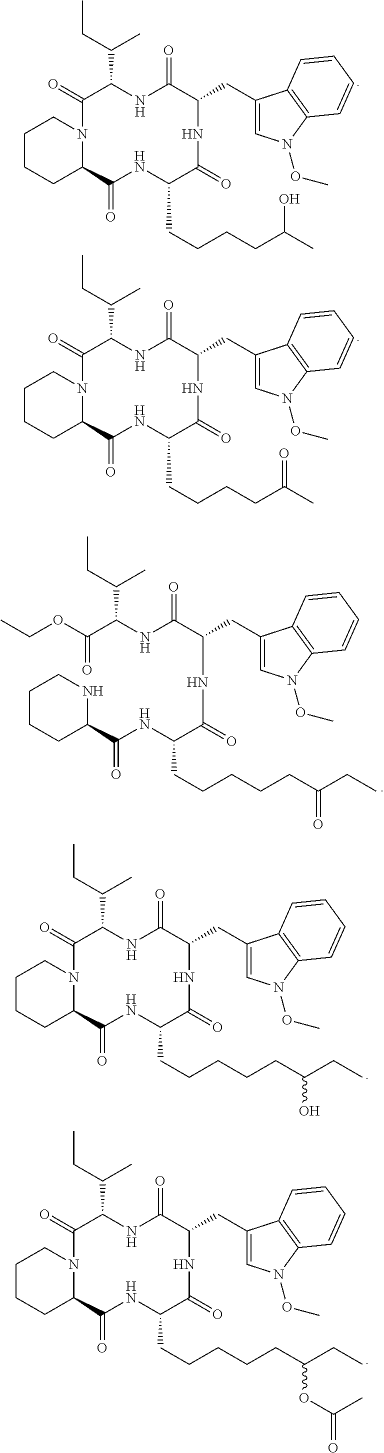 [SMILES: CCC(C)[C@@H]1NC(=O)[C@H](Cc2cn(OC)c3ccccc23)NC(=O)[C@H](CCCCC(C)O)NC(=O)[C@H]2CCCCN2C1=O ; CCC(C)[C@@H]1NC(=O)[C@H](Cc2cn(OC)c3ccccc23)NC(=O)[C@H](CCCCC(C)=O)NC(=O)[C@H]2CCCCN2C1=O ; CCOC(=O)[C@@H](NC(=O)[C@H](Cc1cn(OC)c2ccccc12)NC(=O)[C@H](CCCCCC(=O)CC)NC(=O)[C@H]1CCCCN1)C(C)CC ; CCC(O)CCCCC[C@@H]1NC(=O)[C@H]2CCCCN2C(=O)[C@H](C(C)CC)NC(=O)[C@H](Cc2cn(OC)c3ccccc23)NC1=O ; CCC(CCCCC[C@@H]1NC(=O)[C@H]2CCCCN2C(=O)[C@H](C(C)CC)NC(=O)[C@H](Cc2cn(OC)c3ccccc23)NC1=O)OC(C)=O]

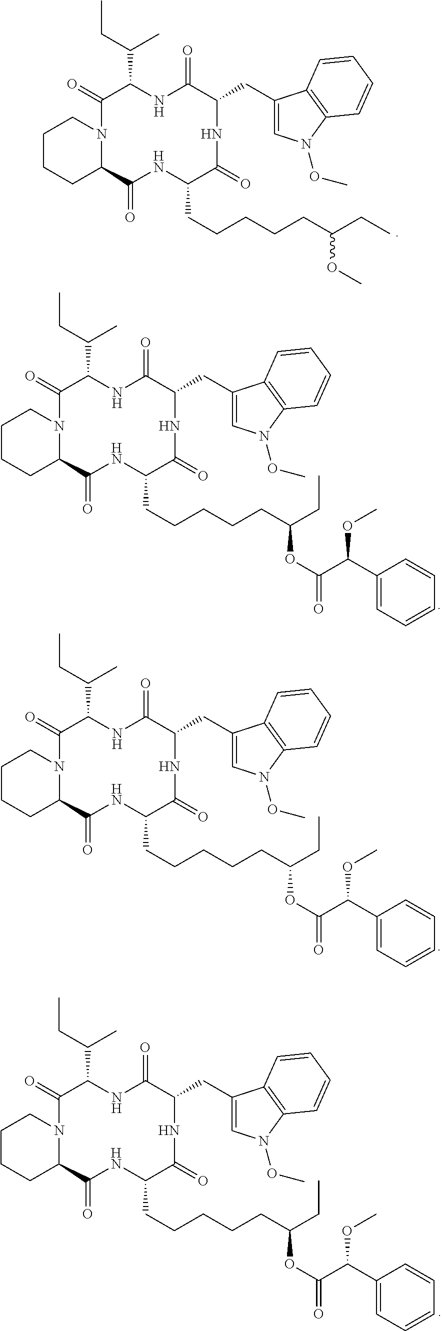 [SMILES: CCC(CCCCC[C@@H]1NC(=O)[C@H]2CCCCN2C(=O)[C@H](C(C)CC)NC(=O)[C@H](Cc2cn(OC)c3ccccc23)NC1=O)OC ; CCC(C)[C@@H]1NC(=O)[C@H](Cc2cn(OC)c3ccccc23)NC(=O)[C@H](CCCCC[C@@H](CC)OC(=O)[C@@H](OC)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCC[C@H](CC)OC(=O)[C@H](OC)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCC[C@@H](CC)OC(=O)[C@H](OC)c2ccccc2)NC(=O)[C@H]2CCCCN2C1=O]

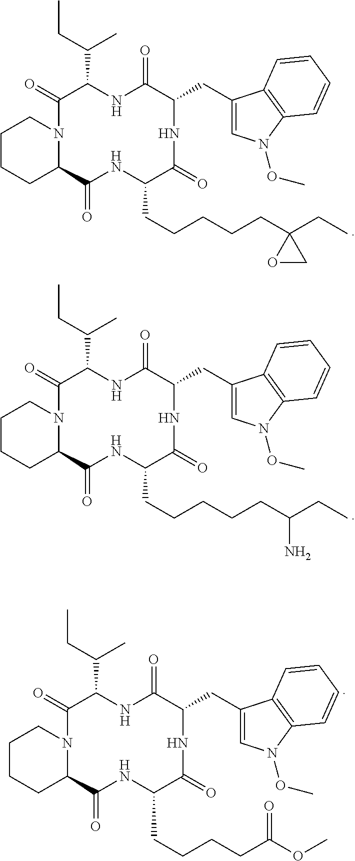 [SMILES: CCC(C)[C@@H]1NC(=O)[C@H](Cc2cn(OC)c3ccccc23)NC(=O)[C@H](CCCCCC2(CC)CO2)NC(=O)[C@H]2CCCCN2C1=O ; CCC(N)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCC(=O)OC)NC(=O)[C@H]2CCCCN2C1=O]

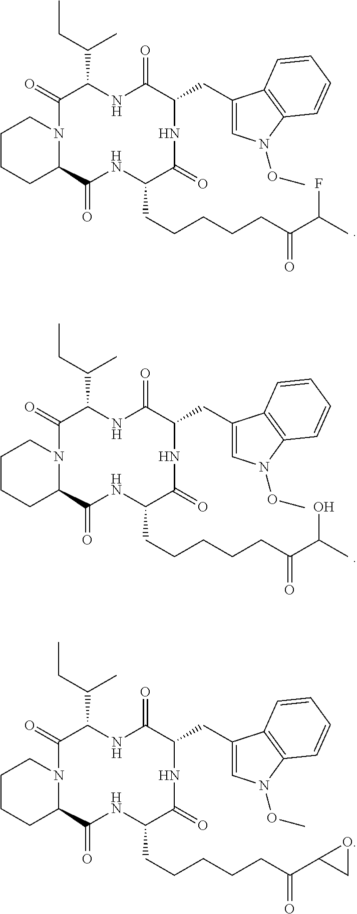 [SMILES: CCC(C)[C@@H]1NC(=O)[C@H](Cc2cn(OC)c3ccccc23)NC(=O)[C@H](CCCCCC(=O)C(C)F)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)C2CO2)NC(=O)[C@H]2CCCCN2C1=O]

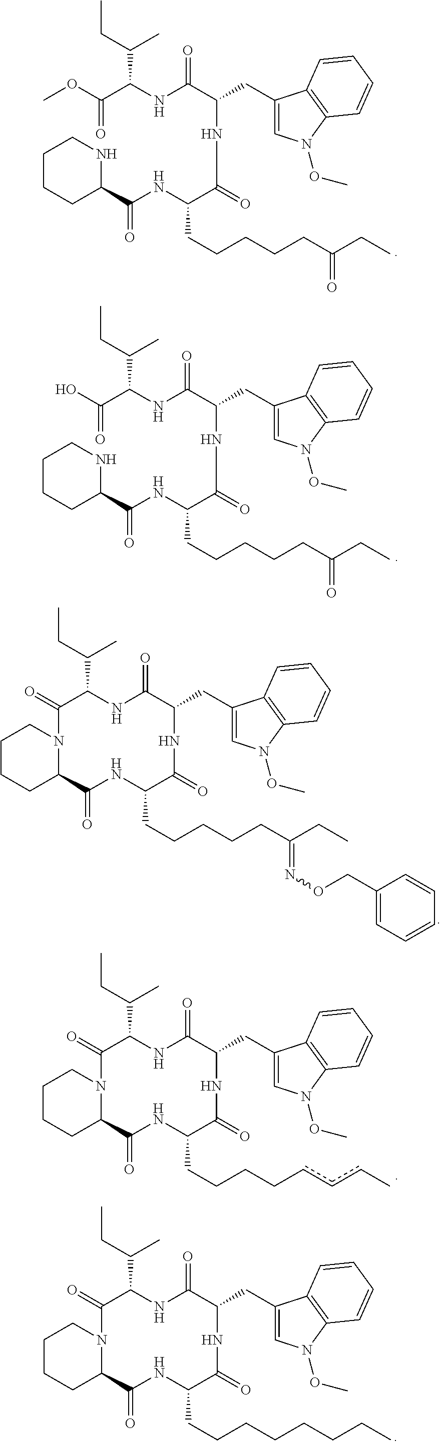

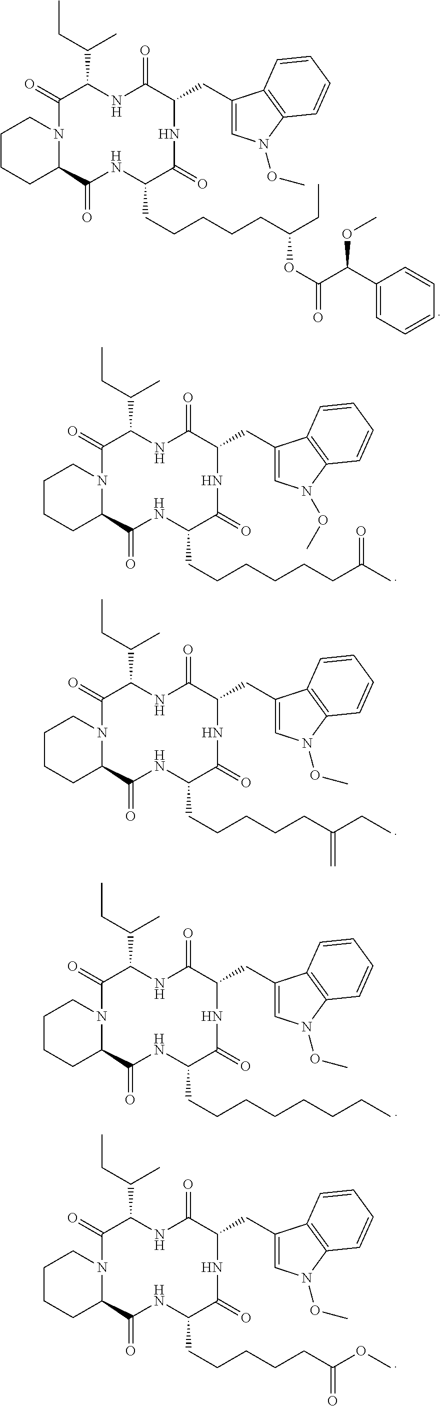

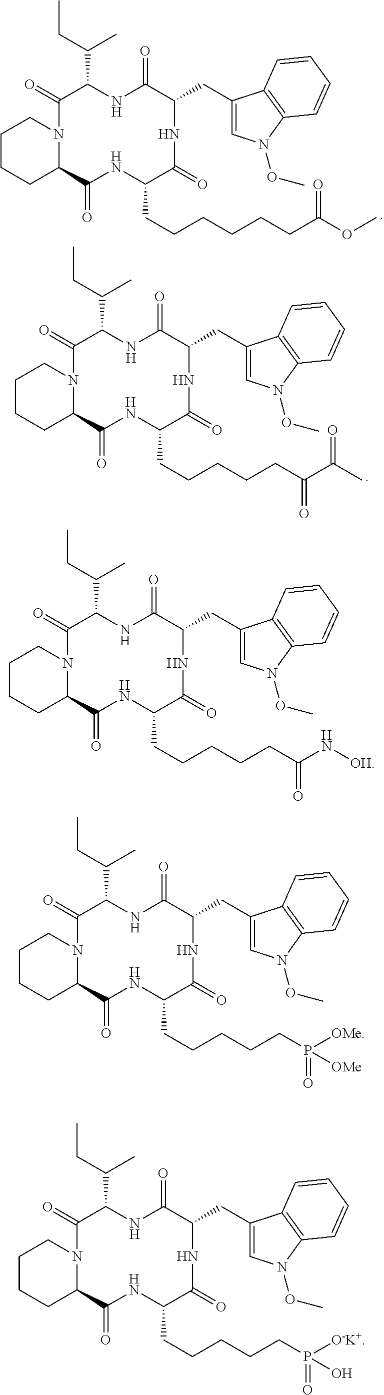

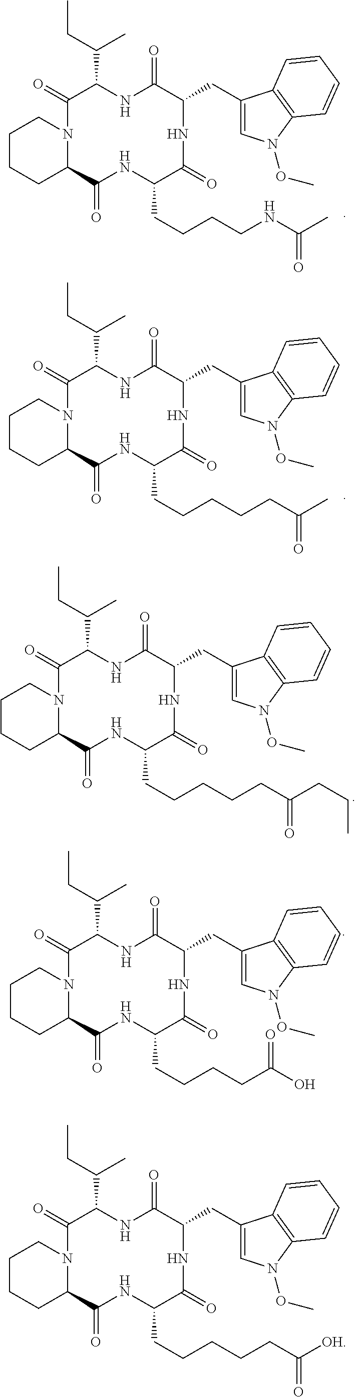 [SMILES: CCC(C)[C@@H]1NC(=O)[C@H](Cc2cn(OC)c3ccccc23)NC(=O)[C@H](CCCCNC(C)=O)NC(=O)[C@H]2CCCCN2C1=O ; CCC(C)[C@@H]1NC(=O)[C@H](Cc2cn(OC)c3ccccc23)NC(=O)[C@H](CCCCCC(C)=O)NC(=O)[C@H]2CCCCN2C1=O ; CCCC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCC(=O)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)O)NC(=O)[C@H]2CCCCN2C1=O]

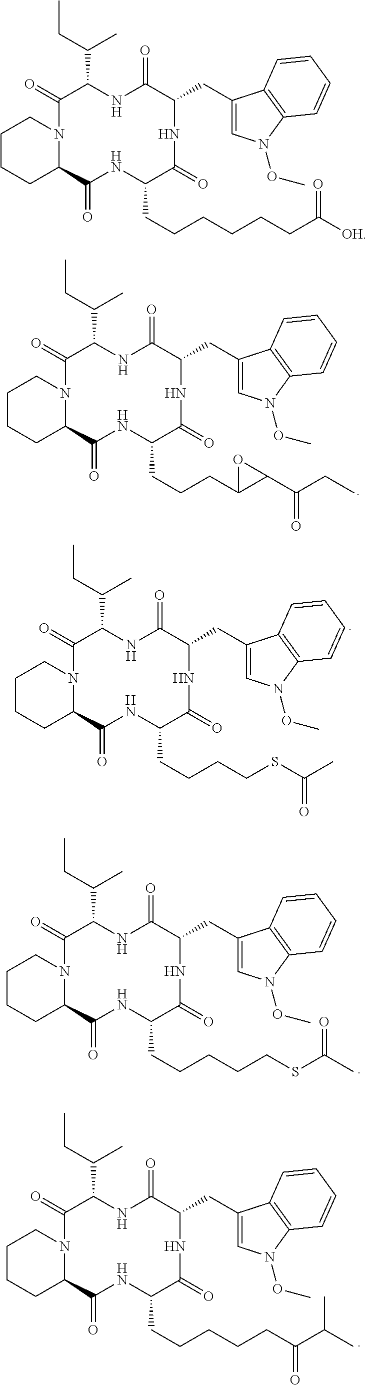 [SMILES: CCC(C)[C@@H]1NC(=O)[C@H](Cc2cn(OC)c3ccccc23)NC(=O)[C@H](CCCCCCC(=O)O)NC(=O)[C@H]2CCCCN2C1=O ; CCC(=O)C1OC1CCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCSC(C)=O)NC(=O)[C@H]2CCCCN2C1=O ; CCC(C)[C@@H]1NC(=O)[C@H](Cc2cn(OC)c3ccccc23)NC(=O)[C@H](CCCCCSC(C)=O)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)C)NC(=O)[C@H]2CCCCN2C1=O]

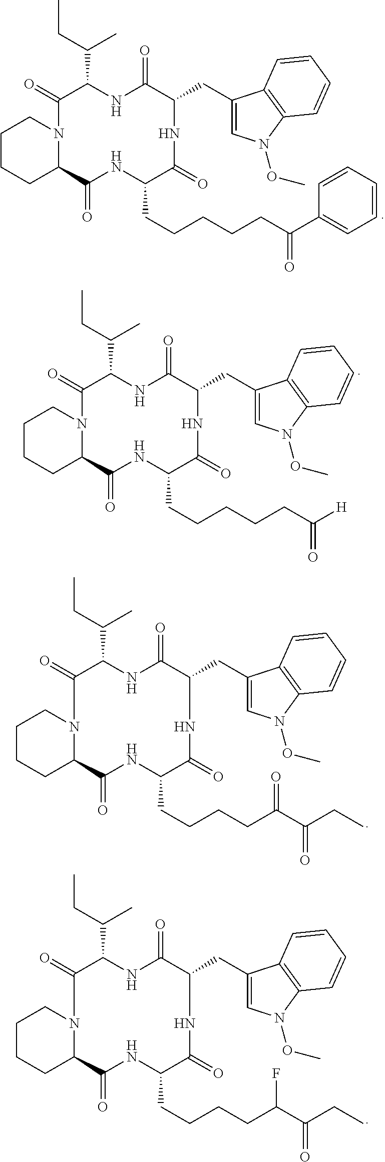 [SMILES: CCC(C)[C@@H]1NC(=O)[C@H](Cc2cn(OC)c3ccccc23)NC(=O)[C@H](CCCCCC(=O)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCCC=O)NC(=O)[C@H]2CCCCN2C1=O ; CCC(=O)C(=O)CCCC[C@@H]1NC(=O)[C@H]2CCCCN2C(=O)[C@H](C(C)CC)NC(=O)[C@H](Cc2cn(OC)c3ccccc23)NC1=O ; CCC(=O)C(F)CCCC[C@@H]1NC(=O)[C@H]2CCCCN2C(=O)[C@H](C(C)CC)NC(=O)[C@H](Cc2cn(OC)c3ccccc23)NC1=O]

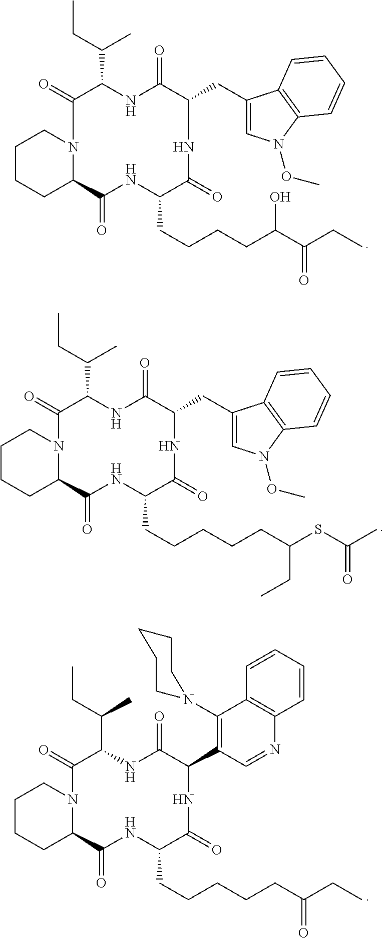 [SMILES: CCC(=O)C(O)CCCC[C@@H]1NC(=O)[C@H]2CCCCN2C(=O)[C@H](C(C)CC)NC(=O)[C@H](Cc2cn(OC)c3ccccc23)NC1=O ; CCC(CCCCC[C@@H]1NC(=O)[C@H]2CCCCN2C(=O)[C@H](C(C)CC)NC(=O)[C@H](Cc2cn(OC)c3ccccc23)NC1=O)SC(C)=O ; CCC(=O)CCCCC[C@@H]1NC(=O)[C@H]2CCCCN2C(=O)[C@H]([C@H](C)CC)NC(=O)[C@@H](c2cnc3ccccc3c2N2CCCCC2)NC1=O]

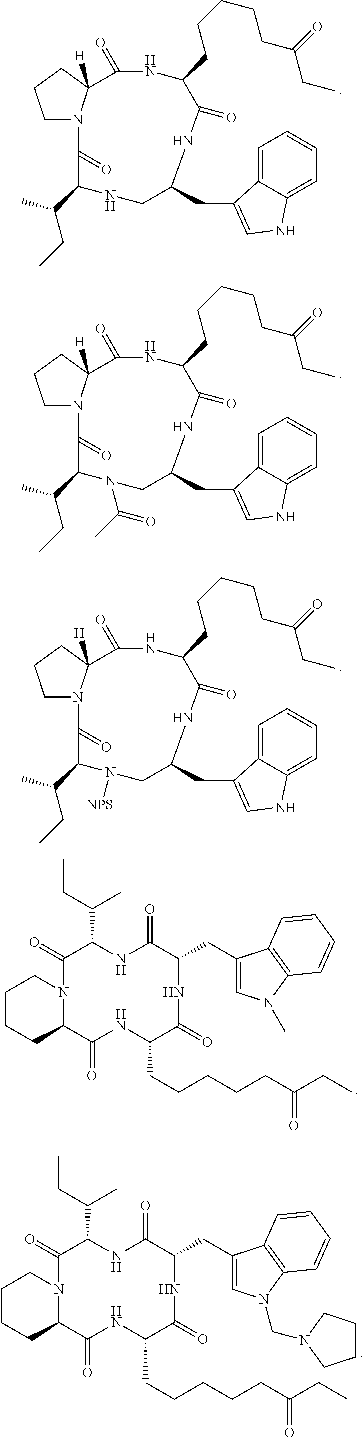

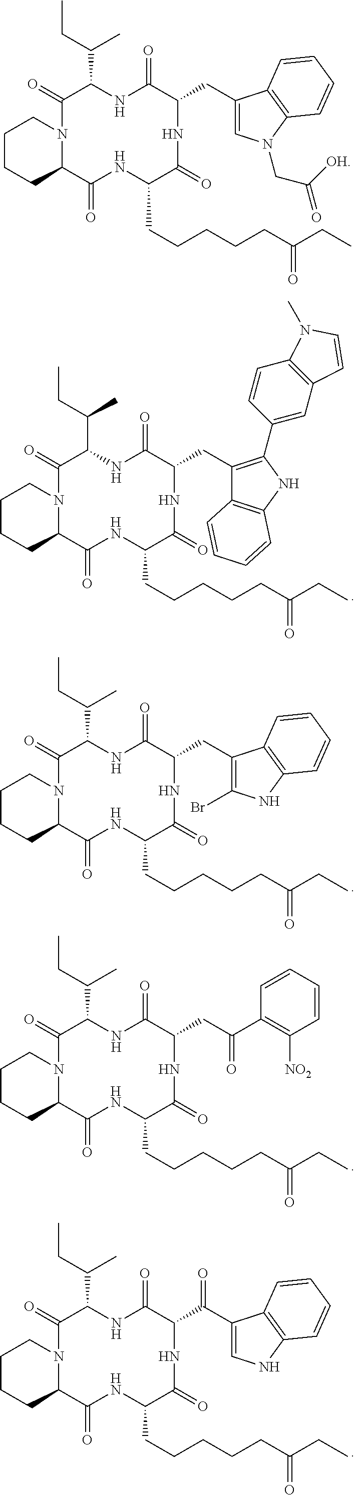

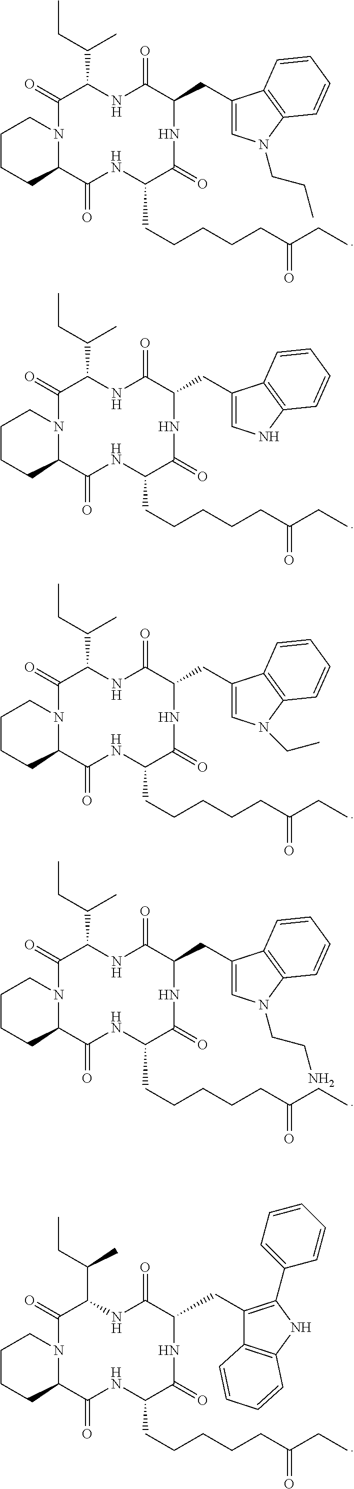 [SMILES: CCCn1cc(C[C@H]2NC(=O)[C@H](CCCCCC(=O)CC)NC(=O)[C@H]3CCCCN3C(=O)[C@H](C(C)CC)NC2=O)c2ccccc21 ; CCC(=O)CCCCC[C@@H]1NC(=O)[C@H]2CCCCN2C(=O)[C@H](C(C)CC)NC(=O)[C@H](Cc2c[nH]c3ccccc23)NC1=O ; CCC(=O)CCCCC[C@@H]1NC(=O)[C@H]2CCCCN2C(=O)[C@H](C(C)CC)NC(=O)[C@H](Cc2cn(CC)c3ccccc23)NC1=O ; CCC(=O)CCCCC[C@@H]1NC(=O)[C@H]2CCCCN2C(=O)[C@H](C(C)CC)NC(=O)[C@@H](Cc2cn(CCN)c3ccccc23)NC1=O ; CCC(=O)CCCCC[C@@H]1NC(=O)[C@H]2CCCCN2C(=O)[C@H]([C@H](C)CC)NC(=O)[C@H](Cc2c(-c3ccccc3)[nH]c3ccccc23)NC1=O]

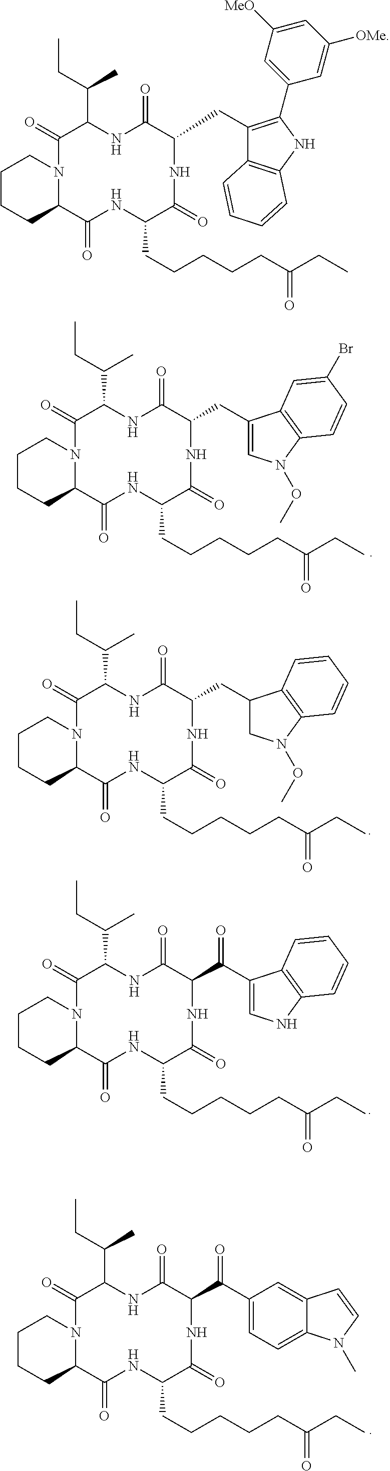 [SMILES: CCC(=O)CCCCC[C@@H]1NC(=O)[C@H]2CCCCN2C(=O)C([C@H](C)CC)NC(=O)[C@H](Cc2c(-c3cc(OC)cc(OC)c3)[nH]c3ccccc23)NC1=O ; CCC(=O)CCCCC[C@@H]1NC(=O)[C@H]2CCCCN2C(=O)[C@H](C(C)CC)NC(=O)[C@H](Cc2cn(OC)c3ccc(Br)cc23)NC1=O ; CCC(=O)CCCCC[C@@H]1NC(=O)[C@H]2CCCCN2C(=O)[C@H](C(C)CC)NC(=O)[C@H](CC2CN(OC)c3ccccc32)NC1=O ; CCC(=O)CCCCC[C@@H]1NC(=O)[C@H]2CCCCN2C(=O)[C@H](C(C)CC)NC(=O)[C@@H](C(=O)c2c[nH]c3ccccc23)NC1=O ; CCC(=O)CCCCC[C@@H]1NC(=O)[C@H]2CCCCN2C(=O)C([C@H](C)CC)NC(=O)[C@@H](C(=O)c2ccc3c(ccn3C)c2)NC1=O]

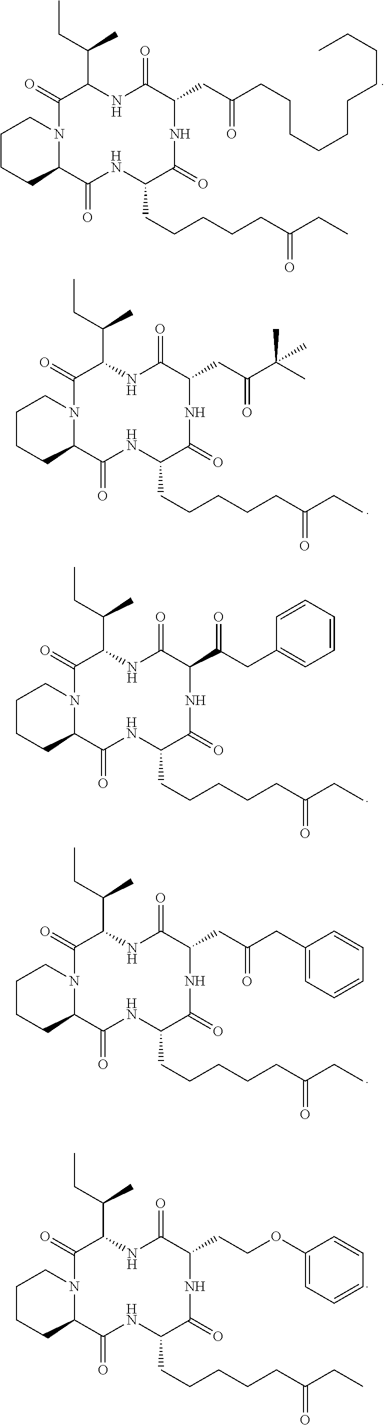

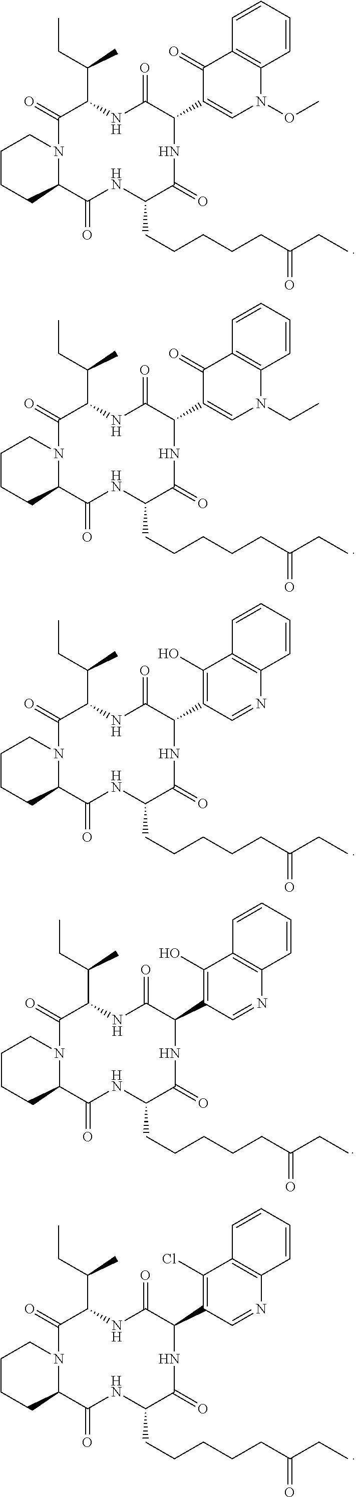

4. The pharmaceutical composition of claim 1, wherein the microbe is Staphylococcus aureus.

5. The pharmaceutical composition of claim 4, wherein the microbe is methicillin-resistant S. aureus or a methicillin-sensitive S. aureus.

6. The pharmaceutical composition of claim 1, wherein the quorum sensing in a microbe comprises regulation of expression of a virulence factor by an accessory gene regulator (agr).

7. The pharmaceutical composition of claim 1, wherein the quorum sensing in a microbe comprises interference of response regulator AgrA activity.

8. A pharmaceutical composition comprising: apicidin, or a pharmaceutically acceptable derivative thereof, in an amount effective to treat a skin and soft tissue infection (SSTI); and a pharmaceutically acceptable carrier.

9. The pharmaceutical composition of claim 8, wherein the apicidin has a structure selected from:

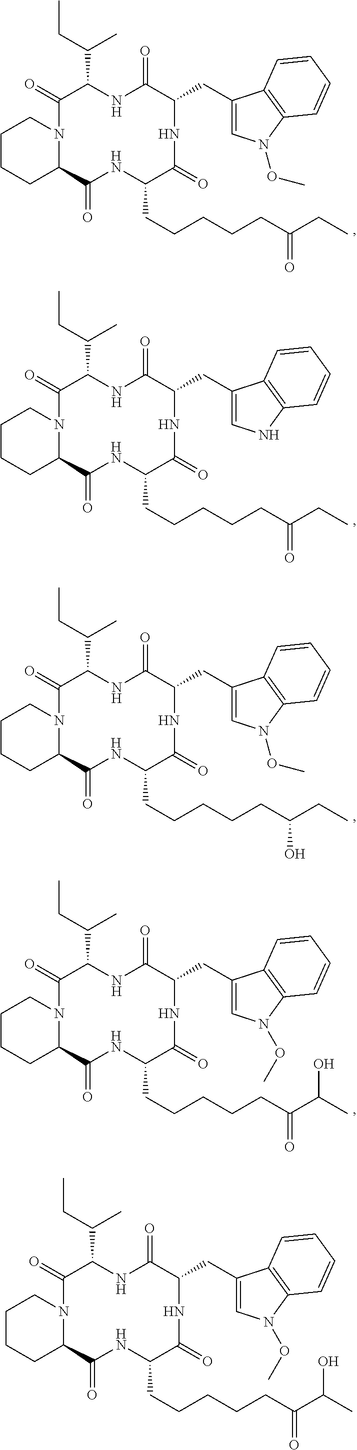 [SMILES: CCC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(=O)CCCCC[C@@H]1NC(=O)[C@H]2CCCCN2C(=O)[C@H](C(C)CC)NC(=O)[C@H](Cc2c[nH]c3ccccc23)NC1=O ; CCC(C)[C@@H]1NC(=O)[C@H](Cc2cn(OC)c3ccccc23)NC(=O)[C@H](CCCCC[C@@H](O)CC)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O]

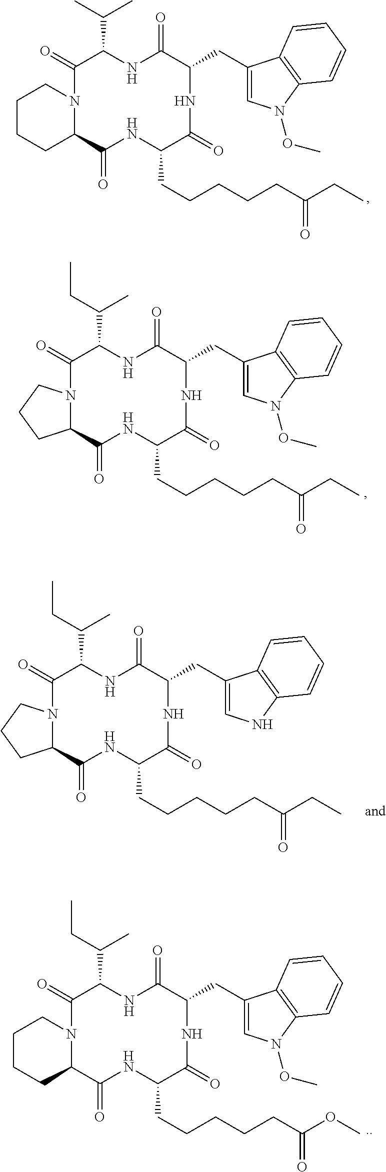 [SMILES: CCC(=O)CCCCC[C@@H]1NC(=O)[C@H]2CCCCN2C(=O)[C@H](C(C)C)NC(=O)[C@H](Cc2cn(OC)c3ccccc23)NC1=O ; CCC(=O)CCCCC[C@@H]1NC(=O)[C@H]2CCCN2C(=O)[C@H](C(C)CC)NC(=O)[C@H](Cc2cn(OC)c3ccccc23)NC1=O ; CCC(=O)CCCCC[C@@H]1NC(=O)[C@H]2CCCN2C(=O)[C@H](C(C)CC)NC(=O)[C@H](Cc2c[nH]c3ccccc23)NC1=O ; CCC(C)[C@@H]1NC(=O)[C@H](Cc2cn(OC)c3ccccc23)NC(=O)[C@H](CCCCCC(=O)OC)NC(=O)[C@H]2CCCCN2C1=O]

10. The pharmaceutical composition of claim 8, wherein apicidin has a structure selected from:

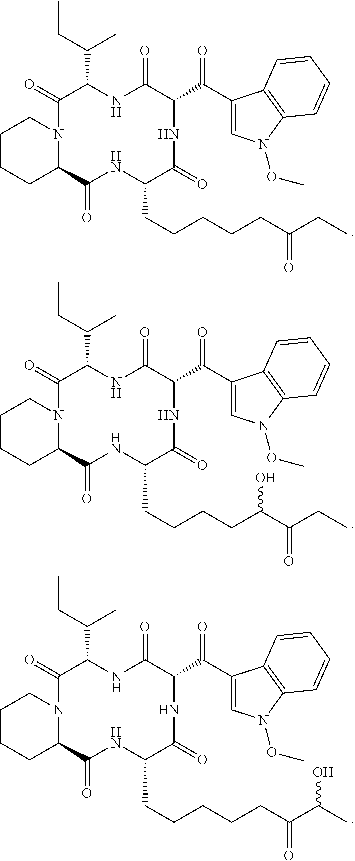 [SMILES: CCC(=O)CCCCC[C@@H]1NC(=O)[C@H]2CCCCN2C(=O)[C@H](C(C)CC)NC(=O)[C@H](C(=O)c2cn(OC)c3ccccc23)NC1=O ; CCC(=O)C(O)CCCC[C@@H]1NC(=O)[C@H]2CCCCN2C(=O)[C@H](C(C)CC)NC(=O)[C@H](C(=O)c2cn(OC)c3ccccc23)NC1=O ; CCC(C)[C@@H]1NC(=O)[C@H](C(=O)c2cn(OC)c3ccccc23)NC(=O)[C@H](CCCCCC(=O)C(C)O)NC(=O)[C@H]2CCCCN2C1=O]

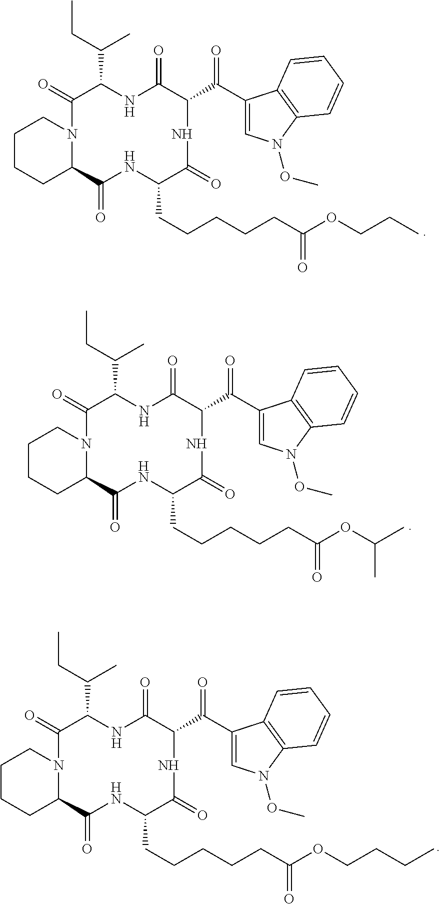 [SMILES: CCCOC(=O)CCCCC[C@@H]1NC(=O)[C@H]2CCCCN2C(=O)[C@H](C(C)CC)NC(=O)[C@H](C(=O)c2cn(OC)c3ccccc23)NC1=O ; CCC(C)[C@@H]1NC(=O)[C@H](C(=O)c2cn(OC)c3ccccc23)NC(=O)[C@H](CCCCCC(=O)OC(C)C)NC(=O)[C@H]2CCCCN2C1=O ; CCCCOC(=O)CCCCC[C@@H]1NC(=O)[C@H]2CCCCN2C(=O)[C@H](C(C)CC)NC(=O)[C@H](C(=O)c2cn(OC)c3ccccc23)NC1=O]

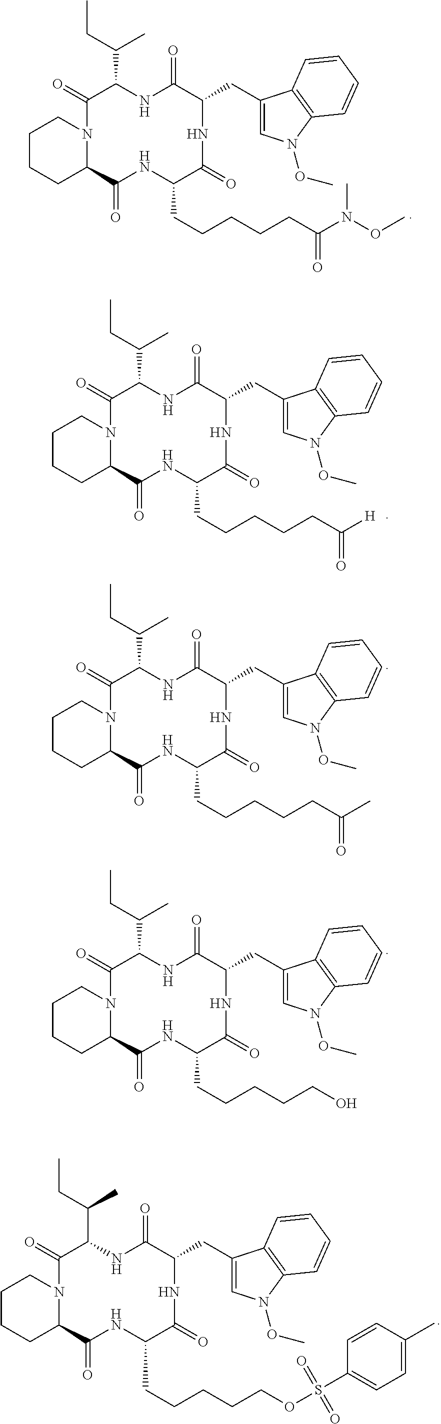 [SMILES: CCC(C)[C@@H]1NC(=O)[C@H](Cc2cn(OC)c3ccccc23)NC(=O)[C@H](CCCCCC(=O)N(C)OC)NC(=O)[C@H]2CCCCN2C1=O ; CCC(C)[C@@H]1NC(=O)[C@H](Cc2cn(OC)c3ccccc23)NC(=O)[C@H](CCCCCC=O)NC(=O)[C@H]2CCCCN2C1=O ; CCC(C)[C@@H]1NC(=O)[C@H](Cc2cn(OC)c3ccccc23)NC(=O)[C@H](CCCCCC(C)=O)NC(=O)[C@H]2CCCCN2C1=O ; CCC(C)[C@@H]1NC(=O)[C@H](Cc2cn(OC)c3ccccc23)NC(=O)[C@H](CCCCCO)NC(=O)[C@H]2CCCCN2C1=O ; CC[C@@H](C)[C@@H]1NC(=O)[C@H](Cc2cn(OC)c3ccccc23)NC(=O)[C@H](CCCCCOS(=O)(=O)c2ccc(C)cc2)NC(=O)[C@H]2CCCCN2C1=O]

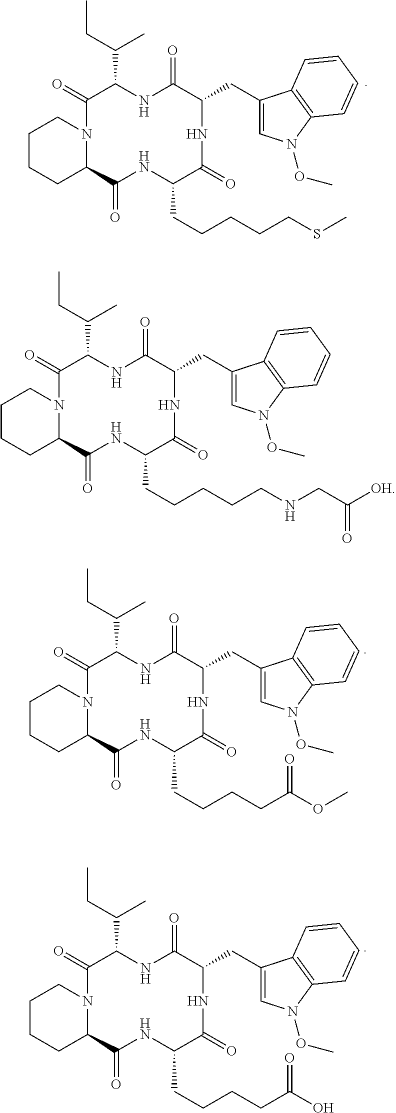 [SMILES: CCC(C)[C@@H]1NC(=O)[C@H](Cc2cn(OC)c3ccccc23)NC(=O)[C@H](CCCCCSC)NC(=O)[C@H]2CCCCN2C1=O ; CCC(C)[C@@H]1NC(=O)[C@H](Cc2cn(OC)c3ccccc23)NC(=O)[C@H](CCCCCNCC(=O)O)NC(=O)[C@H]2CCCCN2C1=O ; CCC(C)[C@@H]1NC(=O)[C@H](Cc2cn(OC)c3ccccc23)NC(=O)[C@H](CCCCC(=O)OC)NC(=O)[C@H]2CCCCN2C1=O ; CCC(C)[C@@H]1NC(=O)[C@H](Cc2cn(OC)c3ccccc23)NC(=O)[C@H](CCCCC(=O)O)NC(=O)[C@H]2CCCCN2C1=O]

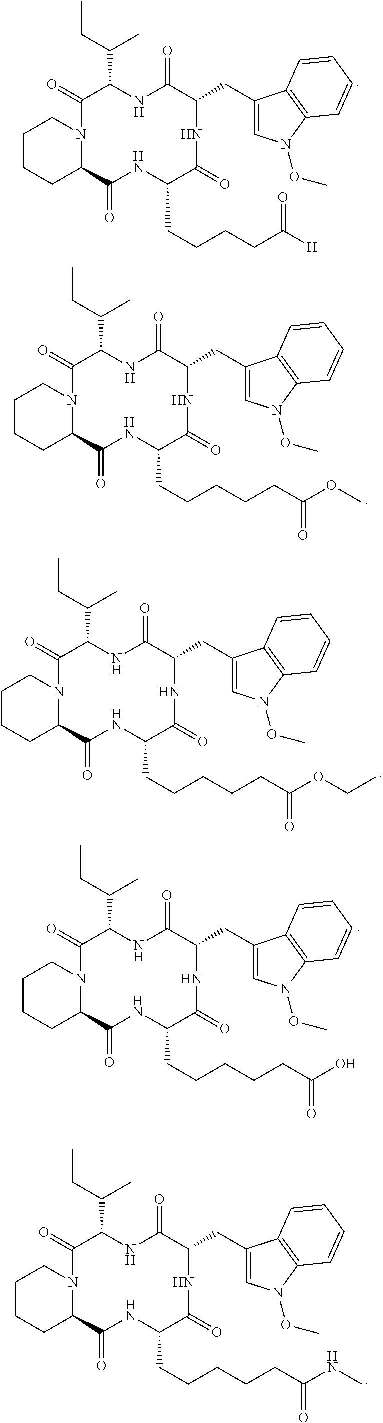 [SMILES: CCC(C)[C@@H]1NC(=O)[C@H](Cc2cn(OC)c3ccccc23)NC(=O)[C@H](CCCCC=O)NC(=O)[C@H]2CCCCN2C1=O ; CCC(C)[C@@H]1NC(=O)[C@H](Cc2cn(OC)c3ccccc23)NC(=O)[C@H](CCCCCC(=O)OC)NC(=O)[C@H]2CCCCN2C1=O ; CCOC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)NC)NC(=O)[C@H]2CCCCN2C1=O]

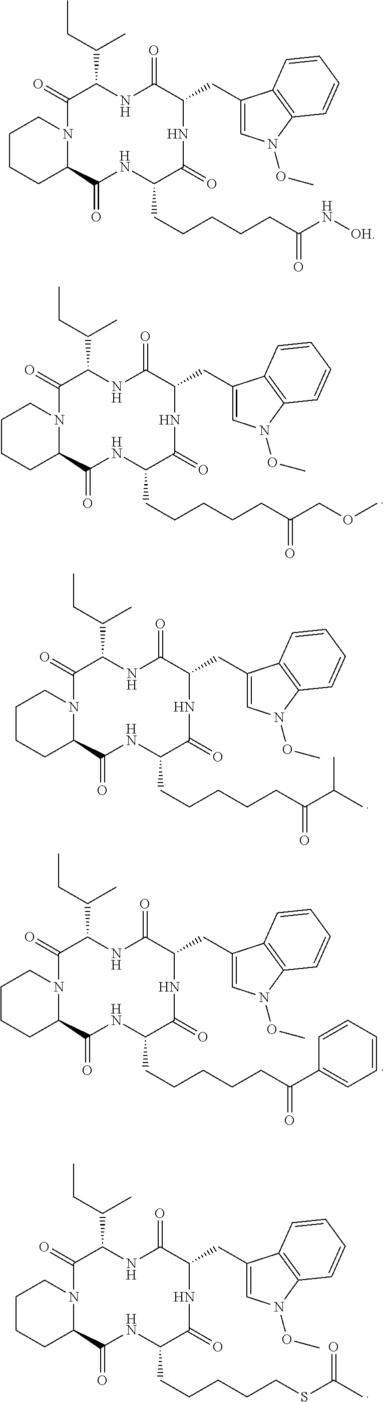 [SMILES: CCC(C)[C@@H]1NC(=O)[C@H](Cc2cn(OC)c3ccccc23)NC(=O)[C@H](CCCCCC(=O)NO)NC(=O)[C@H]2CCCCN2C1=O ; CCC(C)[C@@H]1NC(=O)[C@H](Cc2cn(OC)c3ccccc23)NC(=O)[C@H](CCCCCC(=O)COC)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)C)NC(=O)[C@H]2CCCCN2C1=O ; CCC(C)[C@@H]1NC(=O)[C@H](Cc2cn(OC)c3ccccc23)NC(=O)[C@H](CCCCCC(=O)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCCSC(C)=O)NC(=O)[C@H]2CCCCN2C1=O]

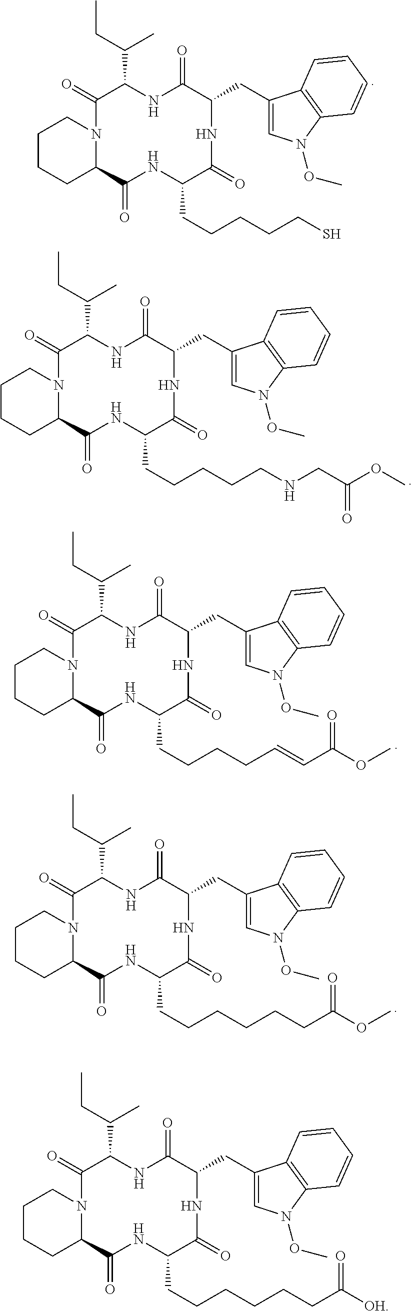 [SMILES: CCC(C)[C@@H]1NC(=O)[C@H](Cc2cn(OC)c3ccccc23)NC(=O)[C@H](CCCCCS)NC(=O)[C@H]2CCCCN2C1=O ; CCC(C)[C@@H]1NC(=O)[C@H](Cc2cn(OC)c3ccccc23)NC(=O)[C@H](CCCCCNCC(=O)OC)NC(=O)[C@H]2CCCCN2C1=O ; CCC(C)[C@@H]1NC(=O)[C@H](Cc2cn(OC)c3ccccc23)NC(=O)[C@H](CCCC/C=C/C(=O)OC)NC(=O)[C@H]2CCCCN2C1=O ; CCC(C)[C@@H]1NC(=O)[C@H](Cc2cn(OC)c3ccccc23)NC(=O)[C@H](CCCCCCC(=O)OC)NC(=O)[C@H]2CCCCN2C1=O ; CCC(C)[C@@H]1NC(=O)[C@H](Cc2cn(OC)c3ccccc23)NC(=O)[C@H](CCCCCCC(=O)O)NC(=O)[C@H]2CCCCN2C1=O]

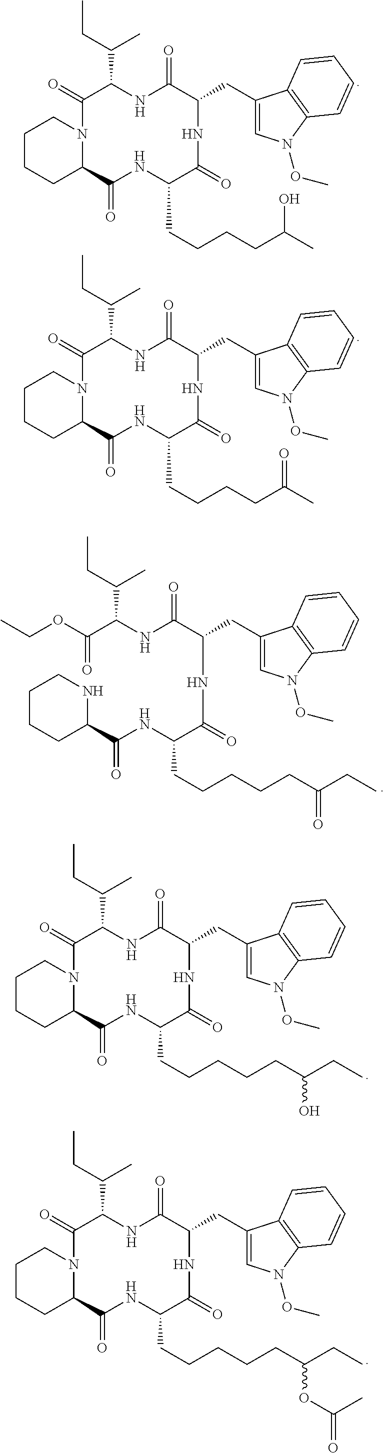 [SMILES: CCC(C)[C@@H]1NC(=O)[C@H](Cc2cn(OC)c3ccccc23)NC(=O)[C@H](CCCCC(C)O)NC(=O)[C@H]2CCCCN2C1=O ; CCC(C)[C@@H]1NC(=O)[C@H](Cc2cn(OC)c3ccccc23)NC(=O)[C@H](CCCCC(C)=O)NC(=O)[C@H]2CCCCN2C1=O ; CCOC(=O)[C@@H](NC(=O)[C@H](Cc1cn(OC)c2ccccc12)NC(=O)[C@H](CCCCCC(=O)CC)NC(=O)[C@H]1CCCCN1)C(C)CC ; CCC(O)CCCCC[C@@H]1NC(=O)[C@H]2CCCCN2C(=O)[C@H](C(C)CC)NC(=O)[C@H](Cc2cn(OC)c3ccccc23)NC1=O ; CCC(CCCCC[C@@H]1NC(=O)[C@H]2CCCCN2C(=O)[C@H](C(C)CC)NC(=O)[C@H](Cc2cn(OC)c3ccccc23)NC1=O)OC(C)=O]

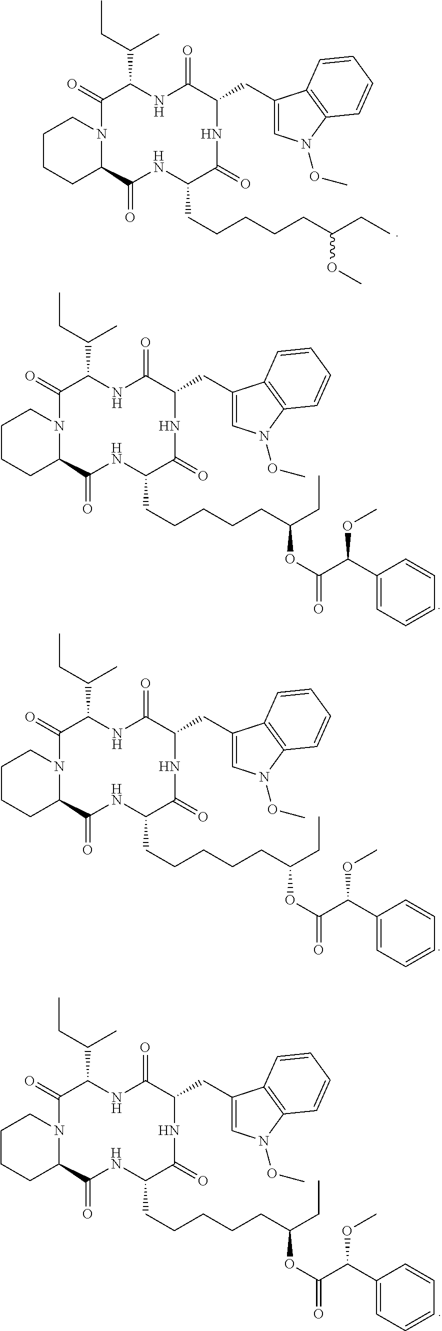 [SMILES: CCC(CCCCC[C@@H]1NC(=O)[C@H]2CCCCN2C(=O)[C@H](C(C)CC)NC(=O)[C@H](Cc2cn(OC)c3ccccc23)NC1=O)OC ; CCC(C)[C@@H]1NC(=O)[C@H](Cc2cn(OC)c3ccccc23)NC(=O)[C@H](CCCCC[C@@H](CC)OC(=O)[C@@H](OC)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCC[C@H](CC)OC(=O)[C@H](OC)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCC[C@@H](CC)OC(=O)[C@H](OC)c2ccccc2)NC(=O)[C@H]2CCCCN2C1=O]

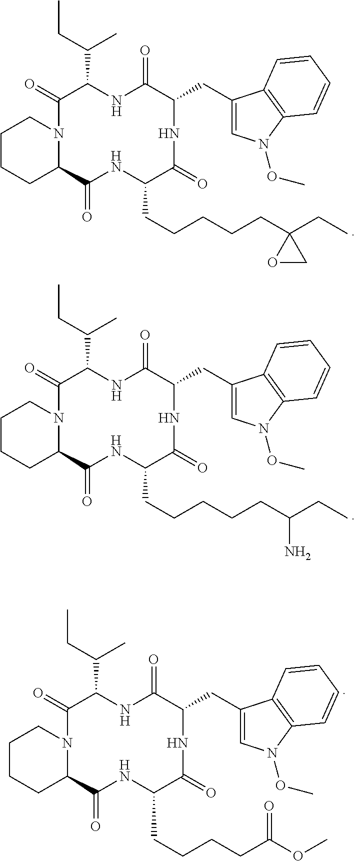 [SMILES: CCC(C)[C@@H]1NC(=O)[C@H](Cc2cn(OC)c3ccccc23)NC(=O)[C@H](CCCCCC2(CC)CO2)NC(=O)[C@H]2CCCCN2C1=O ; CCC(N)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCC(=O)OC)NC(=O)[C@H]2CCCCN2C1=O]

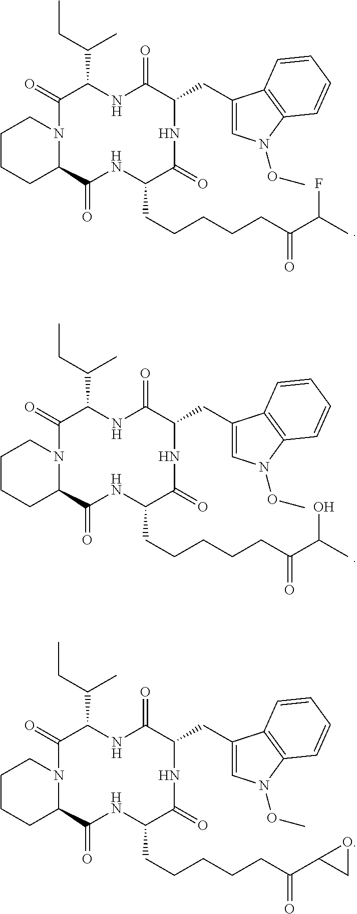 [SMILES: CCC(C)[C@@H]1NC(=O)[C@H](Cc2cn(OC)c3ccccc23)NC(=O)[C@H](CCCCCC(=O)C(C)F)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)C2CO2)NC(=O)[C@H]2CCCCN2C1=O]

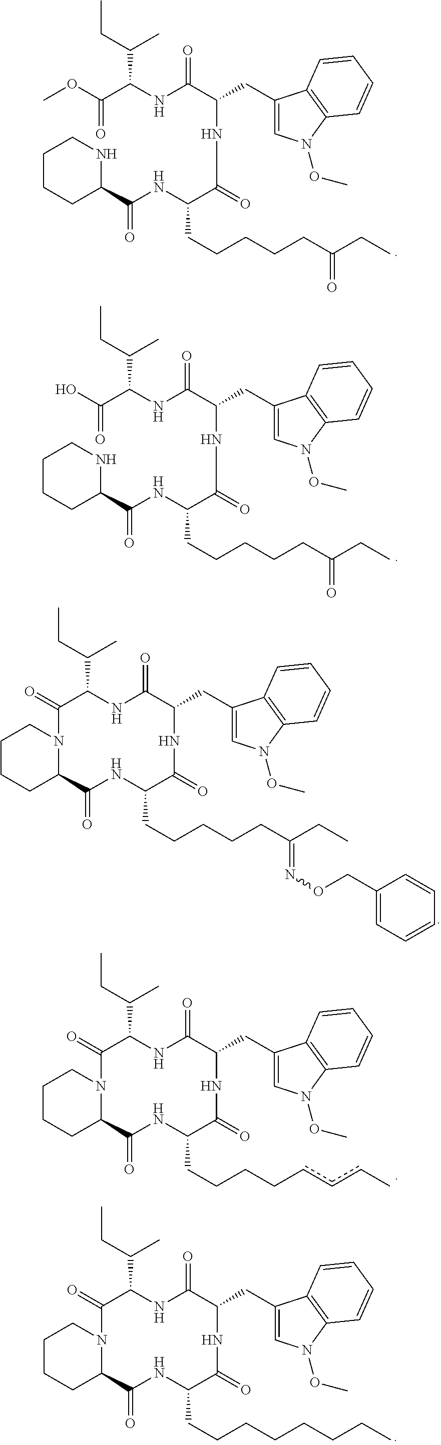

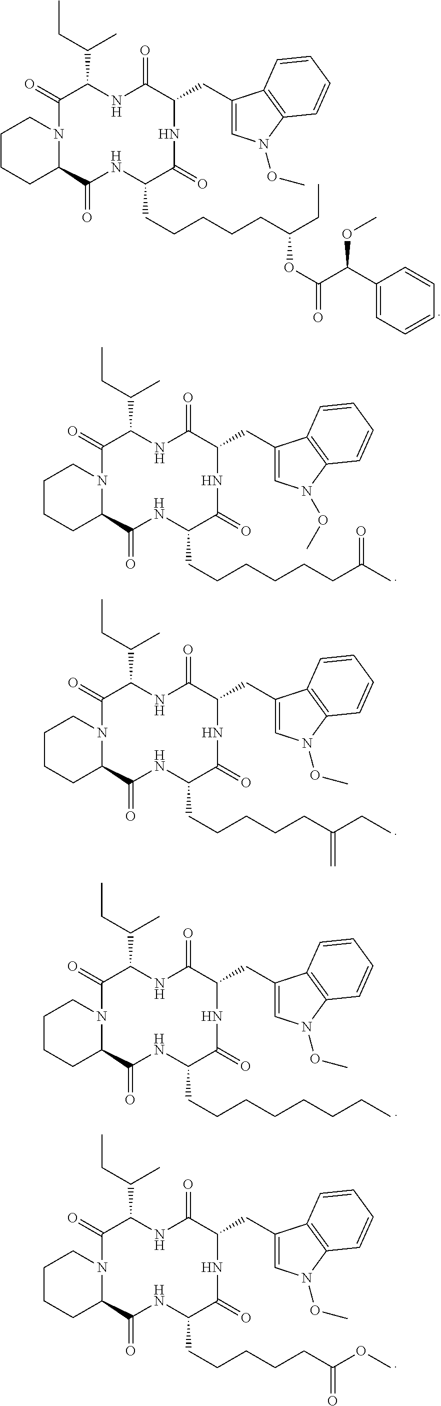

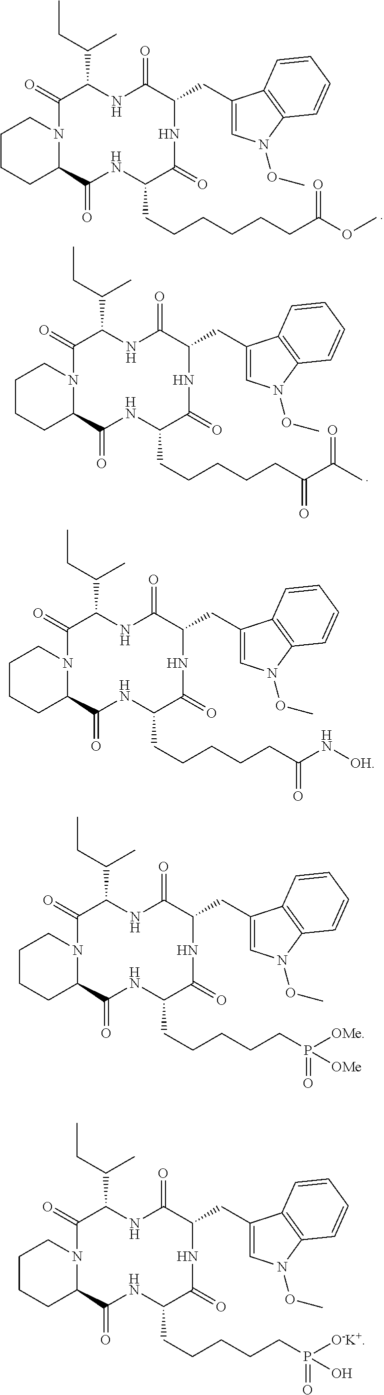

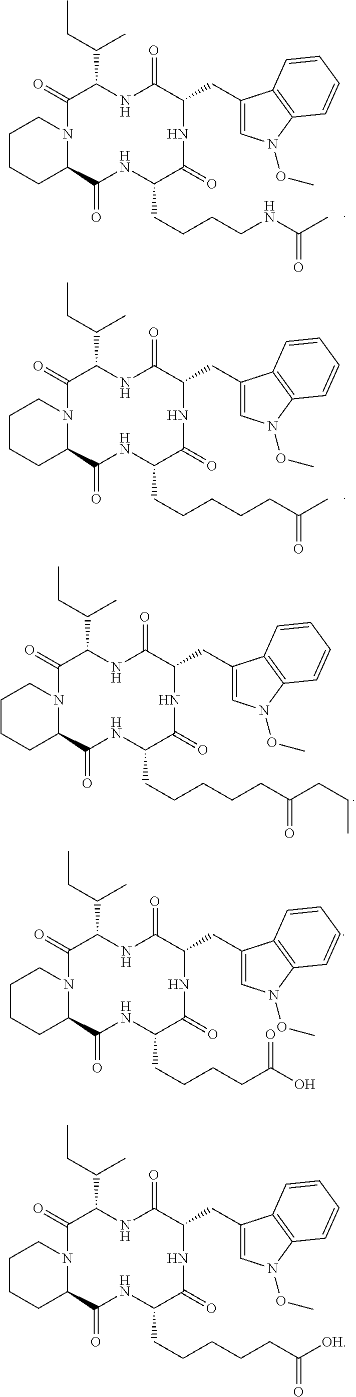 [SMILES: CCC(C)[C@@H]1NC(=O)[C@H](Cc2cn(OC)c3ccccc23)NC(=O)[C@H](CCCCNC(C)=O)NC(=O)[C@H]2CCCCN2C1=O ; CCC(C)[C@@H]1NC(=O)[C@H](Cc2cn(OC)c3ccccc23)NC(=O)[C@H](CCCCCC(C)=O)NC(=O)[C@H]2CCCCN2C1=O ; CCCC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCC(=O)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)O)NC(=O)[C@H]2CCCCN2C1=O]

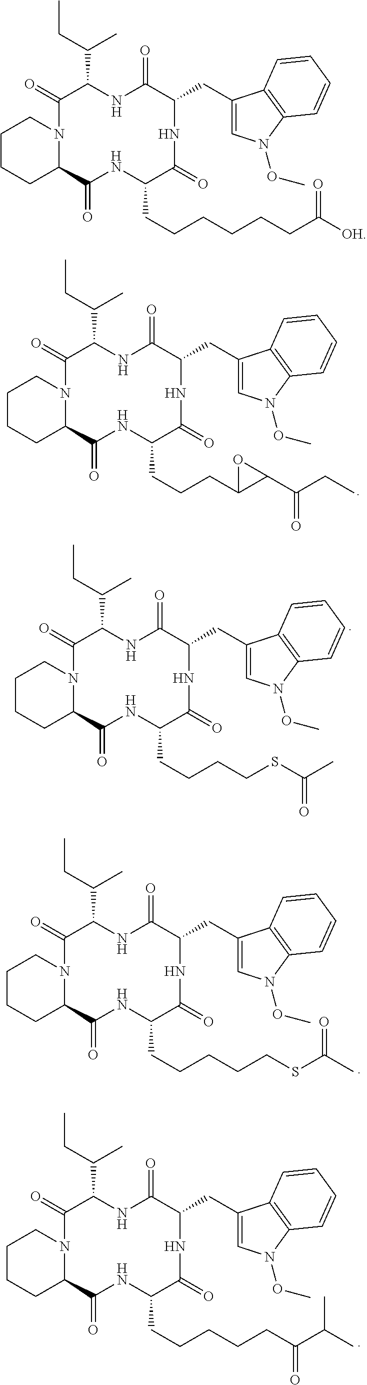 [SMILES: CCC(C)[C@@H]1NC(=O)[C@H](Cc2cn(OC)c3ccccc23)NC(=O)[C@H](CCCCCCC(=O)O)NC(=O)[C@H]2CCCCN2C1=O ; CCC(=O)C1OC1CCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCSC(C)=O)NC(=O)[C@H]2CCCCN2C1=O ; CCC(C)[C@@H]1NC(=O)[C@H](Cc2cn(OC)c3ccccc23)NC(=O)[C@H](CCCCCSC(C)=O)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)C)NC(=O)[C@H]2CCCCN2C1=O]

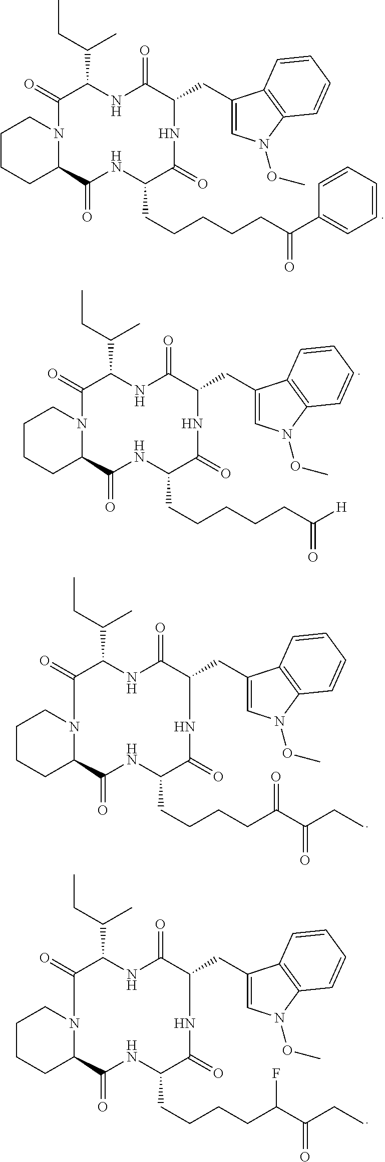 [SMILES: CCC(C)[C@@H]1NC(=O)[C@H](Cc2cn(OC)c3ccccc23)NC(=O)[C@H](CCCCCC(=O)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCCC=O)NC(=O)[C@H]2CCCCN2C1=O ; CCC(=O)C(=O)CCCC[C@@H]1NC(=O)[C@H]2CCCCN2C(=O)[C@H](C(C)CC)NC(=O)[C@H](Cc2cn(OC)c3ccccc23)NC1=O ; CCC(=O)C(F)CCCC[C@@H]1NC(=O)[C@H]2CCCCN2C(=O)[C@H](C(C)CC)NC(=O)[C@H](Cc2cn(OC)c3ccccc23)NC1=O]

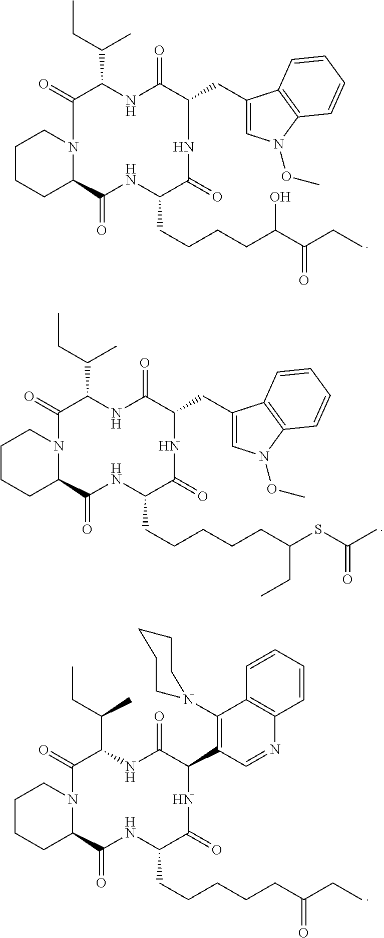 [SMILES: CCC(=O)C(O)CCCC[C@@H]1NC(=O)[C@H]2CCCCN2C(=O)[C@H](C(C)CC)NC(=O)[C@H](Cc2cn(OC)c3ccccc23)NC1=O ; CCC(CCCCC[C@@H]1NC(=O)[C@H]2CCCCN2C(=O)[C@H](C(C)CC)NC(=O)[C@H](Cc2cn(OC)c3ccccc23)NC1=O)SC(C)=O ; CCC(=O)CCCCC[C@@H]1NC(=O)[C@H]2CCCCN2C(=O)[C@H]([C@H](C)CC)NC(=O)[C@@H](c2cnc3ccccc3c2N2CCCCC2)NC1=O]

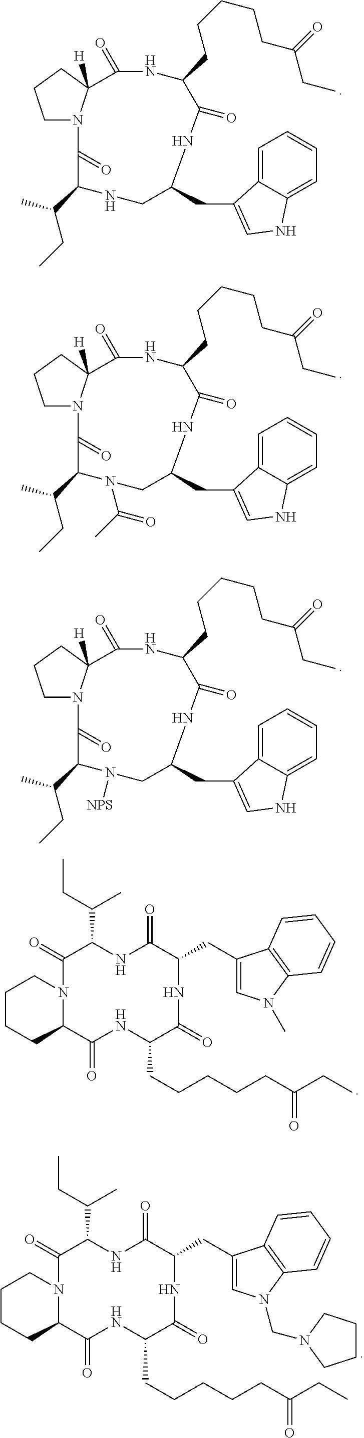

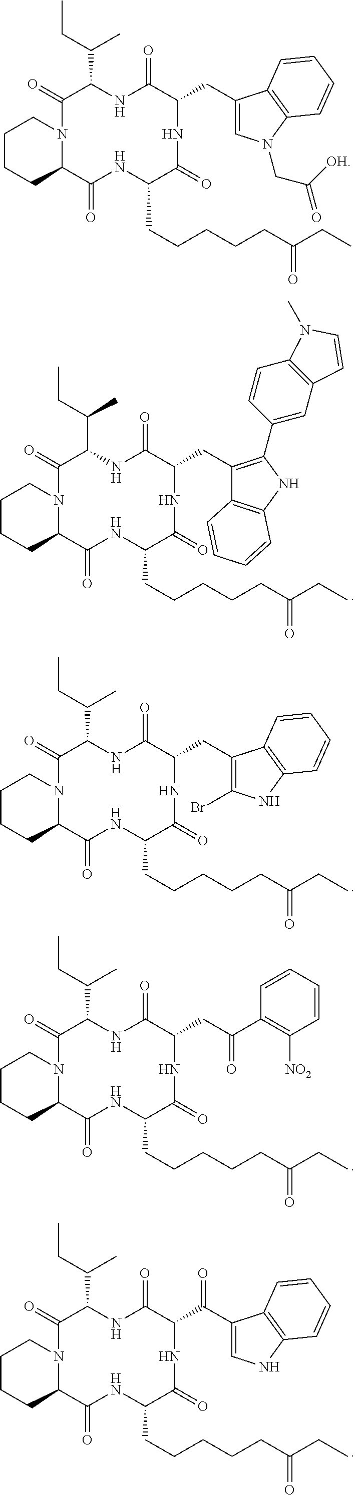

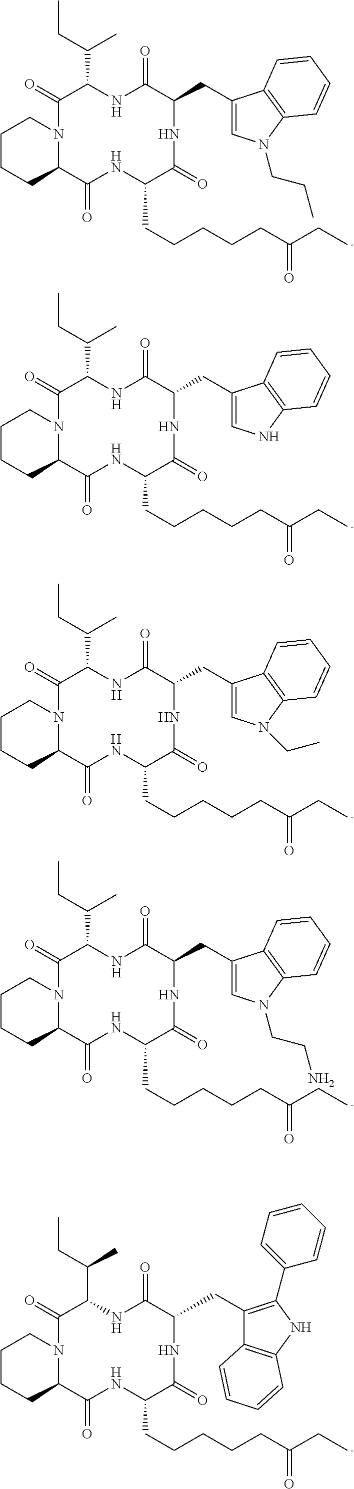 [SMILES: CCCn1cc(C[C@H]2NC(=O)[C@H](CCCCCC(=O)CC)NC(=O)[C@H]3CCCCN3C(=O)[C@H](C(C)CC)NC2=O)c2ccccc21 ; CCC(=O)CCCCC[C@@H]1NC(=O)[C@H]2CCCCN2C(=O)[C@H](C(C)CC)NC(=O)[C@H](Cc2c[nH]c3ccccc23)NC1=O ; CCC(=O)CCCCC[C@@H]1NC(=O)[C@H]2CCCCN2C(=O)[C@H](C(C)CC)NC(=O)[C@H](Cc2cn(CC)c3ccccc23)NC1=O ; CCC(=O)CCCCC[C@@H]1NC(=O)[C@H]2CCCCN2C(=O)[C@H](C(C)CC)NC(=O)[C@@H](Cc2cn(CCN)c3ccccc23)NC1=O ; CCC(=O)CCCCC[C@@H]1NC(=O)[C@H]2CCCCN2C(=O)[C@H]([C@H](C)CC)NC(=O)[C@H](Cc2c(-c3ccccc3)[nH]c3ccccc23)NC1=O]

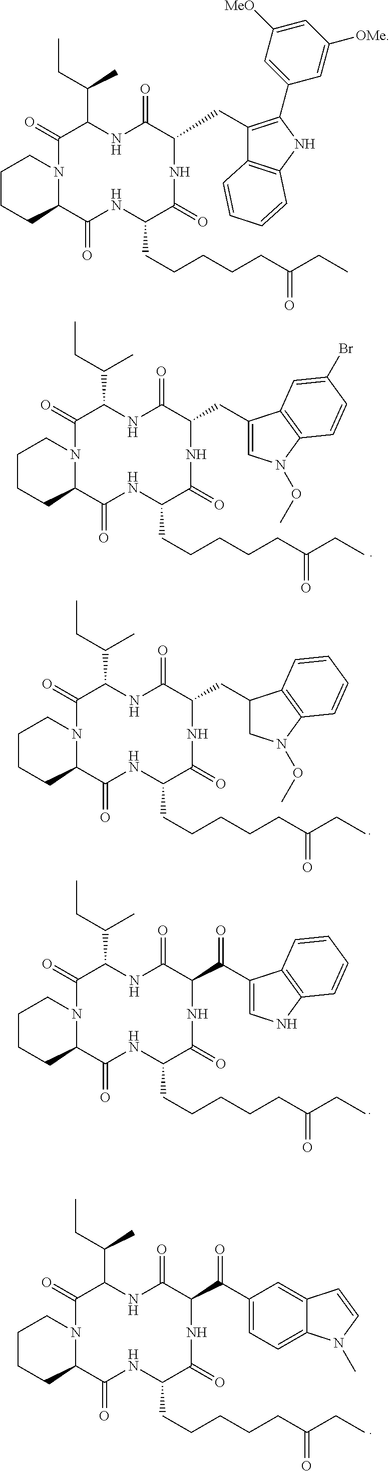 [SMILES: CCC(=O)CCCCC[C@@H]1NC(=O)[C@H]2CCCCN2C(=O)C([C@H](C)CC)NC(=O)[C@H](Cc2c(-c3cc(OC)cc(OC)c3)[nH]c3ccccc23)NC1=O ; CCC(=O)CCCCC[C@@H]1NC(=O)[C@H]2CCCCN2C(=O)[C@H](C(C)CC)NC(=O)[C@H](Cc2cn(OC)c3ccc(Br)cc23)NC1=O ; CCC(=O)CCCCC[C@@H]1NC(=O)[C@H]2CCCCN2C(=O)[C@H](C(C)CC)NC(=O)[C@H](CC2CN(OC)c3ccccc32)NC1=O ; CCC(=O)CCCCC[C@@H]1NC(=O)[C@H]2CCCCN2C(=O)[C@H](C(C)CC)NC(=O)[C@@H](C(=O)c2c[nH]c3ccccc23)NC1=O ; CCC(=O)CCCCC[C@@H]1NC(=O)[C@H]2CCCCN2C(=O)C([C@H](C)CC)NC(=O)[C@@H](C(=O)c2ccc3c(ccn3C)c2)NC1=O]

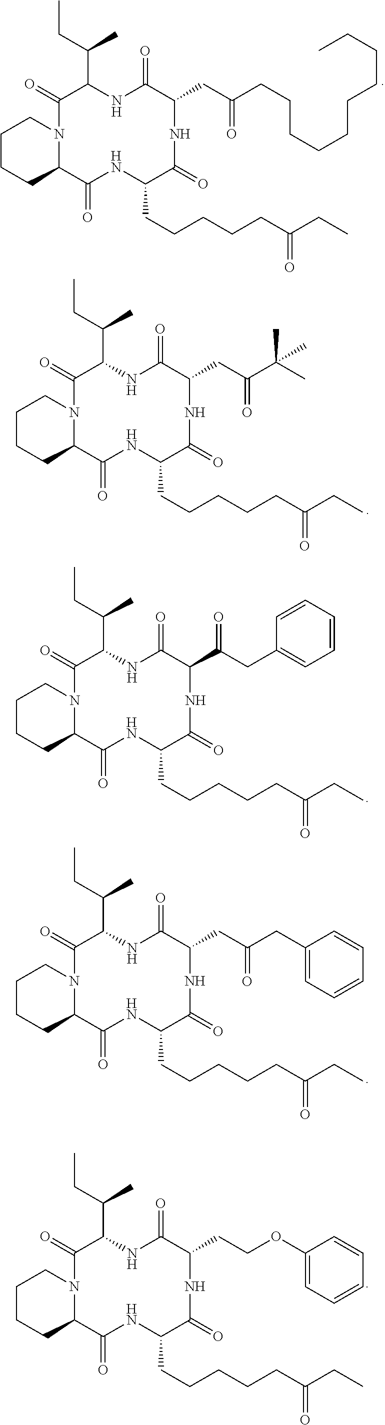

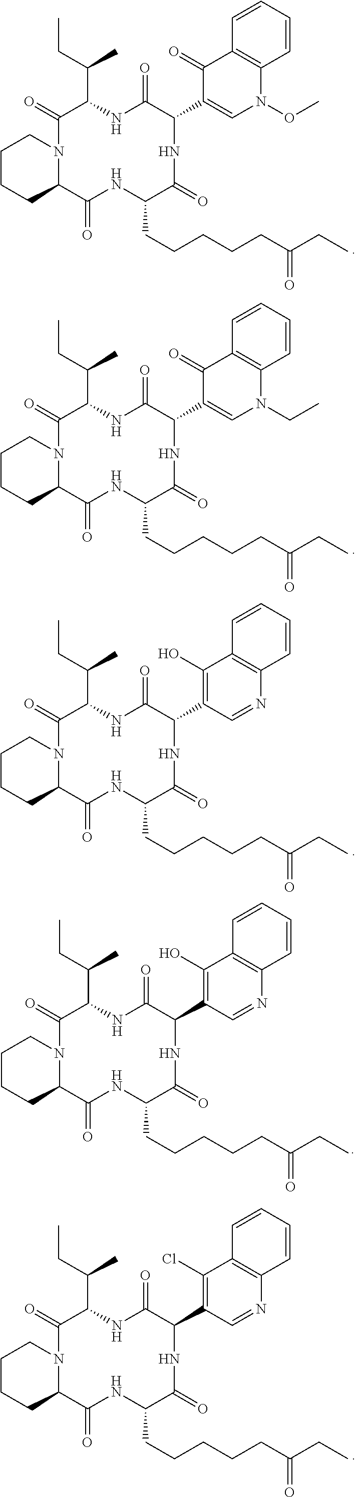

11. The pharmaceutical composition of claim 8, further comprising an antibiotic.

12. The pharmaceutical composition of claim 8, wherein the pharmaceutical composition is a pharmaceutically acceptable topical formulation.

13. The pharmaceutical composition of claim 8, wherein the SSTI is an abscess, furuncles, or cellulitis.

14. A method of treating a skin and soft tissue infection (SSTI) comprising:
administering to a subject in need thereof a therapeutically effective amount of apicidin or a pharmaceutically acceptable derivative thereof, and a pharmaceutically acceptable carrier.

15. The method of claim 14, wherein the apicidin has a structure selected from:

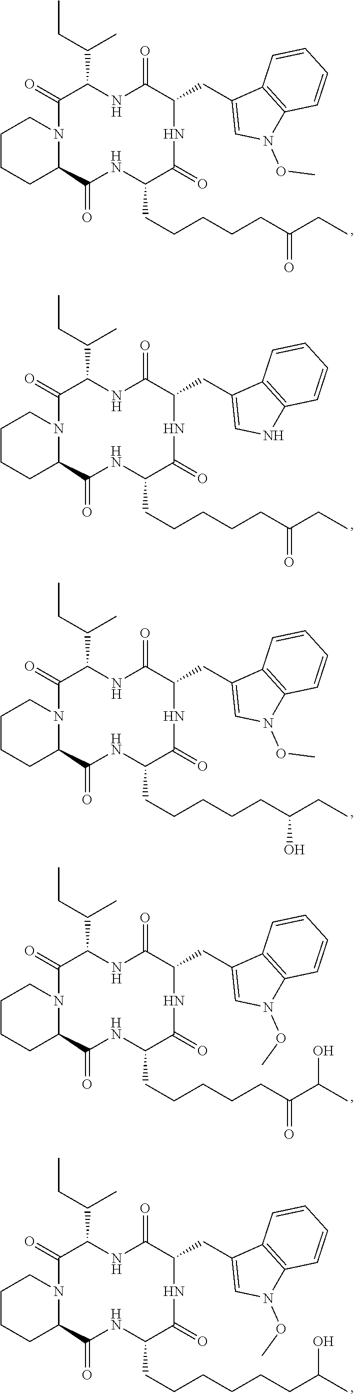 [SMILES: CCC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(=O)CCCCC[C@@H]1NC(=O)[C@H]2CCCCN2C(=O)[C@H](C(C)CC)NC(=O)[C@H](Cc2c[nH]c3ccccc23)NC1=O ; CCC(C)[C@@H]1NC(=O)[C@H](Cc2cn(OC)c3ccccc23)NC(=O)[C@H](CCCCC[C@@H](O)CC)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCC(C)[C@@H]1NC(=O)[C@H](Cc2cn(OC)c3ccccc23)NC(=O)[C@H](CCCCCCC(C)O)NC(=O)[C@H]2CCCCN2C1=O]

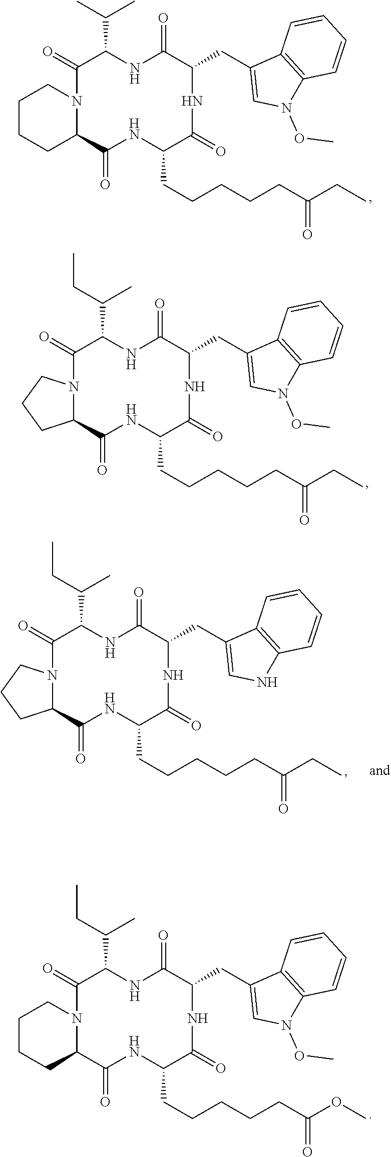 [SMILES: CCC(=O)CCCCC[C@@H]1NC(=O)[C@H]2CCCCN2C(=O)[C@H](C(C)C)NC(=O)[C@H](Cc2cn(OC)c3ccccc23)NC1=O ; CCC(=O)CCCCC[C@@H]1NC(=O)[C@H]2CCCN2C(=O)[C@H](C(C)CC)NC(=O)[C@H](Cc2cn(OC)c3ccccc23)NC1=O ; CCC(=O)CCCCC[C@@H]1NC(=O)[C@H]2CCCN2C(=O)[C@H](C(C)CC)NC(=O)[C@H](Cc2c[nH]c3ccccc23)NC1=O ; CCC(C)[C@@H]1NC(=O)[C@H](Cc2cn(OC)c3ccccc23)NC(=O)[C@H](CCCCCC(=O)OC)NC(=O)[C@H]2CCCCN2C1=O]

16. The method of claim 14, wherein the apicidin has a structure selected from:

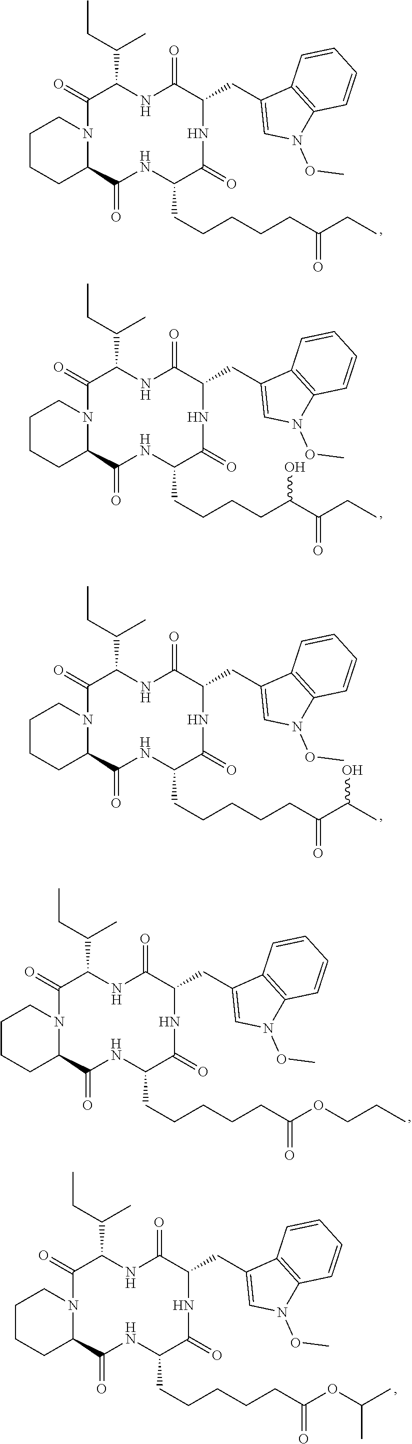 [SMILES: CCC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(=O)C(O)CCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCCOC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)OC(C)C)NC(=O)[C@H]2CCCCN2C1=O]

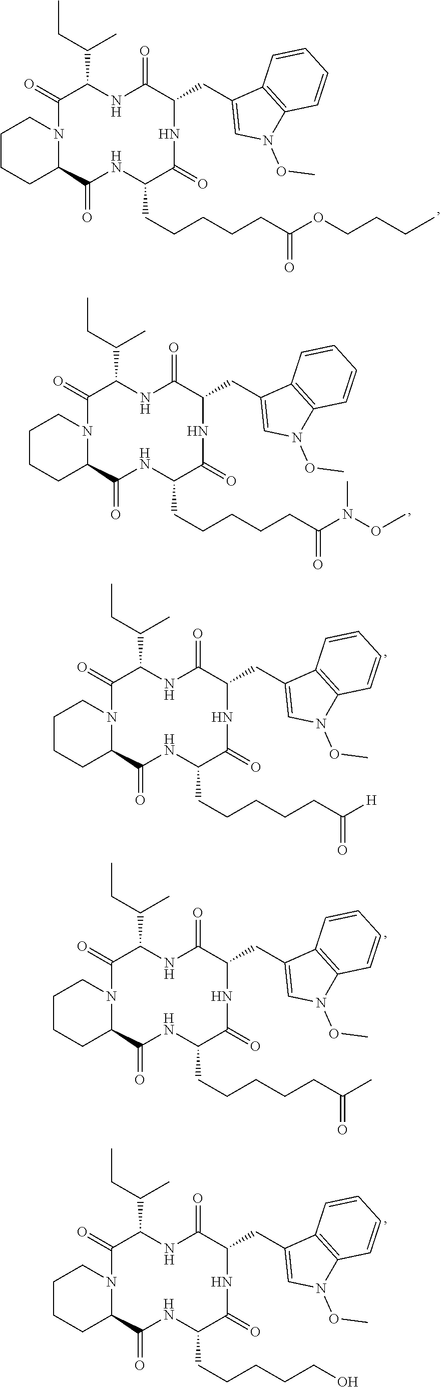 [SMILES: CCCCOC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)N(C)OC)NC(=O)[C@H]2CCCCN2C1=O ; CCC(C)[C@@H]1NC(=O)[C@H](Cc2cn(OC)c3ccccc23)NC(=O)[C@H](CCCCCC=O)NC(=O)[C@H]2CCCCN2C1=O ; CCC(C)[C@@H]1NC(=O)[C@H](Cc2cn(OC)c3ccccc23)NC(=O)[C@H](CCCCCC(C)=O)NC(=O)[C@H]2CCCCN2C1=O ; CCC(C)[C@@H]1NC(=O)[C@H](Cc2cn(OC)c3ccccc23)NC(=O)[C@H](CCCCCO)NC(=O)[C@H]2CCCCN2C1=O]

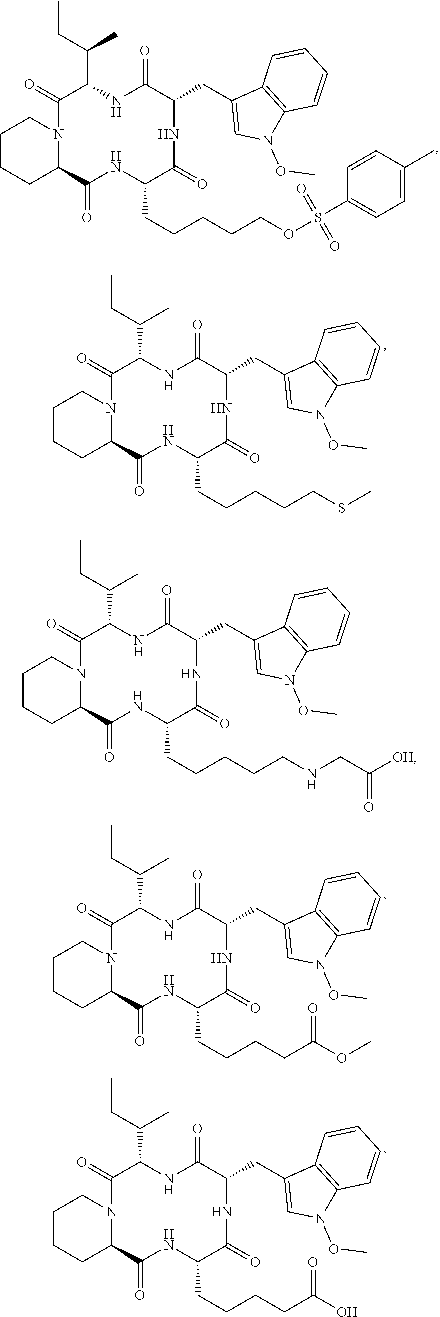 [SMILES: CC[C@@H](C)[C@@H]1NC(=O)[C@H](Cc2cn(OC)c3ccccc23)NC(=O)[C@H](CCCCCOS(=O)(=O)c2ccc(C)cc2)NC(=O)[C@H]2CCCCN2C1=O ; CCC(C)[C@@H]1NC(=O)[C@H](Cc2cn(OC)c3ccccc23)NC(=O)[C@H](CCCCCSC)NC(=O)[C@H]2CCCCN2C1=O ; CCC(C)[C@@H]1NC(=O)[C@H](Cc2cn(OC)c3ccccc23)NC(=O)[C@H](CCCCCNCC(=O)O)NC(=O)[C@H]2CCCCN2C1=O ; CCC(C)[C@@H]1NC(=O)[C@H](Cc2cn(OC)c3ccccc23)NC(=O)[C@H](CCCCC(=O)OC)NC(=O)[C@H]2CCCCN2C1=O ; CCC(C)[C@@H]1NC(=O)[C@H](Cc2cn(OC)c3ccccc23)NC(=O)[C@H](CCCCC(=O)O)NC(=O)[C@H]2CCCCN2C1=O]

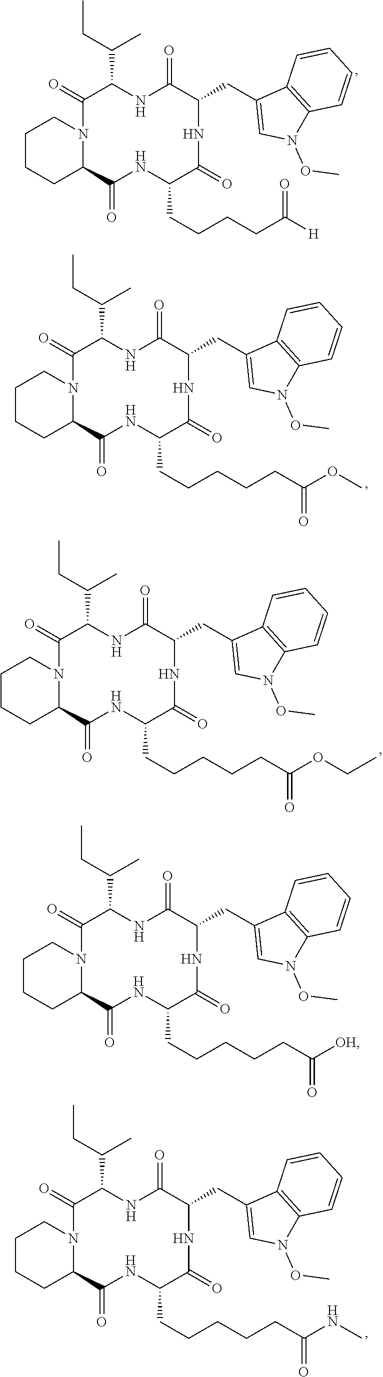 [SMILES: CCC(C)[C@@H]1NC(=O)[C@H](Cc2cn(OC)c3ccccc23)NC(=O)[C@H](CCCCC=O)NC(=O)[C@H]2CCCCN2C1=O ; CCC(C)[C@@H]1NC(=O)[C@H](Cc2cn(OC)c3ccccc23)NC(=O)[C@H](CCCCCC(=O)OC)NC(=O)[C@H]2CCCCN2C1=O ; CCOC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)NC)NC(=O)[C@H]2CCCCN2C1=O]

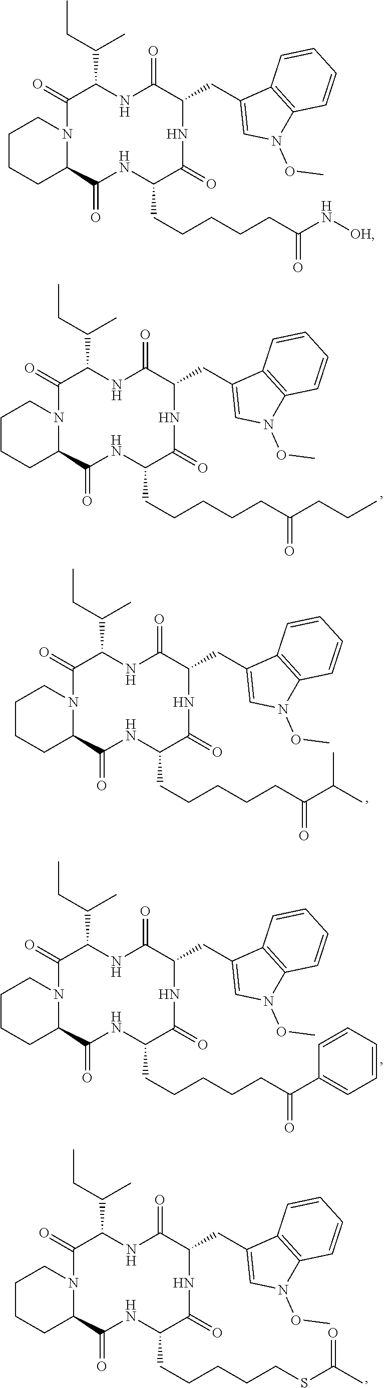 [SMILES: CCC(C)[C@@H]1NC(=O)[C@H](Cc2cn(OC)c3ccccc23)NC(=O)[C@H](CCCCCC(=O)NO)NC(=O)[C@H]2CCCCN2C1=O ; CCCC(=O)CCCCC[C@@H]1NC(=O)[C@H]2CCCCN2C(=O)[C@H](C(C)CC)NC(=O)[C@H](Cc2cn(OC)c3ccccc23)NC1=O ; "," ; CCC(C)[C@@H]1NC(=O)[C@H](Cc2cn(OC)c3ccccc23)NC(=O)[C@H](CCCCCC(=O)C(C)C)NC(=O)[C@H]2CCCCN2C1=O ; CCC(C)[C@@H]1NC(=O)[C@H](Cc2cn(OC)c3ccccc23)NC(=O)[C@H](CCCCCC(=O)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCCSC(C)=O)NC(=O)[C@H]2CCCCN2C1=O]

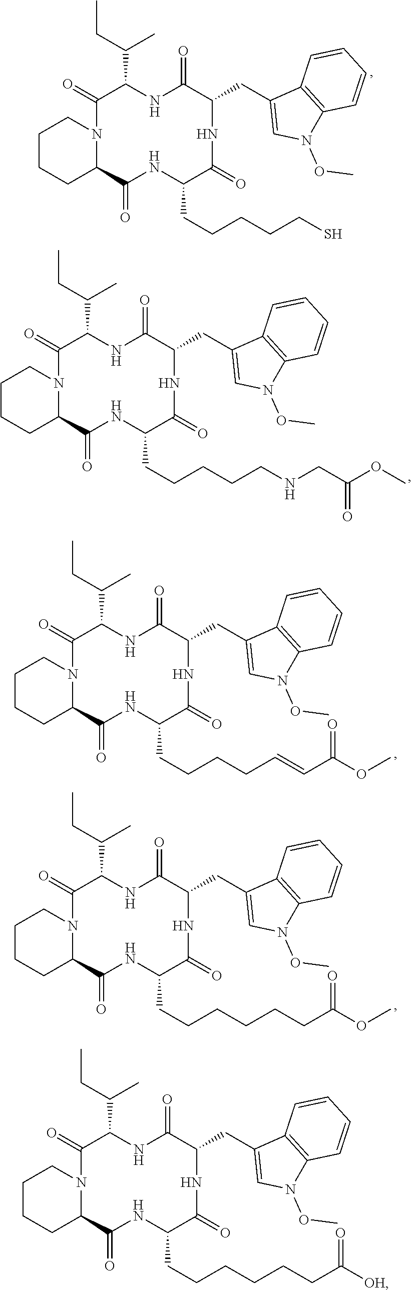

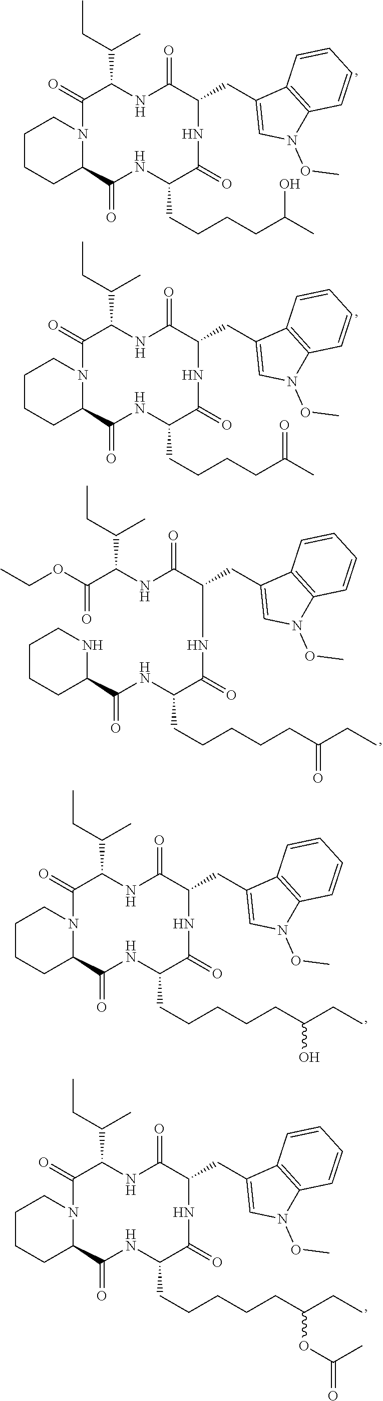

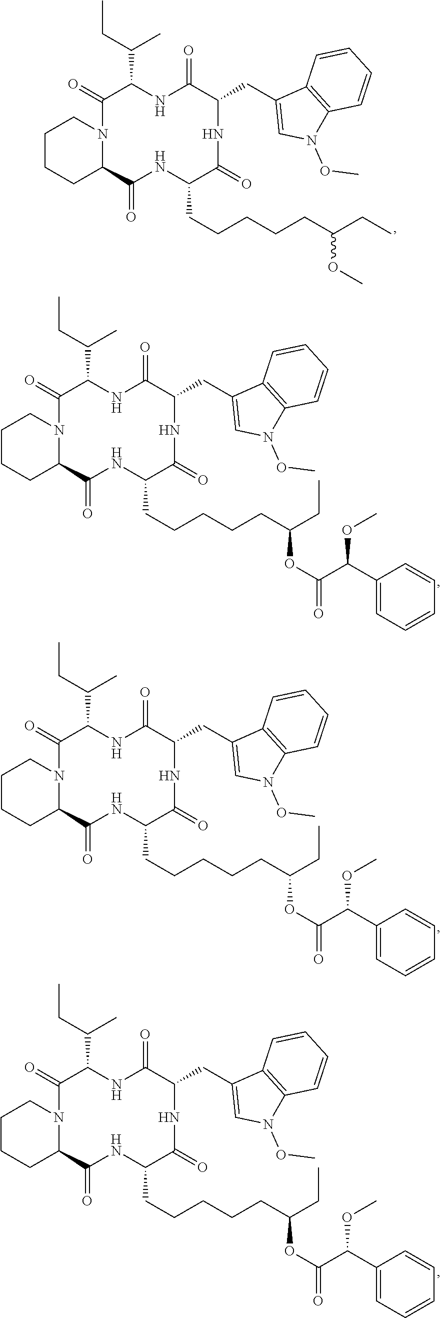 [SMILES: CCC(CCCCC[C@@H]1NC(=O)[C@H]2CCCCN2C(=O)[C@H](C(C)CC)NC(=O)[C@H](Cc2cn(OC)c3ccccc23)NC1=O)OC ; CCC(C)[C@@H]1NC(=O)[C@H](Cc2cn(OC)c3ccccc23)NC(=O)[C@H](CCCCC[C@@H](CC)OC(=O)[C@@H](OC)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCC[C@H](CC)OC(=O)[C@H](OC)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCC[C@@H](CC)OC(=O)[C@H](OC)c2ccccc2)NC(=O)[C@H]2CCCCN2C1=O]

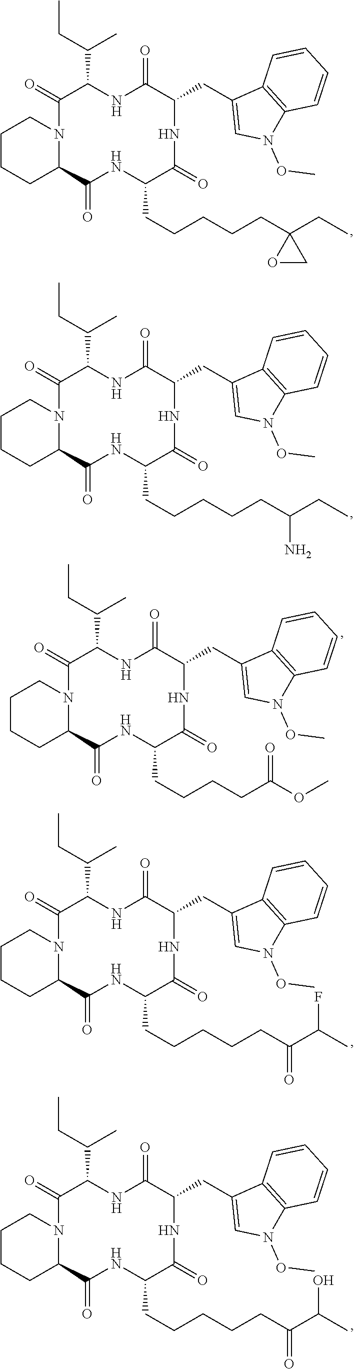 [SMILES: CCC(C)[C@@H]1NC(=O)[C@H](Cc2cn(OC)c3ccccc23)NC(=O)[C@H](CCCCCC2(CC)CO2)NC(=O)[C@H]2CCCCN2C1=O ; CCC(N)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCC(=O)OC)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)F)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O]

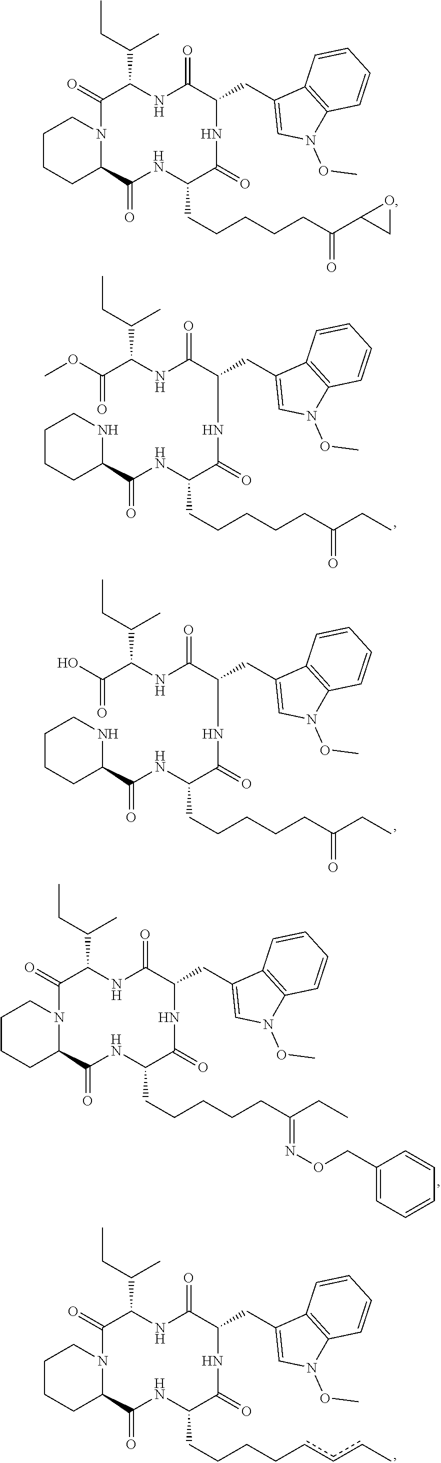

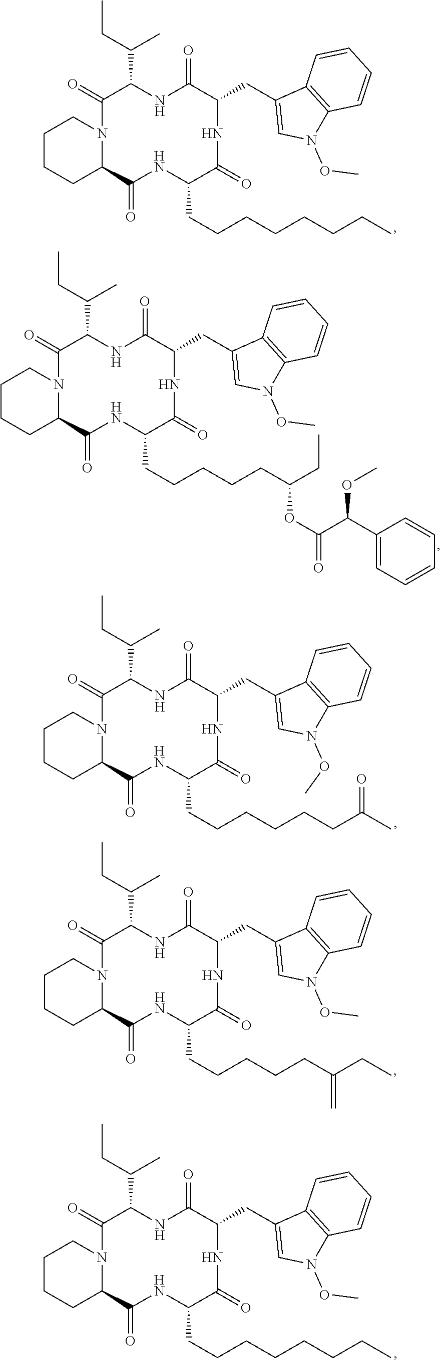

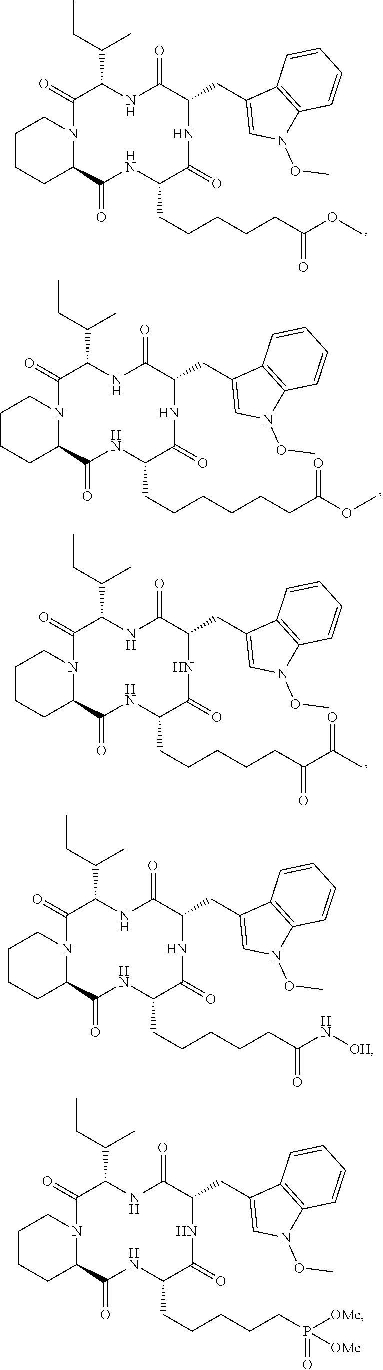

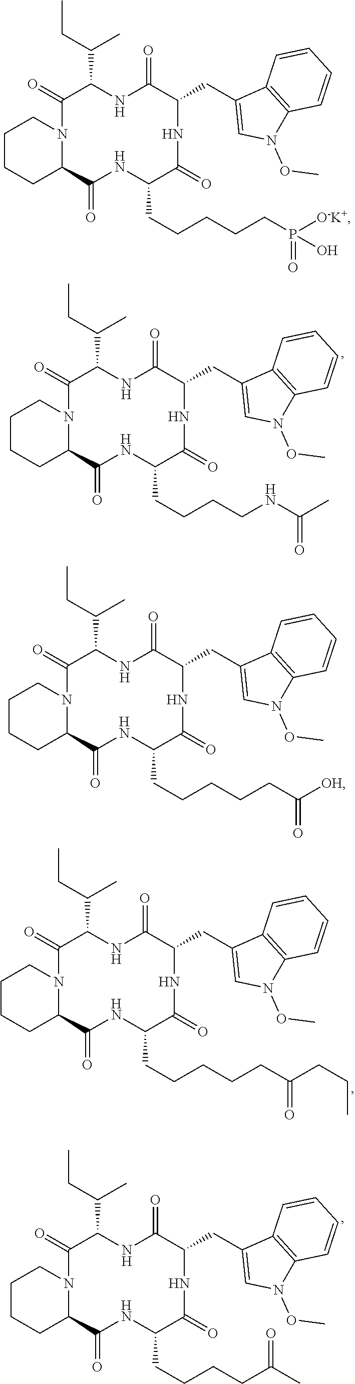

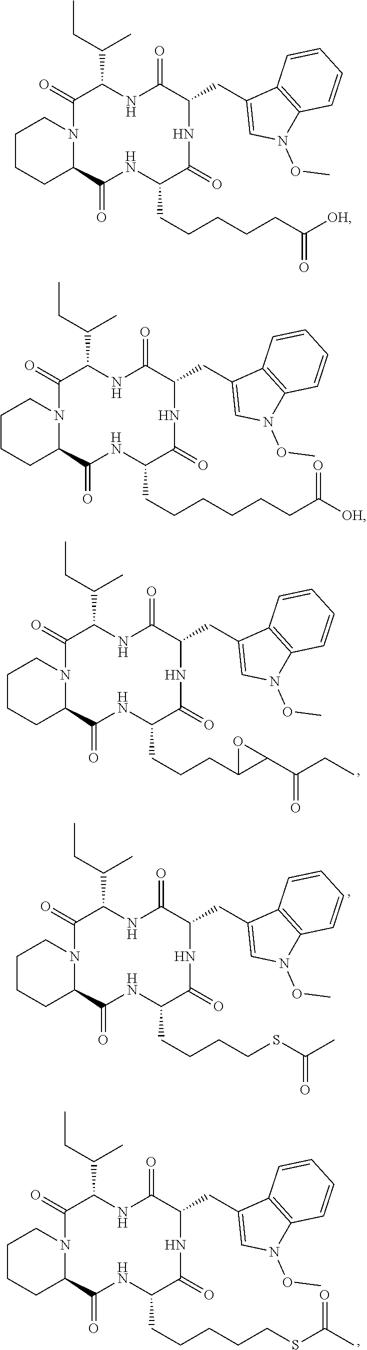 [SMILES: CCC(C)[C@@H]1NC(=O)[C@H](Cc2cn(OC)c3ccccc23)NC(=O)[C@H](CCCCCC(=O)O)NC(=O)[C@H]2CCCCN2C1=O ; CCC(C)[C@@H]1NC(=O)[C@H](Cc2cn(OC)c3ccccc23)NC(=O)[C@H](CCCCCCC(=O)O)NC(=O)[C@H]2CCCCN2C1=O ; CCC(=O)C1OC1CCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCSC(C)=O)NC(=O)[C@H]2CCCCN2C1=O ; CCC(C)[C@@H]1NC(=O)[C@H](Cc2cn(OC)c3ccccc23)NC(=O)[C@H](CCCCCSC(C)=O)NC(=O)[C@H]2CCCCN2C1=O]

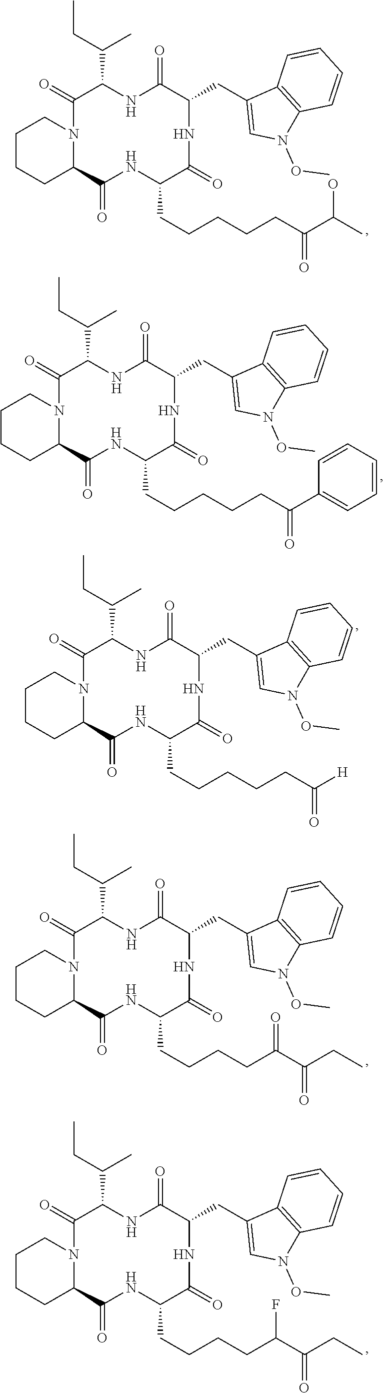 [SMILES: CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCCC=O)NC(=O)[C@H]2CCCCN2C1=O ; CCC(=O)C(=O)CCCC[C@@H]1NC(=O)[C@H]2CCCCN2C(=O)[C@H](C(C)CC)NC(=O)[C@H](Cc2cn(OC)c3ccccc23)NC1=O ; CCC(=O)C(F)CCCC[C@@H]1NC(=O)[C@H]2CCCCN2C(=O)[C@H](C(C)CC)NC(=O)[C@H](Cc2cn(OC)c3ccccc23)NC1=O]

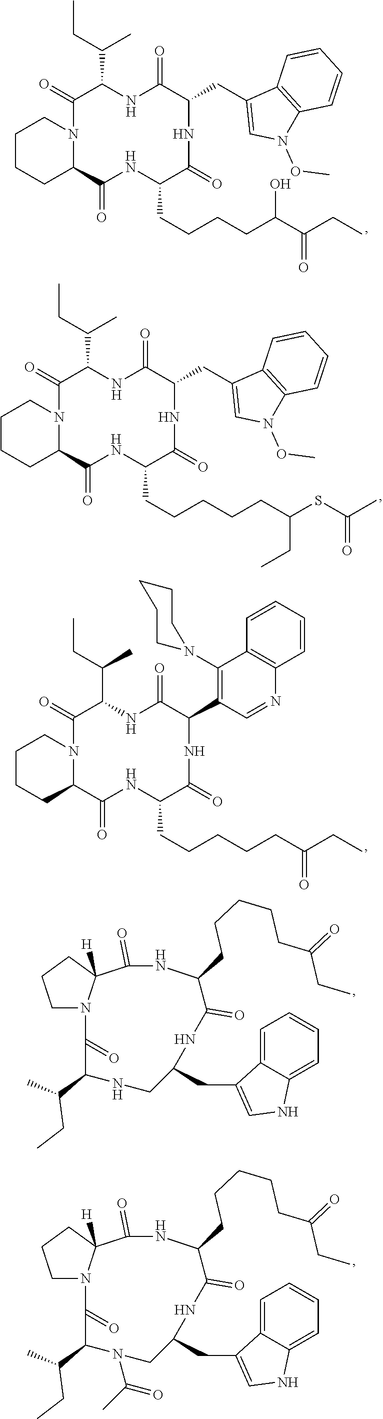

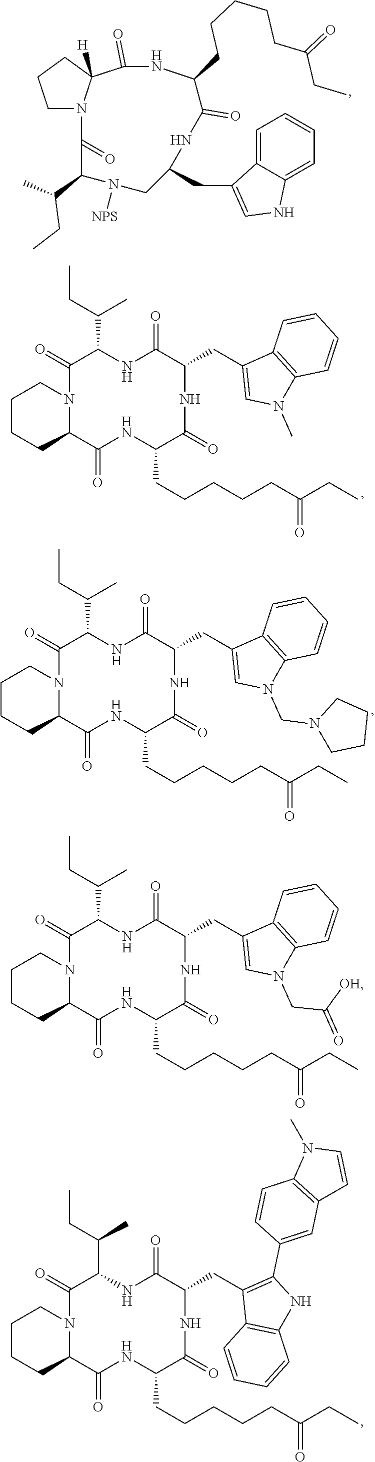

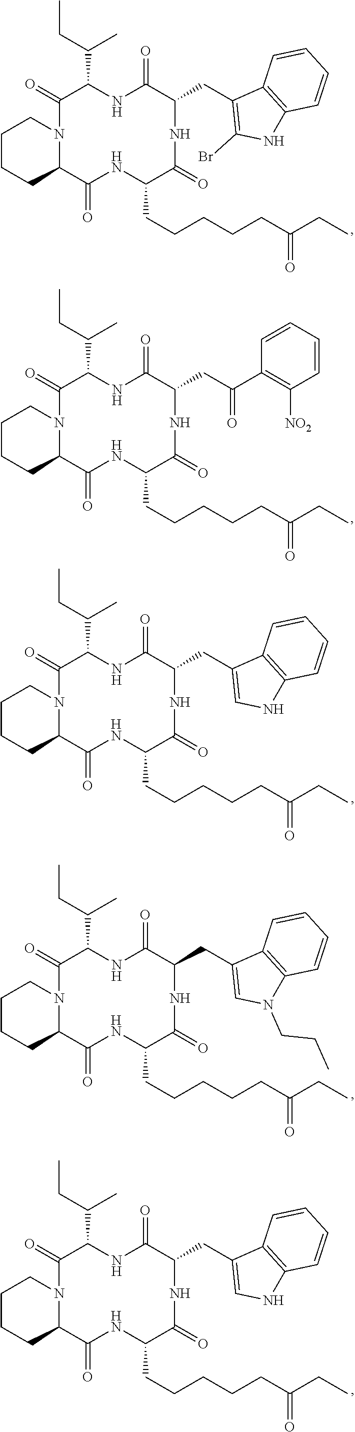 [SMILES: CCC(=O)CCCCC[C@@H]1NC(=O)[C@H]2CCCCN2C(=O)[C@H](C(C)CC)NC(=O)[C@H](Cc2c(Br)[nH]c3ccccc23)NC1=O ; CCC(=O)CCCCC[C@@H]1NC(=O)[C@H]2CCCCN2C(=O)[C@H](C(C)CC)NC(=O)[C@H](CC(=O)c2ccccc2[N+](=O)[O-])NC1=O ; CCC(=O)CCCCC[C@@H]1NC(=O)[C@H]2CCCCN2C(=O)[C@H](C(C)CC)NC(=O)[C@H](Cc2c[nH]c3ccccc23)NC1=O ; CCCn1cc(C[C@H]2NC(=O)[C@H](CCCCCC(=O)CC)NC(=O)[C@H]3CCCCN3C(=O)[C@H](C(C)CC)NC2=O)c2ccccc21 ; CCC(=O)CCCCC[C@@H]1NC(=O)[C@H]2CCCCN2C(=O)[C@H](C(C)CC)NC(=O)[C@H](Cc2c[nH]c3ccccc23)NC1=O]

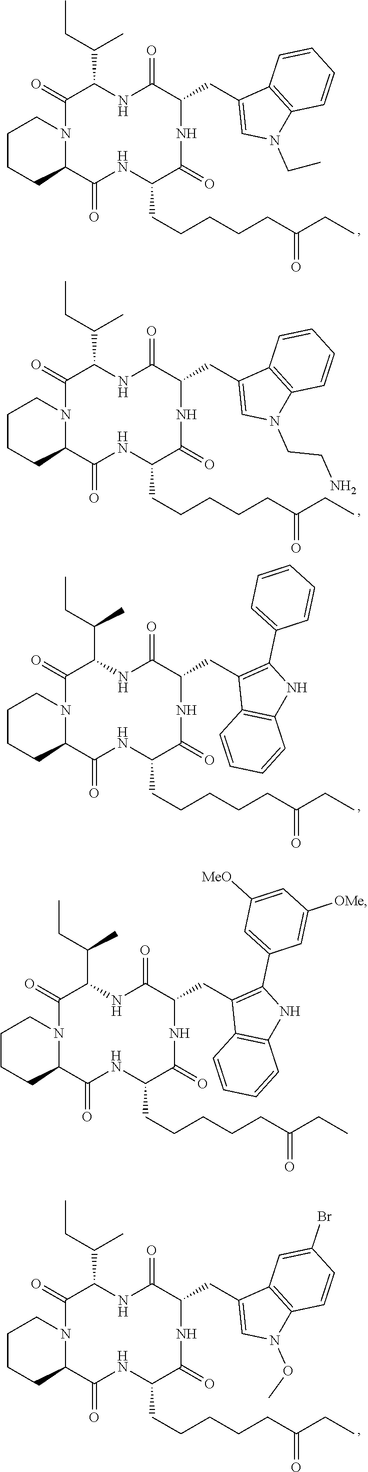

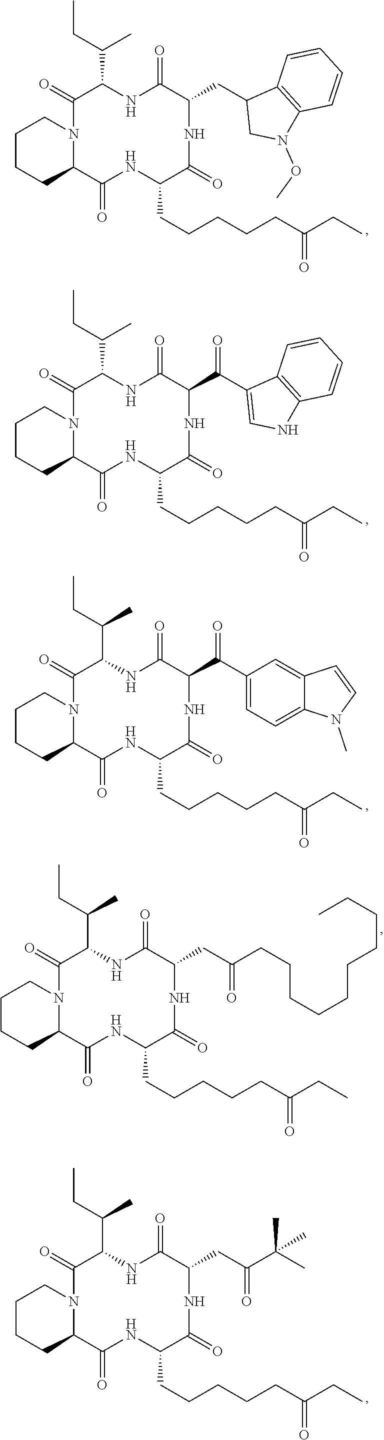

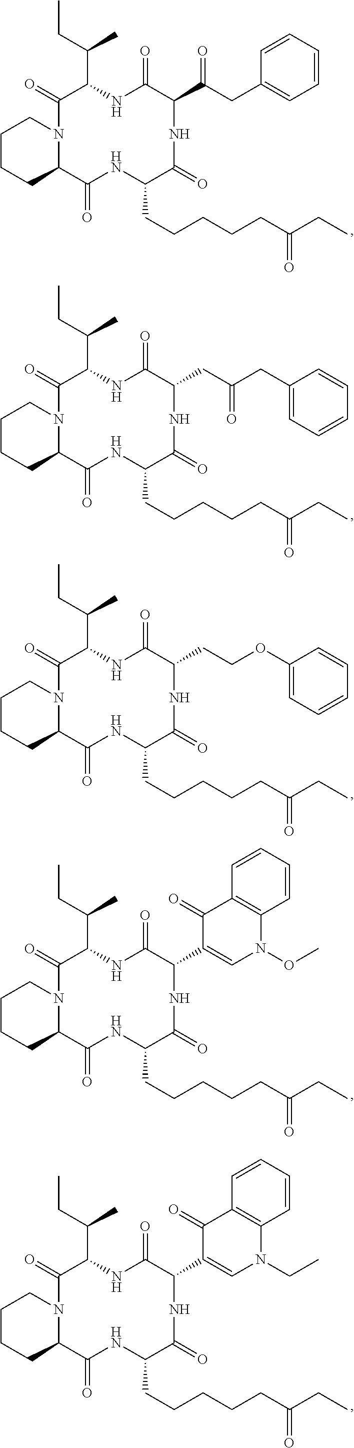

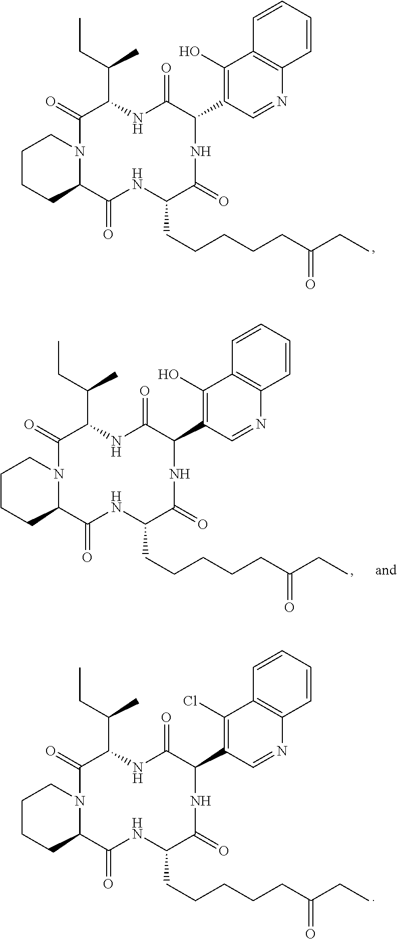

17. The method of claim 14, wherein the SSTI is an abscess, furuncles, or cellulitis.

18. The method of claim 14, wherein weight loss of the subject is reduced.

19. The method of claim 14, wherein dermonecrosis is reduced.

20. The method of claim 19, wherein a skin lesion is reduced in size.

21. The method of claim 14, wherein cutaneous bacterial burden is reduced.

22. The method of claim 14, wherein density of phagocytic polymorphonuclear neutrophils at a site of infection is increased.

23. The method of claim 14, wherein the administering step is topical administration.

24. A pharmaceutical composition comprising: apicidin, or a pharmaceutically acceptable derivative thereof, in an amount effective to treat a staphylococcal infection; and a pharmaceutically acceptable carrier.

25. The pharmaceutical composition of claim 24, wherein the apicidin has a structure selected from:

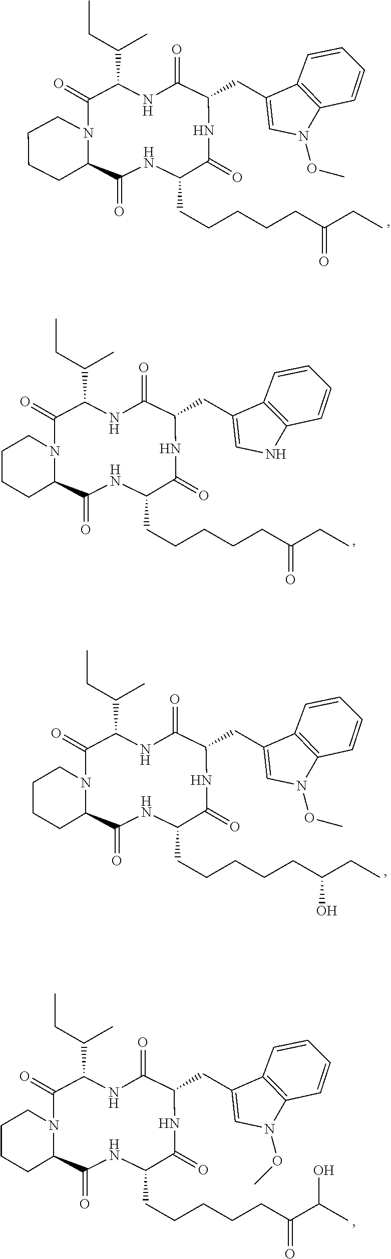 [SMILES: CCC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(=O)CCCCC[C@@H]1NC(=O)[C@H]2CCCCN2C(=O)[C@H](C(C)CC)NC(=O)[C@H](Cc2c[nH]c3ccccc23)NC1=O ; CCC(C)[C@@H]1NC(=O)[C@H](Cc2cn(OC)c3ccccc23)NC(=O)[C@H](CCCCC[C@@H](O)CC)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O]

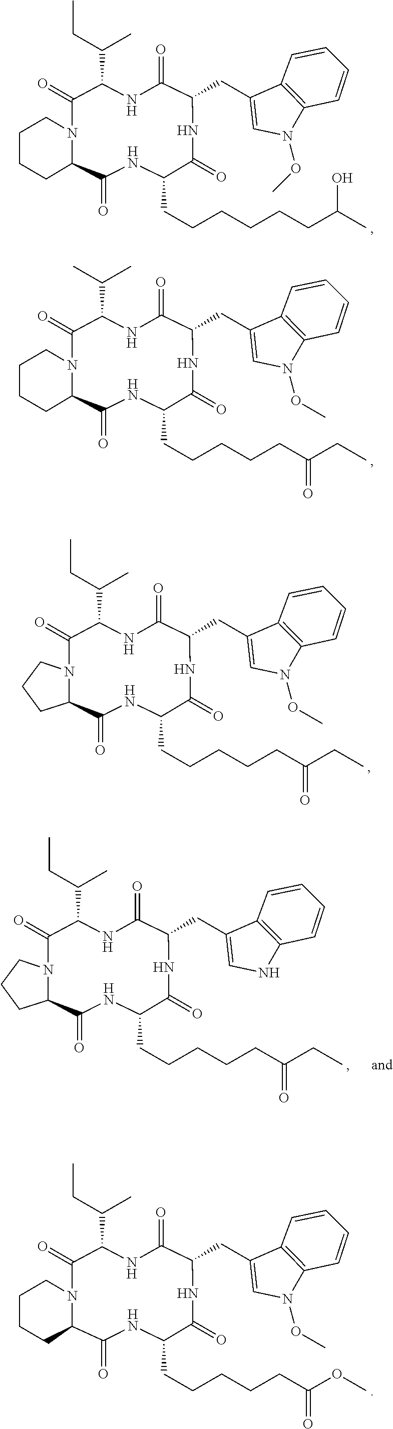 [SMILES: CCC(C)[C@@H]1NC(=O)[C@H](Cc2cn(OC)c3ccccc23)NC(=O)[C@H](CCCCCCC(C)O)NC(=O)[C@H]2CCCCN2C1=O ; CCC(=O)CCCCC[C@@H]1NC(=O)[C@H]2CCCCN2C(=O)[C@H](C(C)C)NC(=O)[C@H](Cc2cn(OC)c3ccccc23)NC1=O ; CCC(=O)CCCCC[C@@H]1NC(=O)[C@H]2CCCN2C(=O)[C@H](C(C)CC)NC(=O)[C@H](Cc2cn(OC)c3ccccc23)NC1=O ; CCC(=O)CCCCC[C@@H]1NC(=O)[C@H]2CCCN2C(=O)[C@H](C(C)CC)NC(=O)[C@H](Cc2c[nH]c3ccccc23)NC1=O ; CCC(C)[C@@H]1NC(=O)[C@H](Cc2cn(OC)c3ccccc23)NC(=O)[C@H](CCCCCC(=O)OC)NC(=O)[C@H]2CCCCN2C1=O]

25. The pharmaceutical composition of 23, wherein the apicidin has a structure selected from:

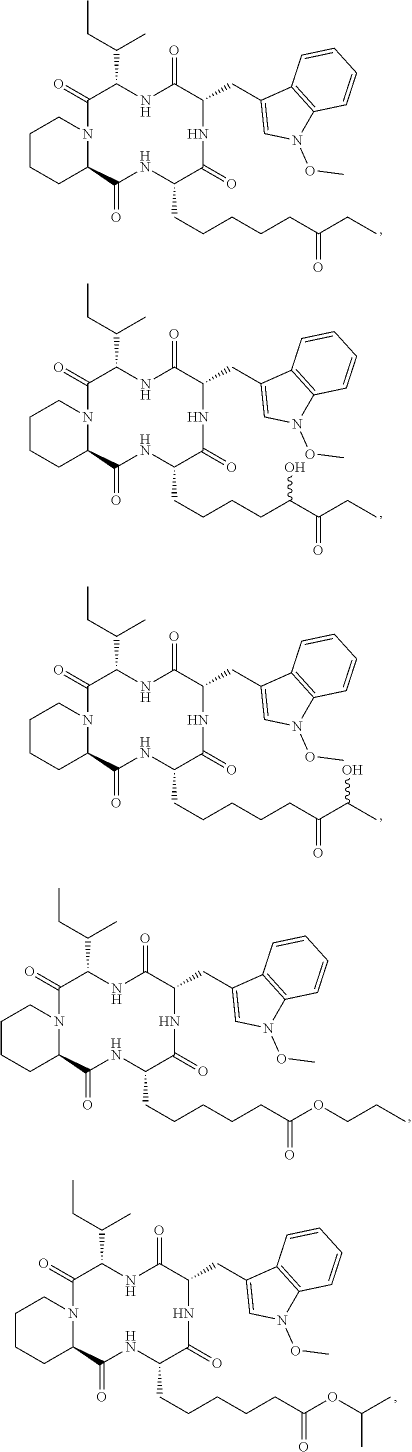 [SMILES: CCC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(=O)C(O)CCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCCOC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)OC(C)C)NC(=O)[C@H]2CCCCN2C1=O]

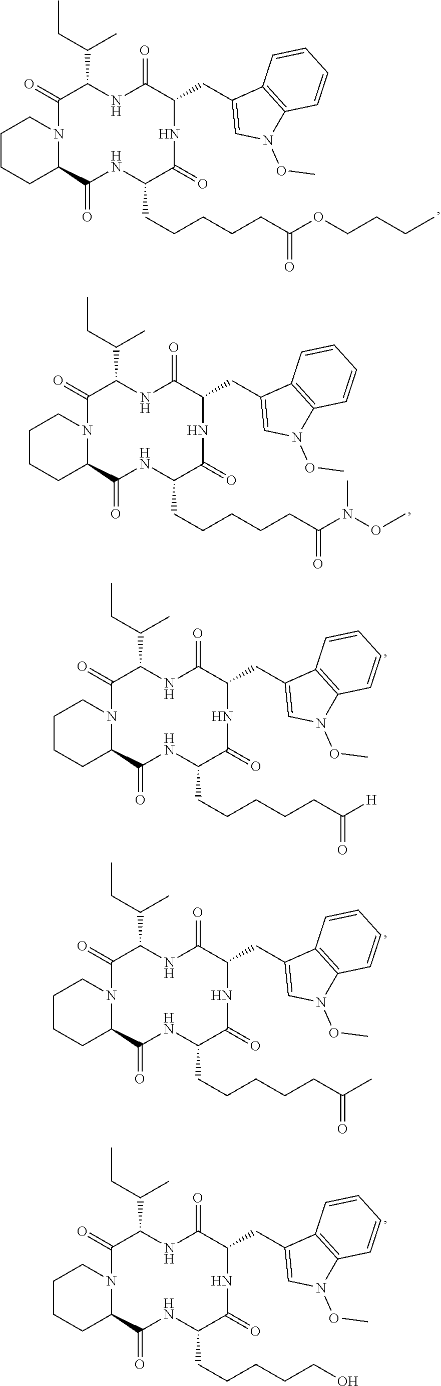 [SMILES: CCCCOC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)N(C)OC)NC(=O)[C@H]2CCCCN2C1=O ; CCC(C)[C@@H]1NC(=O)[C@H](Cc2cn(OC)c3ccccc23)NC(=O)[C@H](CCCCCC=O)NC(=O)[C@H]2CCCCN2C1=O ; CCC(C)[C@@H]1NC(=O)[C@H](Cc2cn(OC)c3ccccc23)NC(=O)[C@H](CCCCCC(C)=O)NC(=O)[C@H]2CCCCN2C1=O ; CCC(C)[C@@H]1NC(=O)[C@H](Cc2cn(OC)c3ccccc23)NC(=O)[C@H](CCCCCO)NC(=O)[C@H]2CCCCN2C1=O]

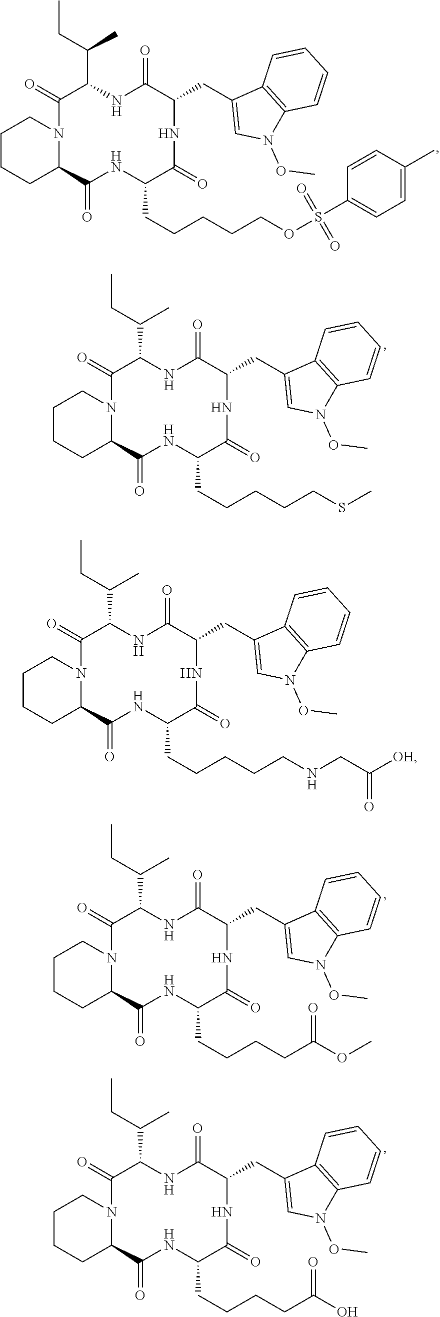 [SMILES: CC[C@@H](C)[C@@H]1NC(=O)[C@H](Cc2cn(OC)c3ccccc23)NC(=O)[C@H](CCCCCOS(=O)(=O)c2ccc(C)cc2)NC(=O)[C@H]2CCCCN2C1=O ; CCC(C)[C@@H]1NC(=O)[C@H](Cc2cn(OC)c3ccccc23)NC(=O)[C@H](CCCCCSC)NC(=O)[C@H]2CCCCN2C1=O ; CCC(C)[C@@H]1NC(=O)[C@H](Cc2cn(OC)c3ccccc23)NC(=O)[C@H](CCCCCNCC(=O)O)NC(=O)[C@H]2CCCCN2C1=O ; CCC(C)[C@@H]1NC(=O)[C@H](Cc2cn(OC)c3ccccc23)NC(=O)[C@H](CCCCC(=O)OC)NC(=O)[C@H]2CCCCN2C1=O ; CCC(C)[C@@H]1NC(=O)[C@H](Cc2cn(OC)c3ccccc23)NC(=O)[C@H](CCCCC(=O)O)NC(=O)[C@H]2CCCCN2C1=O]

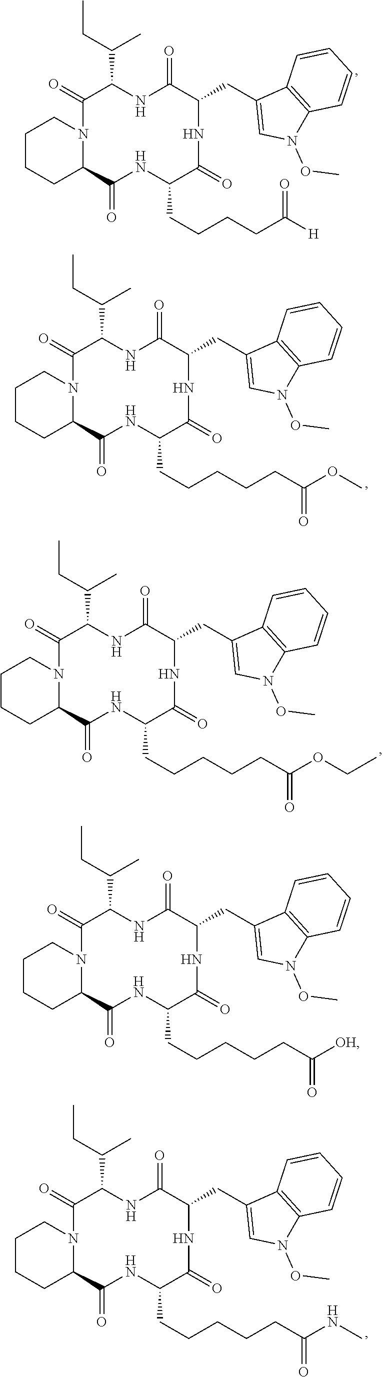 [SMILES: CCC(C)[C@@H]1NC(=O)[C@H](Cc2cn(OC)c3ccccc23)NC(=O)[C@H](CCCCC=O)NC(=O)[C@H]2CCCCN2C1=O ; CCC(C)[C@@H]1NC(=O)[C@H](Cc2cn(OC)c3ccccc23)NC(=O)[C@H](CCCCCC(=O)OC)NC(=O)[C@H]2CCCCN2C1=O ; CCOC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)NC)NC(=O)[C@H]2CCCCN2C1=O]

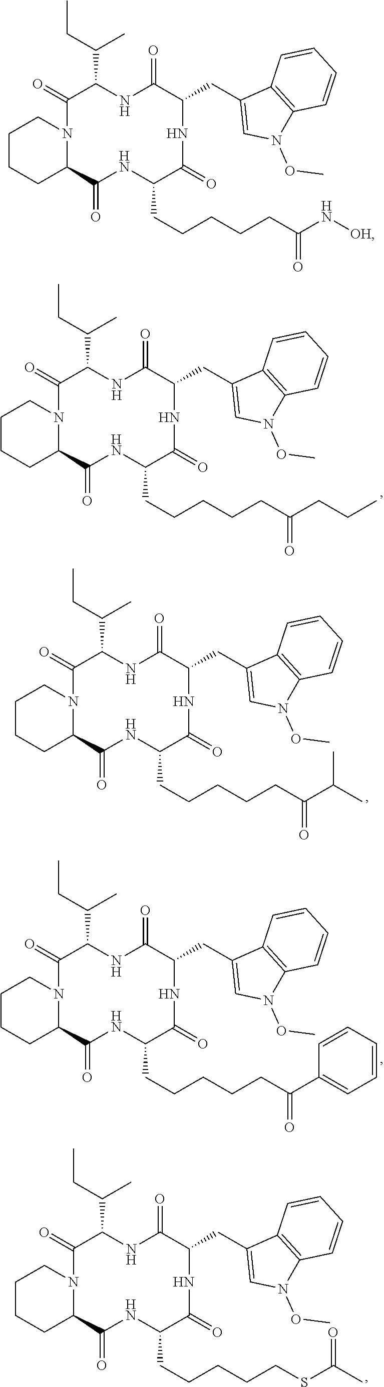 [SMILES: CCC(C)[C@@H]1NC(=O)[C@H](Cc2cn(OC)c3ccccc23)NC(=O)[C@H](CCCCCC(=O)NO)NC(=O)[C@H]2CCCCN2C1=O ; CCCC(=O)CCCCC[C@@H]1NC(=O)[C@H]2CCCCN2C(=O)[C@H](C(C)CC)NC(=O)[C@H](Cc2cn(OC)c3ccccc23)NC1=O ; "," ; CCC(C)[C@@H]1NC(=O)[C@H](Cc2cn(OC)c3ccccc23)NC(=O)[C@H](CCCCCC(=O)C(C)C)NC(=O)[C@H]2CCCCN2C1=O ; CCC(C)[C@@H]1NC(=O)[C@H](Cc2cn(OC)c3ccccc23)NC(=O)[C@H](CCCCCC(=O)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCCSC(C)=O)NC(=O)[C@H]2CCCCN2C1=O]

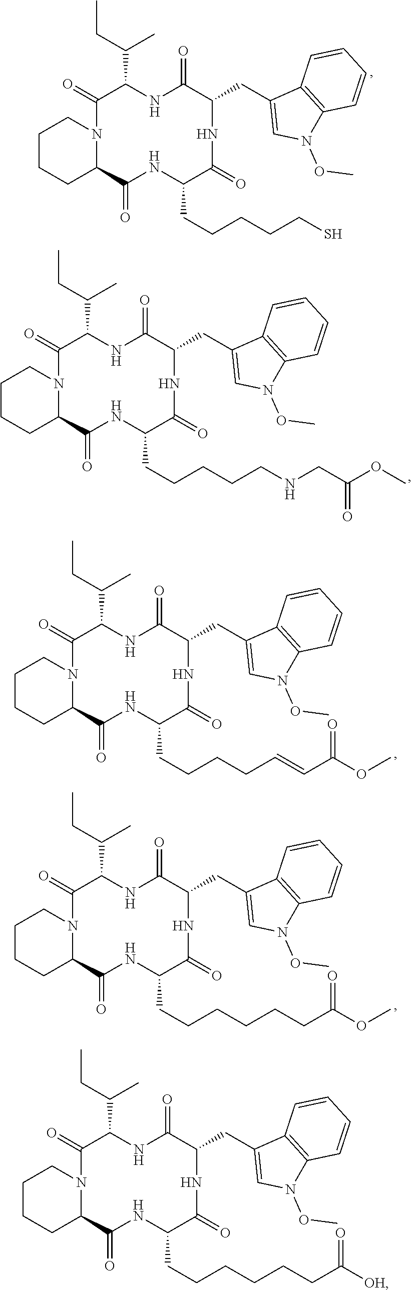

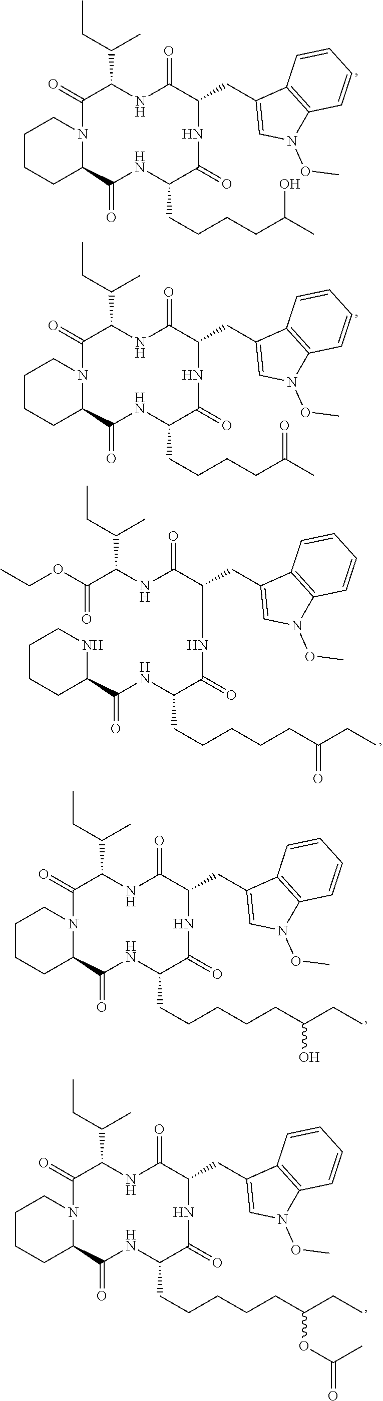

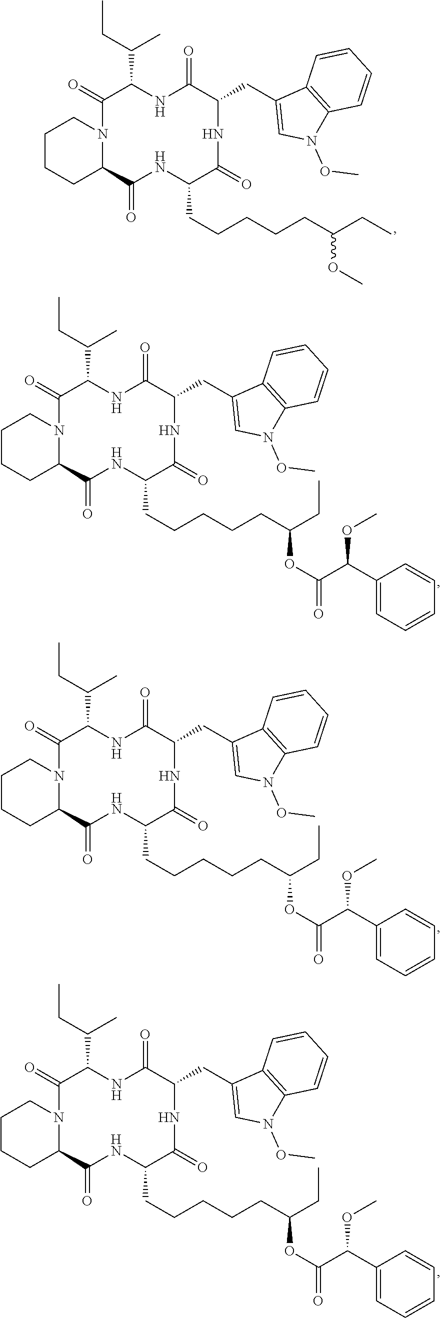 [SMILES: CCC(CCCCC[C@@H]1NC(=O)[C@H]2CCCCN2C(=O)[C@H](C(C)CC)NC(=O)[C@H](Cc2cn(OC)c3ccccc23)NC1=O)OC ; CCC(C)[C@@H]1NC(=O)[C@H](Cc2cn(OC)c3ccccc23)NC(=O)[C@H](CCCCC[C@@H](CC)OC(=O)[C@@H](OC)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCC[C@H](CC)OC(=O)[C@H](OC)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCC[C@@H](CC)OC(=O)[C@H](OC)c2ccccc2)NC(=O)[C@H]2CCCCN2C1=O]

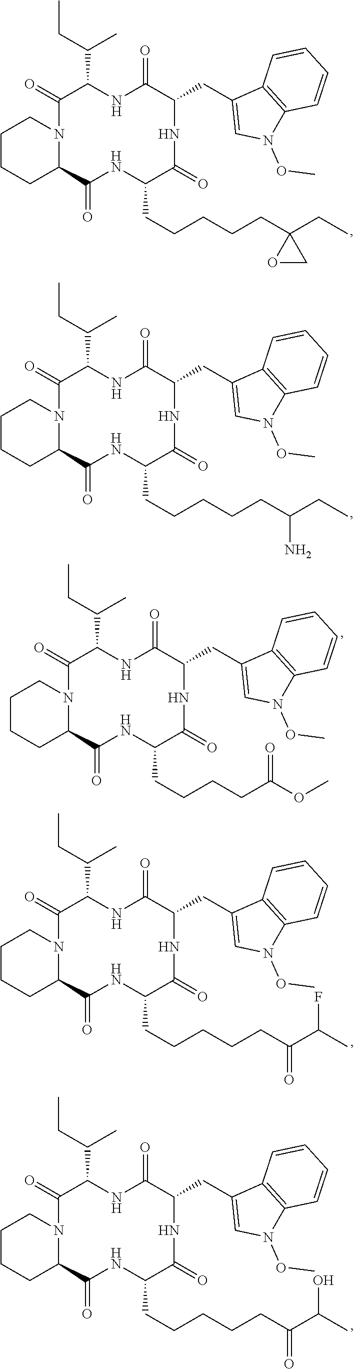 [SMILES: CCC(C)[C@@H]1NC(=O)[C@H](Cc2cn(OC)c3ccccc23)NC(=O)[C@H](CCCCCC2(CC)CO2)NC(=O)[C@H]2CCCCN2C1=O ; CCC(N)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCC(=O)OC)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)F)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O]

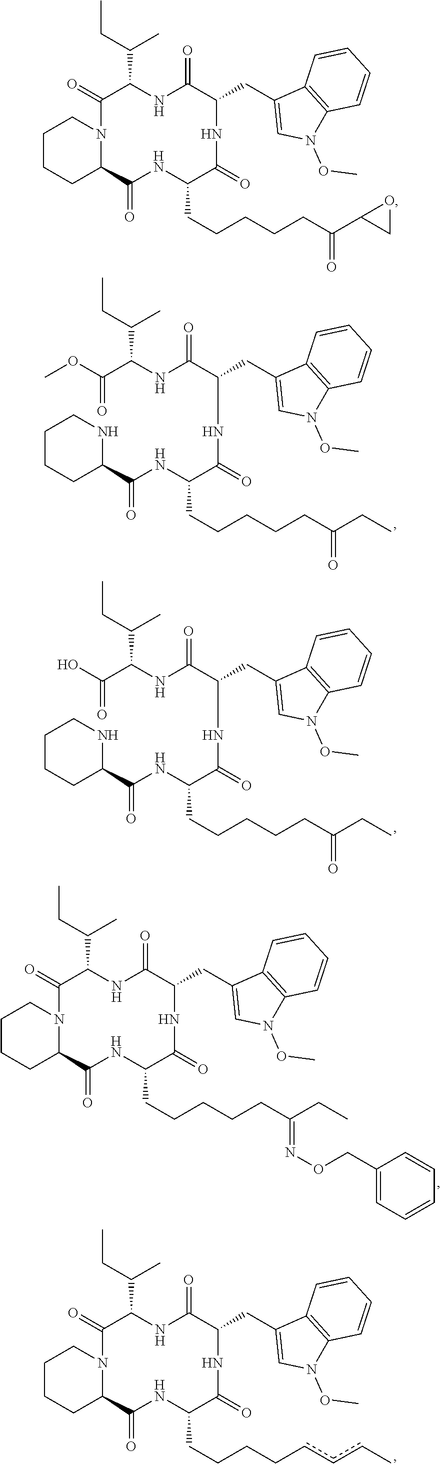

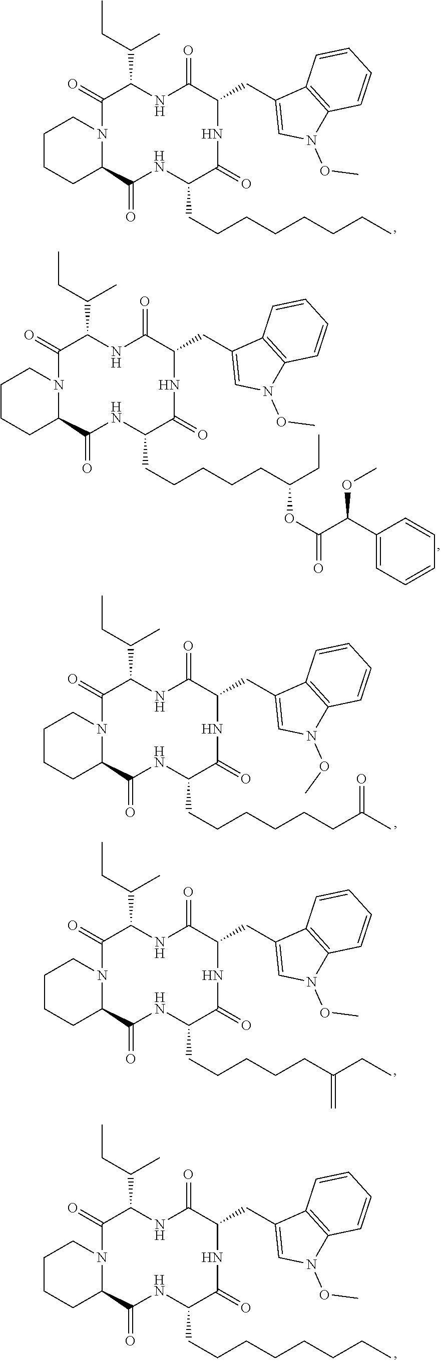

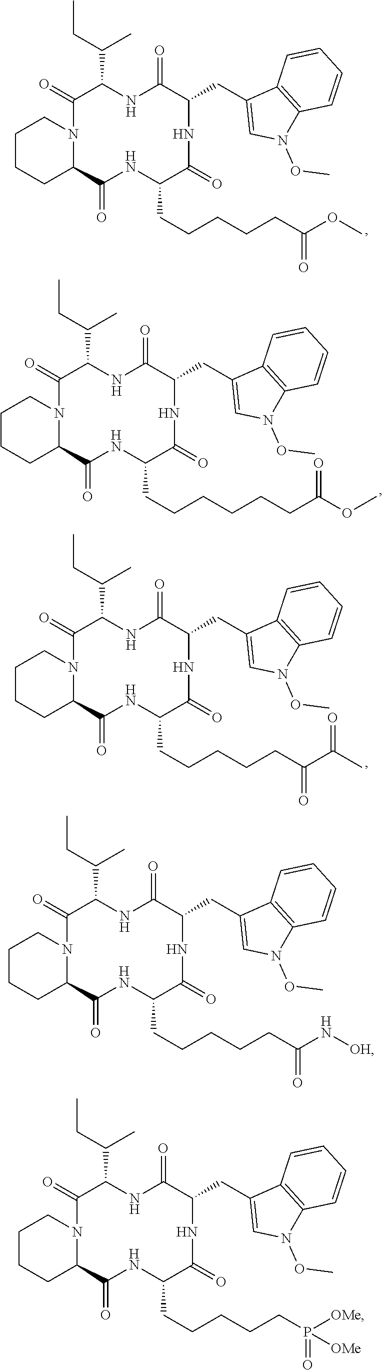

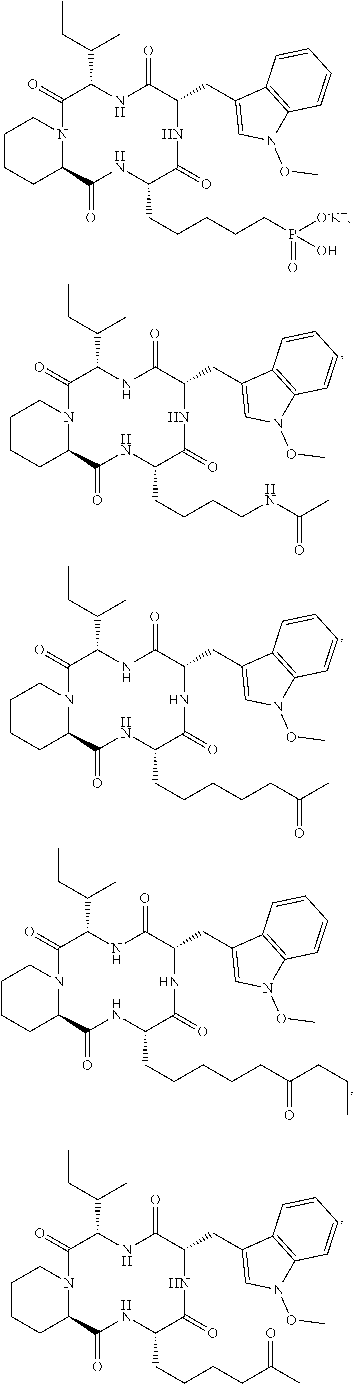

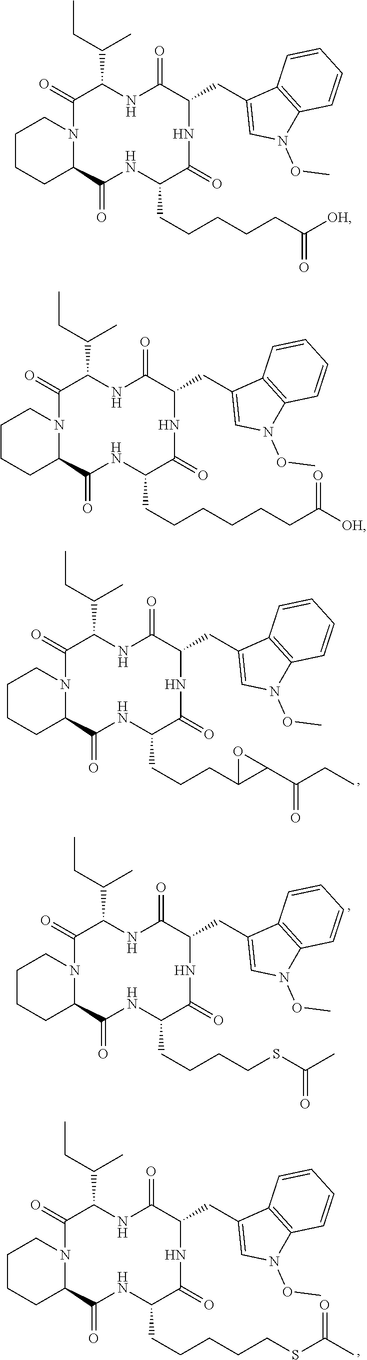 [SMILES: CCC(C)[C@@H]1NC(=O)[C@H](Cc2cn(OC)c3ccccc23)NC(=O)[C@H](CCCCCC(=O)O)NC(=O)[C@H]2CCCCN2C1=O ; CCC(C)[C@@H]1NC(=O)[C@H](Cc2cn(OC)c3ccccc23)NC(=O)[C@H](CCCCCCC(=O)O)NC(=O)[C@H]2CCCCN2C1=O ; CCC(=O)C1OC1CCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCSC(C)=O)NC(=O)[C@H]2CCCCN2C1=O ; CCC(C)[C@@H]1NC(=O)[C@H](Cc2cn(OC)c3ccccc23)NC(=O)[C@H](CCCCCSC(C)=O)NC(=O)[C@H]2CCCCN2C1=O]

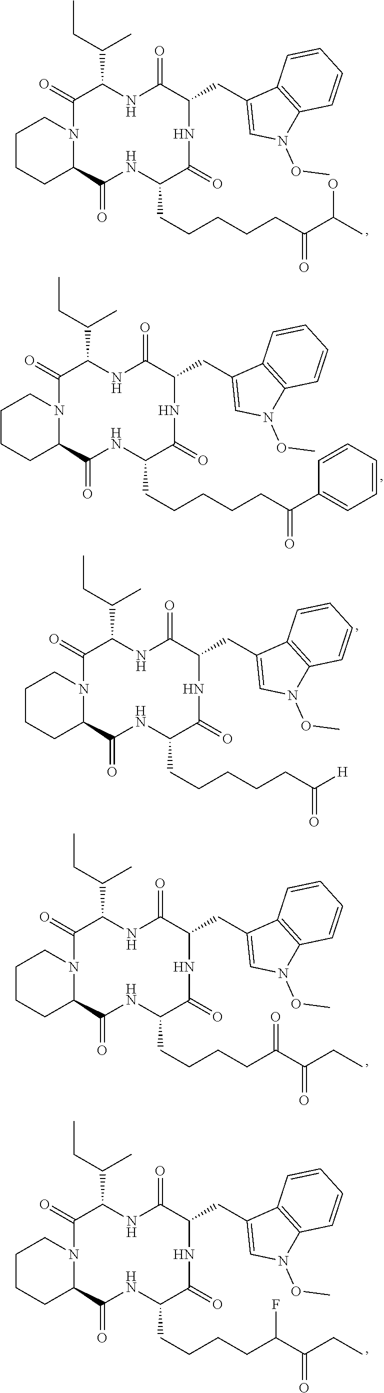 [SMILES: CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCCC=O)NC(=O)[C@H]2CCCCN2C1=O ; CCC(=O)C(=O)CCCC[C@@H]1NC(=O)[C@H]2CCCCN2C(=O)[C@H](C(C)CC)NC(=O)[C@H](Cc2cn(OC)c3ccccc23)NC1=O ; CCC(=O)C(F)CCCC[C@@H]1NC(=O)[C@H]2CCCCN2C(=O)[C@H](C(C)CC)NC(=O)[C@H](Cc2cn(OC)c3ccccc23)NC1=O]

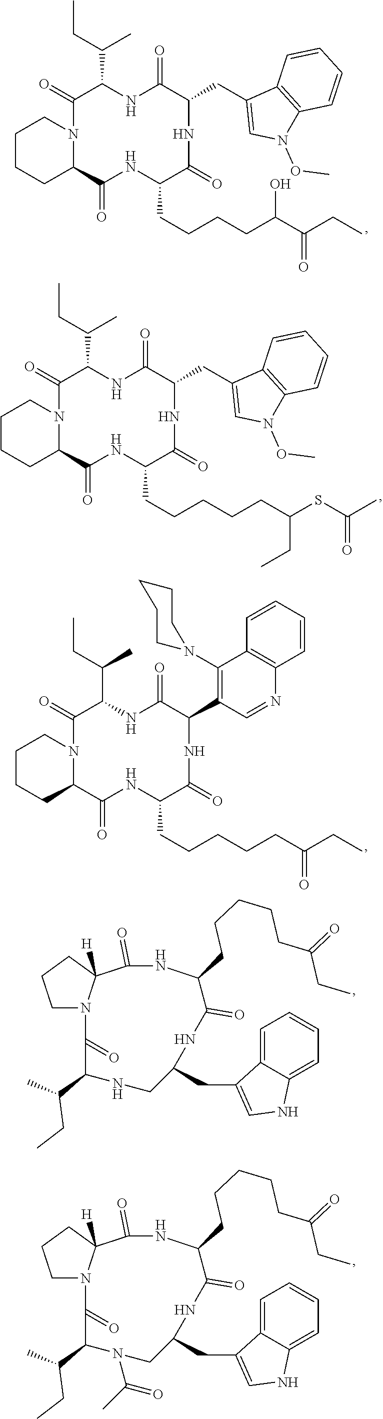

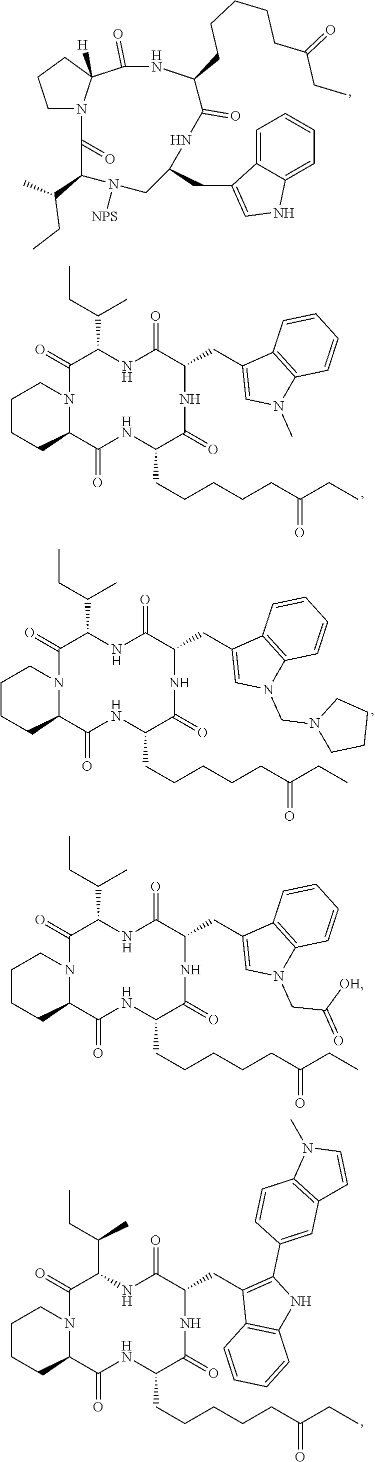

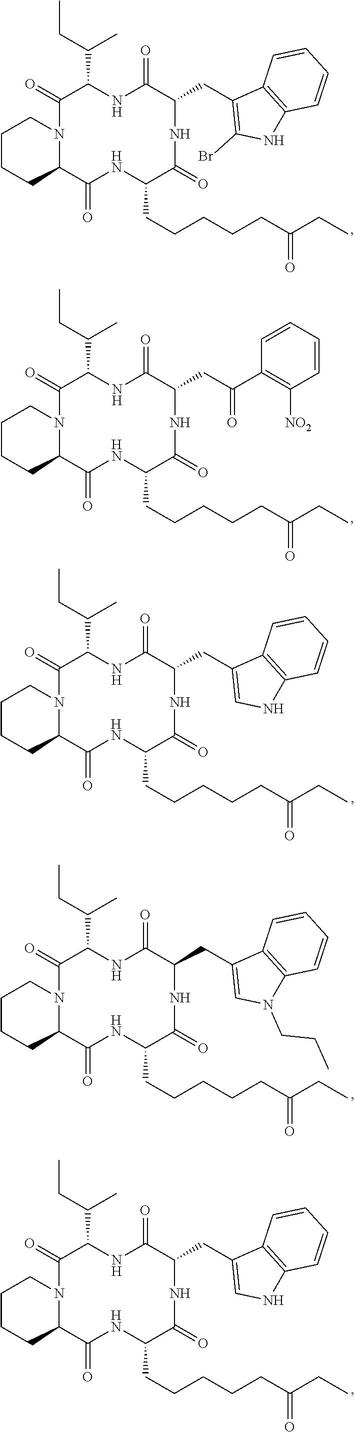 [SMILES: CCC(=O)CCCCC[C@@H]1NC(=O)[C@H]2CCCCN2C(=O)[C@H](C(C)CC)NC(=O)[C@H](Cc2c(Br)[nH]c3ccccc23)NC1=O ; CCC(=O)CCCCC[C@@H]1NC(=O)[C@H]2CCCCN2C(=O)[C@H](C(C)CC)NC(=O)[C@H](CC(=O)c2ccccc2[N+](=O)[O-])NC1=O ; CCC(=O)CCCCC[C@@H]1NC(=O)[C@H]2CCCCN2C(=O)[C@H](C(C)CC)NC(=O)[C@H](Cc2c[nH]c3ccccc23)NC1=O ; CCCn1cc(C[C@H]2NC(=O)[C@H](CCCCCC(=O)CC)NC(=O)[C@H]3CCCCN3C(=O)[C@H](C(C)CC)NC2=O)c2ccccc21 ; CCC(=O)CCCCC[C@@H]1NC(=O)[C@H]2CCCCN2C(=O)[C@H](C(C)CC)NC(=O)[C@H](Cc2c[nH]c3ccccc23)NC1=O]

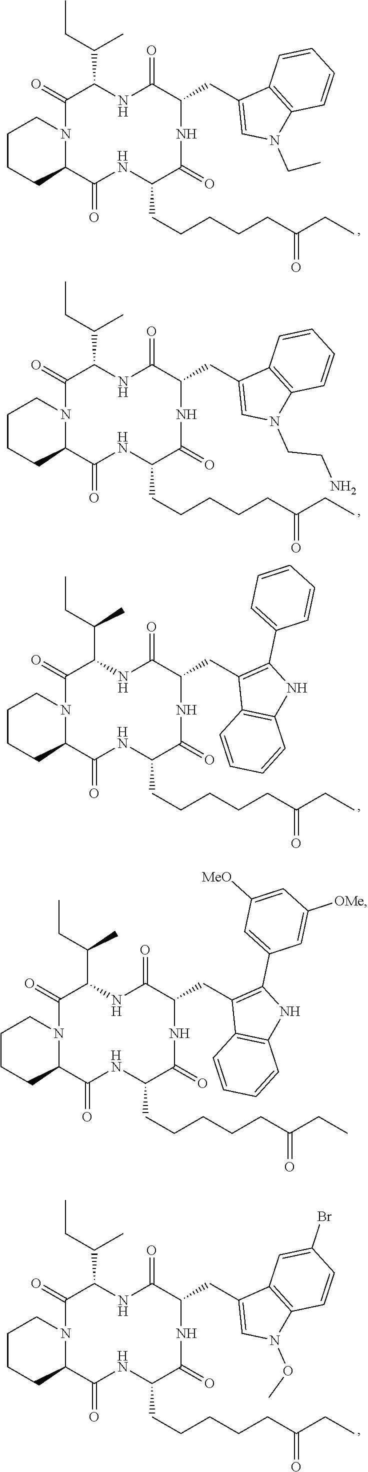

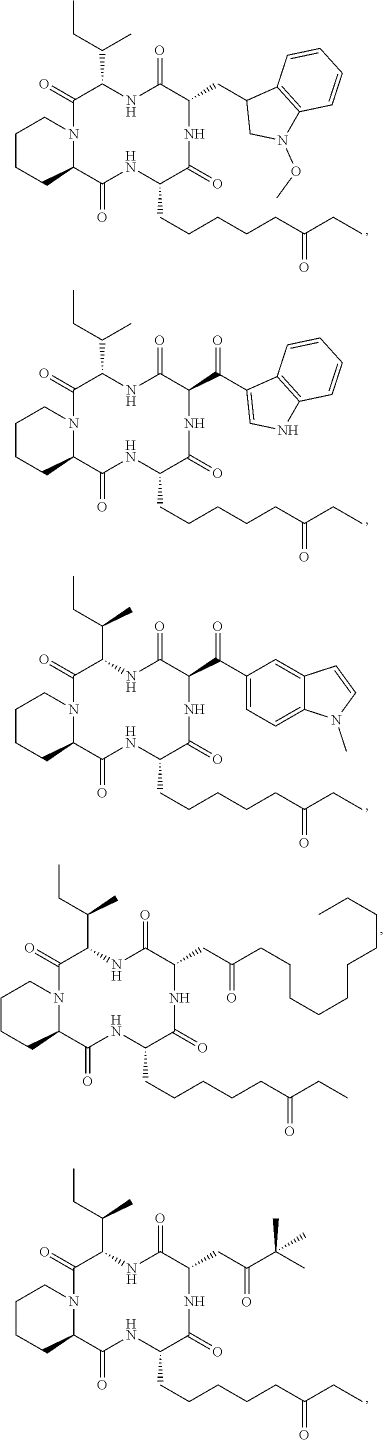

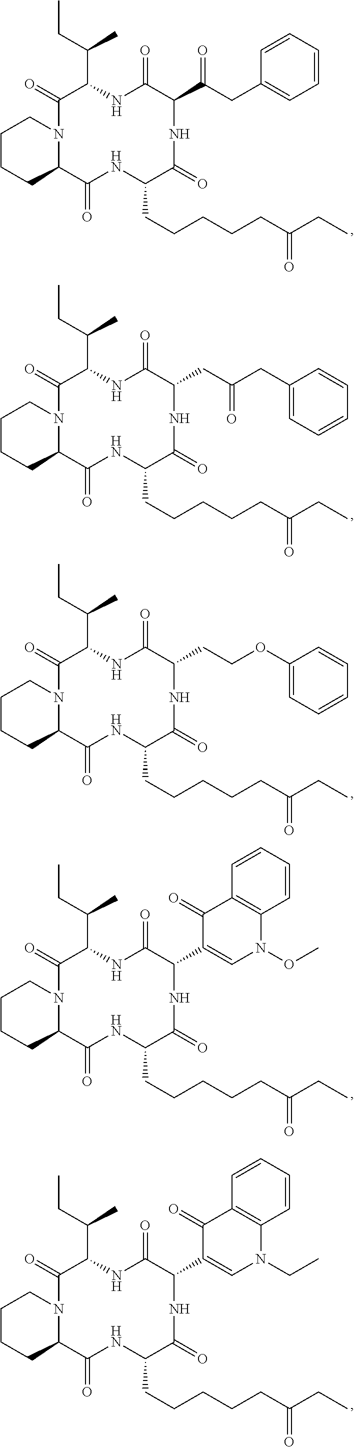

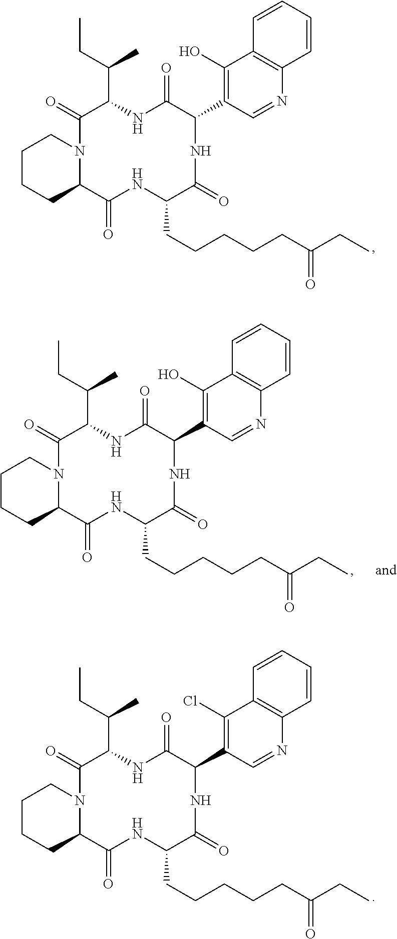

26. The pharmaceutical composition of claim 23, wherein the staphylococcal infection is a skin and soft tissue infection (SSTI), staphylococcal pneumonia, a staphylococcal bone infection, a staphylococcal joint infection, staphylococcal sepsis, staphylococcal endocarditis, staphylococcal osteomyelitis, or staphylococcal meningitis.

27. The pharmaceutical composition of claim 23, wherein the staphylococcal infection is a methicillin-resistant Staphylococcus aureus infection or a methicillin-sensitive Staphylococcus aureus infection.

28. A method of treating a staphylococcal infection comprising:
administering to a subject in need thereof a therapeutically effective amount of apicidin or a pharmaceutically acceptable derivative thereof, and a pharmaceutically acceptable carrier.

29. The method of claim 28, wherein the apicidin has a structure selected from:

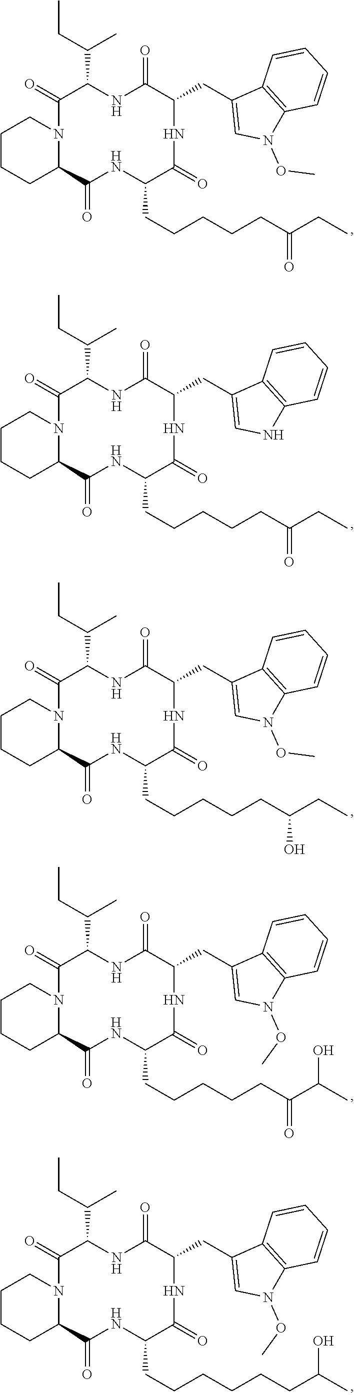 [SMILES: CCC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(=O)CCCCC[C@@H]1NC(=O)[C@H]2CCCCN2C(=O)[C@H](C(C)CC)NC(=O)[C@H](Cc2c[nH]c3ccccc23)NC1=O ; CCC(C)[C@@H]1NC(=O)[C@H](Cc2cn(OC)c3ccccc23)NC(=O)[C@H](CCCCC[C@@H](O)CC)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCC(C)[C@@H]1NC(=O)[C@H](Cc2cn(OC)c3ccccc23)NC(=O)[C@H](CCCCCCC(C)O)NC(=O)[C@H]2CCCCN2C1=O]

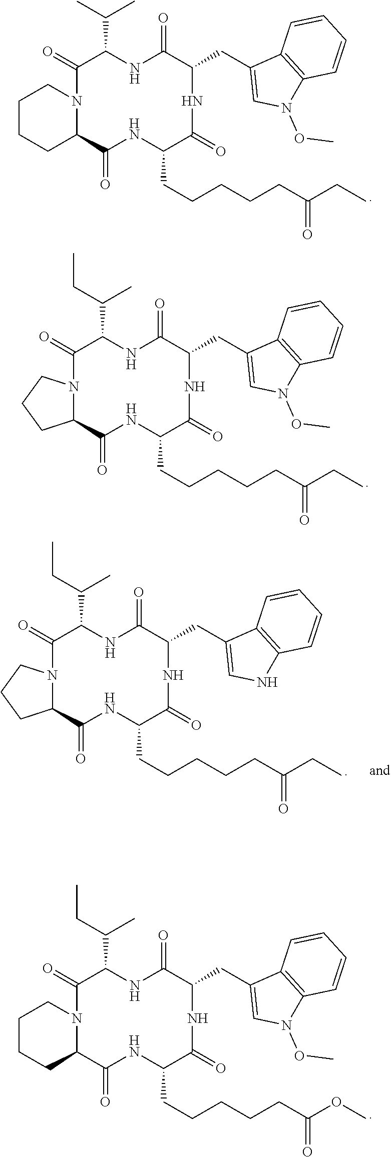 [SMILES: CCC(=O)CCCCC[C@@H]1NC(=O)[C@H]2CCCCN2C(=O)[C@H](C(C)C)NC(=O)[C@H](Cc2cn(OC)c3ccccc23)NC1=O ; CCC(=O)CCCCC[C@@H]1NC(=O)[C@H]2CCCN2C(=O)[C@H](C(C)CC)NC(=O)[C@H](Cc2cn(OC)c3ccccc23)NC1=O ; CCC(=O)CCCCC[C@@H]1NC(=O)[C@H]2CCCN2C(=O)[C@H](C(C)CC)NC(=O)[C@H](Cc2c[nH]c3ccccc23)NC1=O ; CCC(C)[C@@H]1NC(=O)[C@H](Cc2cn(OC)c3ccccc23)NC(=O)[C@H](CCCCCC(=O)OC)NC(=O)[C@H]2CCCCN2C1=O]

30. The method of claim 28, wherein the apicidin has a structure selected from:

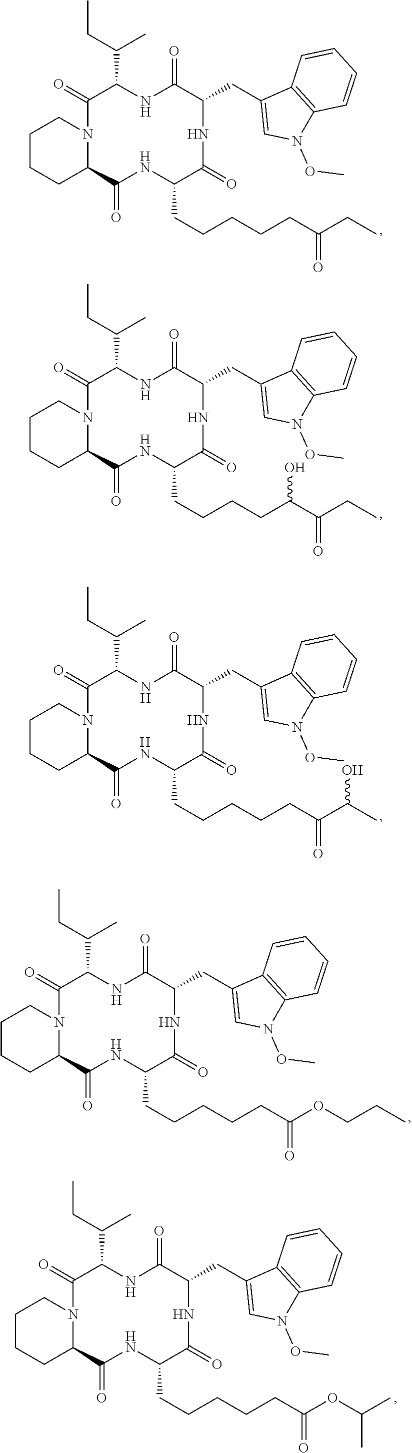 [SMILES: CCC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(=O)C(O)CCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCCOC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)OC(C)C)NC(=O)[C@H]2CCCCN2C1=O]

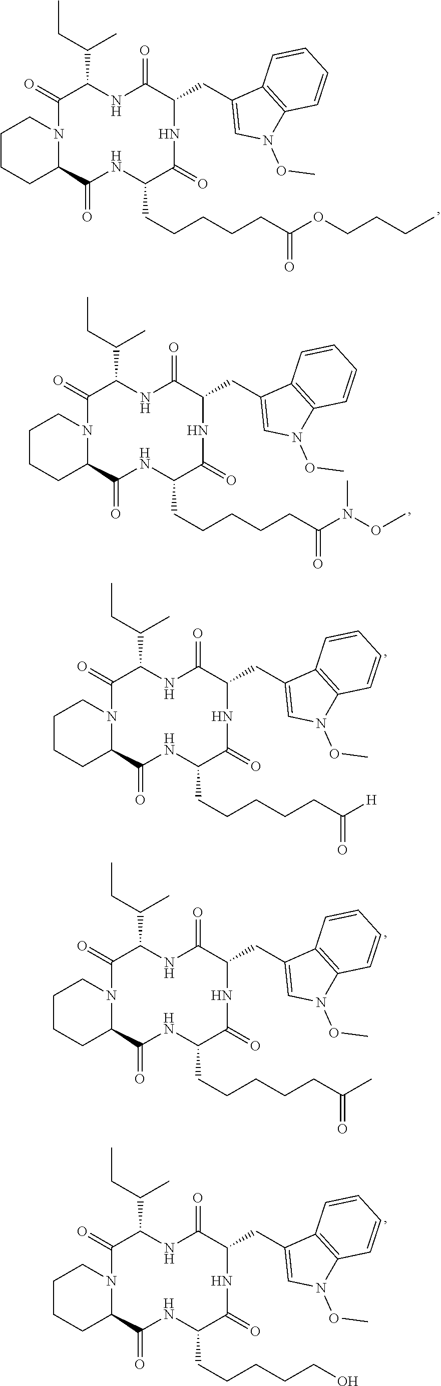 [SMILES: CCCCOC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)N(C)OC)NC(=O)[C@H]2CCCCN2C1=O ; CCC(C)[C@@H]1NC(=O)[C@H](Cc2cn(OC)c3ccccc23)NC(=O)[C@H](CCCCCC=O)NC(=O)[C@H]2CCCCN2C1=O ; CCC(C)[C@@H]1NC(=O)[C@H](Cc2cn(OC)c3ccccc23)NC(=O)[C@H](CCCCCC(C)=O)NC(=O)[C@H]2CCCCN2C1=O ; CCC(C)[C@@H]1NC(=O)[C@H](Cc2cn(OC)c3ccccc23)NC(=O)[C@H](CCCCCO)NC(=O)[C@H]2CCCCN2C1=O]

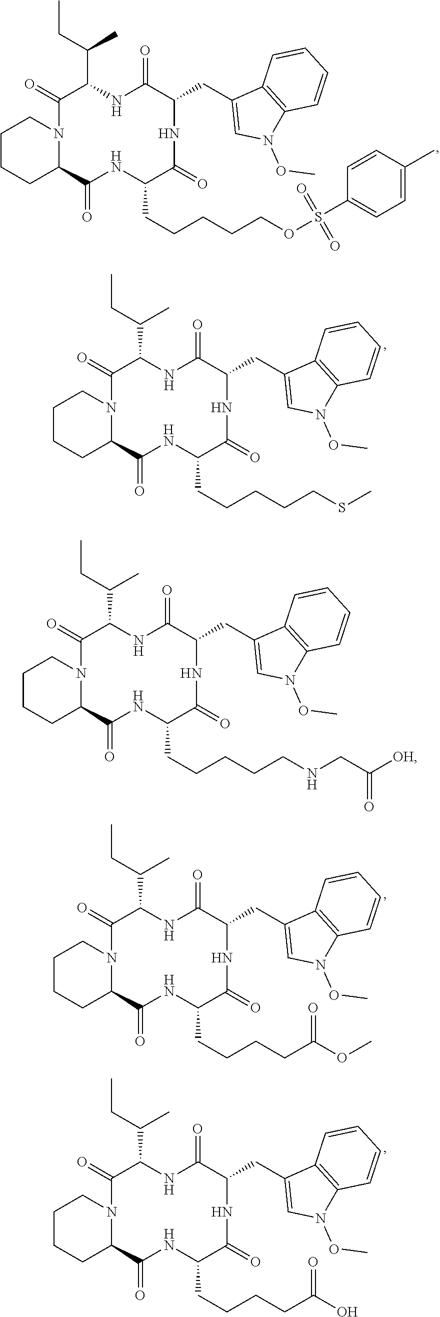 [SMILES: CC[C@@H](C)[C@@H]1NC(=O)[C@H](Cc2cn(OC)c3ccccc23)NC(=O)[C@H](CCCCCOS(=O)(=O)c2ccc(C)cc2)NC(=O)[C@H]2CCCCN2C1=O ; CCC(C)[C@@H]1NC(=O)[C@H](Cc2cn(OC)c3ccccc23)NC(=O)[C@H](CCCCCSC)NC(=O)[C@H]2CCCCN2C1=O ; CCC(C)[C@@H]1NC(=O)[C@H](Cc2cn(OC)c3ccccc23)NC(=O)[C@H](CCCCCNCC(=O)O)NC(=O)[C@H]2CCCCN2C1=O ; CCC(C)[C@@H]1NC(=O)[C@H](Cc2cn(OC)c3ccccc23)NC(=O)[C@H](CCCCC(=O)OC)NC(=O)[C@H]2CCCCN2C1=O ; CCC(C)[C@@H]1NC(=O)[C@H](Cc2cn(OC)c3ccccc23)NC(=O)[C@H](CCCCC(=O)O)NC(=O)[C@H]2CCCCN2C1=O]

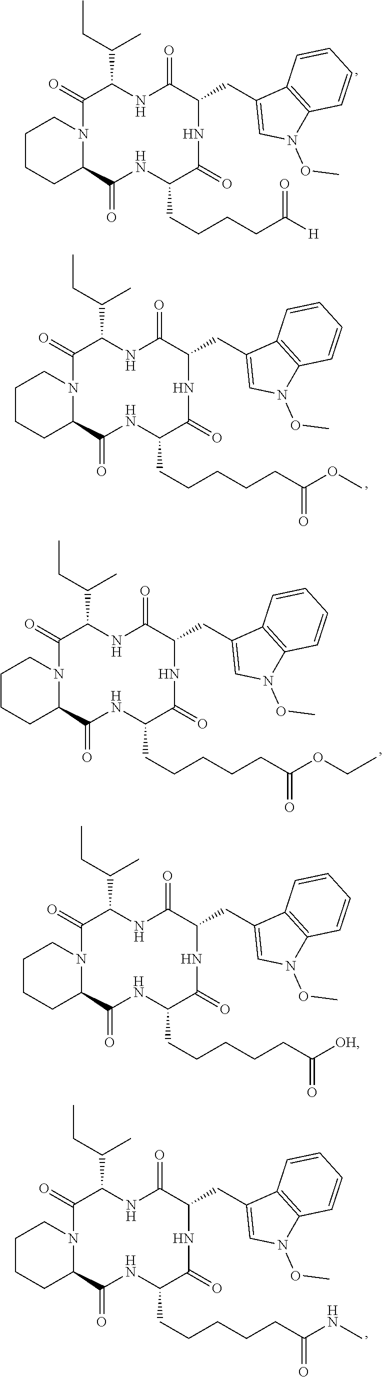 [SMILES: CCC(C)[C@@H]1NC(=O)[C@H](Cc2cn(OC)c3ccccc23)NC(=O)[C@H](CCCCC=O)NC(=O)[C@H]2CCCCN2C1=O ; CCC(C)[C@@H]1NC(=O)[C@H](Cc2cn(OC)c3ccccc23)NC(=O)[C@H](CCCCCC(=O)OC)NC(=O)[C@H]2CCCCN2C1=O ; CCOC(=O)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCCC(=O)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)NC)NC(=O)[C@H]2CCCCN2C1=O]

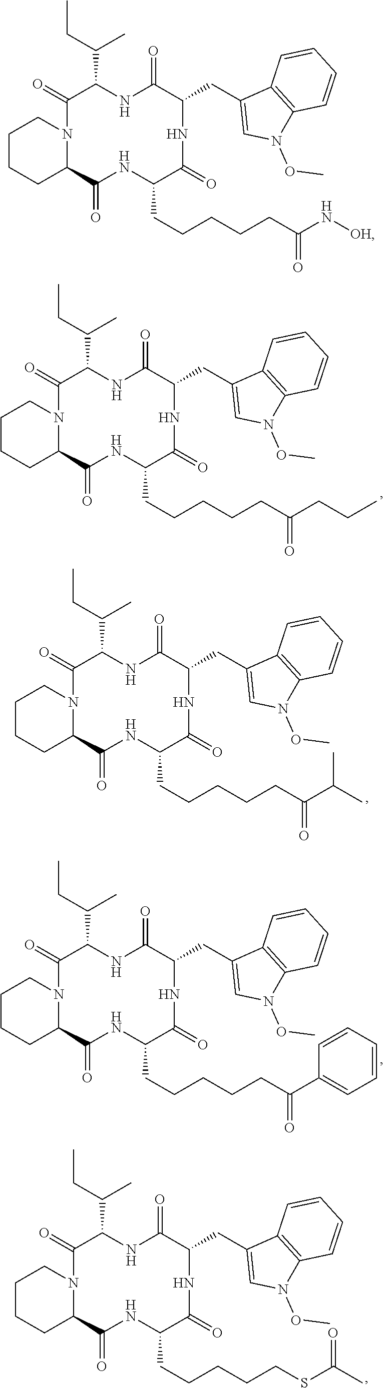 [SMILES: CCC(C)[C@@H]1NC(=O)[C@H](Cc2cn(OC)c3ccccc23)NC(=O)[C@H](CCCCCC(=O)NO)NC(=O)[C@H]2CCCCN2C1=O ; CCCC(=O)CCCCC[C@@H]1NC(=O)[C@H]2CCCCN2C(=O)[C@H](C(C)CC)NC(=O)[C@H](Cc2cn(OC)c3ccccc23)NC1=O ; "," ; CCC(C)[C@@H]1NC(=O)[C@H](Cc2cn(OC)c3ccccc23)NC(=O)[C@H](CCCCCC(=O)C(C)C)NC(=O)[C@H]2CCCCN2C1=O ; CCC(C)[C@@H]1NC(=O)[C@H](Cc2cn(OC)c3ccccc23)NC(=O)[C@H](CCCCCC(=O)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCCSC(C)=O)NC(=O)[C@H]2CCCCN2C1=O]

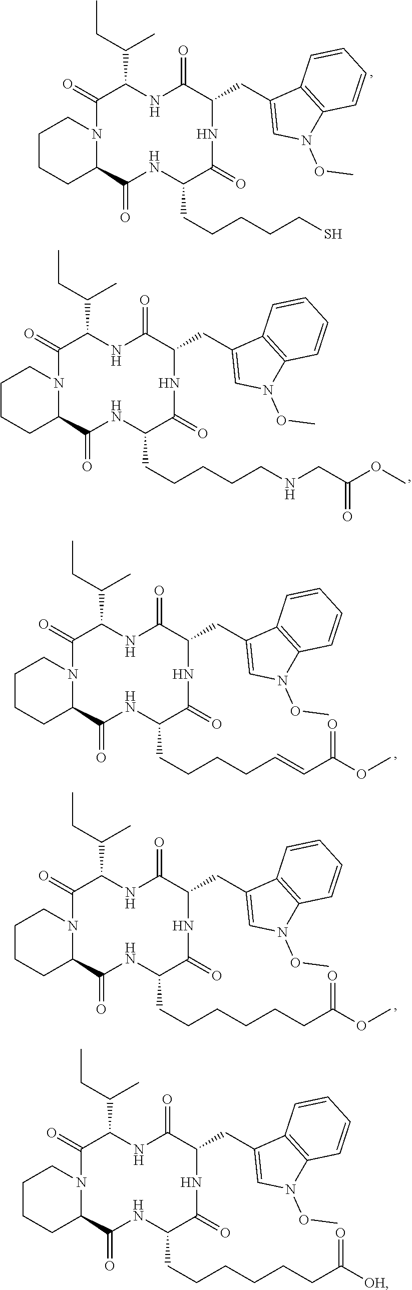

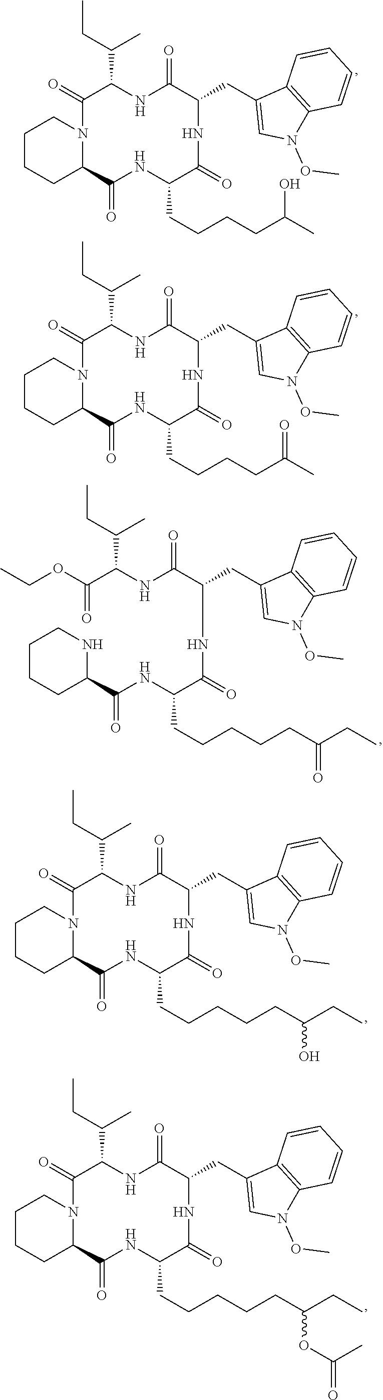

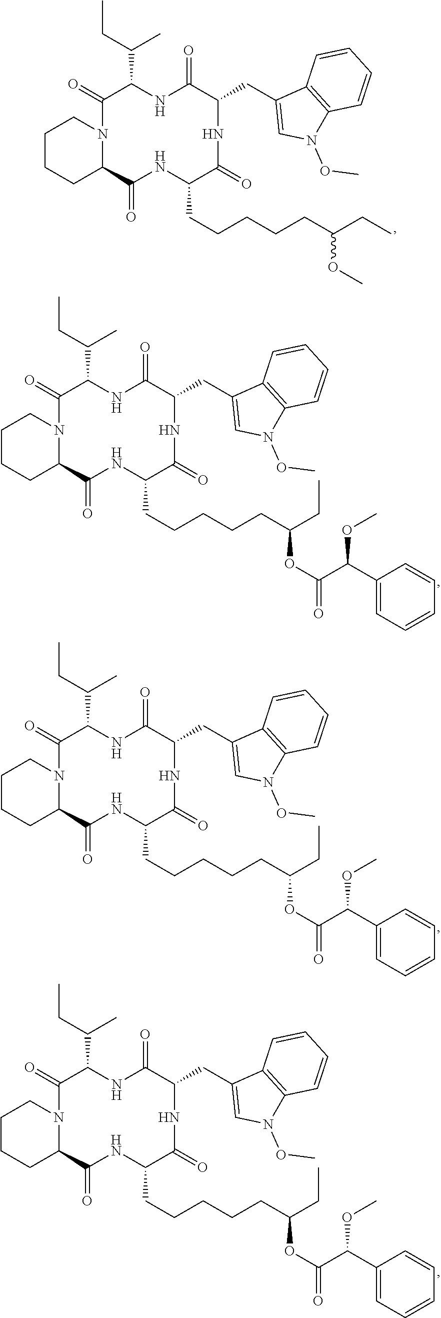 [SMILES: CCC(CCCCC[C@@H]1NC(=O)[C@H]2CCCCN2C(=O)[C@H](C(C)CC)NC(=O)[C@H](Cc2cn(OC)c3ccccc23)NC1=O)OC ; CCC(C)[C@@H]1NC(=O)[C@H](Cc2cn(OC)c3ccccc23)NC(=O)[C@H](CCCCC[C@@H](CC)OC(=O)[C@@H](OC)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCC[C@H](CC)OC(=O)[C@H](OC)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCC[C@@H](CC)OC(=O)[C@H](OC)c2ccccc2)NC(=O)[C@H]2CCCCN2C1=O]

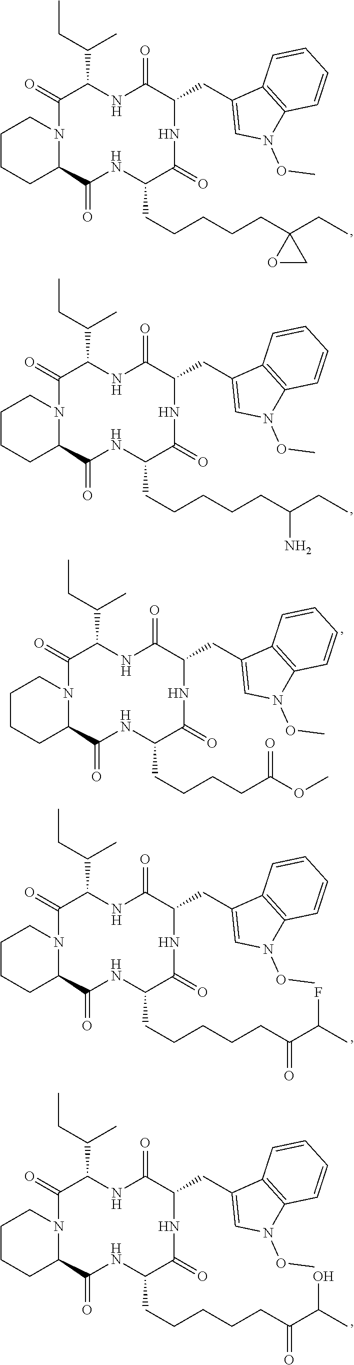 [SMILES: CCC(C)[C@@H]1NC(=O)[C@H](Cc2cn(OC)c3ccccc23)NC(=O)[C@H](CCCCCC2(CC)CO2)NC(=O)[C@H]2CCCCN2C1=O ; CCC(N)CCCCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCC(=O)OC)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)F)NC(=O)[C@H]2CCCCN2C1=O ; CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O]

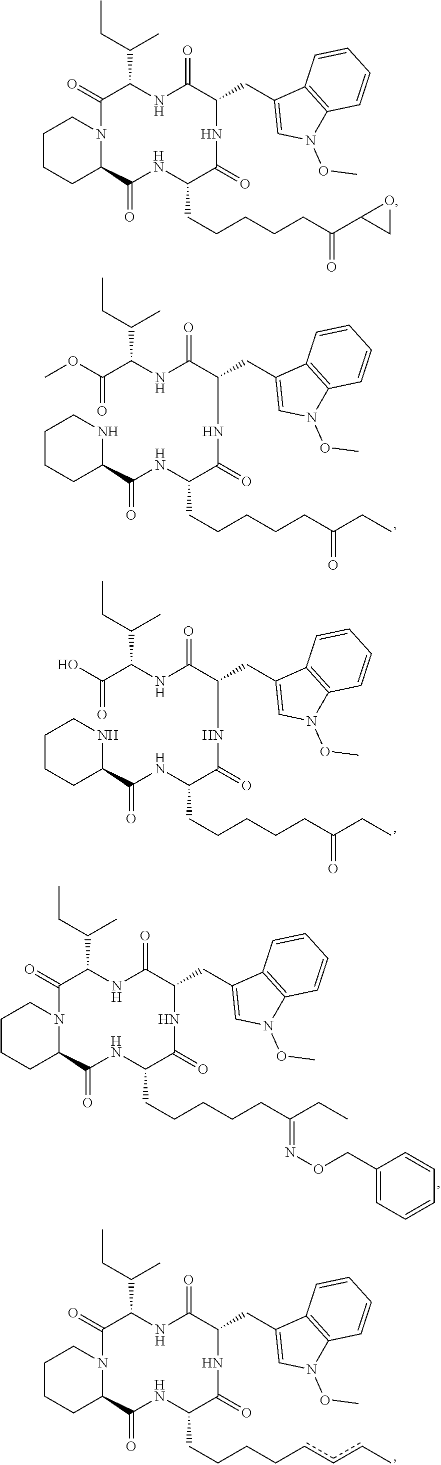

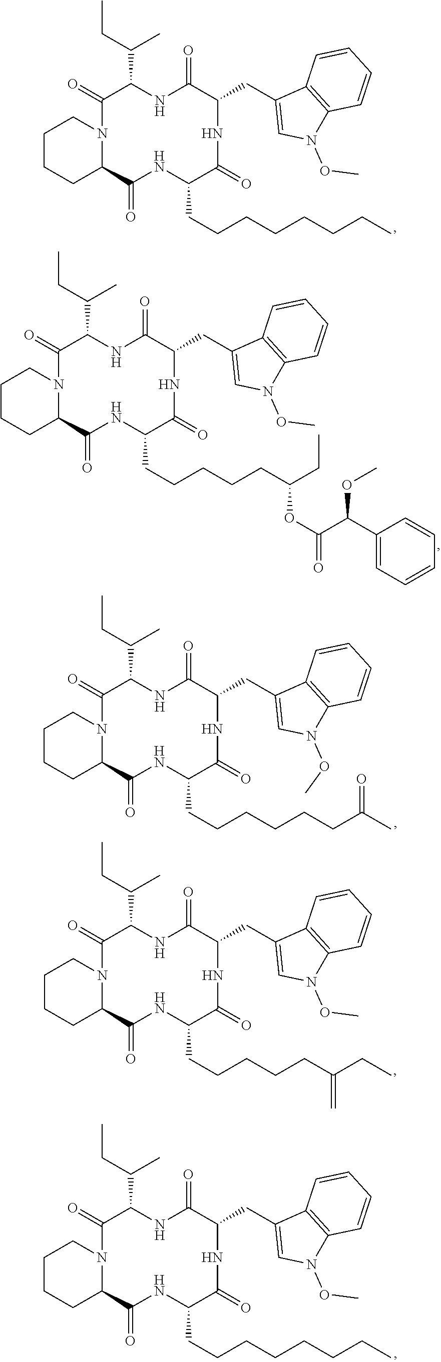

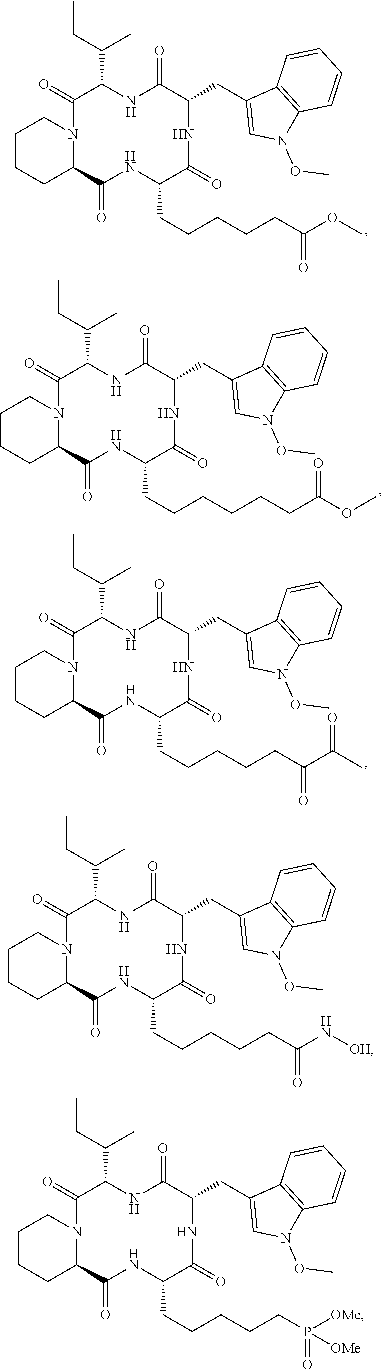

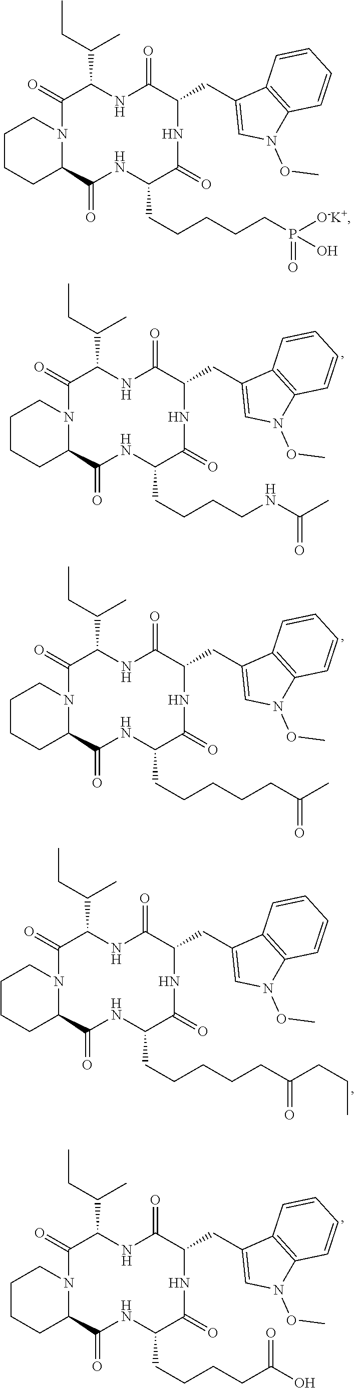

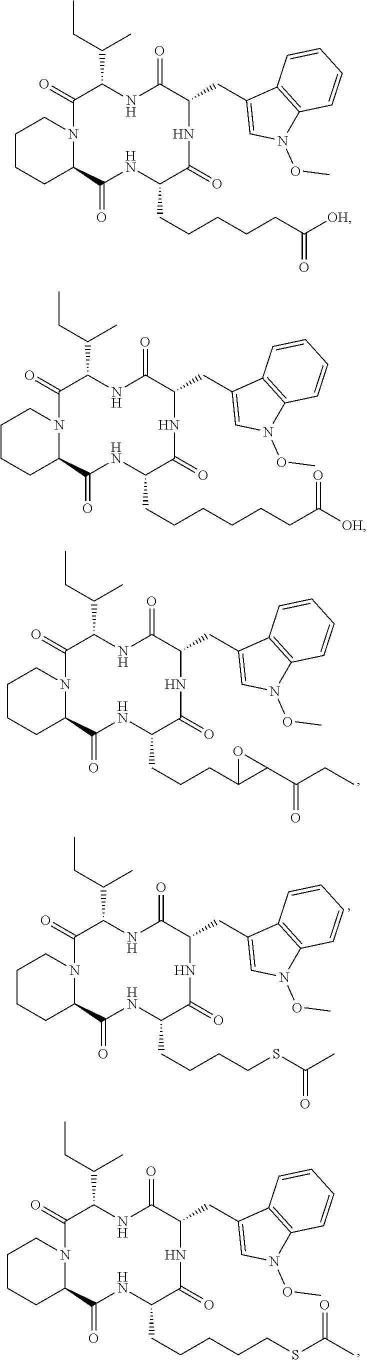 [SMILES: CCC(C)[C@@H]1NC(=O)[C@H](Cc2cn(OC)c3ccccc23)NC(=O)[C@H](CCCCCC(=O)O)NC(=O)[C@H]2CCCCN2C1=O ; CCC(C)[C@@H]1NC(=O)[C@H](Cc2cn(OC)c3ccccc23)NC(=O)[C@H](CCCCCCC(=O)O)NC(=O)[C@H]2CCCCN2C1=O ; CCC(=O)C1OC1CCC[C@@H]1NC(=O)[C@H]2CCCCN2C(=O)[C@H](C(C)CC)NC(=O)[C@H](Cc2cn(OC)c3ccccc23)NC1=O ; CCC(C)[C@@H]1NC(=O)[C@H](Cc2cn(OC)c3ccccc23)NC(=O)[C@H](CCCCSC(C)=O)NC(=O)[C@H]2CCCCN2C1=O ; CCC(C)[C@@H]1NC(=O)[C@H](Cc2cn(OC)c3ccccc23)NC(=O)[C@H](CCCCCSC(C)=O)NC(=O)[C@H]2CCCCN2C1=O]

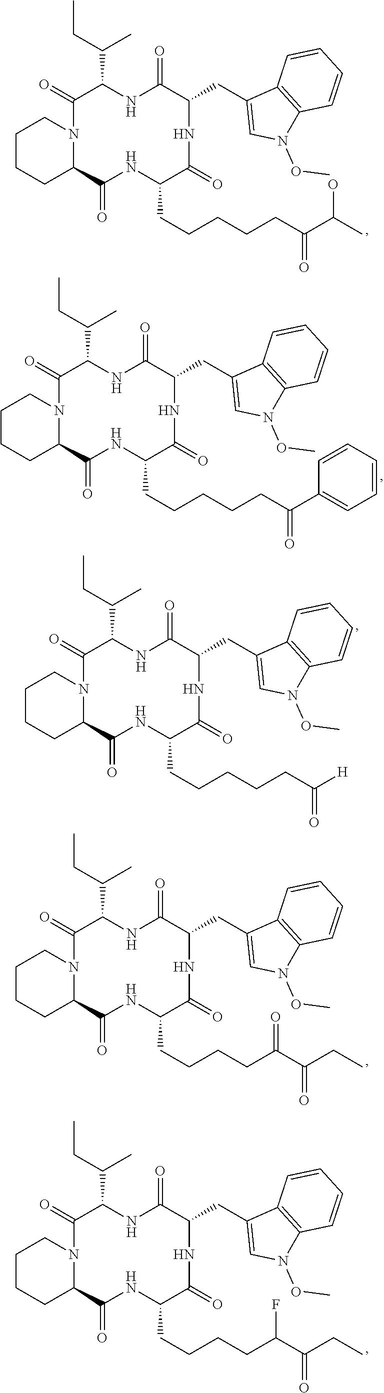 [SMILES: CCC(C)[C@@H]1NC(=O)[C@H](Cc2cn(OC)c3ccccc23)NC(=O)[C@H](CCCCCC(=O)C(C)O)NC(=O)[C@H]2CCCCN2C1=O ; CCC(C)[C@@H]1NC(=O)[C@H](Cc2cn(OC)c3ccccc23)NC(=O)[C@H](CCCCCC(=O)c2ccccc2)NC(=O)[C@H]2CCCCN2C1=O ; CCC(C)[C@@H]1NC(=O)[C@H](Cc2cn(OC)c3ccccc23)NC(=O)[C@H](CCCCCC=O)NC(=O)[C@H]2CCCCN2C1=O ; CCC(=O)C(=O)CCCC[C@@H]1NC(=O)[C@H]2CCCCN2C(=O)[C@H](C(C)CC)NC(=O)[C@H](Cc2cn(OC)c3ccccc23)NC1=O ; CCC(=O)C(F)CCCC[C@@H]1NC(=O)[C@H]2CCCCN2C(=O)[C@H](C(C)CC)NC(=O)[C@H](Cc2cn(OC)c3ccccc23)NC1=O]

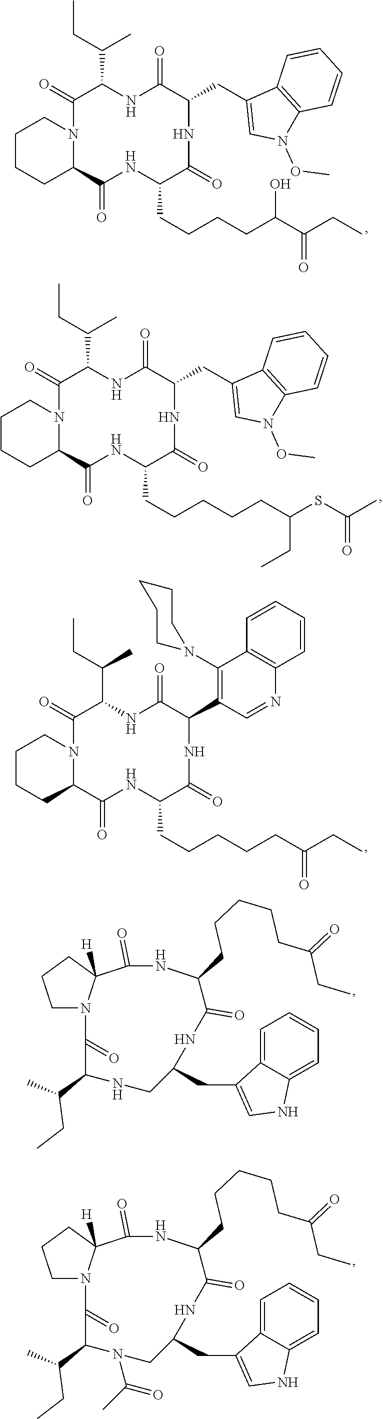

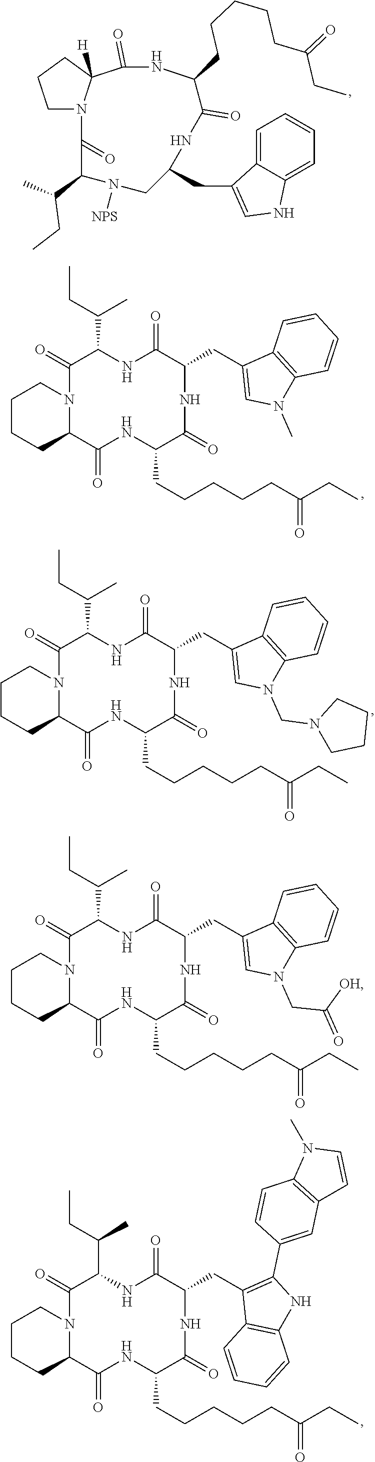

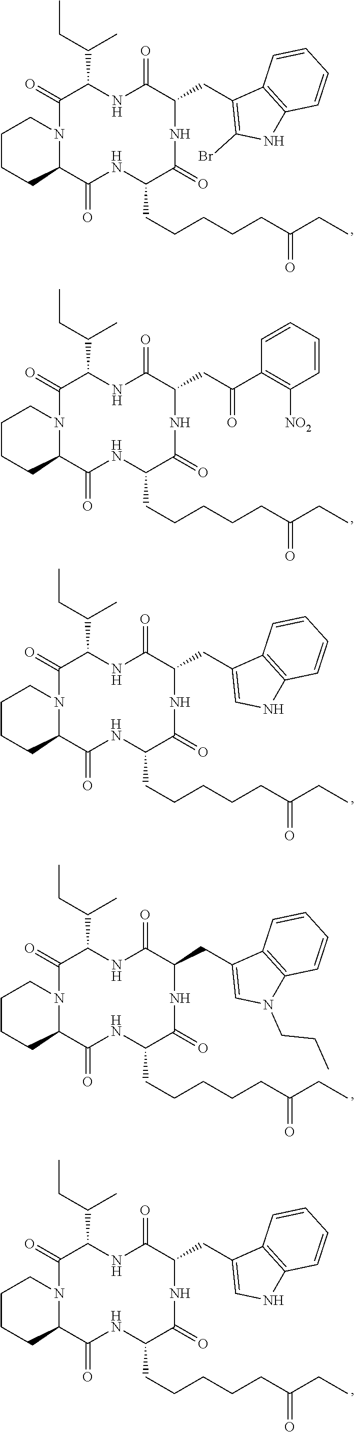 [SMILES: CCC(=O)CCCCC[C@@H]1NC(=O)[C@H]2CCCCN2C(=O)[C@H](C(C)CC)NC(=O)[C@H](Cc2c(Br)[nH]c3ccccc23)NC1=O ; CCC(=O)CCCCC[C@@H]1NC(=O)[C@H]2CCCCN2C(=O)[C@H](C(C)CC)NC(=O)[C@H](CC(=O)c2ccccc2[N+](=O)[O-])NC1=O ; CCC(=O)CCCCC[C@@H]1NC(=O)[C@H]2CCCCN2C(=O)[C@H](C(C)CC)NC(=O)[C@H](Cc2c[nH]c3ccccc23)NC1=O ; CCCn1cc(C[C@H]2NC(=O)[C@H](CCCCCC(=O)CC)NC(=O)[C@H]3CCCCN3C(=O)[C@H](C(C)CC)NC2=O)c2ccccc21 ; CCC(=O)CCCCC[C@@H]1NC(=O)[C@H]2CCCCN2C(=O)[C@H](C(C)CC)NC(=O)[C@H](Cc2c[nH]c3ccccc23)NC1=O]

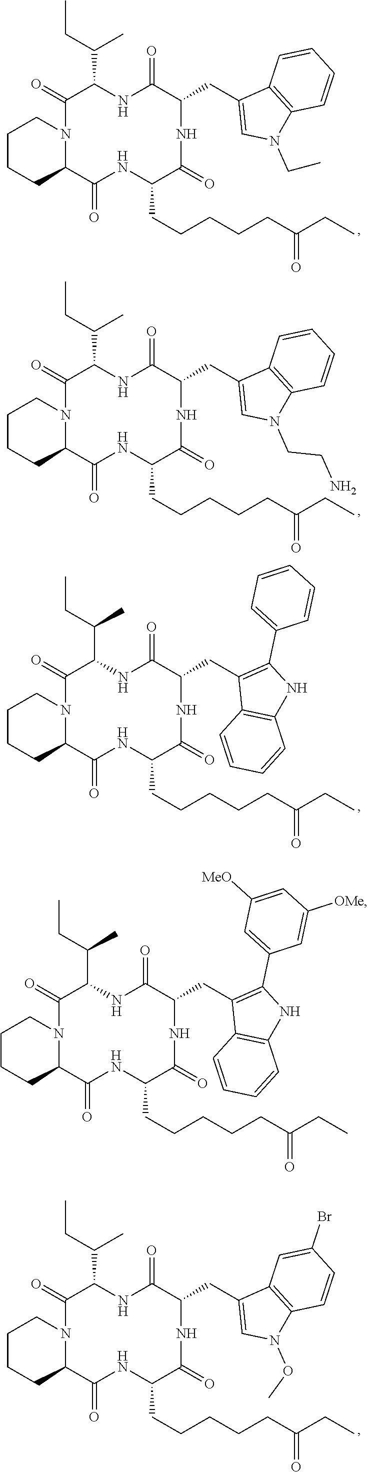

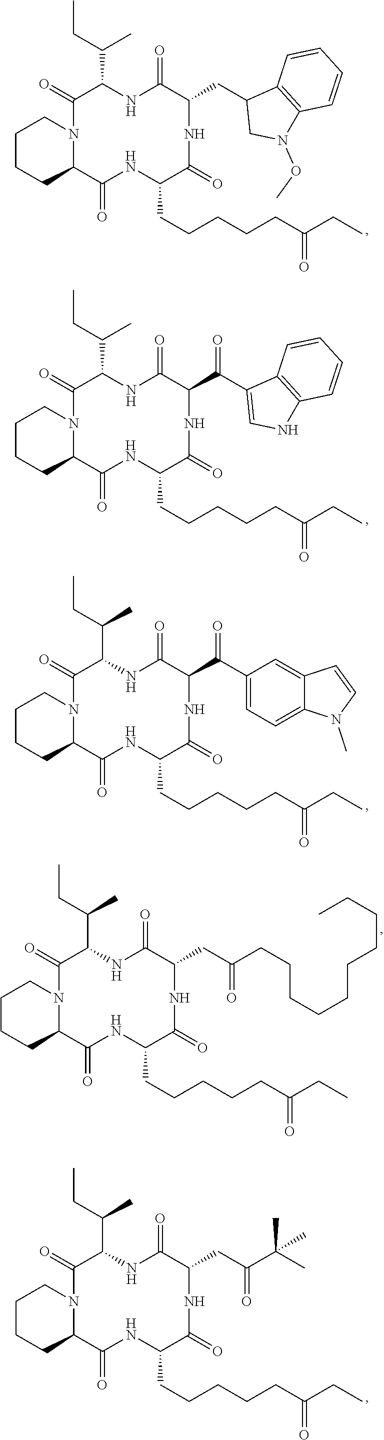

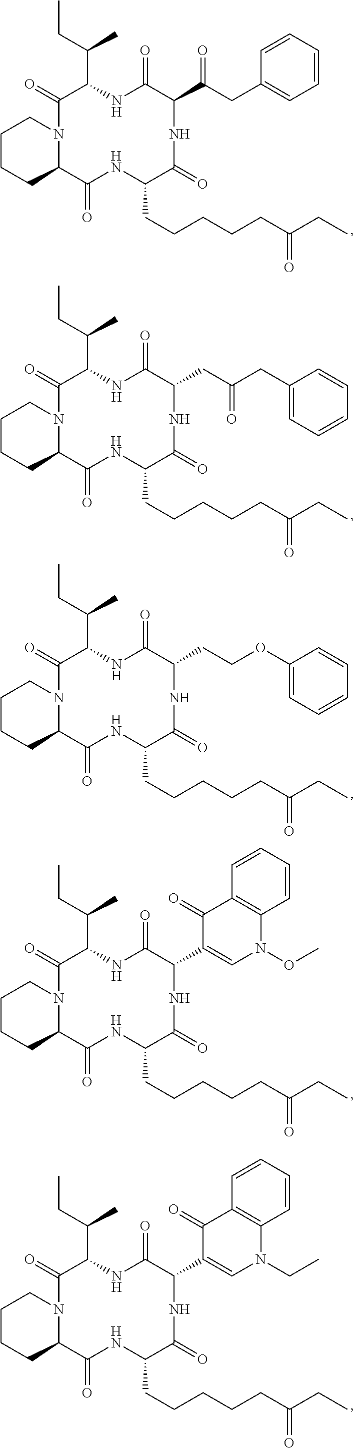

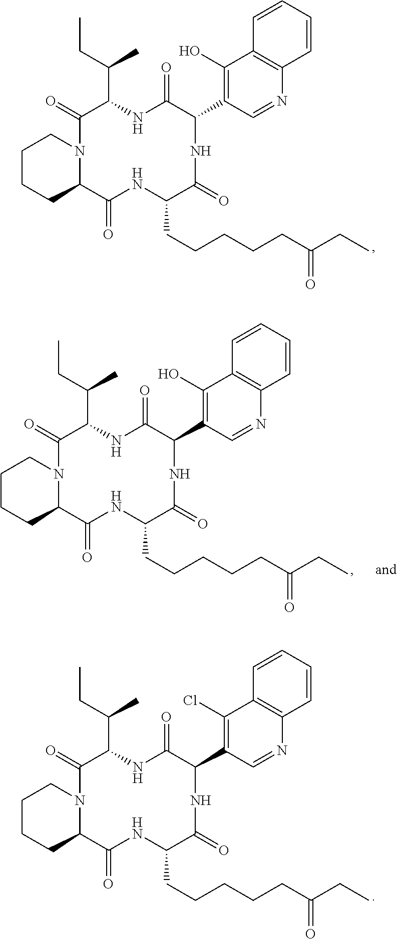

31. The method of claim 28, wherein the administering step is intravenous administration.

32. The method of claim 28, wherein the staphylococcal infection is a skin and soft tissue infection (SSTI), staphylococcal pneumonia, a staphylococcal bone infection, a staphylococcal joint infection, staphylococcal sepsis, or staphylococcal phendocarditis.

33. The method of claim 28, wherein the staphylococcal infection is a methicillin-resistant Staphylococcus aureus infection or a methicillin-sensitive Staphylococcus aureus infection.
